SEQUENCE LISTING (317 sequences)

SEQ ID NO: 1 - protein, 831 aa, synthetic construct
  REGION 1..831: note = synthetic construct
MYPYDVPDYAYPYDVPDYAYPYDVPDYADIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSASGSGGGGDVASDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 2 - protein, 362 aa, synthetic construct
  REGION 1..362: note = synthetic construct
MGELDELVYLLDGPGYDPIHSDGSGTGSGTGSGTGTTSGTGTGGSTGHMMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 3 - protein, 502 aa, synthetic construct
  REGION 1..502: note = synthetic construct
MGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHMMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 4 - protein, 596 aa, synthetic construct
  REGION 1..596: note = synthetic construct
MGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHMMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 5 - protein, 690 aa, synthetic construct
  REGION 1..690: note = synthetic construct
MGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHMMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 6 - protein, 437 aa, synthetic construct
  REGION 1..437: note = synthetic construct
MGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHMASGSGGGGDVDALDDFDLDMLGSDALDDFDLDMLGSDALDDFDLDMLGSDALDDFDLDML

SEQ ID NO: 7 - protein, 481 aa, synthetic construct
  REGION 1..481: note = synthetic construct
MSQSNRELVVDFLSYKLSQKGYSWSQFSDVEENRTEAPEGTESEAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 8 - protein, 268 aa, synthetic construct
  REGION 1..268: note = synthetic construct
MVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASGSGGGGDVMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 9 - protein, 764 aa, synthetic construct
  REGION 1..764: note = synthetic construct
MVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASGSGGGGDVASVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASGSGGGGDVASVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 10 - protein, 822 aa, synthetic construct
  REGION 1..822: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAPASGSGGGGDVMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 11 - protein, 1039 aa, synthetic construct
  REGION 1..1039: note = synthetic construct
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHASGSGGGGDVASMSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHASGSGGGGDVASMSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 12 - protein, 393 aa, synthetic construct
  REGION 1..393: note = synthetic construct
MSTKLYGDVNDDGKVNSTDAVALKRYVLRSGISINTDNADLNEDGRVNSTDLGILKRYILKEIDTLPYKNASGSGGGGDVMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 13 - protein, 477 aa, synthetic construct
  REGION 1..477: note = synthetic construct
MSTKLYGDVNDDGKVNSTDAVALKRYVLRSGISINTDNADLNEDGRVNSTDLGILKRYILKEIDTLPYKNASGSGGGGDVASMSTKLYGDVNDDGKVNSTDAVALKRYVLRSGISINTDNADLNEDGRVNSTDLGILKRYILKEIDTLPYKNASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 14 - protein, 559 aa, synthetic construct
  REGION 1..559: note = synthetic construct
MSTKLYGDVNDDGKVNSTDAVALKRYVLRSGISINTDNADLNEDGRVNSTDLGILKRYILKEIDTLPYKNASGSGGGGDVASMSTKLYGDVNDDGKVNSTDAVALKRYVLRSGISINTDNADLNEDGRVNSTDLGILKRYILKEIDTLPYKNASGSGGGGDVASMSTKLYGDVNDDGKVNSTDAVALKRYVLRSGISINTDNADLNEDGRVNSTDLGILKRYILKEIDTLPYKNASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 15 - protein, 1080 aa, synthetic construct
  REGION 1..1080: note = synthetic construct
MSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFALESAPNLRALAEVATQTVRELTGFDRVMLYKFAPDATGEVIAEARREGLHAFLGHRFPASDIPAQARALYTRHLLRLTADTRAAAVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWPAGADLAPSAAGLLAISVGEGWSECLVWLRPELRLEVAWGGATPDQAKDDLGPRHSFDTYLEEKRGYAEPWHPGEIEEAQDLRDTLTGALGERLSVIRDLNRALTQSNAEWRQYGFVISHHMQEPVRLISQFAELLTRQPRAQDGSPDSPQTERITGFLLRETSRLRSLTQDLHTYTALLSAPPPVRRPTPLGRVVDDVLQDLEPRIADTGASIEVAPELPVIAADAGLLRDLLLHLIGNALTFGGPEPRIAVRTERQGAGWSIAVSDQGAGIAPEYQERIFLLFQRLGSLDEALGNGLGLPLCRKIAELHGGTLTVESAPGEGSTFRCWLPDAGPLPGAADAASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 16 - protein, 121 aa, synthetic construct
  REGION 1..121: note = synthetic construct
DAKSLTAWSRTLVTFKDVFVDFTREEWKLLDTAQQIVYRNVMLENYKNLVSLGYQLTKPDVILRLEKGEEPWLVEREIHQETHPDSETAFEIKSSVSSRSIFKDKQSCDIKMEGMARNDLW

SEQ ID NO: 17 - protein, 449 aa, synthetic construct
  REGION 1..449: note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPAS

SEQ ID NO: 18 - protein, 564 aa, synthetic construct
  REGION 1..564: note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 19 - protein, 483 aa, synthetic construct
  REGION 1..483: note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPASASGSGGGGDVGSGSKMSKDGKKKKKKSKTKCVIM

SEQ ID NO: 20 - protein, 463 aa, synthetic construct
  REGION 1..463: note = synthetic construct
MLQLPPLERLTLGSSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPAS

SEQ ID NO: 21 - protein, 456 aa, synthetic construct
  REGION 1..456: note = synthetic construct
MPKKKRKVVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPAS

SEQ ID NO: 22 - protein, 466 aa, synthetic construct
  REGION 1..466: note = synthetic construct
MLLLLLLLGLRLQLSLGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPAS

SEQ ID NO: 23 - protein, 556 aa, synthetic construct
  REGION 1..556: note = synthetic construct
METDTLLLWVLLLWVPGSTGDEQKLISEEDLNAVGQDTQEVIVVPHSLPFKVVVISAILALVVLTIISLIILIMLWQKKPRGGSGGLEMPASGTMRSASGSGGGGDVMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPAS

SEQ ID NO: 24 - protein, 1061 aa, synthetic construct
  REGION 1..1061: note = synthetic construct
MAAAAAAGAGPEMVRGQVFDVGPRYTNLSYIGEGAYGMVCSAYDNVNKVRVAIKKISPFEHQTYCQRTLREIKILLRFRHENIIGINDIIRAPTIEQMKDVYIVQDLMETDLYKLLKTQHLSNDHICYFLYQILRGLKYIHSANVLHRDLKPSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPGYRSASGSGGGGDVASMAAAAAAGAGPEMVRGQVFDVGPRYTNLSYIGEGAYGMVCSAYDNVNKVRVAIKKISPFEHQTYCQRTLREIKILLRFRHENIIGINDIIRAPTIEQMKDVYIVQDLMETDLYKLLKTQHLSNDHICYFLYQILRGLKYIHSANVLHRDLKPSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPGYRSASGSGGGGDVASGGGSMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 25 - protein, 229 aa, synthetic construct
  REGION 1..229: note = synthetic construct
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGSMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 26 - protein, 433 aa, synthetic construct
  REGION 1..433: note = synthetic construct
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEASGSGGGGDVEFMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 27 - protein, 222 aa, synthetic construct
  REGION 1..222: note = synthetic construct
KRDHHHHHHQDKKTMMMNEEDDGNGMDELLAVLGYKVRSSEMADVAQKLEQLEVMMSNVQEDDLSQLATETVHYNPAELYTWLDSMLTDLNASGSGGGGDVASMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 28 - protein, 559 aa, synthetic construct
  REGION 1..559: note = synthetic construct
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 29 - protein, 805 aa, synthetic construct
  REGION 1..805: note = synthetic construct
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSASGSGGGGDVASMDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 30 - protein, 1049 aa, synthetic construct
  REGION 1..1049: note = synthetic construct
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSASGSGGGGDVASDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSASGSGGGGDVASDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 31 - protein, 1046 aa, synthetic construct
  REGION 1..1046: note = synthetic construct
YVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVGSTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKASGSGGGGSTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKASGSGGGTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 32 - protein, 300 aa, synthetic construct
  REGION 1..300: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKEFGGGGRAEEEVNGAIGGDLLLNFPDMSVLERQRAHLKYLNPTFDSPLAGFFADSSMITGGEMDSYLSTAGLNLPMMYGETTVEGDSRLSISPETTLGTGNFKKRKFDTETKDCNEKKKKMTMNRDDLVEEGEEEKSKITEQNNGSTKSIKKMKHKAKKEENNFSNDSSKVTKELEKTDYI

SEQ ID NO: 33 - protein, 267 aa, synthetic construct
  REGION 1..267: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNV

SEQ ID NO: 34 - protein, 417 aa, synthetic construct
  REGION 1..417: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVAS

SEQ ID NO: 35 - protein, 567 aa, synthetic construct
  REGION 1..567: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVAS

SEQ ID NO: 36 - protein, 364 aa, synthetic construct
  REGION 1..364: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSAS

SEQ ID NO: 37 - protein, 243 aa, synthetic construct
  REGION 1..243: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVASMSMEDPFFVVKGEVQKAVNTAQGLFQRWTELLQGPSAATREEIDWTTNELRNNLRSIEWDLEDLDETISIVEANPRKFNLDATELSIRKAFITSTRQIVRDMKDQMSASSAS

SEQ ID NO: 38 - protein, 282 aa, synthetic construct
  REGION 1..282: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVASGGSQRWELALGRFLEYLSWVSTLSEQVQEELLSSQVTQELRALMDETMKELKAYKSELEEQLTPVAEETRARLSKELQAAQARLGADMEDVRGRLVQYRGEVQAMLGQSTEELRVRLASHLIALQLRLIGDAFDLQKRLAVYQAGAAERAS

SEQ ID NO: 39 - protein, 327 aa, synthetic construct
  REGION 1..327: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPAS

SEQ ID NO: 40 - protein, 535 aa, synthetic construct
  REGION 1..535: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPASASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPAS

SEQ ID NO: 41 - protein, 743 aa, synthetic construct
  REGION 1..743: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPASASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPASASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPAS

SEQ ID NO: 42 - protein, 329 aa, synthetic construct
  REGION 1..329: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPAS

SEQ ID NO: 43 - protein, 444 aa, synthetic construct
  REGION 1..444: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKEFASGSGGGGDVMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 44 - protein, 346 aa, synthetic construct
  REGION 1..346: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVMATVEPETTPTPNPPTTEEEKTESNQEVANPEHYIKHPLQNRWALWFFKNDKSKTWQANLRLISKFDTVEDFWALYNHIQLSSNLMPGCDYSLFKDGIEPMWEDEKNKRGGRWLITLNAQQRRSDLDRFWLETLLCLIGESFDDYSDDVCGAVVNVRAKGDKIAIWTTECENREAVTHIGRVYKERLGLPPKIVIGYQSHADTATKSGSTTKNRFVV

SEQ ID NO: 45 - protein, 442 aa, synthetic construct
  REGION 1..442: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVMLKMESTQQMVSSIINTSFEAAVVAATSTLELMGIQYDYNEVFTRVKSKFDYVMDDSGVKNNLLGKAITIAQALNGKFGSAIRNRNWMTDSKTVAKLDEDVSKLRMTLSSKGIDQKMRVLNACFSVKRIPGKSSSIIKCTRLMKDKIEGGEVEVDDSYVDEKMEIDTIDWKSRYDQLEKRFEALKQRVNEKYNTWVQKAKKVNENMYSLQNVISQQQNQISDLQQYCNKLEADLQGKFSSLVSSVECYLRSMELPDDVKNDIEQQLNSIDAINPINAIDDIESLIRNLIQDYDRTFLMLKGLLKQCNYEYAYE

SEQ ID NO: 46 - protein, 389 aa, synthetic construct
  REGION 1..389: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVDLGKKLLEAARAGQDDEVRILMANGADVNALDEDGLTPLHLAAQLGHLEIVEVLLKYGADVNAEDNFGITPLHLAAIRGHLEIVEVLLKHGADVNAQDKFGKTAFDISIDNGNEDLAEILQKLASASGSGGGGDVDLGKKLLEAARAGQDDEVRILMANGADVNALDEDGLTPLHLAAQLGHLEIVEVLLKYGADVNAEDNFGITPLHLAAIRGHLEIVEVLLKHGADVNAQDKFGKTAFDISIDNGNEDLAEILQKLAS

SEQ ID NO: 47 - protein, 398 aa, synthetic construct
  REGION 1..398: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVMAEVQLQQSGAELVRSGASVKLSCTASGFNIKDYYMHWVKQRPEQGLEWIGWIDPENGDTEYTQKFKGKATLTADKSPSTAYMQLSSLTSEDSAVYYCARITTDYYFDYWGQGTTLTVSSGGGGSGGGGSGGGGSDVVMTQTPLSLPVSLGDQASISCRSSQSLVHSNGNTYLEWYLQKPGQSPKLLIYKVSNRFSGVPDRFSGSGSGTDFTLKISRVEAEDLGVYFCSQSTHVPLTFGAGTKLEIKRAAAGGLNDIFEAQKIEWHEAS

SEQ ID NO: 48 - protein, 765 aa, synthetic construct
  REGION 1..765: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPASASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPASASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPASDYKDHDGDYKDHDIDYKDDDDK

SEQ ID NO: 49 - protein, 466 aa, synthetic construct
  REGION 1..466: note = synthetic construct
MDYKDHDGDYKDHDIDYKDDDDKYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKEFASGSGGGGDVMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 50 - protein, 410 aa, synthetic construct
  REGION 1..410: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSMKFNKEKWQAIKEIAFLPNLNQPQHLAFIVSLLDDPSQSANLLAEAKKLNDAQAPKASASGSGGGGDVGSMKFNKEKWQAIKEIAFLPNLNQPQHLAFIVSLLDDPSQSANLLAEAKKLNDAQAPKASASGSGGGGDVGSMKFNKEKWQAIKEIAFLPNLNQPQHLAFIVSLLDDPSQSANLLAEAKKLNDAQAPKASASGSGGGGDVGSMKFNKEKWQAIKEIAFLPNLNQPQHLAFIVSLLDDPSQSANLLAEAKKLNDAQAPKAS

SEQ ID NO: 51 - protein, 282 aa, synthetic construct
  REGION 1..282: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVAS

SEQ ID NO: 52 - protein, 414 aa, synthetic construct
  REGION 1..414: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGGGGSGGGGSGGGGSMAQVQLVESGGGLVQAGGSLRLSCAASGRTFSNYAMGWFRQAPGKEREFVAAISWTGVSTYYADSVKGRFTISRDNDKNTVYVQMNSLIPEDTAIYYCAAVRARSFSDTYSRVNEYDYWGQGTQVTVAS

SEQ ID NO: 53 - protein, 693 aa, synthetic construct
  REGION 1..693: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGGGGSGGGGSGGGGSMAQVQLVESGGGLVQAGGSLRLSCAASGRTFSNYAMGWFRQAPGKEREFVAAISWTGVSTYYADSVKGRFTISRDNDKNTVYVQMNSLIPEDTAIYYCAAVRARSFSDTYSRVNEYDYWGQGTQVTVASASGSGGGGAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGGGGSGGGGSGGGGSMAQVQLVESGGGLVQAGGSLRLSCAASGRTFSNYAMGWFRQAPGKEREFVAAISWTGVSTYYADSVKGRFTISRDNDKNTVYVQMNSLIPEDTAIYYCAAVRARSFSDTYSRVNEYDYWGQGTQVTVAS

SEQ ID NO: 54 - protein, 338 aa, synthetic construct
  REGION 1..338: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPAS

SEQ ID NO: 55 - protein, 752 aa, synthetic construct
  REGION 1..752: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPASASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPASASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 56 - protein, 340 aa, synthetic construct
  REGION 1..340: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPAS

SEQ ID NO: 57 - protein, 550 aa, synthetic construct
  REGION 1..550: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPASASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPAS

SEQ ID NO: 58 - protein, 760 aa, synthetic construct
  REGION 1..760: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPASASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPASASGSGGGGDVGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPAS

SEQ ID NO: 59 - protein, 453 aa, synthetic construct
  REGION 1..453: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 60 - protein, 453 aa, synthetic construct
  REGION 1..453: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVMLKMESTQQMVSSIINTSFEAAVVAATSTLELMGIQYDYNEVFTRVKSKFDYVMDDSGVKNNLLGKAITIAQALNGKFGSAIRNRNWMTDSKTVAKLDEDVSKLRMTLSSKGIDQKMRVLNACFSVKRIPGKSSSIIKCTRLMKDKIEGGEVEVDDSYVDEKMEIDTIDWKSRYDQLEKRFEALKQRVNEKYNTWVQKAKKVNENMYSLQNVISQQQNQISDLQQYCNKLEADLQGKFSSLVSSVECYLRSMELPDDVKNDIEQQLNSIDAINPINAIDDIESLIRNLIQDYDRTFLMLKGLLKQCNYEYAYE

SEQ ID NO: 61 - protein, 457 aa, synthetic construct
  REGION 1..457: note = synthetic construct
MPKKKRKVAAVDLATHPGHLARRLQQAHYLLWNTMVSEETTSPQYAVLNALVAEPGLDQRTVGERVGLDRSTIAEVVSRLGRRGLLDKVRDPQDGRRSLLRLTDEGLRVHRRLGVRIARMNQVFLAPLAADEQAVFFDLIRRVADAAEGLRNPAEPAVAPGASGSGGGGDVDEMEECSQHGGSGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHGGSGGSDALDDFDLDMLGSDALDDFDLDMLGSDALDDFDLDMLGSDALDDFDLDML

SEQ ID NO: 62 - protein, 522 aa, synthetic construct
  REGION 1..522: note = synthetic construct
MKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 63 - protein, 668 aa, synthetic construct
  REGION 1..668: note = synthetic construct
LKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYEASGSGGGGDVGSAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAEC

SEQ ID NO: 64 - protein, 880 aa, synthetic construct
  REGION 1..880: note = synthetic construct
LKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYEASGSGGGGDVGSGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNGGGGSGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNGGGGSGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 65 - protein, 767 aa, synthetic construct
  REGION 1..767: note = synthetic construct
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTVASGSGGGGDVGSMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 66 - protein, 789 aa, synthetic construct
  REGION 1..789: note = synthetic construct
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTVASGSGGGGDVGSMYVRFEVPEDMQNEALSLLEKVRESGKVKKGTNETTKAVERGLAKLVHIAEDVDPPEIVAHLPLLCEEKNVPYIYVKSKNDLGRAVGIEVPCASAAIINEGELRKELGSLVEKIKGLQKDYKDHDGDYKDHDIDYKDDDDK

SEQ ID NO: 67 - protein, 884 aa, synthetic construct
  REGION 1..884: note = synthetic construct
MGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNASGSGGGGDVASGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNASGSGGGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNASGSGGMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 68 - protein, 490 aa, synthetic construct
  REGION 1..490: note = synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGASGSGGGGDVGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGGGGSGGGGSGGGGSMAQVQLVESGGGLVQAGGSLRLSCAASGRTFSNYAMGWFRQAPGKEREFVAAISWTGVSTYYADSVKGRFTISRDNDKNTVYVQMNSLIPEDTAIYYCAAVRARSFSDTYSRVNEYDYWGQGTQVTVAS

SEQ ID NO: 69 - protein, 1227 aa, synthetic construct
  REGION 1..1227: note = synthetic construct
MGPPRGGPGGELPRGPQAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHNGSMHDQLTEEQIAEFKEAFSLFDKDGDGTITTKELGTVMRSLGQNPTEAELQDMINEVDADGNGTIYFPELLTMLARKMKDTDSEEEIREAFRVFDKDGNGYISAAELRHVMTNLGEKLTDEEVDEMIREADIDGDGQVNYEEFVQMMTAKGGKRRWQKTGHAVRIIGRLKKISSSGALELEFGGGGSMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 70 - protein, 460 aa, synthetic construct
  REGION 1..460: note = synthetic construct
MAEVQVQQSGAELVRSGASVKLSCTASGFNIKDYYMHWVKQRPEQGLEWIGWIDPANGNTKYDPKFQGKATITADTSSNTAYLQLSSLTSEDTAVYYCARWDWYFDVWGAGTTVTVSSGGGGSGGGGSGGGGSVFASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 71 - protein, 300 aa, synthetic construct
  REGION 1..300: note = synthetic construct
TRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 72 - protein, 56 aa, synthetic construct
  REGION 1..56: note = synthetic construct
MKFNKEKWQAIKEIAFLPNLNQPQHLAFIVSLLDDPSQSANLLAEAKKLNDAQAPK

SEQ ID NO: 73 - protein, 21 aa, synthetic construct
  REGION 1..21: note = synthetic construct
GELDELVYLLDGPGYDPIHSD

SEQ ID NO: 74 - protein, 155 aa, synthetic construct
  REGION 1..155: note = synthetic construct
SQSNRELVVDFLSYKLSQKGYSWSQFSDVEENRTEAPEGTESEAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYG

SEQ ID NO: 75 - protein, 24 aa, synthetic construct
  REGION 1..24: note = synthetic construct
GSGSKMSKDGKKKKKKSKTKCVIM

SEQ ID NO: 76 - protein, 169 aa, synthetic construct
  REGION 1..169: note = synthetic construct
NGAIGGDLLLNFPDMSVLERQRAHLKYLNPTFDSPLAGFFADSSMITGGEMDSYLSTAGLNLPMMYGETTVEGDSRLSISPETTLGTGNFKKRKFDTETKDCNEKKKKMTMNRDDLVEEGEEEKSKITEQNNGSTKSIKKMKHKAKKEENNFSNDSSKVTKELEKTDYI

SEQ ID NO: 77 - protein, 138 aa, synthetic construct
  REGION 1..138: note = synthetic construct
VVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNV

SEQ ID NO: 78 - protein, 498 aa, synthetic construct
  REGION 1..498: note = synthetic construct
KMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAP

SEQ ID NO: 79 - protein, 229 aa, synthetic construct
  REGION 1..229: note = synthetic construct
SSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNH

SEQ ID NO: 80 - protein, 69 aa, synthetic construct
  REGION 1..69: note = synthetic construct
STKLYGDVNDDGKVNSTDAVALKRYVLRSGISINTDNADLNEDGRVNSTDLGILKRYILKEIDTLPYKN

SEQ ID NO: 81 - protein, 754 aa, synthetic construct
  REGION 1..754: note = synthetic construct
SRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFALESAPNLRALAEVATQTVRELTGFDRVMLYKFAPDATGEVIAEARREGLHAFLGHRFPASDIPAQARALYTRHLLRLTADTRAAAVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWPAGADLAPSAAGLLAISVGEGWSECLVWLRPELRLEVAWGGATPDQAKDDLGPRHSFDTYLEEKRGYAEPWHPGEIEEAQDLRDTLTGALGERLSVIRDLNRALTQSNAEWRQYGFVISHHMQEPVRLISQFAELLTRQPRAQDGSPDSPQTERITGFLLRETSRLRSLTQDLHTYTALLSAPPPVRRPTPLGRVVDDVLQDLEPRIADTGASIEVAPELPVIAADAGLLRDLLLHLIGNALTFGGPEPRIAVRTERQGAGWSIAVSDQGAGIAPEYQERIFLLFQRLGSLDEALGNGLGLPLCRKIAELHGGTLTVESAPGEGSTFRCWLPDAGPLPGAADA

SEQ ID NO: 82 - protein, 238 aa, synthetic construct
  REGION 1..238: note = synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 83 - protein, 216 aa, synthetic construct
  REGION 1..216: note = synthetic construct
ATVEPETTPTPNPPTTEEEKTESNQEVANPEHYIKHPLQNRWALWFFKNDKSKTWQANLRLISKFDTVEDFWALYNHIQLSSNLMPGCDYSLFKDGIEPMWEDEKNKRGGRWLITLNAQQRRSDLDRFWLETLLCLIGESFDDYSDDVCGAVVNVRAKGDKIAIWTTECENREAVTHIGRVYKERLGLPPKIVIGYQSHADTATKSGSTTKNRFVV

SEQ ID NO: 84 - protein, 919 aa, synthetic construct
  REGION 1..919: note = synthetic construct
GPPRGGPGGELPRGPQAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 85 - protein, 359 aa, synthetic construct
  REGION 1..359: note = synthetic construct
AAAAAAGAGPEMVRGQVFDVGPRYTNLSYIGEGAYGMVCSAYDNVNKVRVAIKKISPFEHQTYCQRTLREIKILLRFRHENIIGINDIIRAPTIEQMKDVYIVQDLMETDLYKLLKTQHLSNDHICYFLYQILRGLKYIHSANVLHRDLKPSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPGYRS

SEQ ID NO: 86 - protein, 107 aa, synthetic construct
  REGION 1..107: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 87 - protein, 96 aa, synthetic construct
  REGION 1..96: note = synthetic construct
ASRILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 88 - protein, 91 aa, synthetic construct
  REGION 1..91: note = synthetic construct
KRDHHHHHHQDKKTMMMNEEDDGNGMDELLAVLGYKVRSSEMADVAQKLEQLEVMMSNVQEDDLSQLATETVHYNPAELYTWLDSMLTDLN

SEQ ID NO: 89 - protein, 344 aa, synthetic construct
  REGION 1..344: note = synthetic construct
AASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAEC

SEQ ID NO: 90 - protein, 233 aa, synthetic construct
  REGION 1..233: note = synthetic construct
DIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKS

SEQ ID NO: 91 - protein, 312 aa, synthetic construct
  REGION 1..312: note = synthetic construct
LKMESTQQMVSSIINTSFEAAVVAATSTLELMGIQYDYNEVFTRVKSKFDYVMDDSGVKNNLLGKAITIAQALNGKFGSAIRNRNWMTDSKTVAKLDEDVSKLRMTLSSKGIDQKMRVLNACFSVKRIPGKSSSIIKCTRLMKDKIEGGEVEVDDSYVDEKMEIDTIDWKSRYDQLEKRFEALKQRVNEKYNTWVQKAKKVNENMYSLQNVISQQQNQISDLQQYCNKLEADLQGKFSSLVSSVECYLRSMELPDDVKNDIEQQLNSIDAINPINAIDDIESLIRNLIQDYDRTFLMLKGLLKQCNYEYAYE

SEQ ID NO: 92 - protein, 118 aa, synthetic construct
  REGION 1..118: note = synthetic construct
YVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 93 - protein, 269 aa, synthetic construct
  REGION 1..269: note = synthetic construct
AQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGGGGSGGGGSGGGGSMAQVQLVESGGGLVQAGGSLRLSCAASGRTFSNYAMGWFRQAPGKEREFVAAISWTGVSTYYADSVKGRFTISRDNDKNTVYVQMNSLIPEDTAIYYCAAVRARSFSDTYSRVNEYDYWGQGTQVTV

SEQ ID NO: 94 - protein, 109 aa, synthetic construct
  REGION 1..109: note = synthetic construct
SMEDPFFVVKGEVQKAVNTAQGLFQRWTELLQGPSAATREEIDWTTNELRNNLRSIEWDLEDLDETISIVEANPRKFNLDATELSIRKAFITSTRQIVRDMKDQMSASS

SEQ ID NO: 95 - protein, 149 aa, synthetic construct
  REGION 1..149: note = synthetic construct
GGSQRWELALGRFLEYLSWVSTLSEQVQEELLSSQVTQELRALMDETMKELKAYKSELEEQLTPVAEETRARLSKELQAAQARLGADMEDVRGRLVQYRGEVQAMLGQSTEELRVRLASHLIALQLRLIGDAFDLQKRLAVYQAGAAER

SEQ ID NO: 96 - protein, 192 aa, synthetic construct
  REGION 1..192: note = synthetic construct
MDGSGEQLGSGGPTSSEQIMKTGAFLLQGFIQDRAGRMAGETPELTLEQPPQDASTKKLSECLRRIGDELDSNMELQRMIADVDTDSPREVFFRVAADMFADGNFNWGRVVALFYFASKLVLKALCTKVPELIRTIMGWTLDFLRERLLVWIQDQGGWEGLLSYFGTPTWQTVTIFVAGVLTASLTIWKKMG

SEQ ID NO: 97 - protein, 236 aa, synthetic construct
  REGION 1..236: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 98 - protein, 129 aa, synthetic construct
  REGION 1..129: note = synthetic construct
ASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 99 - protein, 110 aa, synthetic construct
  REGION 1..110: note = synthetic construct
MALWMRFLPLLALLVLWEPKPAQAFVKQHLCGPHLVEALYLVCGERGFFYTPKSRRKREDPQVPQLELGGGPEAGDLQTLALEVARQKRGIVDQCCTSICSLYQLENYCN

SEQ ID NO: 100 - protein, 36 aa, synthetic construct
  REGION 1..36: note = synthetic construct
MDAQTRRRERRAEKQAQWKAANPLLVGVSAKPVNRP

SEQ ID NO: 101 - protein, 200 aa, synthetic construct
  REGION 1..200: note = synthetic construct
MTSETDTLLLWVLLLWVPGSTGDASGGSGMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 102 - protein, 14 aa, synthetic construct
  REGION 1..14: note = synthetic construct
LQLPPLERLTLGSS

SEQ ID NO: 103 - protein, 7 aa, synthetic construct
  REGION 1..7: note = synthetic construct
PKKKRKV

SEQ ID NO: 104 - protein, 196 aa, synthetic construct
  REGION 1..196: note = synthetic construct
KKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 105 - protein, 196 aa, synthetic construct
  REGION 1..196: note = synthetic construct
KKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 106 - protein, 312 aa, synthetic construct
  REGION 1..312: note = synthetic construct
LKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 107 - protein, 20 aa, synthetic construct
  REGION 1..20: note = synthetic construct
GATNFSLLKQAGDVEENPGP

SEQ ID NO: 108 - protein, 635 aa, synthetic construct
  REGION 1..635: note = synthetic construct
NPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 109 - protein, 153 aa, synthetic construct
  REGION 1..153: note = synthetic construct
AAVDLATHPGHLARRLQQAHYLLWNTMVSEETTSPQYAVLNALVAEPGLDQRTVGERVGLDRSTIAEVVSRLGRRGLLDKVRDPQDGRRSLLRLTDEGLRVHRRLGVRIARMNQVFLAPLAADEQAVFFDLIRRVADAAEGLRNPAEPAVAPG

SEQ ID NO: 110 - protein, 123 aa, synthetic construct
  REGION 1..123: note = synthetic construct
DLGKKLLEAARAGQDDEVRILMANGADVNALDEDGLTPLHLAAQLGHLEIVEVLLKYGADVNAEDNFGITPLHLAAIRGHLEIVEVLLKHGADVNAQDKFGKTAFDISIDNGNEDLAEILQKL

SEQ ID NO: 111 - protein, 181 aa, synthetic construct
  REGION 1..181: note = synthetic construct
GGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 112 - protein, 134 aa, synthetic construct
  REGION 1..134: note = synthetic construct
AEVQVQQSGAELVRSGASVKLSCTASGFNIKDYYMHWVKQRPEQGLEWIGWIDPANGNTKYDPKFQGKATITADTSSNTAYLQLSSLTSEDTAVYYCARWDWYFDVWGAGTTVTVSSGGGGSGGGGSGGGGSVF

SEQ ID NO: 113 - protein, 266 aa, synthetic construct
  REGION 1..266: note = synthetic construct
AEVQLQQSGAELVRSGASVKLSCTASGFNIKDYYMHWVKQRPEQGLEWIGWIDPENGDTEYTQKFKGKATLTADKSPSTAYMQLSSLTSEDSAVYYCARITTDYYFDYWGQGTTLTVSSGGGGSGGGGSGGGGSDVVMTQTPLSLPVSLGDQASISCRSSQSLVHSNGNTYLEWYLQKPGQSPKLLIYKVSNRFSGVPDRFSGSGSGTDFTLKISRVEAEDLGVYFCSQSTHVPLTFGAGTKLEIKRAAAGGLNDIFEAQKIEWHE

SEQ ID NO: 114 - protein, 519 aa, synthetic construct
  REGION 1..519: note = synthetic construct
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGYSRVGAAGRFEQT

SEQ ID NO: 115 - protein, 17 aa, synthetic construct
  REGION 1..17: note = synthetic construct
MLLLLLLLGLRLQLSLG

SEQ ID NO: 116 - protein, 236 aa, synthetic construct
  REGION 1..236: note = synthetic construct
DEMEECSQHGGSGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHGGSGGS

SEQ ID NO: 117 - protein, 205 aa, synthetic construct
  REGION 1..205: note = synthetic construct
SRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESG

SEQ ID NO: 118 - protein, 81 aa, synthetic construct
  REGION 1..81: note = synthetic construct
METDTLLLWVLLLWVPGSTGDEQKLISEEDLNAVGQDTQEVIVVPHSLPFKVVVISAILALVVLTIISLIILIMLWQKKPR

SEQ ID NO: 119 - protein, 50 aa, synthetic construct
  REGION 1..50: note = synthetic construct
DALDDFDLDMLGSDALDDFDLDMLGSDALDDFDLDMLGSDALDDFDLDML

SEQ ID NO: 120 - protein, 117 aa, synthetic construct
  REGION 1..117: note = synthetic construct
AKGKTKSKVGPYRGRGVALTDDEYDEWREHNATRKLDLSVEDFLMLRHRAALGADDADAVKFRSWWNSRSRLADDYEDVTVIGKGGVKHEKIRTNTLRAVDRGYDVSFAEETGVPTV

SEQ ID NO: 121 - RNA, 19 nt, synthetic construct
  misc_feature 1..19: note = synthetic construct
  source 1..19: mol_type = other RNA
gggccctgaagaagggccc

SEQ ID NO: 122 - RNA, 22 nt, synthetic construct
  misc_feature 1..22: note = synthetic construct
  source 1..22: mol_type = other RNA
tcacagttgccagctgagatta

SEQ ID NO: 123 - RNA, 23 nt, synthetic construct
  misc_feature 1..23: note = synthetic construct
  source 1..23: mol_type = other RNA
gggcgtgatccgaaaggtgaccc

SEQ ID NO: 124 - RNA, 76 nt, synthetic construct
  misc_feature 1..76: note = synthetic construct
  source 1..76: mol_type = other RNA
gtttaaaccctcctcgtccggggctgtgtcgccccgctgacgagccctgaaaaagggcgaaacgaggaggactagt

SEQ ID NO: 125 - RNA, 13 nt, synthetic construct
  misc_feature 1..13: note = synthetic construct
  source 1..13: mol_type = other RNA
tgaggatcaccca

SEQ ID NO: 126 - RNA, 60 nt, synthetic construct
  misc_feature 1..60: note = synthetic construct
  source 1..60: mol_type = other RNA
ccggtaatctcagctggcaactgtgactcgagtcacagttgccagctgagattatttttg

SEQ ID NO: 127 - DNA, 588 bp, synthetic construct
  misc_feature 1..588: note = synthetic construct
  source 1..588: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 128 - DNA, 241 bp, synthetic construct
  misc_feature 1..241: note = synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 129 - DNA, 283 bp, synthetic construct
  misc_feature 1..283: note = synthetic construct
  source 1..283: mol_type = other DNA
aggggaaataatctatttgaacagtttagatggtgaagaacatttgcagcatttgcaagatttttttccactctgaagtggtctttgtccttgaacataggatacaagtgacccctgctctgttaattattggcaaattgcctaacttcaacgtaaggaaatagagtcatatgtttgctcactgaaggttactagttaacaggcatcccttaaacaggatataaaaggacttcagcaggactgctcgaaacatcccacttccagcactgcctgcggtgaagga

SEQ ID NO: 130 - DNA, 1617 bp, synthetic construct
  misc_feature 1..1617: note = synthetic construct
  source 1..1617: mol_type = other DNA
tgacgtcagctgccagatcccatggccgtcatactgtgacgtctttcagacaccccattgacgtcaatgggagaacagatctgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagaagcttgccaccatgcccaagaagaagaggaaggtagcagcggtcgatctggccacccaccccggacacctggcaagaaggctgcaacaagcccactacctgctgtggaatacaatggtcagcgaggagaccacatccccccagtacgccgtgctgaacgccctggtggctgaacctggcctggaccaaaggactgtgggcgagagggtggggctggatagaagcaccatcgccgaggtggtgagcagactggggagaaggggcctgctggacaaggtgagggacccccaggacggcaggaggtccctgctgaggctgaccgacgagggcctgagggtgcacaggaggctgggcgtgaggatcgccaggatgaaccaggtcttcctggccccactggcagccgatgaacaggccgtgttcttcgacctgattaggagagtcgccgatgccgcagaggggctgaggaaccctgcagaaccggcggtggcaccgggcgccagcggaagtggcggaggaggcgacgtcgatgagatggaagagtgctctcagcacggcggatctggaggttctacaggctgcgtggtcatagtgggcaggatcgtcttgtccggatctggcactagtgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctgggcagtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctctgggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctctccaccagcagtcaccctgacgcacgggggttcaggcggtagtgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtga

SEQ ID NO: 131 - RNA, 2208 nt, synthetic construct
  misc_feature 1..2208: note = synthetic construct
  source 1..2208: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgatggatccgctagctaatagcaaccacacaaacaatcaaagtttaaacgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaacttgaaccggtaagaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgaattcacagatgcagatcacagttgccagctgagattatacagtaaattggagacgagtccgtctcactagacagatgcagatcacagttgccagctgagattatacagtatctagagggcccgtttaaacccgctgatcagcct

SEQ ID NO: 132 - RNA, 2709 nt, synthetic construct
  misc_feature 1..2709: note = synthetic construct
  source 1..2709: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgatggatccgctagctaatagcaaccacacaaacaatcaaagtttaaacgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaacttgaaccgggcaaccacacaaacaatcaaagtttaaacgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaacttgaaccggtaagaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgaattcacagatgccggtaagaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgaattcacagatgcagatcacagttgccagctgagattatacagtaaattggagacgagtccgtctcactagacagatgcagatcacagttgccagctgagattatacagtatctagagggcccgtttaaacccgctgatcagcct

SEQ ID NO: 133 - RNA, 2424 nt, synthetic construct
  misc_feature 1..2424: note = synthetic construct
  source 1..2424: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgatggatccgctagctaatagcaaccacacaaacaatcaaagtttaaacgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaacttgaaccgggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactccggtaagaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgaattcacagatgcagatcacagttgccagctgagattatacagtaaattggagacgagtccgtctcactagacagatgcagatcacagttgccagctgagattatacagtatctagagggcccgtttaaacccgctgatcagcct

SEQ ID NO: 134 - RNA, 3058 nt, synthetic construct
  misc_feature 1..3058: note = synthetic construct
  source 1..3058: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgagcggccgctcaatctcgaggggatcccgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcctgatccaacttgaaccggtaagaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgatcccgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcctgatcggagacgagtccgtctcaaattgccgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatgaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgaattgccgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatgaattcacagatgcagatcacagttgccagctgagattatacagtaaattggagacgagtccgtctcactagacagatgcagatcacagttgccagctgagattatacagta

SEQ ID NO: 135 - RNA, 2135 nt, synthetic construct
  misc_feature 1..2135: note = synthetic construct
  source 1..2135: mol_type = other RNA
atgactagtgagacagacacactcctgctatgggtactgctgctctgggttccaggttccactggtgacgctagtggtggttctggtatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtgagcggccgctcaatctcgaggggatcccgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcctgatccaacttgaaccggtaagaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgatcccgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcctgatcggagacgagtccgtctcaaattgccgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatgaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgaattgccgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatgaattcacagatgcagatcacagttgccagctgagattatacagtaaattggagacgagtccgtctcactagacagatgcagatcacagttgccagctgagattatacagtatctagagggcccgtttaaacccgctgatcagcct

SEQ ID NO: 136 - RNA, 3056 nt, synthetic construct
  misc_feature 1..3056: note = synthetic construct
  source 1..3056: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgagcggccgctcaatctcgaggggatcccgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcctgatccaacttgaaccggtaagaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgatcccgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcctgatcggagacgagtccgtctcaaattgccgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatgaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgaattgccgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatgaattcgtttaaaccctcctcgtccggggctgtgtcgccccgctgacgagccctgaaaaagggcgaaacgaggaggactagtgggcccgtttaaacccgctgatcagcct

SEQ ID NO: 137 - RNA, 2959 nt, synthetic construct
  misc_feature 1..2959: note = synthetic construct
  source 1..2959: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgagcggccgctcaatctcgagggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagtgggcccgtttaaacccgctgatcagcct

SEQ ID NO: 138 - RNA, 2549 nt, synthetic construct
  misc_feature 1..2549: note = synthetic construct
  source 1..2549: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgagcggccgctcaatctcgagttatgaggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagtgggcccgtttaaacccgctgatcagcct

SEQ ID NO: 139 - RNA, 2763 nt, synthetic construct
  misc_feature 1..2763: note = synthetic construct
  source 1..2763: mol_type = other RNA
atgactagtgagacagacacactcctgctatgggtactgctgctctgggttccaggttccactggtgacgctagtggtggttctggtatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcggagcaaccaacttttccctgctgaagcaggcaggcgacgtggaggagaatcctggacccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgactcgagggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagtgggcccgtttaaacccgctgatcagcct

SEQ ID NO: 140 - RNA, 2385 nt, synthetic construct
  misc_feature 1..2385: note = synthetic construct
  source 1..2385: mol_type = other RNA
atgactagtgagacagacacactcctgctatgggtactgctgctctgggttccaggttccactggtgacgctagtggtggttctggtatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcggagcaaccaacttttccctgctgaagcaggcaggcgacgtggaggagaatcctggacccatggccctgtggatgcgcttcctgcccctgctggccctgctcgtcctctgggagcccaagcctgcccaggcttttgtcaaacagcacctttgtggtcctcacctggtggaggctctgtacctggtgtgtggggaacgtggtttcttctacacacccaagtcccgtcgtaaaagggaggacccgcaagtgccacaactggagctgggtggaggcccggaggccggggatcttcagaccttggcactggaggttgcccggcagaagcgtggcattgtggatcagtgctgcaccagcatctgctccctctaccaactggagaactactgcaactgactcgagggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagtgggcccgtttaaacccgctgatcagcct

SEQ ID NO: 141 - DNA, 987 bp, synthetic construct
  misc_feature 1..987: note = synthetic construct
  source 1..987: mol_type = other DNA
cgcgtatactcagtgccctgactatatactcagtgccctgactatatactcagtgccctgactatatactcagtgccctgactatatactcagtgccctgactatgctagctcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 142 - RNA, 1820 nt, synthetic construct
  misc_feature 1..1820: note = synthetic construct
  source 1..1820: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccagaatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagaccatcatgttcgacgacgccattgagagggcgggccagctcaccagcgaggaggacacgctgagcctcgtcactgccgaccactcccacgtgttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttacagcagagtcggggcggccggccgcttcgagcagacatgagcggccgctcaatctcgaggggatccactagtccagtgtggtggaattcgtttaaaccctcctcgtccggggctgtgtcgccccgctgacgagccctgaaaaagggcgaaacgaggaggactagtaattggagacgagtccgtctcactaggtttaaaccctcctcgtccggggctgtgtcgccccgctgacgagccctgaaaaagggcgaaacgaggaggactagtctagagggcccgtttaaacccgctgatcagcct

SEQ ID NO: 143 - RNA, 1981 nt, synthetic construct
  misc_feature 1..1981: note = synthetic construct
  source 1..1981: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccagaatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagaccatcatgttcgacgacgccattgagagggcgggccagctcaccagcgaggaggacacgctgagcctcgtcactgccgaccactcccacgtgttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttacagcagagtcggggcggccggccgcttcgagcagacatgagcggccgctcaatctcgaggggatccactagtccagtgtggtggaattcgtttaaaccctcctcgtccggggctgtgtcgccccgctgacgagccctgaaaaagggcgaaacgaggaggactagtaattcgtttaaaccctcctcgtccggggctgtgtcgccccgctgacgagccctgaaaaagggcgaaacgaggaggactagtaattggagacgagtccgtctcactaggtttaaaccctcctcgtccggggctgtgtcgccccgctgacgagccctgaaaaagggcgaaacgaggaggactagtctaggtttaaaccctcctcgtccggggctgtgtcgccccgctgacgagccctgaaaaagggcgaaacgaggaggactagtctagagggcccgtttaaacccgctgatcagcct

SEQ ID NO: 144 - DNA, 3050 bp, synthetic construct
  misc_feature 1..3050: note = synthetic construct
  source 1..3050: mol_type = other DNA
aggggaaataatctatttgaacagtttagatggtgaagaacatttgcagcatttgcaagatttttttccactctgaagtggtctttgtccttgaacataggatacaagtgacccctgctctgttaattattggcaaattgcctaacttcaacgtaaggaaatagagtcatatgtttgctcactgaaggttactagttaacaggcatcccttaaacaggatataaaaggacttcagcaggactgctcgaaacatcccacttccagcactgcctgcggtgaaggataatacgactcactatagggaagcttgccaccatgactagtgagacagacacactcctgctatgggtactgctgctctgggttccaggttccactggtgacgctagtggtggttctggtatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcggagcaaccaacttttccctgctgaagcaggcaggcgacgtggaggagaatcctggacccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgactcgagggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagt

SEQ ID NO: 145 - RNA, 1965 nt, synthetic construct
  misc_feature 1..1965: note = synthetic construct
  source 1..1965: mol_type = other RNA
atggacgggtccggggagcagcttgggagcggcgggcccaccagctctgaacagatcatgaagacaggggcctttttgctacagggtttcatccaggatcgagcagggaggatggctggggagacacctgagctgaccttggagcagccgccccaggatgcgtccaccaagaagctgagcgagtgtctccggcgaattggagatgaactggacagcaatatggagctgcagaggatgattgctgacgtggacacggactccccccgagaggtcttcttccgggtggcagctgacatgtttgctgatggcaacttcaactggggccgcgtggttgccctcttctactttgctagcaaactggtgctcaaggccctgtgcactaaagtgcccgagctgatcagaaccatcatgggctggacactggacttcctccgtgagcggctgcttgtctggatccaagaccagggtggctgggaaggcctcctctcctacttcgggacccccacatggcagacagtgaccatctttgtggctggagtcctcaccgcctcactcaccatctggaagaagatgggctgactcgagggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagtgggcccgtttaaacccgctgatcagcct

SEQ ID NO: 146 - DNA, 3265 bp, synthetic construct
  misc_feature 1..3265: note = synthetic construct
  source 1..3265: mol_type = other DNA
aggggaaataatctatttgaacagtttagatggtgaagaacatttgcagcatttgcaagatttttttccactctgaagtggtctttgtccttgaacataggatacaagtgacccctgctctgttaattattggcaaattgcctaacttcaacgtaaggaaatagagtcatatgtttgctcactgaaggttactagttaacaggcatcccttaaacaggatataaaaggacttcagcaggactgctcgaaacatcccacttccagcactgcctgcggtgaaggataatacgactcactataggggctagcgtttaaacttaagcttagcgccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgagcggccgctcaatctcgagggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagt

SEQ ID NO: 147 - DNA, 2268 bp, synthetic construct
  misc_feature 1..2268: note = synthetic construct
  source 1..2268: mol_type = other DNA
aggggaaataatctatttgaacagtttagatggtgaagaacatttgcagcatttgcaagatttttttccactctgaagtggtctttgtccttgaacataggatacaagtgacccctgctctgttaattattggcaaattgcctaacttcaacgtaaggaaatagagtcatatgtttgctcactgaaggttactagttaacaggcatcccttaaacaggatataaaaggacttcagcaggactgctcgaaacatcccacttccagcactgcctgcggtgaaggataatacgactcactataggggctagcgtttaaacttaagcttgccaccatggacgggtccggggagcagcttgggagcggcgggcccaccagctctgaacagatcatgaagacaggggcctttttgctacagggtttcatccaggatcgagcagggaggatggctggggagacacctgagctgaccttggagcagccgccccaggatgcgtccaccaagaagctgagcgagtgtctccggcgaattggagatgaactggacagcaatatggagctgcagaggatgattgctgacgtggacacggactccccccgagaggtcttcttccgggtggcagctgacatgtttgctgatggcaacttcaactggggccgcgtggttgccctcttctactttgctagcaaactggtgctcaaggccctgtgcactaaagtgcccgagctgatcagaaccatcatgggctggacactggacttcctccgtgagcggctgcttgtctggatccaagaccagggtggctgggaaggcctcctctcctacttcgggacccccacatggcagacagtgaccatctttgtggctggagtcctcaccgcctcgctcaccatctggaagaagatgggctgactcgagggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagt

SEQ ID NO: 148 - RNA, 1713 nt, synthetic construct
  misc_feature 1..1713: note = synthetic construct
  source 1..1713: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccagaatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagaccatcatgttcgacgacgccattgagagggcgggccagctcaccagcgaggaggacacgctgagcctcgtcactgccgaccactcccacgtgttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttacagcagagtcggggcggccggccgcttcgagcagacatgagcggccgctcaatctcgaggggatccactagtccagtgtggtggaattcgtttaaaccctcctcgtccggggctgtgtcgccccgctgacgagccctgaaaaagggcgaaacgaggaggactagtgggcccgtttaaacccgctgatcagcct

SEQ ID NO: 149 - RNA, 2106 nt, synthetic construct
  misc_feature 1..2106: note = synthetic construct
  source 1..2106: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcgagggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagtgggcccgtttaaacccgctgatcagcct

SEQ ID NO: 150 - DNA, 1135 bp, synthetic construct
  misc_feature 1..1135: note = synthetic construct
  source 1..1135: mol_type = other DNA
gtcgacatttcccgtaaatcgtcgagtcgacatttcccgtaaatcgtcgagggcccatcgatgtcgacatttcccgtaaatcgtcgagtcgacatttcccgtaaatcgtcgagggcccgtcgacatttcccgtaaatcgtcgagtcgacatttcccgtaaatcgtcgaatcgatgtcgacatttcccgtaaatcgtcgagtcgacatttcccgtaaatcgtcgaacgcgtcctgcaggtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggaattcgagctcgcccggggatcctctagagccacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 151 - DNA, 2223 bp, synthetic construct
  misc_feature 1..2223: note = synthetic construct
  source 1..2223: mol_type = other DNA
attggatccaatgcattggatccaatggattggatccaatcgattggatccaattgattggatccaatttaattaagcagctcctgcaggtagagggtatataatggaagctcgacttccaggctagcgtttaaacttaagcttgccaccatgggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccatatgatgctcaagatggagtctacacagcagatggctagttcgattattaacacttcttttgaagccgcagtcgttgctgccacttcaactcttgaacttatgggcattcaatatgattataatgaagtttatactagagttaagagcaaatttgattacgtaatggatgattcaggtgttaaaaacaatctgttgggtaaagcggcaactattgatcaggcactgaatggaaaatttggttcagcagcaagaaatagaaattggatggctgatactagaactacagctagactagatgaagatgtgaacaaacttagaatgatgctatcatctaaaggaattgatcaaaaaatgagagtacttaatgcatgtttcaatgtgaagcgagttccaggtaaatcatcatcaataattaaatgtactagattaatgagagataagattgaacgtggtgaagtcgaagtggatgattcgtttgttgaagaaaaaatggaagttgacacaattgattggaaatctagatatgaacaattggaaaaacgatttgaatcgctgaaacaaagggtgaatgaaaaatatacaagttgggtgcaaaaagcaaaaaaggtaaatgaaaatatgtactctctccaaaacgttatatcacaacagcagagccagattgctgatttacaaaattactgtaataaattggaagttgatctacaaaataaaattagttcacttgtgtcttcagtcgaatggtacctgaaatcaatggaattgcctgatgaaattaagacagatattgaacaacaactaaattccattgatgtaatcaatccaattaatgctatagatgattttgaatcactgatcagaaatattatactcgactatgacagaatatttttaatgtttaaaggattaatgaggcaatgcaactatgaatatacgtatgaatag

SEQ ID NO: 152 - DNA, 3365 bp, synthetic construct
  misc_feature 1..3365: note = synthetic construct
  source 1..3365: mol_type = other DNA
attggatccaatgcattggatccaatggattggatccaatcgattggatccaattgattggatccaatttaattaagcagctcctgcaggtagagggtatataatggaagctcgacttccagggagctctctggctaactagagaacccactgcttactggcttatcgaaattaatacgactcactatagggagacccaagctggctaggccaccatggacatcgagaagctgtgcaagaaggcggaagaggaggccaaggaggcccaggagaaggccgacgagctgagacaacgacaccccgattcgcaggcggcggaagacgcggaggatctcgcaaatctggctgtcgccgccgtgctgaccgcttgtctgctggcccaggagcatcccaacgccgatatcgctaagctgtgcatcaaggccgcgagcgaagctgcggaggccgcgtcaaaagccgccgagctggcccaacggcacccagacagtcaagcagccagagatgctatcaaactcgcctctcaagccgcccgcgccgtgatcctggccatcatgctcgccgcagagaatcccaacgctgatattgcaaagctatgtattaaggcggccagcgaagccgcagaggctgcctcgaaagctgcggaacttgcacagcggcaccccgactcccaggccgcccgagatgcaattaaactggccagtcaggccgctgaggcggtggagagagccatctggctggccgccgagaaccccaatgccgatatcgccaaaaaatgcatcaaagcggcctccgaagcagccgaggaggcctctaaagctgccgaagaggctcaaagacacccagacagccagaaggcacgcgacgagatcaaggaggcctctcagaaggccgaggaggtgaaggagagatgcaagagcgccagcggaagtggcggaggaggcgacgtcgctagtgacatcgagaagctgtgcaagaaggcggaagaggaggccaaggaggcccaggagaaggccgacgagctgagacaacgacaccccgattcgcaggcggcggaagacgcggaggatctcgcaaatctggctgtcgccgccgtgctgaccgcttgtctgctggcccaggagcatcccaacgccgatatcgctaagctgtgcatcaaggccgcgagcgaagctgcggaggccgcgtcaaaagccgccgagctggcccaacggcacccagacagtcaagcagccagagatgctatcaaactcgcctctcaagccgcccgcgccgtgatcctggccatcatgctcgccgcagagaatcccaacgctgatattgcaaagctatgtattaaggcggccagcgaagccgcagaggctgcctcgaaagctgcggaacttgcacagcggcaccccgactcccaggccgcccgagatgcaattaaactggccagtcaggccgctgaggcggtggagagagccatctggctggccgccgagaaccccaatgccgatatcgccaaaaaatgcatcaaagcggcctccgaagcagccgaggaggcctctaaagctgccgaagaggctcaaagacacccagacagccagaaggcacgcgacgagatcaaggaggcctctcagaaggccgaggaggtgaaggagagatgcaagagcgccagcggaagtggcggaggaggcgacgtcgctagtgacatcgagaagctgtgcaagaaggcggaagaggaggccaaggaggcccaggagaaggccgacgagctgagacaacgacaccccgattcgcaggcggcggaagacgcggaggatctcgcaaatctggctgtcgccgccgtgctgaccgcttgtctgctggcccaggagcatcccaacgccgatatcgctaagctgtgcatcaaggccgcgagcgaagctgcggaggccgcgtcaaaagccgccgagctggcccaacggcacccagacagtcaagcagccagagatgctatcaaactcgcctctcaagccgcccgcgccgtgatcctggccatcatgctcgccgcagagaatcccaacgctgatattgcaaagctatgtattaaggcggccagcgaagccgcagaggctgcctcgaaagctgcggaacttgcacagcggcaccccgactcccaggccgcccgagatgcaattaaactggccagtcaggccgctgaggcggtggagagagccatctggctggccgccgagaaccccaatgccgatatcgccaaaaaatgcatcaaagcggcctccgaagcagccgaggaggcctctaaagctgccgaagaggctcaaagacacccagacagccagaaggcacgcgacgagatcaaggaggcctctcagaaggccgaggaggtgaaggagagatgcaagagcgccagcggaagtggcggaggaggcgacgtcgctagcatgctcaagatggagtctacacagcagatggctagttcgattattaacacttcttttgaagccgcagtcgttgctgccacttcaactcttgaacttatgggcattcaatatgattataatgaagtttatactagagttaagagcaaatttgattacgtaatggatgattcaggtgttaaaaacaatctgttgggtaaagcggcaactattgatcaggcactgaatggaaaatttggttcagcagcaagaaatagaaattggatggctgatactagaactacagctagactagatgaagatgtgaacaaacttagaatgatgctatcatctaaaggaattgatcaaaaaatgagagtacttaatgcatgtttcaatgtgaagcgagttccaggtaaatcatcatcaataattaaatgtactagattaatgagagataagattgaacgtggtgaagtcgaagtggatgattcgtttgttgaagaaaaaatggaagttgacacaattgattggaaatctagatatgaacaattggaaaaacgatttgaatcgctgaaacaaagggtgaatgaaaaatatacaagttgggtgcaaaaagcaaaaaaggtaaatgaaaatatgtactctctccaaaacgttatatcacaacagcagagccagattgctgatttacaaaattactgtaataaattggaagttgatctacaaaataaaattagttcacttgtgtcttcagtcgaatggtacctgaaatcaatggaattgcctgatgaaattaagacagatattgaacaacaactaaattccattgatgtaatcaatccaattaatgctatagatgattttgaatcactgatcagaaatattatactcgactatgacagaatatttttaatgtttaaaggattaatgaggcaatgcaactatgaatatacgtatgaatag

SEQ ID NO: 153 - DNA, 2316 bp, synthetic construct
  misc_feature 1..2316: note = synthetic construct
  source 1..2316: mol_type = other DNA
tgacgtcagctgccagatcccatggccgtcatactgtgacgtctttcagacaccccattgacgtcaatgggagaacagatctgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagaagcttgccaccatgggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccatatgatgctcaagatggagtctacacagcagatggctagttcgattattaacacttcttttgaagccgcagtcgttgctgccacttcaactcttgaacttatgggcattcaatatgattataatgaagtttatactagagttaagagcaaatttgattacgtaatggatgattcaggtgttaaaaacaatctgttgggtaaagcggcaactattgatcaggcactgaatggaaaatttggttcagcagcaagaaatagaaattggatggctgatactagaactacagctagactagatgaagatgtgaacaaacttagaatgatgctatcatctaaaggaattgatcaaaaaatgagagtacttaatgcatgtttcaatgtgaagcgagttccaggtaaatcatcatcaataattaaatgtactagattaatgagagataagattgaacgtggtgaagtcgaagtggatgattcgtttgttgaagaaaaaatggaagttgacacaattgattggaaatctagatatgaacaattggaaaaacgatttgaatcgctgaaacaaagggtgaatgaaaaatatacaagttgggtgcaaaaagcaaaaaaggtaaatgaaaatatgtactctctccaaaacgttatatcacaacagcagagccagattgctgatttacaaaattactgtaataaattggaagttgatctacaaaataaaattagttcacttgtgtcttcagtcgaatggtacctgaaatcaatggaattgcctgatgaaattaagacagatattgaacaacaactaaattccattgatgtaatcaatccaattaatgctatagatgattttgaatcactgatcagaaatattatactcgactatgacagaatatttttaatgtttaaaggattaatgaggcaatgcaactatgaatatacgtatgaatag

SEQ ID NO: 154 - DNA, 2963 bp, synthetic construct
  misc_feature 1..2963: note = synthetic construct
  source 1..2963: mol_type = other DNA
tgacgtccgagagcctgacgtccgagagcctgacgtccgagagcctgacgtccgagatctctcgaggtcgacagcggagactctagagggtatataatggaagctcgacttccagcttggcaatccggtactgttggtaaagaaagctgccggccaccatggagaccgataccttattactgtgggtgcttcttctgtgggttcccggtagcaccggagacggatccaagcttgccaccatgggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccatatggaattcgcaccttcacccagcctcccggaccccaagtttgagagcaaagcggccctgctggcatcccggggctccgaagaacttctgtgcttcacccaacgcttggaagacttggtgtgtttctgggaggaagcggcgagctccgggatggacttcaactacagcttctcataccagctcgagggtgagtcacgaaagtcatgtagcctgcaccaggctcccaccgtccgcggctccgtgcgtttctggtgttcactgccaacagcggacacatcgagtgctgtgccgctggagctgcaggtgacggaggcgtccggttctcctcgctatcaccgcatcatccatatcaatgaagtagtgctcctggacgcccccgcggggctgctggcgcgccgggcagaagagggcagccacgtggtgctgcgctggctgccacctcctggagcacctatgaccacccacatccgatatgaagtggacgtgtcggcaggcaaccgggcaggagggacacaaagggtggaggtcctggaaggccgcactgagtgtgttctgagcaacctgcggggcgggacgcgctacaccttcgctgttcgagcgcgcatggccgagccgagcttcagcggattctggagtgcctggtctgagcccgcgtcactactgaccgctagcgacctggaccctctcatcttgacgctgtctctcattctggtcctcatctcgttgttgctgacggttctggccctgctgtccgcagcagcaaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccaggcttctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtaa

SEQ ID NO: 155 - DNA, 3038 bp, synthetic construct
  misc_feature 1..3038: note = synthetic construct
  source 1..3038: mol_type = other DNA
attggatccaatgcattggatccaatggattggatccaatcgattggatccaattgattggatccaatctagctcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcgggccggccaccatggagaccgataccttattactgtgggtgcttcttctgtgggttcccggtagcaccggagacggatccaagcttgccaccatgggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccatatggaattcgcaccttcacccagcctcccggaccccaagtttgagagcaaagcggccctgctggcatcccggggctccgaagaacttctgtgcttcacccaacgcttggaagacttggtgtgtttctgggaggaagcggcgagctccgggatggacttcaactacagcttctcataccagctcgagggtgagtcacgaaagtcatgtagcctgcaccaggctcccaccgtccgcggctccgtgcgtttctggtgttcactgccaacagcggacacatcgagtgctgtgccgctggagctgcaggtgacggaggcgtccggttctcctcgctatcaccgcatcatccatatcaatgaagtagtgctcctggacgcccccgcggggctgctggcgcgccgggcagaagagggcagccacgtggtgctgcgctggctgccacctcctggagcacctatgaccacccacatccgatatgaagtggacgtgtcggcaggcaaccgggcaggagggacacaaagggtggaggtcctggaaggccgcactgagtgtgttctgagcaacctgcggggcgggacgcgctacaccttcgctgttcgagcgcgcatggccgagccgagcttcagcggattctggagtgcctggtctgagcccgcgtcactactgaccgctagcgacctggaccctctcatcttgacgctgtctctcattctggtcctcatctcgttgttgctgacggttctggccctgctgtccgcagcagcaaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccaggcttctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtaa

SEQ ID NO: 156 - DNA, 2483 bp, synthetic construct
  misc_feature 1..2483: note = synthetic construct
  source 1..2483: mol_type = other DNA
attggatccaatgcattggatccaatggattggatccaatcgattggatccaattgattggatccaatctagctcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcgggccggccaccatggagaccgataccttattactgtgggtgcttcttctgtgggttcccggtagcaccggagacggatccaagaagaagggcagcgtggtgatcgtgggcagaatcaacctgagcggcgacaccgcctacagccagcagaccagaggcctggagggctgccaggagaccagccagaccggcagagacaagaaccaggtggagggcgaggtgcaggtggtgagcaccgccacccagagcttcctggccaccagcatcaacggcgtgctgtggaccgtgtaccacggcgccggcaccagaaccatcgccagccccaagggccccgtgacccagatgtacacgaacgtggacaaggacttagtgggctggcaggccccccaaggcagcagaagcctgaccccctgcacctgcggcagcagcgacctgtacctggtgaccagacacgccgacgtgatccccgtgagaagaagaggcgacagcagaggcagcctgctgagccccagacccatcagctatctgaaaggctcaagcggcggccccctgttgtgtcccgccggccatgccgtgggcatcttcagagccgccgtgagcaccagaggcgtggccaaggccgtggacttcatccccgtggagagcctggagaccaccatgagaagccccgaattcgcaccttcacccagcctcccggaccccaagtttgagagcaaagcggccctgctggcatcccggggctccgaagaacttctgtgcttcacccaacgcttggaagacttggtgtgtttctgggaggaagcggcgagctccgggatggacttcaactacagcttctcataccagctcgagggtgagtcacgaaagtcatgtagcctgcaccaggctcccaccgtccgcggctccgtgcgtttctggtgttcactgccaacagcggacacatcgagtgctgtgccgctggagctgcaggtgacggaggcgtccggttctcctcgctatcaccgcatcatccatatcaatgaagtagtgctcctggacgcccccgcggggctgctggcgcgccgggcagaagagggcagccacgtggtgctgcgctggctgccacctcctggagcacctatgaccacccacatccgatatgaagtggacgtgtcggcaggcaaccgggcaggagggacacaaagggtggaggtcctggaaggccgcactgagtgtgttctgagcaacctgcggggcgggacgcgctacaccttcgctgttcgagcgcgcatggccgagccgagcttcagcggattctggagtgcctggtctgagcccgcgtcactactgaccgctagcgacctggaccctctcatcttgacgctgtctctcattctggtcctcatctcgttgttgctgacggttctggccctgctgtccgcagcagcaaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccaggcttctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtaa

SEQ ID NO: 157 - DNA, 2405 bp, synthetic construct
  misc_feature 1..2405: note = synthetic construct
  source 1..2405: mol_type = other DNA
tgacgtccgagagcctgacgtccgagagcctgacgtccgagagcctgacgtccgagatctctcgaggtcgacagcggagactctagagggtatataatggaagctcgacttccagcttggcaatccggtactgttggtaaagaaagcttgccaccatggagaccgataccttattactgtgggtgcttcttctgtgggttcccggtagcaccggagacggatccaagaagaagggcagcgtggtgatcgtgggcagaatcaacctgagcggcgacaccgcctacagccagcagaccagaggcctggagggctgccaggagaccagccagaccggcagagacaagaaccaggtggagggcgaggtgcaggtggtgagcaccgccacccagagcttcctggccaccagcatcaacggcgtgctgtggaccgtgtaccacggcgccggcaccagaaccatcgccagccccaagggccccgtgacccagatgtacacgaacgtggacaaggacttagtgggctggcaggccccccaaggcagcagaagcctgaccccctgcacctgcggcagcagcgacctgtacctggtgaccagacacgccgacgtgatccccgtgagaagaagaggcgacagcagaggcagcctgctgagccccagacccatcagctatctgaaaggctcaagcggcggccccctgttgtgtcccgccggccatgccgtgggcatcttcagagccgccgtgagcaccagaggcgtggccaaggccgtggacttcatccccgtggagagcctggagaccaccatgagaagccccgaattcgcaccttcacccagcctcccggaccccaagtttgagagcaaagcggccctgctggcatcccggggctccgaagaacttctgtgcttcacccaacgcttggaagacttggtgtgtttctgggaggaagcggcgagctccgggatggacttcaactacagcttctcataccagctcgagggtgagtcacgaaagtcatgtagcctgcaccaggctcccaccgtccgcggctccgtgcgtttctggtgttcactgccaacagcggacacatcgagtgctgtgccgctggagctgcaggtgacggaggcgtccggttctcctcgctatcaccgcatcatccatatcaatgaagtagtgctcctggacgcccccgcggggctgctggcgcgccgggcagaagagggcagccacgtggtgctgcgctggctgccacctcctggagcacctatgaccacccacatccgatatgaagtggacgtgtcggcaggcaaccgggcaggagggacacaaagggtggaggtcctggaaggccgcactgagtgtgttctgagcaacctgcggggcgggacgcgctacaccttcgctgttcgagcgcgcatggccgagccgagcttcagcggattctggagtgcctggtctgagcccgcgtcactactgaccgctagcgacctggaccctctcatcttgacgctgtctctcattctggtcctcatctcgttgttgctgacggttctggccctgctgtccgcagcagcaaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccaggcttctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtaa

SEQ ID NO: 158 - DNA, 2516 bp, synthetic construct
  misc_feature 1..2516: note = synthetic construct
  source 1..2516: mol_type = other DNA
tgacgtccgagagcctgacgtccgagagcctgacgtccgagagcctgacgtccgagatctctcgaggtcgacagcggagactctagagggtatataatggaagctcgacttccagcttggcaatccggtactgttggtaaagaaagcttgccaccatggagaccgataccttattactgtgggtgcttcttctgtgggttcccggtagcaccggagacggatccgacatcgagaagctgtgcaagaaggcggaagaggaggccaaggaggcccaggagaaggccgacgagctgagacaacgacaccccgattcgcaggcggcggaagacgcggaggatctcgcaaatctggctgtcgccgccgtgctgaccgcttgtctgctggcccaggagcatcccaacgccgatatcgctaagctgtgcatcaaggccgcgagcgaagctgcggaggccgcgtcaaaagccgccgagctggcccaacggcacccagacagtcaagcagccagagatgctatcaaactcgcctctcaagccgcccgcgccgtgatcctggccatcatgctcgccgcagagaatcccaacgctgatattgcaaagctatgtattaaggcggccagcgaagccgcagaggctgcctcgaaagctgcggaacttgcacagcggcaccccgactcccaggccgcccgagatgcaattaaactggccagtcaggccgctgaggcggtggagagagccatctggctggccgccgagaaccccaatgccgatatcgccaaaaaatgcatcaaagcggcctccgaagcagccgaggaggcctctaaagctgccgaagaggctcaaagacacccagacagccagaaggcacgcgacgagatcaaggaggcctctcagaaggccgaggaggtgaaggagagatgcaagagcgaattcgcaccttcacccagcctcccggaccccaagtttgagagcaaagcggccctgctggcatcccggggctccgaagaacttctgtgcttcacccaacgcttggaagacttggtgtgtttctgggaggaagcggcgagctccgggatggacttcaactacagcttctcataccagctcgagggtgagtcacgaaagtcatgtagcctgcaccaggctcccaccgtccgcggctccgtgcgtttctggtgttcactgccaacagcggacacatcgagtgctgtgccgctggagctgcaggtgacggaggcgtccggttctcctcgctatcaccgcatcatccatatcaatgaagtagtgctcctggacgcccccgcggggctgctggcgcgccgggcagaagagggcagccacgtggtgctgcgctggctgccacctcctggagcacctatgaccacccacatccgatatgaagtggacgtgtcggcaggcaaccgggcaggagggacacaaagggtggaggtcctggaaggccgcactgagtgtgttctgagcaacctgcggggcgggacgcgctacaccttcgctgttcgagcgcgcatggccgagccgagcttcagcggattctggagtgcctggtctgagcccgcgtcactactgaccgctagcgacctggaccctctcatcttgacgctgtctctcattctggtcctcatctcgttgttgctgacggttctggccctgctgtccgcagcagcaaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccaggcttctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtaa

SEQ ID NO: 159 - DNA, 3510 bp, synthetic construct
  misc_feature 1..3510: note = synthetic construct
  source 1..3510: mol_type = other DNA
attggatccaatgcattggatccaatggattggatccaatcgattggatccaattgattggatccaatctagctcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcgcccggggatcctctagagtcagcttctgcgagctctctggctaactagagaacccactgcttactggcttatcgaaattaatacgactcactatagggagacccaagctggctaggccaccatggacatcgagaagctgtgcaagaaggcggaagaggaggccaaggaggcccaggagaaggccgacgagctgagacaacgacaccccgattcgcaggcggcggaagacgcggaggatctcgcaaatctggctgtcgccgccgtgctgaccgcttgtctgctggcccaggagcatcccaacgccgatatcgctaagctgtgcatcaaggccgcgagcgaagctgcggaggccgcgtcaaaagccgccgagctggcccaacggcacccagacagtcaagcagccagagatgctatcaaactcgcctctcaagccgcccgcgccgtgatcctggccatcatgctcgccgcagagaatcccaacgctgatattgcaaagctatgtattaaggcggccagcgaagccgcagaggctgcctcgaaagctgcggaacttgcacagcggcaccccgactcccaggccgcccgagatgcaattaaactggccagtcaggccgctgaggcggtggagagagccatctggctggccgccgagaaccccaatgccgatatcgccaaaaaatgcatcaaagcggcctccgaagcagccgaggaggcctctaaagctgccgaagaggctcaaagacacccagacagccagaaggcacgcgacgagatcaaggaggcctctcagaaggccgaggaggtgaaggagagatgcaagagcgccagcggaagtggcggaggaggcgacgtcgctagtgacatcgagaagctgtgcaagaaggcggaagaggaggccaaggaggcccaggagaaggccgacgagctgagacaacgacaccccgattcgcaggcggcggaagacgcggaggatctcgcaaatctggctgtcgccgccgtgctgaccgcttgtctgctggcccaggagcatcccaacgccgatatcgctaagctgtgcatcaaggccgcgagcgaagctgcggaggccgcgtcaaaagccgccgagctggcccaacggcacccagacagtcaagcagccagagatgctatcaaactcgcctctcaagccgcccgcgccgtgatcctggccatcatgctcgccgcagagaatcccaacgctgatattgcaaagctatgtattaaggcggccagcgaagccgcagaggctgcctcgaaagctgcggaacttgcacagcggcaccccgactcccaggccgcccgagatgcaattaaactggccagtcaggccgctgaggcggtggagagagccatctggctggccgccgagaaccccaatgccgatatcgccaaaaaatgcatcaaagcggcctccgaagcagccgaggaggcctctaaagctgccgaagaggctcaaagacacccagacagccagaaggcacgcgacgagatcaaggaggcctctcagaaggccgaggaggtgaaggagagatgcaagagcgccagcggaagtggcggaggaggcgacgtcgctagtgacatcgagaagctgtgcaagaaggcggaagaggaggccaaggaggcccaggagaaggccgacgagctgagacaacgacaccccgattcgcaggcggcggaagacgcggaggatctcgcaaatctggctgtcgccgccgtgctgaccgcttgtctgctggcccaggagcatcccaacgccgatatcgctaagctgtgcatcaaggccgcgagcgaagctgcggaggccgcgtcaaaagccgccgagctggcccaacggcacccagacagtcaagcagccagagatgctatcaaactcgcctctcaagccgcccgcgccgtgatcctggccatcatgctcgccgcagagaatcccaacgctgatattgcaaagctatgtattaaggcggccagcgaagccgcagaggctgcctcgaaagctgcggaacttgcacagcggcaccccgactcccaggccgcccgagatgcaattaaactggccagtcaggccgctgaggcggtggagagagccatctggctggccgccgagaaccccaatgccgatatcgccaaaaaatgcatcaaagcggcctccgaagcagccgaggaggcctctaaagctgccgaagaggctcaaagacacccagacagccagaaggcacgcgacgagatcaaggaggcctctcagaaggccgaggaggtgaaggagagatgcaagagcgccagcggaagtggcggaggaggcgacgtcgctagcatgctcaagatggagtctacacagcagatggctagttcgattattaacacttcttttgaagccgcagtcgttgctgccacttcaactcttgaacttatgggcattcaatatgattataatgaagtttatactagagttaagagcaaatttgattacgtaatggatgattcaggtgttaaaaacaatctgttgggtaaagcggcaactattgatcaggcactgaatggaaaatttggttcagcagcaagaaatagaaattggatggctgatactagaactacagctagactagatgaagatgtgaacaaacttagaatgatgctatcatctaaaggaattgatcaaaaaatgagagtacttaatgcatgtttcaatgtgaagcgagttccaggtaaatcatcatcaataattaaatgtactagattaatgagagataagattgaacgtggtgaagtcgaagtggatgattcgtttgttgaagaaaaaatggaagttgacacaattgattggaaatctagatatgaacaattggaaaaacgatttgaatcgctgaaacaaagggtgaatgaaaaatatacaagttgggtgcaaaaagcaaaaaaggtaaatgaaaatatgtactctctccaaaacgttatatcacaacagcagagccagattgctgatttacaaaattactgtaataaattggaagttgatctacaaaataaaattagttcacttgtgtcttcagtcgaatggtacctgaaatcaatggaattgcctgatgaaattaagacagatattgaacaacaactaaattccattgatgtaatcaatccaattaatgctatagatgattttgaatcactgatcagaaatattatactcgactatgacagaatatttttaatgtttaaaggattaatgaggcaatgcaactatgaatatacgtatgaatag

SEQ ID NO: 160 - DNA, 1752 bp, synthetic construct
  misc_feature 1..1752: note = synthetic construct
  source 1..1752: mol_type = other DNA
tgacgtcagctgccagatcccatggccgtcatactgtgacgtctttcagacaccccattgacgtcaatgggagaacagatctgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagaagcttgccaccatgggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccatatgatgctcaagatggagtctacacagcagatggctagttcgattattaacacttcttttgaagccgcagtcgttgctgccacttcaactcttgaacttatgggcattcaatatgattataatgaagtttatactagagttaagagcaaatttgattacgtaatggatgattcaggtgttaaaaacaatctgttgggtaaagcggcaactattgatcaggcactgaatggaaaatttggttcagcagcaagaaatagaaattggatggctgatactagaactacagctagactagatgaagatgtgaacaaacttagaatgatgctatcatctaaaggaattgatcaaaaaatgagagtacttaatgcatgtttcaatgtgaagcgagttccaggtaaatcatcatcaataattaaatgtactagattaatgagagataagattgaacgtggtgaagtcgaagtggatgattcgtttgttgaagaaaaaatggaagttgacacaattgattggaaatctagatatgaacaattggaaaaacgatttgaatcgctgaaacaaagggtgaatgaaaaatatacaagttgggtgcaaaaagcaaaaaaggtaaatgaaaatatgtactctctccaaaacgttatatcacaacagcagagccagattgctgatttacaaaattactgtaataaattggaagttgatctacaaaataaaattagttcacttgtgtcttcagtcgaatggtacctgaaatcaatggaattgcctgatgaaattaagacagatattgaacaacaactaaattccattgatgtaatcaatccaattaatgctatagatgattttgaatcactgatcagaaatattatactcgactatgacagaatatttttaatgtttaaaggattaatgaggcaatgcaactatgaatatacgtatgaatag

SEQ ID NO: 161 - DNA, 1659 bp, synthetic construct
  misc_feature 1..1659: note = synthetic construct
  source 1..1659: mol_type = other DNA
attggatccaatgcattggatccaatggattggatccaatcgattggatccaattgattggatccaatttaattaagcagctcctgcaggtagagggtatataatggaagctcgacttccaggctagcgtttaaacttaagcttgccaccatgggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccataccggggagctcgacgagttggtttatctcttagacggacccggatatgaccccattcattcagatggctccggaacgggcagcgggaccggaagtggcaccggaaccacttccggaaccggtactggcgggagtaccggcggtgagctagacgaactcgtgtacctgttggatggccccggttacgacccaatccacagcgatggaagtggaactgggtctgggactgggtccggcaccgggactacttcaggcaccggcactgggggctccactggccatatgatgctcaagatggagtctacacagcagatggctagttcgattattaacacttcttttgaagccgcagtcgttgctgccacttcaactcttgaacttatgggcattcaatatgattataatgaagtttatactagagttaagagcaaatttgattacgtaatggatgattcaggtgttaaaaacaatctgttgggtaaagcggcaactattgatcaggcactgaatggaaaatttggttcagcagcaagaaatagaaattggatggctgatactagaactacagctagactagatgaagatgtgaacaaacttagaatgatgctatcatctaaaggaattgatcaaaaaatgagagtacttaatgcatgtttcaatgtgaagcgagttccaggtaaatcatcatcaataattaaatgtactagattaatgagagataagattgaacgtggtgaagtcgaagtggatgattcgtttgttgaagaaaaaatggaagttgacacaattgattggaaatctagatatgaacaattggaaaaacgatttgaatcgctgaaacaaagggtgaatgaaaaatatacaagttgggtgcaaaaagcaaaaaaggtaaatgaaaatatgtactctctccaaaacgttatatcacaacagcagagccagattgctgatttacaaaattactgtaataaattggaagttgatctacaaaataaaattagttcacttgtgtcttcagtcgaatggtacctgaaatcaatggaattgcctgatgaaattaagacagatattgaacaacaactaaattccattgatgtaatcaatccaattaatgctatagatgattttgaatcactgatcagaaatattatactcgactatgacagaatatttttaatgtttaaaggattaatgaggcaatgcaactatgaatatacgtatgaatag

SEQ ID NO: 162 - DNA, 3502 bp, synthetic construct
  misc_feature 1..3502: note = synthetic construct
  source 1..3502: mol_type = other DNA
gcaccagacagtgacgtcagctgccagatcccatggccgtcatactgtgacgtctttcagacaccccattgacgtcaatgggagaacagatctgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagaagcttcgaatcgcgaattcggagctctctggctaactagagaacccactgcttactggcttatcgaaattaatacgactcactatagggagacccaagctggctaggccaccatggacatcgagaagctgtgcaagaaggcggaagaggaggccaaggaggcccaggagaaggccgacgagctgagacaacgacaccccgattcgcaggcggcggaagacgcggaggatctcgcaaatctggctgtcgccgccgtgctgaccgcttgtctgctggcccaggagcatcccaacgccgatatcgctaagctgtgcatcaaggccgcgagcgaagctgcggaggccgcgtcaaaagccgccgagctggcccaacggcacccagacagtcaagcagccagagatgctatcaaactcgcctctcaagccgcccgcgccgtgatcctggccatcatgctcgccgcagagaatcccaacgctgatattgcaaagctatgtattaaggcggccagcgaagccgcagaggctgcctcgaaagctgcggaacttgcacagcggcaccccgactcccaggccgcccgagatgcaattaaactggccagtcaggccgctgaggcggtggagagagccatctggctggccgccgagaaccccaatgccgatatcgccaaaaaatgcatcaaagcggcctccgaagcagccgaggaggcctctaaagctgccgaagaggctcaaagacacccagacagccagaaggcacgcgacgagatcaaggaggcctctcagaaggccgaggaggtgaaggagagatgcaagagcgccagcggaagtggcggaggaggcgacgtcgctagtgacatcgagaagctgtgcaagaaggcggaagaggaggccaaggaggcccaggagaaggccgacgagctgagacaacgacaccccgattcgcaggcggcggaagacgcggaggatctcgcaaatctggctgtcgccgccgtgctgaccgcttgtctgctggcccaggagcatcccaacgccgatatcgctaagctgtgcatcaaggccgcgagcgaagctgcggaggccgcgtcaaaagccgccgagctggcccaacggcacccagacagtcaagcagccagagatgctatcaaactcgcctctcaagccgcccgcgccgtgatcctggccatcatgctcgccgcagagaatcccaacgctgatattgcaaagctatgtattaaggcggccagcgaagccgcagaggctgcctcgaaagctgcggaacttgcacagcggcaccccgactcccaggccgcccgagatgcaattaaactggccagtcaggccgctgaggcggtggagagagccatctggctggccgccgagaaccccaatgccgatatcgccaaaaaatgcatcaaagcggcctccgaagcagccgaggaggcctctaaagctgccgaagaggctcaaagacacccagacagccagaaggcacgcgacgagatcaaggaggcctctcagaaggccgaggaggtgaaggagagatgcaagagcgccagcggaagtggcggaggaggcgacgtcgctagtgacatcgagaagctgtgcaagaaggcggaagaggaggccaaggaggcccaggagaaggccgacgagctgagacaacgacaccccgattcgcaggcggcggaagacgcggaggatctcgcaaatctggctgtcgccgccgtgctgaccgcttgtctgctggcccaggagcatcccaacgccgatatcgctaagctgtgcatcaaggccgcgagcgaagctgcggaggccgcgtcaaaagccgccgagctggcccaacggcacccagacagtcaagcagccagagatgctatcaaactcgcctctcaagccgcccgcgccgtgatcctggccatcatgctcgccgcagagaatcccaacgctgatattgcaaagctatgtattaaggcggccagcgaagccgcagaggctgcctcgaaagctgcggaacttgcacagcggcaccccgactcccaggccgcccgagatgcaattaaactggccagtcaggccgctgaggcggtggagagagccatctggctggccgccgagaaccccaatgccgatatcgccaaaaaatgcatcaaagcggcctccgaagcagccgaggaggcctctaaagctgccgaagaggctcaaagacacccagacagccagaaggcacgcgacgagatcaaggaggcctctcagaaggccgaggaggtgaaggagagatgcaagagcgccagcggaagtggcggaggaggcgacgtcgctagcatgctcaagatggagtctacacagcagatggctagttcgattattaacacttcttttgaagccgcagtcgttgctgccacttcaactcttgaacttatgggcattcaatatgattataatgaagtttatactagagttaagagcaaatttgattacgtaatggatgattcaggtgttaaaaacaatctgttgggtaaagcggcaactattgatcaggcactgaatggaaaatttggttcagcagcaagaaatagaaattggatggctgatactagaactacagctagactagatgaagatgtgaacaaacttagaatgatgctatcatctaaaggaattgatcaaaaaatgagagtacttaatgcatgtttcaatgtgaagcgagttccaggtaaatcatcatcaataattaaatgtactagattaatgagagataagattgaacgtggtgaagtcgaagtggatgattcgtttgttgaagaaaaaatggaagttgacacaattgattggaaatctagatatgaacaattggaaaaacgatttgaatcgctgaaacaaagggtgaatgaaaaatatacaagttgggtgcaaaaagcaaaaaaggtaaatgaaaatatgtactctctccaaaacgttatatcacaacagcagagccagattgctgatttacaaaattactgtaataaattggaagttgatctacaaaataaaattagttcacttgtgtcttcagtcgaatggtacctgaaatcaatggaattgcctgatgaaattaagacagatattgaacaacaactaaattccattgatgtaatcaatccaattaatgctatagatgattttgaatcactgatcagaaatattatactcgactatgacagaatatttttaatgtttaaaggattaatgaggcaatgcaactatgaatatacgtatgaa

SEQ ID NO: 163 - RNA, 1989 nt, synthetic construct
  misc_feature 1..1989: note = synthetic construct
  source 1..1989: mol_type = other RNA
atgactagtgagacagacacactcctgctatgggtactgctgctctgggttccaggttccactggtgacgctagtggtggttctggtatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtgactcgagggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagtgggcccgtttaaacccgctgatcagcct

SEQ ID NO: 164 - DNA, 1003 bp, synthetic construct
  misc_feature 1..1003: note = synthetic construct
  source 1..1003: mol_type = other DNA
gtcgacatttcccgtaaatcgtcgagtcgacatttcccgtaaatcgtcgagggcccatcgatgtcgacatttcccgtaaatcgtcgagtcgacatttcccgtaaatcgtcgagggcccgtcgacatttcccgtaaatcgtcgagtcgacatttcccgtaaatcgtcgaatcgatgtcgacatttcccgtaaatcgtcgagtcgacatttcccgtaaatcgtcgaacgcgtcctgcaggtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggaattcgccacctatgactagtgagacagacacactcctgctatgggtactgctgctctgggttccaggttccactggtgacgctagtggtggttctggtatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 165 - DNA, 1823 bp, synthetic construct
  misc_feature 1..1823: note = synthetic construct
  source 1..1823: mol_type = other DNA
atactcagtgccctgactatatactcagtgccctgactatatactcagtgccctgactatatactcagtgccctgactatatactcagtgccctgactatgctagctcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcgcccggggatcctctagagtcagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaa

SEQ ID NO: 166 - DNA, 1911 bp, synthetic construct
  misc_feature 1..1911: note = synthetic construct
  source 1..1911: mol_type = other DNA
atactcagtgccctgactatatactcagtgccctgactatatactcagtgccctgactatatactcagtgccctgactatatactcagtgccctgactatgctagcatacgcgtgatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagctacgaatcgcgctagtggccggaattcgagctcgcccggggatcctctagagtcagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaa

SEQ ID NO: 167 - DNA, 1801 bp, synthetic construct
  misc_feature 1..1801: note = synthetic construct
  source 1..1801: mol_type = other DNA
ggtacctgagctcgctagctacattggaaaattttatacacgttctagctacattggaaaattttatacacgttctagctacattggaaaattttatacacgttctagctacattggaaaattttatacacgttctagctacattggaaaattttatacacgttagactctagagggtatataatggaagctcgacttccagcttggcaatccggtactgttggtaaagaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatga

SEQ ID NO: 168 - DNA, 2034 bp, synthetic construct
  misc_feature 1..2034: note = synthetic construct
  source 1..2034: mol_type = other DNA
tcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcgggctagcgtttaaacttaagcttagcgccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatga

SEQ ID NO: 169 - DNA, 1950 bp, synthetic construct
  misc_feature 1..1950: note = synthetic construct
  source 1..1950: mol_type = other DNA
gtcgacatttcccgtaaatcgtcgagtcgacatttcccgtaaatcgtcgagggcccatcgatgtcgacatttcccgtaaatcgtcgagtcgacatttcccgtaaatcgtcgagggcccgtcgacatttcccgtaaatcgtcgagtcgacatttcccgtaaatcgtcgaatcgatgtcgacatttcccgtaaatcgtcgagtcgacatttcccgtaaatcgtcgaacgcgtcctgcaggtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggaattcgagctcgcccggggatcctctagagtcagcttctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaa

SEQ ID NO: 170 - DNA, 850 bp, synthetic construct
  misc_feature 1..850: note = synthetic construct
  source 1..850: mol_type = other DNA
cgcgtattggatccaatgcattggatccaatggattggatccaatcgattggatccaattgattggatccaatctagctcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaattcgccacctatgactagtgagacagacacactcctgctatgggtactgctgctctgggttccaggttccactggtgacgctagtggtggttctggtatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagc

SEQ ID NO: 171 - DNA, 1789 bp, synthetic construct
  misc_feature 1..1789: note = synthetic construct
  source 1..1789: mol_type = other DNA
attggatccaatgcattggatccaatggattggatccaatcgattggatccaattgattggatccaatctagctcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcgcccggggatcctctagagtcagcttctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaa

SEQ ID NO: 172 - DNA, 1854 bp, synthetic construct
  misc_feature 1..1854: note = synthetic construct
  source 1..1854: mol_type = other DNA
gtaccgagctcttacgcgtgctagcgcaccagacagtgacgtcagctgccagatcccatggccgtcatactgtgacgtctttcagacaccccattgacgtcaatgggagaacagatctgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagaagcttcgaatcgcgaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatga

SEQ ID NO: 173 - DNA, 897 bp, synthetic construct
  misc_feature 1..897: note = synthetic construct
  source 1..897: mol_type = other DNA
aggggaaataatctatttgaacagtttagatggtgaagaacatttgcagcatttgcaagatttttttccactctgaagtggtctttgtccttgaacataggatacaagtgacccctgctctgttaattattggcaaattgcctaacttcaacgtaaggaaatagagtcatatgtttgctcactgaaggttactagttaacaggcatcccttaaacaggatataaaaggacttcagcaggactgctcgaaacatcccacttccagcactgcctgcggtgaaggaagcttgccaccatgactagtgagacagacacactcctgctatgggtactgctgctctgggttccaggttccactggtgacgctagtggtggttctggtatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtga

SEQ ID NO: 174 - RNA, 1987 nt, synthetic construct
  misc_feature 1..1987: note = synthetic construct
  source 1..1987: mol_type = other RNA
atgactagtgagacagacacactcctgctatgggtactgctgctctgggttccaggttccactggtgacgctagtggtggttctggtatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcggagcaaccaacttttccctgctgaagcaggcaggcgacgtggaggagaatcctggacccatggccctgtggatgcgcttcctgcccctgctggccctgctcgtcctctgggagcccaagcctgcccaggcttttgtcaaacagcacctttgtggtcctcacctggtggaggctctgtacctggtgtgtggggaacgtggtttcttctacacacccaagtcccgtcgtaaaagggaggacccgcaagtgccacaactggagctgggtggaggcccggaggccggggatcttcagaccttggcactggaggttgcccggcagaagcgtggcattgtggatcagtgctgcaccagcatctgctccctctaccaactggagaactactgcaactgacggccgctcaatctcgagttatgaggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagtgggcccgtttaaacccgctgatcagcct

SEQ ID NO: 175 - RNA, 3579 nt, synthetic construct
  misc_feature 1..3579: note = synthetic construct
  source 1..3579: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgagcggccgctcaatctcgaggggatcccgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcctgatccaacttgaaccggtaagaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgatcccgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcctgatcggagacgagtccgtctcaaattgccgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatgaattgcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaactgaattgccgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatggccaccatgactagtgagacagacacactcctgctatgggtactgctgctctgggttccaggttccactggtgacgctagtggtggttctggtatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagcgtttaaacccgctgatcagcct

SEQ ID NO: 176 - RNA, 1921 nt, synthetic construct
  misc_feature 1..1921: note = synthetic construct
  source 1..1921: mol_type = other RNA
agcttcaacaactactagtttaacgtaacatcgacagaccagtggaagtaatactgcacgctagctaatagcaaccacacaaacaatcaaagtttaaacgacaacgaaacttaggcaacaaacaaatcaccaggggcgtgatccgaaaggtgaccctggtgaaagccttagcctaggggcgtgatccgaaaggtgaccctaggcacaagaaattctacggggcgtgatccgaaaggtgacccgtagaatcacaaagactacagggggcgtgatccgaaaggtgacccctgtagactcaaacttaaaaaggatatcttcagcaaacttgaaccggtaagaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacatt

SEQ ID NO: 177 - RNA, 3105 nt, synthetic construct
  misc_feature 1..3105: note = synthetic construct
  source 1..3105: mol_type = other RNA
atggactacaaagaccatgacggtgattataaagatcatgacatcgattacaaggatgacgatgacaaggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagtgggcccgtttaaacccgctgatcagcct

SEQ ID NO: 178 - RNA, 2339 nt, synthetic construct
  misc_feature 1..2339: note = synthetic construct
  source 1..2339: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccagaatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagaccatcatgttcgacgacgccattgagagggcgggccagctcaccagcgaggaggacacgctgagcctcgtcactgccgaccactcccacgtgttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttacagcagagtcggggcggccggccgcttcgagcagacatgagcggccgctcaatctcgaggggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtgaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagtgggcccgtttaaacccgctgatcagcct

SEQ ID NO: 179 - RNA, 2128 nt, synthetic construct
  misc_feature 1..2128: note = synthetic construct
  source 1..2128: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccagaatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagaccatcatgttcgacgacgccattgagagggcgggccagctcaccagcgaggaggacacgctgagcctcgtcactgccgaccactcccacgtgttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttacagcagagtcggggcggccggccgcttcgagcagacatgagcggccgctcaatctcgaggggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcgtttaaaccctcctcgtccggggctgtgtcgccccgctgacgagccctgaaaaagggcgaaacgaggaggactagtgggcccgtttaaacccgctgatcagcct

SEQ ID NO: 180 - RNA, 2164 nt, synthetic construct
  misc_feature 1..2164: note = synthetic construct
  source 1..2164: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgagcggccgctcaatctcgaggggatccaacttgaaccggtaagaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgatcggagacgagtccgtctcaaattgcaacaaacaaatcaccaccgtgaggatcacccacggtggtgaaagccttagcctagccgtgaggatcacccacggtaggcacaagaaattctacccgtgaggatcacccacgggtagaatcacaaagactacagccgtgaggatcacccacggctgtagactcaaacttaaaaaggatatcttcagcaaactgaattcacagatgcagatcacagttgccagctgagattatacagtaaattggagacgagtccgtctcactagacagatgcagatcacagttgccagctgagattatacagtatctagagggcccgtttaaacccgctgatcagcct

SEQ ID NO: 181 - protein, 266 aa, synthetic construct
  REGION 1..266: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSEVQLLESGGGLVQPGGSLRLSCAASGFTFSTFSMNWVRQAPGKGLEWVSYISKCGHVIYYADSVKGRFTISRDNSKNTLYLQMNSLRAEDTAVYYCARGPLWSFVRMSFDYWGQGTLVTVSSAS

SEQ ID NO: 182 - protein, 251 aa, synthetic construct
  REGION 1..251: note = synthetic construct
APSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSAAA

SEQ ID NO: 183 - protein, 248 aa, synthetic construct
  REGION 1..248: note = synthetic construct
APSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLS

SEQ ID NO: 184 - protein, 276 aa, synthetic construct
  REGION 1..276: note = synthetic construct
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQASTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMP

SEQ ID NO: 185 - protein, 571 aa, synthetic construct
  REGION 1..571: note = synthetic construct
LRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 186 - protein, 423 aa, synthetic construct
  REGION 1..423: note = synthetic construct
KSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSIPLDQYSPSFPDTRSSTCSSGEDSVFSHEPLPEEPCLPRHPTQLANSGLKRR

SEQ ID NO: 187 - protein, 746 aa, synthetic construct
  REGION 1..746: note = synthetic construct
METDTLLLWVLLLWVPGSTGDGSKLATMGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHMEFAPSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSAAANKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQASTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 188 - protein, 934 aa, synthetic construct
  REGION 1..934: note = synthetic construct
METDTLLLWVLLLWVPGSTGDGSKLATMGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHMEFAPSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSAAANKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQASTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 189 - protein, 890 aa, synthetic construct
  REGION 1..890: note = synthetic construct
METDTLLLWVLLLWVPGSTGDGSKLATMGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHMEFAPSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSIPLDQYSPSFPDTRSSTCSSGEDSVFSHEPLPEEPCLPRHPTQLANSGLKRR

SEQ ID NO: 190 - protein, 1033 aa, synthetic construct
  REGION 1..1033: note = synthetic construct
METDTLLLWVLLLWVPGSTGDKLATMGELDELVYLLDGPGYDPIHSDGSGTGSGTGSGTGTTSGTGTGGSTGGELDELVYLLDGPGYDPIHSDGSGTGSGTGSGTGTTSGTGTGGSTGHTGELDELVYLLDGPGYDPIHSDGSGTGSGTGSGTGTTSGTGTGGSTGGELDELVYLLDGPGYDPIHSDGSGTGSGTGSGTGTTSGTGTGGSTGHMAPSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSLRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 191 - protein, 786 aa, synthetic construct
  REGION 1..786: note = synthetic construct
METDTLLLWVLLLWVPGSTGDGSDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSEFAPSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSAAANKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQASTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 192 - protein, 749 aa, synthetic construct
  REGION 1..749: note = synthetic construct
METDTLLLWVLLLWVPGSTGDGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPEFAPSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSAAANKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQASTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 193 - protein, 930 aa, synthetic construct
  REGION 1..930: note = synthetic construct
METDTLLLWVLLLWVPGSTGDGSDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSEFAPSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSIPLDQYSPSFPDTRSSTCSSGEDSVFSHEPLPEEPCLPRHPTQLANSGLKRR

SEQ ID NO: 194 - protein, 893 aa, synthetic construct
  REGION 1..893: note = synthetic construct
METDTLLLWVLLLWVPGSTGDGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPEFAPSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSIPLDQYSPSFPDTRSSTCSSGEDSVFSHEPLPEEPCLPRHPTQLANSGLKRR

SEQ ID NO: 195 - protein, 1077 aa, synthetic construct
  REGION 1..1077: note = synthetic construct
METDTLLLWVLLLWVPGSTGDGSDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSEFAPSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSLRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 196 - protein, 1040 aa, synthetic construct
  REGION 1..1040: note = synthetic construct
METDTLLLWVLLLWVPGSTGDGSKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPEFAPSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSLRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 197 - protein, 555 aa, synthetic construct
  REGION 1..555: note = synthetic construct
PKKKRKVAAVDLATHPGHLARRLQQAHYLLWNTMVSEETTSPQYAVLNALVAEPGLDQRTVGERVGLDRSTIAEVVSRLGRRGLLDKVRDPQDGRRSLLRLTDEGLRVHRRLGVRIARMNQVFLAPLAADEQAVFFDLIRRVADAAEGLRNPAEPAVAPGASGSGGGGDVDEMEECSQHGGSGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHASGSGGGAPDPKKKRKVDPKKKRKVDPKKKRKVMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVSSRSIFKDKQSCDIKMEGMARNDLW

SEQ ID NO: 198 - protein, 456 aa, synthetic construct
  REGION 1..456: note = synthetic construct
PKKKRKVAAVDLATHPGHLARRLQQAHYLLWNTMVSEETTSPQYAVLNALVAEPGLDQRTVGERVGLDRSTIAEVVSRLGRRGLLDKVRDPQDGRRSLLRLTDEGLRVHRRLGVRIARMNQVFLAPLAADEQAVFFDLIRRVADAAEGLRNPAEPAVAPGASGSGGGGDVDEMEECSQHGGSGGSTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHGGSGGSDALDDFDLDMLGSDALDDFDLDMLGSDALDDFDLDMLGSDALDDFDLDML

SEQ ID NO: 199 - protein, 338 aa, synthetic construct
  REGION 1..338: note = synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPAVRSQPQKGRKPRDLELPLSPSLLGGPGPERTPGSGSGSGLQAPGPALTPSLLPTHTLTPVLLTPSSLPPSIHFWSTLSPIAPRSPAKLSFQFPSSGSAQVHIPSISVDGLSTPVVLSQGPRSHEPVPGN

SEQ ID NO: 200 - protein, 116 aa, synthetic construct
  REGION 1..116: note = synthetic construct
ASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 201 - protein, 424 aa, synthetic construct
  REGION 1..424: note = synthetic construct
MVDPVGFAEAWKAQFPDSEPPRMELRSVGDIEQELERAEARIRRNEQRVNQERFRMIYLETLLAKEKKSYDRASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYEYPYDVPDYAYPYDVPDYAYPYDVPDYA

SEQ ID NO: 202 - protein, 288 aa, synthetic construct
  REGION 1..288: note = synthetic construct
MDYKDHDGDYKDHDIDYKDDDDKASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSEVQLLESGGGLVQPGGSLRLSCAASGFTFSTFSMNWVRQAPGKGLEWVSYISKCGHVIYYADSVKGRFTISRDNSKNTLYLQMNSLRAEDTAVYYCARGPLWSFVRMSFDYWGQGTLVTVSSAS

SEQ ID NO: 203 - protein, 312 aa, synthetic construct
  REGION 1..312: note = synthetic construct
YVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVGSGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 204 - protein, 297 aa, synthetic construct
  REGION 1..297: note = synthetic construct
MDAQTRRRERRAEKQAQWKAANPLLVGVSAKPVNRPGGGSGGGGSKLVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGILQISSTVAAARV

SEQ ID NO: 205 - protein, 170 aa, synthetic construct
  REGION 1..170: note = synthetic construct
MDAQTRRRERRAEKQAQWKAANASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNV

SEQ ID NO: 206 - protein, 775 aa, synthetic construct
  REGION 1..775: note = synthetic construct
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTVASGSGGGGDVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 207 - protein, 718 aa, synthetic construct
  REGION 1..718: note = synthetic construct
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTVASGSGGGGDVGSMSTKLYGDVNDDGKVNSTDAVALKRYVLRSGISINTDNADLNEDGRVNSTDLGILKRYILKEIDTLPYKN

SEQ ID NO: 208 - protein, 344 aa, synthetic construct
  REGION 1..344: note = synthetic construct
MNGTEGPNFYVPFSNATGVVREFTRMSYSNHSSTSFFLTGLPGLETVYLWLSIPLCTMYIASLAGNGLILWVVKSEPSLHQPMYYFLSMLAVTDLGLSVSTLPTMLTIYMMGVSEVALDMCLAQLFFIHTFSIMESSVLLTMAFDRVVAISSPLHYATILTNPRVASLGMVILVRSIGLHIPAPIMLKKLPYCQKRHLSHSYCLHPDVMKLACTDTRINSAYGLFVVLSTLGVDSVLIVLSYGLILYTVLSIASKTERLKALNTCVSHICSVLLFYTPMIGLSMIHRFGKWASPCSRVLLSYLHFLTPPVLNPVVYTIKTKQIRQRIWCIFRCGGRSIGHIQGH

SEQ ID NO: 209 - DNA, 50 bp, synthetic construct
  misc_feature 1..50: note = synthetic construct
  source 1..50: mol_type = other DNA
gtcgacatttcccgtaaatcgtcgagtcgacatttcccgtaaatcgtcga

SEQ ID NO: 210 - DNA, 241 bp, synthetic construct
  misc_feature 1..241: note = synthetic construct
  source 1..241: mol_type = other DNA
ggtacctgagctcgctagctacattggaaaattttatacacgttctagctacattggaaaattttatacacgttctagctacattggaaaattttatacacgttctagctacattggaaaattttatacacgttctagctacattggaaaattttatacacgttagactctagagggtatataatggaagctcgacttccagcttggcaatccggtactgttggtaaagaattcgcccacc

SEQ ID NO: 211 - protein, 132 aa, synthetic construct
  REGION 1..132: note = synthetic construct
GPAVRSQPQKGRKPRDLELPLSPSLLGGPGPERTPGSGSGSGLQAPGPALTPSLLPTHTLTPVLLTPSSLPPSIHFWSTLSPIAPRSPAKLSFQFPSSGSAQVHIPSISVDGLSTPVVLSQGPRSHEPVPGN

SEQ ID NO: 212 - DNA, 130 bp, synthetic construct
  misc_feature 1..130: note = synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 213 - skipped

SEQ ID NO: 214 - DNA, 147 bp, synthetic construct
  misc_feature 1..147: note = synthetic construct
  source 1..147: mol_type = other DNA
gccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagc

SEQ ID NO: 215 - DNA, 634 bp, synthetic construct
  misc_feature 1..634: note = synthetic construct
  source 1..634: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 216 - RNA, 574 nt, synthetic construct
  misc_feature 1..574: note = synthetic construct
  source 1..574: mol_type = other RNA
cccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 217 - DNA, 19 bp, synthetic construct
  misc_feature 1..19: note = synthetic construct
  source 1..19: mol_type = other DNA
tctctatcactgataggga

SEQ ID NO: 218 - DNA, 18 bp, synthetic construct
  misc_feature 1..18: note = synthetic construct
  source 1..18: mol_type = other DNA
tacattggaaaattttat

SEQ ID NO: 219 - protein, 243 aa, synthetic construct
  REGION 1..243: note = synthetic construct
DMPRIKPGQRVMMALRKMIASGEIKSGERIAEIPTAAALGVSRMPVRTALRSLEQEGLVVRLGARGYAARGVSSDQIRDAIEVRGVLEGFAARRLAERGMTAETHARFVALIAEGEALFAAGRLNGEDLDRYAAYNQAFHDTLVSAAGNGAVESALARNGFEPFAAAGALALDLMDLPAEYEHLLAAHRQHQAVLDAVSCGDAEGAERIMRDHALAAIRNAKVFEAAASAGAPLGAAWSIRAD

SEQ ID NO: 220 - protein, 265 aa, synthetic construct
  REGION 1..265: note = synthetic construct
AQVQLVESGGGLVQAGGSLRLSCAVSGRPFSEYNLGWFRQAPGKEREFVARIRSSGTTVYTDSVKGRFSASRDNAGLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAGKNMGYLQLNSLEPEDTAVYYCAMSRVDTDSPAFYDYWGQGTQVTVSTPRSAS

SEQ ID NO: 221 - protein, 22 aa, synthetic construct
  REGION 1..22: note = synthetic construct
DYKDHDGDYKDHDIDYKDDDDK

SEQ ID NO: 222 - protein, 71 aa, synthetic construct
  REGION 1..71: note = synthetic construct
VDPVGFAEAWKAQFPDSEPPRMELRSVGDIEQELERAEARIRRNEQRVNQERFRMIYLETLLAKEKKSYDR

SEQ ID NO: 223 - protein, 122 aa, synthetic construct
  REGION 1..122: note = synthetic construct
EVQLLESGGGLVQPGGSLRLSCAASGFTFSTFSMNWVRQAPGKGLEWVSYISKCGHVIYYADSVKGRFTISRDNSKNTLYLQMNSLRAEDTAVYYCARGPLWSFVRMSFDYWGQGTLVTVSS

SEQ ID NO: 224 - protein, 27 aa, synthetic construct
  REGION 1..27: note = synthetic construct
YPYDVPDYAYPYDVPDYAYPYDVPDYA

SEQ ID NO: 225 - DNA, 20 bp, synthetic construct
  misc_feature 1..20: note = synthetic construct
  source 1..20: mol_type = other DNA
atactcagtgccctgactat

SEQ ID NO: 226 - protein, 1130 aa, synthetic construct
  REGION 1..1130: note = synthetic construct
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 227 - protein, 1271 aa, synthetic construct
  REGION 1..1271: note = synthetic construct
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSNLYCTLEVDSFGYFVNKAKTRVYRDTAEPNWNEEFEIELEGSQTLRILCYEKCYNKTKIPKEDGESTDRLMGKGQVQLDPQALQDRDWQRTVIAMNGIEVKLSVKFNSREFSLKRMPSRKQTGVFGVKIAVVTKRERSKVPYIVRQCVEEIERRGMEEVGIYRVSGVATDIQALKAAFDVNNKDVSVMMSEMDVNAIAGTLKLYFRELPEPLFTDEFYPNFAEGIALSDPVAKESCMLNLLLSLPEANLLTFLFLLDHLKRVAEKEAVNKMSLHNLATVFGPTLLRPSEKESKLPANPSQPITMTDSWSLEVMSQVQVLLYFLQLEAIPAPDSKRQSILFSTEV

SEQ ID NO: 228 - protein, 124 aa, synthetic construct
  REGION 1..124: note = synthetic construct
MAKLTSAVPVLTARDVAGAVEFWTDRLGFSRDFVEDDFAGVVRDDVTLFISAVQDQVVPDNTLAWVLVRGLDELYAEWSEVVSTNFRDASGPAMTEIGEQPWGREFALRDPAGNCVHFVAEEQD

SEQ ID NO: 229 - protein, 199 aa, synthetic construct
  REGION 1..199: note = synthetic construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVECPKDRATWCMTRKPGA

SEQ ID NO: 230 - DNA, 12 bp, synthetic construct
  misc_feature 1..12: note = synthetic construct
  source 1..12: mol_type = other DNA
attggatccaat

SEQ ID NO: 231 - DNA, 32 bp, synthetic construct
  misc_feature 1..32: note = synthetic construct
  source 1..32: mol_type = other DNA
tagagggtatataatggaagctcgacttccag

SEQ ID NO: 232 - DNA, 20 bp, synthetic construct
  misc_feature 1..20: note = synthetic construct
  source 1..20: mol_type = other DNA
taatacgactcactataggg

SEQ ID NO: 233 - DNA, 330 bp, synthetic construct
  misc_feature 1..330: note = synthetic construct
  source 1..330: mol_type = other DNA
gtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaa

SEQ ID NO: 234 - DNA, 612 bp, synthetic construct
  misc_feature 1..612: note = synthetic construct
  source 1..612: mol_type = other DNA
cgagaccctgtctcacaaaataaagtaagcccggactgagtgcggaaaggcgggcctggcgggtctggtctccccatgcgggccaccagaggccctgcagccttcagtcgcttgaaggggtaatggcgcttccactcacaaacatggcggacagagcgtgtgaacgagatgaacagcccctcaaaaatatggccgccgaggctggacggccgtgccccagcagcaccgcctccgcgccccacgtgatctctcgccgggcacagcgctgaccgcggaggtccaaccggaagaatgtccggattggacattcggaagagggcccgccttccctggggaatctctgcgcacgcgcagaacgcttcgaccaatgaaaacacaggaagccgtccgcgcaaccgcgttgcgtcacttctgccgcccctgtttcaaggtatatagccgtagacggaacttcgcctttctctcggccttagcgccatttttttgggtgagtgttttttggttcctgcgttgggattccgtgtacaatccatagacatctgacctcggcacttagcatcatcacagcaaactaactgtagcctttctctctttccctgtagaaacctctgc

SEQ ID NO: 235 - DNA, 501 bp, synthetic construct
  misc_feature 1..501: note = synthetic construct
  source 1..501: mol_type = other DNA
ttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 236 - DNA, 1059 bp, synthetic construct
  misc_feature 1..1059: note = synthetic construct
  source 1..1059: mol_type = other DNA
gggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcttggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccatactgagtgggtggagactgaagttaggccagcttggcacttgatgtaattctccttggaatttgccctttttgagtttggatcttggttcattctcaagcctcagacagtggttcaaagtttttttcttccattt

SEQ ID NO: 237 - DNA, 144 bp, synthetic construct
  misc_feature 1..144: note = synthetic construct
  source 1..144: mol_type = other DNA
ctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcg

SEQ ID NO: 238 - DNA, 437 bp, synthetic construct
  misc_feature 1..437: note = synthetic construct
  source 1..437: mol_type = other DNA
tttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcg

SEQ ID NO: 239 - DNA, 240 bp, synthetic construct
  misc_feature 1..240: note = synthetic construct
  source 1..240: mol_type = other DNA
gcaccagacagtgacgtcagctgccagatcccatggccgtcatactgtgacgtctttcagacaccccattgacgtcaatgggagaacagatctgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagc

SEQ ID NO: 240 - DNA, 584 bp, synthetic construct
  misc_feature 1..584: note = synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 241 - protein, 408 aa, synthetic construct
  REGION 1..408: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSMAQVQLVESGGGLVQAGGSLRLSCAVSGRPFSEYNLGWFRQAPGKEREFVARIRSSGTTVYTDSVKGRFSASRDNAGLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAGKNMGYLQLNSLEPEDTAVYYCAMSRVDTDSPAFYDYWGQGTQVTVSTPRSAS

SEQ ID NO: 242 - protein, 486 aa, synthetic construct
  REGION 1..486: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAEC

SEQ ID NO: 243 - protein, 236 aa, synthetic construct
  REGION 1..236: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVASRILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 244 - protein, 358 aa, synthetic construct
  REGION 1..358: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSATVEPETTPTPNPPTTEEEKTESNQEVANPEHYIKHPLQNRWALWFFKNDKSKTWQANLRLISKFDTVEDFWALYNHIQLSSNLMPGCDYSLFKDGIEPMWEDEKNKRGGRWLITLNKQQRRSDLDRFWLETLLCLIGESFDDYSDDVCGAVVNVRAKGDKIAIWTTECENREAVTHIGRVYKERLGLPPKIVIGYQSHADTATKSGSTTKNRFVV

SEQ ID NO: 245 - protein, 379 aa, synthetic construct
  REGION 1..379: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 246 - protein, 214 aa, synthetic construct
  REGION 1..214: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSMSTKLYGDVNDDGKVNSTDAVALKRYVLRSGISINTDNADLNEDGRVNSTDLGILKRYILKEIDTLPYKNAS

SEQ ID NO: 247 - protein, 309 aa, synthetic construct
  REGION 1..309: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVNGAIGGDLLLNFPDMSVLERQRAHLKYLNPTFDSPLAGFFADSSMITGGEMDSYLSTAGLNLPMMYGETTVEGDSRLSISPETTLGTGNFKKRKFDTETKDCNEKKKKMTMNRDDLVEEGEEEKSKITEQNNGSTKSIKKMKHKAKKEENNFSNDSSKVTKELEKTDYI

SEQ ID NO: 248 - protein, 21 aa, synthetic construct
  REGION 1..21: note = synthetic construct
METDTLLLWVLLLWVPGSTGD

SEQ ID NO: 249 - protein, 402 aa, synthetic construct
  REGION 1..402: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSDLGKKLLEAARAGQDDEVRILMANGADVNALDEDGLTPLHLAAQLGHLEIVEVLLKYGADVNAEDNFGITPLHLAAIRGHLEIVEVLLKHGADVNAQDKFGKTAFDISIDNGNEDLAEILQKLASASGSGGGGDVDLGKKLLEAARAGQDDEVRILMANGADVNALDEDGLTPLHLAAQLGHLEIVEVLLKYGADVNAEDNFGITPLHLAAIRGHLEIVEVLLKHGADVNAQDKFGKTAFDISIDNGNEDLAEILQKLAS

SEQ ID NO: 250 - protein, 561 aa, synthetic construct
  REGION 1..561: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 251 - protein, 370 aa, synthetic construct
  REGION 1..370: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKEFASGSGGGGDVMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 252 - protein, 433 aa, synthetic construct
  REGION 1..433: note = synthetic construct
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 253 - protein, 1059 aa, synthetic construct
  REGION 1..1059: note = synthetic construct
MGPPRGGPGGELPRGPQAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHNASGSGGGGDVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 254 - protein, 192 aa, synthetic construct
  REGION 1..192: note = synthetic construct
MSTKLYGDVNDDGKVNSTDAVALKRYVLRSGISINTDNADLNEDGRVNSTDLGILKRYILKEIDTLPYKNGSGGGAKGKTKSKVGPYRGRGVALTDDEYDEWREHNATRKLDLSVEDFLMLRHRAALGADDADAVKFRSWWNSRSRLADDYEDVTVIGKGGVKHEKIRTNTLRAVDRGYDVSFAEETGVPTV

SEQ ID NO: 255 - protein, 319 aa, synthetic construct
  REGION 1..319: note = synthetic construct
MSTKLYGDVNDDGKVNSTDAVALKRYVLRSGISINTDNADLNEDGRVNSTDLGILKRYILKEIDTLPYKNASGSGGGGDVGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKAS

SEQ ID NO: 256 - protein, 999 aa, synthetic construct
  REGION 1..999: note = synthetic construct
MSTKLYGDVNDDGKVNSTDAVALKRYVLRSGISINTDNADLNEDGRVNSTDLGILKRYILKEIDTLPYKNASGSGGGGDVGPPRGGPGGELPRGPQAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 257 - protein, 291 aa, synthetic construct
  REGION 1..291: note = synthetic construct
MSTKLYGDVNDDGKVNSTDAVALKRYVLRSGISINTDNADLNEDGRVNSTDLGILKRYILKEIDTLPYKNGSGGGATVEPETTPTPNPPTTEEEKTESNQEVANPEHYIKHPLQNRWALWFFKNDKSKTWQANLRLISKFDTVEDFWALYNHIQLSSNLMPGCDYSLFKDGIEPMWEDEKNKRGGRWLITLNKQQRRSDLDRFWLETLLCLIGESFDDYSDDVCGAVVNVRAKGDKIAIWTTECENREAVTHIGRVYKERLGLPPKIVIGYQSHADTATKSGSTTKNRFVV

SEQ ID NO: 258 - protein, 397 aa, synthetic construct
  REGION 1..397: note = synthetic construct
MVDPVGFAEAWKAQFPDSEPPRMELRSVGDIEQELERAEARIRRNEQRVNQERFRMIYLETLLAKEKKSYDRASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 259 - protein, 442 aa, synthetic construct
  REGION 1..442: note = synthetic construct
MTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKASGSGGGGDVASASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 260 - protein, 475 aa, synthetic construct
  REGION 1..475: note = synthetic construct
MDYKDHDGDYKDHDIDYKDDDDKASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 261 - protein, 607 aa, synthetic construct
  REGION 1..607: note = synthetic construct
MAEVQVQQSGAELVRSGASVKLSCTASGFNIKDYYMHWVKQRPEQGLEWIGWIDPANGNTKYDPKFQGKATITADTSSNTAYLQLSSLTSEDTAVYYCARWDWYFDVWGAGTTVTVSSGGGGSGGGGSGGGGSVFASGSGGGGDVASMAEVQVQQSGAELVRSGASVKLSCTASGFNIKDYYMHWVKQRPEQGLEWIGWIDPANGNTKYDPKFQGKATITADTSSNTAYLQLSSLTSEDTAVYYCARWDWYFDVWGAGTTVTVSSGGGGSGGGGSGGGGSVFASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 262 - protein, 754 aa, synthetic construct
  REGION 1..754: note = synthetic construct
MAEVQVQQSGAELVRSGASVKLSCTASGFNIKDYYMHWVKQRPEQGLEWIGWIDPANGNTKYDPKFQGKATITADTSSNTAYLQLSSLTSEDTAVYYCARWDWYFDVWGAGTTVTVSSGGGGSGGGGSGGGGSVFASGSGGGGDVASMAEVQVQQSGAELVRSGASVKLSCTASGFNIKDYYMHWVKQRPEQGLEWIGWIDPANGNTKYDPKFQGKATITADTSSNTAYLQLSSLTSEDTAVYYCARWDWYFDVWGAGTTVTVSSGGGGSGGGGSGGGGSVFASGSGGGGDVASMAEVQVQQSGAELVRSGASVKLSCTASGFNIKDYYMHWVKQRPEQGLEWIGWIDPANGNTKYDPKFQGKATITADTSSNTAYLQLSSLTSEDTAVYYCARWDWYFDVWGAGTTVTVSSGGGGSGGGGSGGGGSVFASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 263 - protein, 360 aa, synthetic construct
  REGION 1..360: note = synthetic construct
MDYKDHDGDYKDHDIDYKDDDDKASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPAS

SEQ ID NO: 264 - protein, 152 aa, synthetic construct
  REGION 1..152: note = synthetic construct
MDYKDHDGDYKDHDIDYKDDDDKASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 265 - protein, 431 aa, synthetic construct
  REGION 1..431: note = synthetic construct
TRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKASGSGGGGDVASMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 266 - protein, 625 aa, synthetic construct
  REGION 1..625: note = synthetic construct
TRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 267 - protein, 408 aa, synthetic construct
  REGION 1..408: note = synthetic construct
MGELDELVYLLDGPGYDPIHSDGSGTGSGTGSGTGTTSGTGTGGSTGGELDELVYLLDGPGYDPIHSDGSGTGSGTGSGTGTTSGTGTGGSTGHMMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 268 - protein, 464 aa, synthetic construct
  REGION 1..464: note = synthetic construct
MVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 269 - protein, 614 aa, synthetic construct
  REGION 1..614: note = synthetic construct
MVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASGSGGGGDVASVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 270 - protein, 555 aa, synthetic construct
  REGION 1..555: note = synthetic construct
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 271 - protein, 797 aa, synthetic construct
  REGION 1..797: note = synthetic construct
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHASGSGGGGDVASMSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 272 - protein, 432 aa, synthetic construct
  REGION 1..432: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVAS

SEQ ID NO: 273 - protein, 582 aa, synthetic construct
  REGION 1..582: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVGSVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVAS

SEQ ID NO: 274 - protein, 586 aa, synthetic construct
  REGION 1..586: note = synthetic construct
MYPYDVPDYAYPYDVPDYAYPYDVPDYASTKLYGDVNDDGKVNSTDAVALKRYVLRSGISINTDNADLNEDGRVNSTDLGILKRYILKEIDTLPYKNASGSGGGGDVASMSTKLYGDVNDDGKVNSTDAVALKRYVLRSGISINTDNADLNEDGRVNSTDLGILKRYILKEIDTLPYKNASGSGGGGDVASMSTKLYGDVNDDGKVNSTDAVALKRYVLRSGISINTDNADLNEDGRVNSTDLGILKRYILKEIDTLPYKNASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 275 - protein, 243 aa, synthetic construct
  REGION 1..243: note = synthetic construct
ASNFTQFVLVDNGGTGDVTVAPSNFANGVAEWISSNSRSQAYKVTCSVRQSSAQNRKYTIKVEVPKGAWRSYLNMELTIPIFATNSDCELIVKAMQGLLKDGNPIPSAIAANSGIYASGSGGGGDVAKGKTKSKVGPYRGRGVALTDDEYDEWREHNATRKLDLSVEDFLMLRHRAALGADDADAVKFRSWWNSRSRLADDYEDVTVIGKGGVKHEKIRTNTLRAVDRGYDVSFAEETGVPTV

SEQ ID NO: 276 - protein, 235 aa, synthetic construct
  REGION 1..235: note = synthetic construct
ASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSAKGKTKSKVGPYRGRGVALTDDEYDEWREHNATRKLDLSVEDFLMLRHRAALGADDADAVKFRSWWNSRSRLADDYEDVTVIGKGGVKHEKIRTNTLRAVDRGYDVSFAEETGVPTV

SEQ ID NO: 277 - protein, 685 aa, synthetic construct
  REGION 1..685: note = synthetic construct
MAAAAAAGAGPEMVRGQVFDVGPRYTNLSYIGEGAYGMVCSAYDNVNKVRVAIKKISPFEHQTYCQRTLREIKILLRFRHENIIGINDIIRAPTIEQMKDVYIVQDLMETDLYKLLKTQHLSNDHICYFLYQILRGLKYIHSANVLHRDLKPSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPGYRSASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 278 - protein, 507 aa, synthetic construct
  REGION 1..507: note = synthetic construct
GGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 279 - protein, 313 aa, synthetic construct
  REGION 1..313: note = synthetic construct
GGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNASGSGGGGDVASMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 280 - protein, 254 aa, synthetic construct
  REGION 1..254: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVDLGKKLLEAARAGQDDEVRILMANGADVNALDEDGLTPLHLAAQLGHLEIVEVLLKYGADVNAEDNFGITPLHLAAIRGHLEIVEVLLKHGADVNAQDKFGKTAFDISIDNGNEDLAEILQKLAS

SEQ ID NO: 281 - protein, 245 aa, synthetic construct
  REGION 1..245: note = synthetic construct
YVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVAKGKTKSKVGPYRGRGVALTDDEYDEWREHNATRKLDLSVEDFLMLRHRAALGADDADAVKFRSWWNSRSRLADDYEDVTVIGKGGVKHEKIRTNTLRAVDRGYDVSFAEETGVPTV

SEQ ID NO: 282 - protein, 474 aa, synthetic construct
  REGION 1..474: note = synthetic construct
YVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVGSAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAEC

SEQ ID NO: 283 - protein, 219 aa, synthetic construct
  REGION 1..219: note = synthetic construct
YVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGGGGDVGSKRDHHHHHHQDKKTMMMNEEDDGNGMDELLAVLGYKVRSSEMADVAQKLEQLEVMMSNVQEDDLSQLATETVHYNPAELYTWLDSMLTDLN

SEQ ID NO: 284 - protein, 430 aa, synthetic construct
  REGION 1..430: note = synthetic construct
YVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVGSTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 285 - protein, 397 aa, synthetic construct
  REGION 1..397: note = synthetic construct
KRDHHHHHHQDKKTMMMNEEDDGNGMDELLAVLGYKVRSSEMADVAQKLEQLEVMMSNVQEDDLSQLATETVHYNPAELYTWLDSMLTDLNASGSGGGGDVASQDKKTMMMNEEDDGNGMDELLAVLGYKVRSSEMADVAQKLEQLEVMMSNVQEDDLSQLATETVHYNPAELYTWLDSMLTDLNGGGGSGQDKKTMMMNEEDDGNGMDELLAVLGYKVRSSEMADVAQKLEQLEVMMSNVQEDDLSQLATETVHYNPAELYTWLDSMLTDLNGGGGSMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 286 - protein, 475 aa, synthetic construct
  REGION 1..475: note = synthetic construct
AASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECASGSGGGGDVASMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 287 - protein, 669 aa, synthetic construct
  REGION 1..669: note = synthetic construct
AASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 288 - protein, 439 aa, synthetic construct
  REGION 1..439: note = synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASGSGGGGDVVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVASDYKDHDGDYKDHDIDYKDDDDK

SEQ ID NO: 289 - protein, 416 aa, synthetic construct
  REGION 1..416: note = synthetic construct
KRDHHHHHHQDKKTMMMNEEDDGNGMDELLAVLGYKVRSSEMADVAQKLEQLEVMMSNVQEDDLSQLATETVHYNPAELYTWLDSMLTDLNASGSGGGGDVASMLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYE

SEQ ID NO: 290 - protein, 546 aa, synthetic construct
  REGION 1..546: note = synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPASASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPAS

SEQ ID NO: 291 - protein, 507 aa, synthetic construct
  REGION 1..507: note = synthetic construct
MLKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYEASGSGGGGDVGSGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 292 - protein, 1368 aa, synthetic construct
  REGION 1..1368: note = synthetic construct
LKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYEASGSGGGGDVGSAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECGGGGSGSAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECGGGGSAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAEC

SEQ ID NO: 293 - protein, 415 aa, synthetic construct
  REGION 1..415: note = synthetic construct
LKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYEASGSGGGGDVGSKRDHHHHHHQDKKTMMMNEEDDGNGMDELLAVLGYKVRSSEMADVAQKLEQLEVMMSNVQEDDLSQLATETVHYNPAELYTWLDSMLTDLN

SEQ ID NO: 294 - protein, 624 aa, synthetic construct
  REGION 1..624: note = synthetic construct
LKMESTQQMASSIINTSFEAAVVAATSTLELMGIQYDYNEVYTRVKSKFDYVMDDSGVKNNLLGKAATIDQALNGKFGSAARNRNWMADTRTTARLDEDVNKLRMMLSSKGIDQKMRVLNACFNVKRVPGKSSSIIKCTRLMRDKIERGEVEVDDSFVEEKMEVDTIDWKSRYEQLEKRFESLKQRVNEKYTSWVQKAKKVNENMYSLQNVISQQQSQIADLQNYCNKLEVDLQNKISSLVSSVEWYLKSMELPDEIKTDIEQQLNSIDVINPINAIDDFESLIRNIILDYDRIFLMFKGLMRQCNYEYTYEASGSGGGGDVGSTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 295 - protein, 572 aa, synthetic construct
  REGION 1..572: note = synthetic construct
MDYKDHDGDYKDHDIDYKDDDDKEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 296 - protein, 304 aa, synthetic construct
  REGION 1..304: note = synthetic construct
MDMPRIKPGQRVMMALRKMIASGEIKSGERIAEIPTAAALGVSRMPVRTALRSLEQEGLVVRLGARGYAARGVSSDQIRDAIEVRGVLEGFAARRLAERGMTAETHARFVALIAEGEALFAAGRLNGEDLDRYAAYNQAFHDTLVSAAGNGAVESALARNGFEPFAAAGALALDLMDLPAEYEHLLAAHRQHQAVLDAVSCGDAEGAERIMRDHALAAIRNAKVFEAAASAGAPLGAAWSIRADASGSGGGGDVDALDDFDLDMLGSDALDDFDLDMLGSDALDDFDLDMLGSDALDDFDLDML

SEQ ID NO: 297 - protein, 266 aa, synthetic construct
  REGION 1..266: note = synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGASGSGGGGDVDALDDFDLDMLGSDALDDFDLDMLGSDALDDFDLDMLGSDALDDFDLDML

SEQ ID NO: 298 - protein, 214 aa, synthetic construct
  REGION 1..214: note = synthetic construct
MAAVDLATHPGHLARRLQQAHYLLWNTMVSEETTSPQYAVLNALVAEPGLDQRTVGERVGLDRSTIAEVVSRLGRRGLLDKVRDPQDGRRSLLRLTDEGLRVHRRLGVRIARMNQVFLAPLAADEQAVFFDLIRRVADAAEGLRNPAEPAVAPGASGSGGGGDVDALDDFDLDMLGSDALDDFDLDMLGSDALDDFDLDMLGSDALDDFDLDML

SEQ ID NO: 299 - protein, 414 aa, synthetic construct
  REGION 1..414: note = synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGASGSGGGGDVKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPAS

SEQ ID NO: 300 - protein, 804 aa, synthetic construct
  REGION 1..804: note = synthetic construct
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSASGSGGGGDVASDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSASGSGGGGDVASDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSASGSGGGGDVASASPKKKRKVEASGRADALDDFDLDMLGSDALDDFDLDMLGSDALDDFDLDMLGSDALDDFDLDMLYID

SEQ ID NO: 301 - protein, 2031 aa, synthetic construct
  REGION 1..2031: note = synthetic construct
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSKALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 302 - protein, 234 aa, synthetic construct
  REGION 1..234: note = synthetic construct
MSTKLYGDVNDDGKVNSTDAVALKRYVLRSGISINTDNADLNEDGRVNSTDLGILKRYILKEIDTLPYKNASGSGGGGDVASMSTKLYGDVNDDGKVNSTDAVALKRYVLRSGISINTDNADLNEDGRVNSTDLGILKRYILKEIDTLPYKNASGSGGGGDVASMSTKLYGDVNDDGKVNSTDAVALKRYVLRSGISINTDNADLNEDGRVNSTDLGILKRYILKEIDTLPYKN

SEQ ID NO: 303 - protein, 201 aa, synthetic construct
  REGION 1..201: note = synthetic construct
MAPIGSVVIVGRIILSGRGGPITAYSQQTRGLLGCIITSLTGRDKNQVEGEVQVVSTATQSFLATCVNGACWTVFHGAGSKTLAGPKGPITQMYTNVDQDLVGWPAPPGARSLTPCTCGSSDLYLVTRHADVIPVRRRGDTRGSLLSPRPISYLKGSSGGPLLCPSGHVVGIFRAAVCTRGVAKAVDFIPVESMETTMRGS

SEQ ID NO: 304 - protein, 221 aa, synthetic construct
  REGION 1..221: note = synthetic construct
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWGLPLAESLKRLMSLSPGRPPLLLWDAHVADRDHLCGGSAHRLTHHLEEDGLRPPAALDCVFPP

SEQ ID NO: 305 - protein, 550 aa, synthetic construct
  REGION 1..550: note = synthetic construct
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 306 - protein, 232 aa, synthetic construct
  REGION 1..232: note = synthetic construct
SELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 307 - protein, 131 aa, synthetic construct
  REGION 1..131: note = synthetic construct
AMPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 308 - RNA, 110 nt, synthetic construct
  misc_feature 1..110: note = synthetic construct
  source 1..110: mol_type = other RNA
gattcgtcagtagggttgtaaaggtttttcttttcctgagaaaacaaccttttgttttctcaggttttgctttttggcctttccctagctttaaaaaaaaaaaagcaaaa

SEQ ID NO: 309 - RNA, 93 nt, synthetic construct
  misc_feature 1..93: note = synthetic construct
  source 1..93: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 310 - RNA, 278 nt, synthetic construct
  misc_feature 1..278: note = synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 311 - RNA, 132 nt, synthetic construct
  misc_feature 1..132: note = synthetic construct
  source 1..132: mol_type = other RNA
tctcaacacaacatatacaaaacaaacgaatctcaagcaatcaagcattctacttctattgcagcaatttaaatcatttcttttaaagcaaaagcaattttctgaaaattttcaccatttacgaacgatagc

SEQ ID NO: 312 - protein, 180 aa, synthetic construct
  REGION 1..180: note = synthetic construct
HDQLTEEQIAEFKEAFSLFDKDGDGTITTKELGTVMRSLGQNPTEAELQDMINEVDADGNGTIYFPELLTMLARKMKDTDSEEEIREAFRVFDKDGNGYISAAELRHVMTNLGEKLTDEEVDEMIREADIDGDGQVNYEEFVQMMTAKGGKRRWQKTGHAVRIIGRLKKISSSGALELEF

SEQ ID NO: 313 - DNA, 2628 bp, synthetic construct
  misc_feature 1..2628: note = synthetic construct
  source 1..2628: mol_type = other DNA
attggatccaatgcattggatccaatggattggatccaatcgattggatccaattgattggatccaatctagctcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcgggccggccaccatggagaccgataccttattactgtgggtgcttcttctgtgggttcccggtagcaccggagacggatccgacatcgagaagctgtgcaagaaggcggaagaggaggccaaggaggcccaggagaaggccgacgagctgagacaacgacaccccgattcgcaggcggcggaagacgcggaggatctcgcaaatctggctgtcgccgccgtgctgaccgcttgtctgctggcccaggagcatcccaacgccgatatcgctaagctgtgcatcaaggccgcgagcgaagctgcggaggccgcgtcaaaagccgccgagctggcccaacggcacccagacagtcaagcagccagagatgctatcaaactcgcctctcaagccgcccgcgccgtgatcctggccatcatgctcgccgcagagaatcccaacgctgatattgcaaagctatgtattaaggcggccagcgaagccgcagaggctgcctcgaaagctgcggaacttgcacagcggcaccccgactcccaggccgcccgagatgcaattaaactggccagtcaggccgctgaggcggtggagagagccatctggctggccgccgagaaccccaatgccgatatcgccaaaaaatgcatcaaagcggcctccgaagcagccgaggaggcctctaaagctgccgaagaggctcaaagacacccagacagccagaaggcacgcgacgagatcaaggaggcctctcagaaggccgaggaggtgaaggagagatgcaagagcgaattcgcaccttcacccagcctcccggaccccaagtttgagagcaaagcggccctgctggcatcccggggctccgaagaacttctgtgcttcacccaacgcttggaagacttggtgtgtttctgggaggaagcggcgagctccgggatggacttcaactacagcttctcataccagctcgagggtgagtcacgaaagtcatgtagcctgcaccaggctcccaccgtccgcggctccgtgcgtttctggtgttcactgccaacagcggacacatcgagtgctgtgccgctggagctgcaggtgacggaggcgtccggttctcctcgctatcaccgcatcatccatatcaatgaagtagtgctcctggacgcccccgcggggctgctggcgcgccgggcagaagagggcagccacgtggtgctgcgctggctgccacctcctggagcacctatgaccacccacatccgatatgaagtggacgtgtcggcaggcaaccgggcaggagggacacaaagggtggaggtcctggaaggccgcactgagtgtgttctgagcaacctgcggggcgggacgcgctacaccttcgctgttcgagcgcgcatggccgagccgagcttcagcggattctggagtgcctggtctgagcccgcgtcactactgaccgctagcgacctggaccctctcatcttgacgctgtctctcattctggtcctcatctcgttgttgctgacggttctggccctgctgtccgcagcagcaaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccaggcttctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtaatctagagggcccgtttaaacccgctgatcagcct

SEQ ID NO: 314 - protein, 216 aa, synthetic construct
  REGION 1..216: note = synthetic construct
ATVEPETTPTPNPPTTEEEKTESNQEVANPEHYIKHPLQNRWALWFFKNDKSKTWQANLRLISKFDTVEDFWALYNHIQLSSNLMPGCDYSLFKDGIEPMWEDEKNKRGGRWLITLNKQQRRSDLDRFWLETLLCLIGESFDDYSDDVCGAVVNVRAKGDKIAIWTTECENREAVTHIGRVYKERLGLPPKIVIGYQSHADTATKSGSTTKNRFVV

SEQ ID NO: 315 - RNA, 19 nt, synthetic construct
  misc_feature 1..19: note = synthetic construct
  source 1..19: mol_type = other RNA
acatgaggattacccatgt

SEQ ID NO: 316 - DNA, 130 bp, synthetic construct
  misc_feature 1..130: note = synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 317 - skipped

(19) United States
(12) Patent Application Publication
(10) Pub. No.: US 2026/0209789 A1
(43) Pub. Date: Jul. 23, 2026

(54) TRIGGER-INDUCIBLE MRNA CIRCULARIZATION

(71) Applicant: WESTLAKE UNIVERSITY, Hangzhou, Zhejiang (CN)

(72) Inventors: Jiawei SHAO, Hangzhou, Zhejiang (CN); Shichao LI, Hangzhou, Zhejiang (CN); Hui WANG, Hangzhou, Zhejiang (CN); Xinyuan QIU, Hangzhou, Zhejiang (CN); Mingqi XIE, Hangzhou, Zhejiang (CN)

(21) Appl. No.: 19/138,067

(22) PCT Filed: Oct. 14, 2023
(86) PCT No.: PCT/CN2023/124626

§ 371 Date: Jun. 11, 2025

(30) Foreign Application Priority Data
Oct. 14, 2022 (WO) PCT/CN2022/125433

(51) Int. Cl. C12N 15/85; A61K 31/4995; A61K 38/16; A61K 38/17; A61K 38/28; A61K 48/00; C07K 14/005; C07K 14/47; C07K 14/62; C12N 15/86; G01N 33/68

(52) U.S. Cl. CPC C12N 15/85; A61K 31/4995; A61K 38/162; A61K 38/1709; A61K 38/28; A61K 48/005; C07K 14/005; C07K 14/47; C07K 14/4705; C07K 14/62; C12N 15/86; G01N 33/6893; C07K 2319/00; C12N 2720/12322; C12N 2750/14143; C12N 2770/16022; C12N 2795/00022

(57) ABSTRACT

Provided is a gene regulation strategy enabling programmable control over eukaryotic translational initiation and use of said gene regulation strategy for various biomedical purposes including but not limited to therapeutic transgene delivery, intracellular sensing, molecular diagnostics and gene- and cell-based therapies. Also provided is a method to detect and eliminate cancer cells harboring fusion proteins

### FIELD OF THE INVENTION

The present invention relates to a gene regulation system enabling programmable control over eukaryotic translational initiation and a method using such system e.g., to detect and eliminate cancer cells harboring fusion proteins. The present invention further relates to use of said gene regulation system e.g., for various biomedical purposes including but not limited to therapeutic transgene delivery, intracellular sensing, biocomputation, molecular diagnostics and gene- and cell-based therapies.

### BACKGROUND OF THE INVENTION

One central focus of synthetic biology is the engineering of biocomputational gene circuits capable of driving self-sufficient therapeutic activities through time- and context-specific regulation of mammalian cell activities. These circuits typically comprise interconnected trigger-inducible gene switches and intracellular sensors where expression of target genes is engineered to depend on various user-defined exogenous signals and/or specific intracellular states, respectively. In particular, gene switches are often used to experimentally study specific cellular events with high spatiotemporal precision, to monitor critical bioprocess activities during industrial production, or to remotely control therapeutic transgene expression during gene- and cell-based therapies. Likewise, genetically encoded sensors enable cells to detect and respond to critical biological states that may hardly be accessible for conventional diagnostic tools. For such purposes, most gene switches and sensors developed to date act at the transcriptional level, which have relatively slow response rates and are inherently limited to the detection of signals transduced into the nucleus. A variety of fluorescent sensors have been developed on the basis of protein-protein interaction-dependent reconstitution of split fluorescent proteins, but fluorescence-based sensors acting on the protein-level are limited to visualization-based applications. Also, programming of more customized sense-and-response activities, such as initiation of therapeutic activity, would require complicated redesign of protein structures when using protein-level sensors.

Overall, regulation systems operating at the translational level of gene expression that directly couple real-time detection of various intracellular target compounds to the production of user-defined proteins of interest would be advantageous in many aspects, but engineering of inducible translational regulation systems with good therapeutic efficacy in vivo has remained challenging.

### DISCLOSURE OF THE INVENTION

Precise regulation of (trans) gene activities is critical for achieving optimal efficacy and safety of gene- and cell-based therapies. In recent years, many trigger-inducible gene regulation systems have been developed for use in mammalian cells, enabling the expression of target genes to be controlled by various user-defined exogenous signals and/or intracellular states (Slomovic et al., 2015). For example, optogenetics can provide traceless, non-invasive, long-distance communication between electronic devices and biological systems, allowing portable electronics-such as smartphones-to regulate cellular activities (Shao et al., 2017). Also, human cells can be programmed to sense extracellular disease markers such as blood glucose (Xie et al., 2016) or pH (D. Ausländer et al., 2014), or internal signals such as redox states (Weber et al., 2006) or miRNA signatures (Xie et al., 2011) to respond with customized therapeutic actions, thus enabling the development of disease-specific control devices that provide automated diagnosis and treatment.

Most gene regulation systems reported so far act at the transcriptional level, and therefore have relatively slow sense-and-response dynamics (Ausländer et al., 2012). In contrast, regulation systems operating at the level of protein translation are faster-acting and have attracted increasing interest in recent years (Dykstra et al., 2022). Translation-based systems would also be applicable to sense a wider variety of intracellular signals, since transcription-based sensors are inherently limited to the detection of signals transduced into the nucleus (Nakanishi et al., 2022). However, engineering of inducible translational regulation in mammalian cells has been hampered by the limited information on “convergent” molecular mechanisms of translation initiation (Jackson et al., 2010). As a result, most translational regulation devices developed to date are inhibitory rather than inducible in nature (Saito et al., 2010; Wroblewska et al., 2015), where ligand responsiveness is primarily achieved through prevention of the interruption and/or termination of mRNA activity (Cafferty et al., 2021; Cella et al., 2018). Inducible protein translation, which would allow a trigger signal to directly activate translation of target mRNA, has remained largely elusive.

In eukaryotic cells, protein translation is initiated when a preinitiation complex consisting of a 40S ribosome and initiation factors (eIFs) is recruited to the untranslated region (UTR) at the guanine-rich 5′-cap of mature mRNA molecules that have been exported to the cytoplasm (Jackson et al., 2010; Mitchell and Parker, 2015). Cooperative activity by cap-binding protein eIF4E, RNA helicase eIF4A, central scaffolding protein eIF4G and helicase enhancers eIF4B and eIF4H then subsequently triggers RNA unwinding, ribosome attachment and codon scanning (Jackson et al., 2010). It is well known that the 3′-poly-adenine tail (poly (A)) enhances mRNA stability in living cells, but the underlying mechanism remains a subject of debate (Jackson et al., 2010; Passmore and Coller, 2021). Nevertheless, numerous studies support a “closed-loop” model in which poly-A-binding protein (PABP) is regarded as another canonical eIF capable of simultaneously binding both poly (A) and eIF4G to form a circularized mRNA configuration that is favorable for mRNA scanning, ribosome recycling and protein translation (Gray et al., 2000; Jackson et al., 2010).

To engineer trigger-inducible translational devices, conventional strategies rely on the incorporation of RNA-binding protein (RBP)-specific aptamers into the UTRs of target gene mRNA for the recruitment of RBP-containing regulatory proteins designed to control mRNA stability or eIF4E recruitment (S. Ausländer et al., 2014; Nakanishi et al., 2022). However, these approaches showed only limited efficacy in vivo, which restricts their clinical relevance.

In this work, we show that genetically encoded removal of the poly (A) signal from the target mRNA is the important to overcoming the poor induction fold that has hampered earlier attempts to achieve efficient translational control in mammalian cells. This in turn sets the stage for a new design approach to the systematic engineering of gene switches and intracellular sensors with therapeutic utility. For example, we show that a custom-designed gene switch triggered by the FDA-approved drug grazoprevir could effectively control insulin expression and restore glucose homeostasis in diabetic mice, while remaining compatible with various DNA- and RNA-centered gene therapy delivery strategies currently used in the clinic. In addition, we show that a unique advantage of the STIF architecture is the ability to custom-develop genetically encoded sensors to detect various subcellularly (mis) localized proteins in a quantitative manner, such as the BCR-ABL fusion protein of chronic myelogenous leukemia (CML). In line with this, we describe various designs of intracellular protein sensors with the unique potential to either displace or substantially enhance state-of-the-art cell-state classifier circuits to create next-generation “therapeutic biocomputers” for future precision therapies. By illustrating complexity and specificity issues that could become relevant in a clinical context, we eventually demonstrate self-sufficient elimination of cancer cells in mice mediated by such protein-responsive gene therapies.

Therefore, in a specific aspect, the present invention relates to the following embodiments:
1. A nucleic acids construct comprising an mRNA whose translation (i.e. the event of protein synthesis) is regulated in a trigger-inducible manner.
2. The nucleic acids construct of embodiment 1, wherein initiation of protein translation occurs through trigger-inducible circularization of said mRNA.
3. The nucleic acids construct of embodiments 1-2, wherein mRNA circularization occurs through ectopic overexpression of one or several eIF4F-interacting moieties that can the bind both the 3′-UTR and 5′-UTR of said mRNA.
4. The mRNA circularization strategy of embodiments 2-3, wherein the eIF4F-interacting moiety (also known as eIFBP) consists of a single protein that binds both eIF4F and a specific site on said mRNA.
5. The mRNA circularization strategy of embodiment 4, wherein the single eIF4F-interacting moiety is PABP and its mutants or derivates or a fragment thereof.
6. The mRNA circularization strategy of embodiment 4, wherein the single eIF4F-interacting moiety is NSP3 and its mutants or derivates or a fragment thereof.
7. The mRNA circularization strategy of embodiment 4, wherein the single eIF4F-interacting moiety is VPg and its mutants or derivates or a fragment thereof.
8. The mRNA circularization strategy of embodiments 2-3, wherein the single eIF4F-interacting moiety is a chimeric fusion between anyone protein of the embodiments 5-7 and an RNA-binding protein (RBP).
9. The mRNA circularization strategy of embodiment 8, wherein the RNA-binding protein is L7Ae and its mutants or derivates or a fragment thereof.
10. The mRNA circularization strategy of embodiment 8, wherein the RNA-binding protein is MCP and its mutants or derivates or a fragment thereof.
11. The mRNA circularization strategy of embodiment 8, wherein the RNA-binding protein is λ-N and its mutants or derivates or a fragment thereof.
12. The mRNA circularization strategy of embodiments 2-3, wherein the eIF4F-interacting moiety consist of two recombinant fusion proteins A (comprising the general formula X-Y) and B (comprising the general formula Y′-Z).
13. The mRNA circularization strategy of embodiment 12, wherein X of protein A is anyone protein of the embodiments 5-7 and wherein Z of protein B is anyone protein of the embodiments 9-11; X is genetically fused to protein Y while Z is genetically fused to protein Y′ to allow proteins A and B to associate through a specific Y:Y′-mediated interaction.
14. The mRNA circularization strategy of embodiment 13, wherein protein Y is DocS and protein Y′ is Coh2 or wherein protein Y is Coh2 and protein Y′ is DocS.
15. The mRNA circularization strategy of embodiment 13, wherein protein Y is FKBP and protein Y′ is FRB or wherein protein Y is FRB and protein Y′ is FKBP.
16. The mRNA circularization strategy of embodiment 13, wherein protein Y is ABI and protein Y′ is PYL1 or wherein protein Y is PYL1 and protein Y′ is ABI.
17. The mRNA circularization strategy of embodiment 13, wherein protein Y is GAI and protein Y′ is GID or wherein protein Y is GID and protein Y′ is GAI.
18. The mRNA circularization strategy of embodiment 13, wherein protein Y is NS3a and protein Y′ is GNCR or wherein protein Y is GNCR and protein Y′ is NS3a.
19. The mRNA circularization strategy of embodiment 13, wherein protein Y is NS3a and protein Y′ is DNCR or wherein protein Y is DNCR and protein Y′ is NS3a.
20. The mRNA circularization strategy of embodiment 13, wherein protein Y is NS3a and protein Y′ is ANR or wherein protein Y is ANR and protein Y′ is NS3a.
21. The mRNA circularization strategy of embodiment 13, wherein protein Y is ERK2 and protein Y′ is pE59 or wherein protein Y is pE59 and protein Y′ is ERK2.
22. The mRNA circularization strategy of embodiment 12, wherein X of protein A is anyone protein of the embodiments 9-11 and wherein Z of protein B is anyone protein of the embodiments 5-7; X is genetically fused to protein Y while Z is genetically fused to protein Y′ to allow proteins A and B to associate through a specific Y:Y′-mediated interaction.
23. The mRNA circularization strategy of embodiment 22, wherein protein Y is DocS and protein Y′ is Coh2 or wherein protein Y is Coh2 and protein Y′ is DocS.
24. The mRNA circularization strategy of embodiment 22, wherein protein Y is FKBP and protein Y′ is FRB or wherein protein Y is FRB and protein Y′ is FKBP.
25. The mRNA circularization strategy of embodiment 22, wherein protein Y is ABI and protein Y′ is PYL1 or wherein protein Y is PYL1 and protein Y′ is ABI.
26. The mRNA circularization strategy of embodiment 22, wherein protein Y is GAI and protein Y′ is GID or wherein protein Y is GID and protein Y′ is GAI.
27. The mRNA circularization strategy of embodiment 22, wherein protein Y is NS3a and protein Y′ is GNCR or wherein protein Y is GNCR and protein Y′ is NS3a.
28. The mRNA circularization strategy of embodiment 22, wherein protein Y is NS3a and protein Y′ is DNCR or wherein protein Y is DNCR and protein Y′ is NS3a.
29. The mRNA circularization strategy of embodiment 22, wherein protein Y is NS3a and protein Y′ is ANR or wherein protein Y is ANR and protein Y′ is NS3a.
30. The mRNA circularization strategy of embodiment 22, wherein protein Y is ERK2 and protein Y′ is pE59 or wherein protein Y is pE59 and protein Y′ is ERK2.
31. The mRNA circularization strategy of embodiment 12, wherein X of protein A is anyone protein of the embodiments 5-7 and wherein Z of protein B is anyone protein of the embodiments 9-11; X is genetically fused to protein Y while Z is genetically fused to protein Y′ to allow proteins A and B to associate with target protein Y″ through a specific Y:Y′:Y″-mediated interaction.
32. The mRNA circularization strategy of embodiment 31, wherein protein Y″ is a fusion gene product, an RNA-binding protein or any other intracellular or secreted protein containing one or multiple domains.
33. The mRNA circularization strategy of embodiment 31, wherein protein Y and protein Y′ are two different scFvs.
34. The mRNA circularization strategy of embodiment 31, wherein protein Y and protein Y′ are two different nanobodies.
35. The mRNA circularization strategy of embodiment 31, wherein protein Y is a nanobody and protein Y′ is an scFv, or wherein protein Y is an scFv and protein Y′ is a nanobody.
36. The mRNA circularization strategy of embodiment 31, wherein proteins Y and Y′ are any natural or synthetic proteins that bind to protein Y″ with high affinity; or wherein protein Y is any natural or synthetic protein that binds to protein Y″ with high affinity and protein Y′ is an scFv or a nanobody; or wherein protein Y is an scFv or a nanobody and protein Y′ is any natural or synthetic protein that binds to protein Y″ with high affinity.
37. The mRNA circularization strategy of embodiment 12, wherein X of protein A is anyone protein of the embodiments 9-11 and wherein Z of protein B is anyone protein of the embodiments 5-7; X is genetically fused to protein Y while Z is genetically fused to protein Y′ to allow proteins A and B to associate with specific target protein Y″ through a specific Y:Y′:Y″-mediated interaction.
38. The mRNA circularization strategy of embodiment 37, wherein protein Y″ is a fusion gene product, an RNA-binding protein or any other intracellular or secreted protein containing one or multiple domains.
39. The mRNA circularization strategy of embodiment 37, wherein protein Y and protein Y′ are two different scFvs.
40. The mRNA circularization strategy of embodiment 37, wherein protein Y and protein Y′ are two different nanobodies.
41. The mRNA circularization strategy of embodiment 37, wherein protein Y is a nanobody and protein Y′ is an scFv, or wherein protein Y is an scFv and protein Y′ is a nanobody.
42. The mRNA circularization strategy of embodiment 37, wherein proteins Y and Y′ are any natural or synthetic proteins that bind to protein Y″ with high affinity, or wherein protein Y is any natural or synthetic protein that binds to protein Y″ with high affinity and protein Y′ is an scFv or a nanobody, or wherein protein Y is an scFv or a nanobody and protein Y′ is any natural or synthetic protein that binds to protein Y″ with high affinity.
43. The mRNA circularization strategy of embodiments 3-28, wherein the nucleic acids sequence bound by any said eIF4F-interacting moiety in the 3′-UTR or 5′-UTR of said mRNA comprises or a poly-A signal.
44. The mRNA circularization strategy of embodiments 3-28, wherein the nucleic acids sequence bound by any said eIF4F-interacting moiety in the 3′-UTR or 5′-UTR of said mRNA contains an RNA aptamer.
45. The mRNA circularization strategy of embodiment 44, wherein the RNA aptamer comprises one or multiple tandem copies and combinations of anyone of the sequences C/D-box, MS2-box, boxB, or other aptamers placed into the 3′-UTR or 5′-UTR of said mRNA.
46. The mRNA circularization strategy of embodiment 44, wherein the RNA aptamer is placed into the 3′-UTR or 5′-UTR of said mRNA but the natural poly-A signal of said mRNA is cleaved and removed by a nuclease.
47. The mRNA circularization strategy of embodiment 46, wherein the nuclease accounting for mRNA cleavage belongs to the RNase or CRISPR family of proteins.
48. The mRNA circularization strategy of embodiment 46, wherein cleavage of the natural poly-A signal occurs through RNA interference.
49. The mRNA circularization strategy of embodiment 48, wherein cleavage of the natural poly-A signal occurs through placement of a siRNA binding site or multiple copies thereof into the 3′-UTR.
50. The mRNA circularization strategy of embodiment 48, wherein cleavage of the natural poly-A signal occurs through placement of a shRNA binding site or multiple copies thereof into the 3′-UTR.
51. The mRNA circularization strategy of embodiment 48, wherein cleavage of the natural poly-A signal occurs through placement of a miRNA binding site or multiple copies thereof into the 3′-UTR.
52. The mRNA circularization strategy of embodiment 44, wherein the RNA aptamer is placed into the 3′-UTR or 5′-UTR of said mRNA but the natural poly-A signal of said mRNA is cleaved and removed by a ribozyme.
53. The mRNA circularization strategy of embodiment 52, wherein the ribozyme accounting for mRNA cleavage occurs through placement of a self-cleaving ribozyme or multiple copies thereof or a fragment thereof into the 3′-UTR.
54. The mRNA circularization strategy of embodiment 53, wherein the self-cleaving ribozyme is the hammerhead ribozyme (HHR).
55. The mRNA circularization strategy of embodiment 44, wherein the RNA aptamer is placed into the 3′-UTR or 5′-UTR of said mRNA but the natural 5′-cap of said mRNA is cleaved and removed by a nuclease.
56. The mRNA circularization strategy of embodiment 55, wherein the nuclease accounting for mRNA cleavage belongs to the RNase or CRISPR family of proteins.
57. The mRNA circularization strategy of embodiment 56, wherein cleavage of the natural 5′-cap occurs through RNA interference.
58. The mRNA circularization strategy of embodiment 57, wherein cleavage of the natural 5′-cap signal occurs through placement of a siRNA binding site or multiple copies thereof into the 5′-UTR.
59. The mRNA circularization strategy of embodiment 57, wherein cleavage of the natural 5′-cap signal occurs through placement of a shRNA binding site or multiple copies thereof into the 5′-UTR.
60. The mRNA circularization strategy of embodiment 57, wherein cleavage of the natural 5′-cap signal occurs through placement of a miRNA binding site or multiple copies thereof into the 5′-UTR.
61. The mRNA circularization strategy of embodiment 44, wherein the RNA aptamer is placed into the 3′-UTR or 5′-UTR of said mRNA but the natural 5′-cap of said mRNA is cleaved and removed by a ribozyme.
62. The mRNA circularization strategy of embodiment 61, wherein the ribozyme accounting for mRNA cleavage occurs through placement of a self-cleaving ribozyme or multiple copies thereof or a fragment thereof into the 5′-UTR.
63. The mRNA circularization strategy of embodiment 62, wherein the self-cleaving ribozyme is the hammerhead ribozyme (HHR).
64. The nucleic acids construct of embodiments 1-63, wherein the coding region flanked by the 5′-UTR and 3′-UTR of said mRNA encodes one or multiple proteins or peptides.
65. The nucleic acids construct of embodiment 64, wherein the coding region flanked by the 5′-UTR and 3′-UTR of said mRNA starts with the nucleotide sequence AUG and terminates with the nucleotide sequences UAG, UAA or UGA.
66. The nucleic acids construct of embodiments 1-65, wherein protein translation (mRNA circularization upon binding between mRNA and eIF4F-interacting moieties) occurs outside of a living cell.
67. The nucleic acids construct of embodiments 1-65, wherein protein translation (mRNA circularization upon binding between mRNA and eIF4F-interacting moieties) occurs inside of a living cell.
68. The nucleic acids construct of embodiment 67, wherein the living cell is of mammalian origin.
69. The nucleic acids construct of embodiment 68, wherein the living cell is of human origin.
70. The nucleic acids construct of embodiment 69, wherein the living cell is part of an organism's live tissue.
71. The nucleic acids construct of embodiments 1-70, wherein said mRNA is delivered into the living cell directly in the form of RNA and said proteins are delivered into the living cell directly in the form of proteins.
72. The nucleic acids construct of embodiments 1-70, wherein said mRNA is delivered into the living cell through any form of encoding DNA-based vectors and said proteins are delivered into the living cell through any form of encoding DNA- or RNA-based vectors.
73. A genetically-modified living cell of embodiments 67-72 for use as or in part of a medicament.
74. A genetically-modified living cell of embodiments 67-72 for use in medical diagnostics and/or real-time monitoring of cellular processes.
75. The gene regulation system of embodiments 1-66 for use in point-of-care testing.

### DETAILED DESCRIPTION

### (I) Definitions

For the purpose of explaining this specification, the following definitions will be used, and wherever appropriate, terms used in the singular may also include the plural and vice versa. It is understood that the terminology used herein is for the purpose of describing particular embodiments only and is not intended to be limiting.

As used herein and unless otherwise indicated, the terms “a” and “an” are taken to mean “one”, “at least one” or “one or more”. Unless otherwise required by context, singular terms used herein shall include pluralities and plural terms shall include the singular.

The term “comprise” or “include” as used herein means including the elements, integers or steps described, but does not exclude any other elements, integers or steps. The term also covers the combination of the elements, integers or steps mentioned herein when the term “comprises” or “include” is used, unless otherwise specified. In some embodiments, the term can also mean “consisting of the elements, integers or steps described”.

The term “about” used in combination with a numerical value is intended to encompass the numerical values in a range from a lower limit less than the specified numerical value by 5% to an upper limit greater than the specified numerical value by 5%.

The term “and/or” as used herein, means any of the options or two or more of the options.

Additionally, the words “herein,” “above” and “below” and words of similar import, when used in this application, shall refer to this application as a whole and not to any particular portions of this application.

The term “Nucleic Acids Construct” refers to a specific type of biopolymers made of nucleotide monomers. “Nucleotides” are defined as a chemical structure comprising a 5-carbon sugar, a phosphate group and a nitrogenous base. The two main classes of nucleic acids are deoxyribonucleic acid (DNA) and ribonucleic acid (RNA).

The term “Translation” refers to the event of protein synthesis from mRNA molecules carried out by the ribosome.

The term “messenger ribonucleic acid (mRNA)” refers to a single-stranded RNA molecule that corresponds to the genetic sequence of a gene, which is read by a ribosome in the process of translation.

In the present invention, the term “eIF4F-interacting moieties” refers to any protein or fragment thereof that can interact with anyone member of the eukaryotic eIF4F complex (also known as eIFBP (eIF4F-binding proteins) through the examples of this invention). In particular embodiments, possible eIF4F-interacting moieties include but are not limited to PABP, NSP3, VPg and anyone member of eIF4F.

The term “RNA-binding proteins (RBPs)” describes proteins that are capable of binding a specific sequence or structure within RNA molecules with sufficient affinity. In some embodiment, the RBP can be found in any natural (e.g. listed in (Gerstberger et al., 2014)) or synthetic contexts (e.g. through directed evolution, (Fukunaga and Yokobayashi, 2021)). In particular embodiments, possible RBPs include but are not limited to L7Ae, MCP or λ-N.

The term “ectopic overexpression” describes an event based on forced expression of a particular gene in a cell type in which the gene is usually not expressed at the desired level.

In the present invention, the term “mRNA circularization” relates to a specific state of translational initiation, where the 5′-cap and 3′-tail of mRNA molecules are brought into close proximity by one or several proteins simultaneously binding 5′-UTR and 3′-UTR of said mRNA.

The term “eIF4F” refers to a heterotrimeric protein complex comprising eIF4A, eIF4B, eIF4E and eIF4G that binds the 5′ cap of messenger RNAs (mRNAs) to promote eukaryotic translation initiation.

The term “PABP” refers to the poly (A) binding protein (NCBI-ID: XP_004402403.1) capable of binding poly (A) signals and eIF4G (Passmore and Coller, 2021). In the present invention, PABP is regarded as an eIF4F-interacting moiety and/or an RNA-binding protein.

The term “NSP3” refers to rotaviral nonstructural protein 3 (Groft and Burley, 2002; Piron et al., 1999). In some embodiments, NSP3 derives from bovine rotavirus strain RF (NSP3; UniProtKB/Swiss-Prot: Q86504.1) or human rotavirus strain WA (hNSP3; UniProtKB/Swiss-Prot: Q82054.1). In the present invention, NSP3 is regarded as an eIF4F-interacting moiety and/or an RNA-binding protein.

The term “VPg” refers to Calicivirus-derived VPg Protein (Royall and Locker, 2016). In the present invention, VPg is regarded as an eIF4F-interacting moiety and/or an RNA-binding protein.

The term “fusion protein” refers to a class of hybrid proteins created through the joining of two or more genes that were originally coding for separate proteins.

The term “fusion gene product” refers to one type of fusion proteins that was naturally formed through gene mutation and/or chromosomal translocation, resulting in a novel coding sequence containing parts of the coding sequences from two different genes.

The term “chimeric fusion” refers to one type of synthetic fusion proteins engineered to retain key functions or physico-chemical patterns of each individual protein that were naturally unrelated.

The term “L7Ae” refers to archaeal ribosomal protein (Saito et al., 2010). In the present invention, L7Ae is regarded as an RNA-binding protein.

The term “MCP” refers to bacteriophage MS2 coat protein (GenBank: ASW25882.1). In the present invention, MCP is regarded as an RNA-binding protein.

The term “λ-N” refers to Bacteriophageλ-derived N-peptide (Schoenberg et al., 2004). In the present invention, λ-N is regarded as an RNA-binding protein.

The term “specific protein interaction” refers to a conventional measure to assess binding affinity between two different proteins (e.g. between proteins Y and Y′, between proteins Y and Y″ or between proteins Y′ and Y″) with a dissociation constant lower than 1 μM (K_{D}<1 μM).

The term “high binding affinity” refers to a conventional measure to assess binding strength between multiple moieties, such as between two proteins (dissociation constant K_{D}<1 μM) or between proteins and small molecules (inhibitor constant Kᵢ<1 μM).

The term “Coh2” refers Clostridium thermocellum cohesin (Wu et al., 2020). The term “DocS” refers to Clostridium thermocellum dockerin (Wu et al., 2020). In particular embodiments, both Coh2 and DocS can be either Y, Y′ or Y″ to form specific protein interactions.

The term “FRB” refers to the FKBP-rapamycin binding domain of the mammalian target of rapamycin (mTOR) kinase (Scheller et al., 2018). The term “FKBP” refers to FK506-binding protein (Scheller et al., 2018). In particular embodiments, both FRB and FKBP can be either Y, Y′ or Y″ to form specific protein interactions.

The term “ABI” refers to Abscisic acid-responsive PYL1-binding protein (Gao et al., 2016). The term “PYL1” refers to pyrabactin resistance (PYR)-like protein (Gao et al., 2016). In particular embodiments, both ABI and PYL1 can be either Y, Y′ or Y″ to form specific protein interactions.

The term “GID1” refers to gibberellin insensitive dwarf1 (Gao et al., 2016). The term “GAI” refers to gibberellin insensitive (Gao et al., 2016). In particular embodiments, both GID1 and GAI can be either Y, Y′ or Y″ to form specific protein interactions.

The term “GNCR” refers to grazoprevir/NS3a complex reader (Foight et al., 2019). The term “DNCR” refers to danoprevir/NS3a complex reader (Foight et al., 2019). The term “ANR” refers to apo NS3a reader (Cunningham-Bryant et al., 2019). The term “NS3a” refers to hepatitis C virus protease or a mutant or fragment thereof such as NS3a (H1) (WO2020117778A2). In particular embodiments, both NS3a and GNCR can be either Y, Y′ or Y″ to form specific protein interactions. In other embodiments, both NS3a and DNCR can be either Y, Y′ or Y″ to form specific protein interactions. In other embodiments, both NS3a and ANR can be either Y, Y′ or Y″ to form specific protein interactions.

The term “pE59” refers to a DARPin targeting phosphorylated ERK2 (Kummer et al., 2012). The term “ERK2” refers to extracellular regulated protein kinase 2 (NCBI-ID: NM_138957). In particular embodiments, both pE59 and ERK2 can be either Y, Y′ or Y″ to form specific protein interactions.

The term “protein domain” refers to any functional and/or structural unit of a protein's polypeptide chain that is self-stabilizing and folds independently from the remainder.

The term “secreted protein” refers to any protein that is secreted outside of its producer cell upon translation.

The term “intracellular protein” refers to any protein that resides within its producer cell upon translation.

The term “single-chain variable fragment (scFv)” refers to a specific type of fusion protein between the variable regions of the heavy and light chains of immunoglobulins connected with a short linker peptide.

The term “nanobody” (also known as single-domain antibody) refers to an antibody fragment consisting of a single monomeric variable antibody domain.

The term “poly (A) signal” or “pA” refers to a stretch of an RNA molecule (usually at the 3′-UTR of mRNA) that primarily consists of adenine bases.

The term “aptamer(s)” refers to single-stranded RNA or DNA sequences that form a secondary structure that undergoes a considerable conformational change upon binding to a specific ligand (small molecule, ions or protein) with high affinity. In particular embodiments, possible aptamers include but are not limited to MS2-box, C/D-box or boxB.

The term “MS2-box” refers to an MCP-specific aptamer. In specific embodiments, the RNA-sequence of MS2-box is 5′-UGAGGAUCACCCA-3′.

The term “C/D-box” refers to an L7Ae-specific aptamer. In specific embodiments, the RNA-sequence of C/D-box is 5′-GGGCGUGAUCCGAAAGGUGACCC-3′.

The term “boxB” refers to aλ-N-specific aptamer. In specific embodiments, the RNA-sequence of boxB is 5′-GGGCCCUGAAGAAGGGCCC-3′.

The term “UTR” refers to the untranslated region(s) of an mRNA molecule, which do not contain nucleotide sequences that encode for proteins.

The term “nuclease” refers to an enzyme capable of cleaving the phosphodiester bonds between nucleotides of nucleic acids.

The term “Clustered Regularly Interspaced Short Palindromic Repeats (CRISPR)” refers to a family of DNA sequences found in the genomes of prokaryotic organisms encoding proteins capable of destroying foreign DNA during infections.

The term “Ribonuclease (RNase)” refers to a type of nuclease that catalyzes the degradation of RNA into smaller components.

The term “RNA interference (RNAi)” refers to a biological process in which gene expression from mRNA is repressed (knocked-down) by small regulatory RNA (srRNA) molecules binding to any one site of said target mRNA.

The terms “siRNA”, “shRNA” and “miRNA” refer to different types of srRNA molecules involved in RNA interference.

The term “ribozyme” refers to RNA molecules with enzymatic functions.

The term “hammerhead ribozyme (HHR)” refers to a ribozyme motif that catalyzes reversible cleavage and ligation reactions at a specific site within a (same) RNA molecule. In some embodiment, the HHR-like self-cleaving ribozymes can be found in (Peng et al., 2021; Roberts et al., 2023; Zhong et al., 2020).

The term “5′-cap” refers to a specially altered nucleotide (such as addition of multiple guanine nucleotides) on the 5′ end of some primary transcripts such as precursor messenger RNA.

The term “Peptide” refers to chains of interconnected amino acids that form the basic building blocks of proteins.

The term “virulence factors” refers to representative molecules of pathogenic microorganisms and viruses that could cause diseases upon infection of eukaryotic hosts such as humans.

The term “disease metabolites” refers to representative molecules within body fluids (such as blood, sweat or urine) of a eukaryotic host such as humans reflecting critical health states.

The term “disease signature” refers os any molecules such as proteins that can be representative for a particular disease or abnormal cellular condition. For example, virulence factors, e.g., some virus-specific antigens such as NS3, or oncoproteins, e.g., cancer-specific fusion gene products such as BCR-ABL, any other representative cytosolic biomarker of chronic myelogenous leukemia (CML), or any other disease metabolites.

The term “environmental pollutants” refers to representative molecules within specific biotopes (such as water, air or soil) reflecting particular risks for environmental and/or human health.

The term “trigger-inducible gene regulation” refers to any system that allows the expression of a particular gene of interest to only initiate upon exposure to a specific user-defined signal (i.e., trigger).

The term “trigger-repressible gene regulation” refers to any system that allows the expression of a particular gene of interest to repress upon exposure to a specific user-defined signal (i.e., trigger).

The term “poly (A)-surrogate” refers to a synthetic protein-binding motif engineered into the 3′-UTR that can operate instead or in parallel to native poly (A) signals to bind proteins that contain eIF4F-interacting moieties. In some embodiments, possible poly (A)-surrogates comprise protein-specific aptamers such as MS2-box, C/D-box or boxB.

The term “5′-cap-surrogate” refers to a synthetic protein-binding motif engineered into the 5′-UTR that can operate instead or in parallel to native 5′-cap to bind proteins that contain eIF4F-interacting moieties. In some embodiments, possible 5′-cap-surrogates comprise protein-specific aptamers such as MS2-box, C/D-box or boxB.

The terms “tumor” and “cancer” are used interchangeably herein, covering solid tumors and liquid tumors.

The terms “cancer” and “cancerous” refer to or describe physiological diseases in mammals characterized by unregulated cell growth.

The term “tumor” refers to the growth and proliferation of all neoplastic cells, whether malignant or benign, as well as all pre-cancerous and cancerous cells and tissues. The terms “cancer”, “cancerous” and “tumor” are not mutually exclusive when mentioned herein.

As used herein, “oncoprotein” refers to the antigenic determinant exhibited in the target cell, where the target cell is the cell in the tumor, such as cancer cells and tumor matrix cells.

The term “pharmaceutical supplementary material” refers to diluents, adjuvants (e.g., Freund's adjuvants (complete and incomplete)), excipients, carriers, or stabilizers, etc., which are co-administered with active substance.

The term “pharmaceutical composition” refers to such a composition that exists in a form which allows the biological activity of the active ingredient contained therein to be effective, and does not comprise additional ingredients having unacceptable toxicity to a subject to which the composition is administered.

The term “pharmaceutical combination” refers to non-fixed combination products or fixed combination products, including but not limited to drug kits and drug compositions. The term “unfixed combination” means that the active ingredients (for example, (i) the system in the invention, and (ii) other therapeutic agents) are administered to patients simultaneously, without specific time limits or at the same or different time intervals, in sequence, in separate entities, where these two or more active agents are administered to provide effective levels of prevention or treatment in patients. In some embodiments, the system of the invention used in the pharmaceutical combination are administered at a level not exceeding the level when they are used alone. The term “fixed combination” means that two or more active agents are administered simultaneously to patients in the form of a single entity. It is applicable to select the dose and/or time interval of two or more active agents, so that the combined use of each component can produce greater effect than the single use of any one component in the treatment of disease or disorder. Each component can take its own form of formulation, which can be the same or different.

The term “combination therapy” refers to the application of two or more therapeutic agents or therapeutic modes (such as radiotherapy or surgery) to treat the diseases described herein. Such administration includes the co-administration of these therapeutic agents in a substantially simultaneous manner, such as in a single capsule with a fixed proportion of active ingredients. Alternatively, such application includes the joint application of each active ingredient in multiple or separate containers (such as tablets, capsules, powders and liquids). The powder and/or liquid can be reconstituted or diluted to the required dose before application. In addition, this application also includes the use of each type of therapeutic agent at approximately the same time or at different times in a sequential manner. In either case, the treatment plan will provide the beneficial effect of pharmaceutical combination in treating the disease or condition described herein.

“Individuals” or “subjects” include mammals. Mammals include, but are not limited to, domestic animals (such as cattle, sheep, cats, dogs and horses), primates (such as human and non-human primates, such as monkeys), rabbits, and rodents (such as mice and rats). In some embodiments, the individuals or subjects are human.

As used herein, “treatment” (or “treat” or “treating”) refers to slowing, interrupting, arresting, alleviating, stopping, reducing, or reversing the progression or severity of an existing symptom, disorder, condition, or disease.

As used herein, “prevention” (or “prevent” or “preventing”) includes the inhibition of the onset or progression of a disease or disorder or a symptom of a particular disease or disorder. In some embodiments, subjects with family history of cancer are candidates for preventive regimens. Generally, in the context of cancer, the term “prevention” refers to the administration of a drug prior to the onset of signs or symptoms of a cancer, particularly in subjects at risk of cancer.

The term “effective amount” refers to the amount or dose of the antibody or fragment or conjugate or composition or combination of the invention, which will produce the expected effect in patients needing such treatment or prevention after being administered to patients in a single or multiple dose.

“Therapeutically effective amount” refers to the amount that can effectively achieve the desired results at the required dose and for the required period of time. The therapeutically effective amount is also such an amount, where any toxic or harmful effect of antibody or antibody fragment or conjugate or composition or combination is less than the therapeutic beneficial effect. “Therapeutically effective amount” preferably inhibits measurable parameters (such as tumor volume) by at least about 20%, more preferably by at least about 40%, or even more preferably by at least 50%, 60%, or 70% compared to untreated objects.

“Preventively effective amount” refers to the amount that can effectively achieve the desired prevention results at the required dose and for the required period of time. Generally, since the preventive dose is used before or at an earlier stage of the disease in the objects, the preventively effective amount will be less than the therapeutically effective amount.

The term “vector” as used herein refers to a nucleic acid molecule capable of delivering and/or proliferating another nucleic acid to which it is linked. The term includes vectors that serve as self-replicating nucleic acid structures as well as episomal vectors delivered into the nucleus of a host cell into which they have been introduced. Some vectors are capable of directing the expression of a nucleic acid to which they are operably linked. Such vectors are called “expression vectors” herein.

“Subject/patient/individual sample” refers to a collection of cells or fluids obtained from a patient or subject. The source of the tissue or cell samples can be solid tissues, e.g., from fresh, frozen and/or preserved organ or tissue samples or biopsy samples or puncture samples; blood or any blood component; body fluids such as cerebrospinal fluids, amniotic fluids, peritoneal fluids, or interstitial fluids; cells from a subject at any time during pregnancy or development. Tissue samples may comprise compounds which are naturally not mixed with tissues, such as preservatives, anticoagulants, buffers, fixatives, nutrients, antibiotics, and the like.

### (II.) Gene Regulation System

In a first aspect, the present invention relates to a gene regulation strategy involving a nucleic acids construct that comprises an mRNA whose translation is regulated in a trigger-inducible manner. Preferably, initiation of protein translation occurs through trigger-inducible circularization of said mRNA. Preferably, said mRNA circularization is achieved through ectopic overexpression of one or several eIF4F-interacting moieties that can the bind both the 3′-UTR and 5′-UTR of said mRNA.

In an embodiment, the gene regulation strategy relates to a gene regulation system that can express the target gene mRNA by regulation, which comprises
(i) a synthetic translation initiation Factor (STIF), and mRNA constructs that comprises the mRNA encoding the target protein; or
(ii) a nucleic acid that encodes STIF and a nucleic acid that encodes the mRNA constructs that comprises the mRNA encoding the target protein.

In some further embodiments, the system further comprises a poly (A)-removal module.

In some embodiments, the nucleic acid is DNA or RNA.

In one embodiment, the STIF comprises one or several eIF4F-interacting moieties (or known as “eIFBP”) that can bind either the 3′-UTR or 5′-UTR of said mRNA. In an embodiment, the STIF comprises or consists of at least one eIFBP (eIF4F-binding proteins), and at least one RBP (RNA binding proteins). In some embodiments, the eIFBP and the RBP can be in one protein or in separate protein. In one embodiment, the eIF4F-interacting moiety further comprises other arbitrary protein domains which in some embodiments inserted between RBP and eIFBP domains, e.g., a calmodulin-like motif2CaM-M13. In some embodiments, the STIF further comprises a tag, such as FLAG tag. In some embodiment, the STIF is a fusion protein comprising the aforesaid moieties/proteins.

In a particular embodiment, the eIFBP is selected from PABP, NSP3, VPg and anyone member of eIF4F, such as eIF4A, eIF4B, eIF4E or eIF4G.

In one embodiment, the eIFBP is PABP and its mutants or derivates or a fragment thereof having the function of PABP. In a further embodiment, the PABP is human PABP. In a further embodiment, the PABP comprises the amino acid sequence of SEQ ID NO: 108 or an amino acid sequence having at least 85%, 90%, 91%, 92%, 93%, 94%, 95%, 96%, 97%, 98% or 99% sequence identity to SEQ ID NO: 108, or consists of said amino acid sequence.

In another embodiment, the eIFBP is NSP3 and its mutants or derivates or a fragment thereof having the function of NSP3. In a further embodiment, the NSP3 is derived from bovine or human rotavirus strains. In a further embodiment, the NSP3 comprises the amino acid sequence of SEQ ID NO: 91 or 106 or an amino acid sequence having at least 85%, 90%, 91%, 92%, 93%, 94%, 95%, 96%, 97%, 98% or 99% sequence identity to SEQ ID NO:91 or 106, or consists of said amino acid sequence.

In another embodiment, the eIFBP is VPg and its mutants or derivates or a fragment thereof having the function of VPg. In a further embodiment, the VPg is caliciviral VPg. In a further embodiment, the VPg comprises the amino acid sequence of SEQ ID NO: 120 or an amino acid sequence having at least 85%, 90%, 91%, 92%, 93%, 94%, 95%, 96%, 97%, 98% or 99% sequence identity to SEQ ID NO: 120, or consists of said amino acid sequence.

In another embodiment, the eIFBP is eIF4G and its mutants or derivates or a fragment thereof having the function of eIF4G. In a further embodiment, the eIF4G is human eIF4G. In a further embodiment, the eIF4G comprises the amino acid sequence of SEQ ID NO: 84 or an amino acid sequence having at least 85%, 90%, 91%, 92%, 93%, 94%, 95%, 96%, 97%, 98% or 99% sequence identity to SEQ ID NO: 84, or consists of said amino acid sequence.

In another embodiment, the eIFBP is eIF4E and its mutants or derivates or a fragment thereof having the function of eIF4E. In a further embodiment, the eIF4G is human eIF4E. In a further embodiment, the eIF4G is a variant having a substitution of K119A. In a further embodiment, the eIF4E comprises the amino acid sequence of SEQ ID NO: 83 or 314 or an amino acid sequence having at least 85%, 90%, 91%, 92%, 93%, 94%, 95%, 96%, 97%, 98% or 99% sequence identity to SEQ ID NO: 83 or 314, or an amino acid sequence having at least 85%, 90%, 91%, 92%, 93%, 94%, 95%, 96%, 97%, 98% or 99% sequence identity to SEQ ID NO: 83 and having a substitution of K119A or consists of said amino acid sequence.

In a particular embodiment, the RBP is selected from L7Ae or MCP or λ-N.

In one embodiment, the RBP is L7Ae and its mutants or derivates or a fragment thereof having the function of L7Ae. In a further embodiment, the L7Ae is archeal ribosomal protein L7Ae. In a further embodiment, the L7Ae comprises the amino acid sequence of SEQ ID NO: 92 or an amino acid sequence having at least 85%, 90%, 91%, 92%, 93%, 94%, 95%, 96%, 97%, 98% or 99% sequence identity to SEQ ID NO:
92, or consists of said amino acid sequence.

In another embodiment, the RBP is MCP and its mutants or derivates or a fragment thereof having the function of MCP. In a further embodiment, the MCP is bacteriophage-derived MCP. In a further embodiment, the MCP comprises the amino acid sequence of SEQ ID NO: 98 or an amino acid sequence having at least 85%, 90%, 91%, 92%, 93%, 94%, 95%, 96%, 97%, 98% or 99% sequence identity to SEQ ID NO: 98, or consists of said amino acid sequence. In a further embodiment, the MCP is an MCP variant, which has V29I substitution compared to the MCP. In a further embodiment, the MCP having V29I comprises the amino acid sequence of SEQ ID NO: 200 or an amino acid sequence having at least 85%, 90%, 91%, 92%, 93%, 94%, 95%, 96%, 97%, 98% or 99% sequence identity to SEQ ID NO: 200, or consists of said amino acid sequence.

In another embodiment, the RBP is λ-N and its mutants or derivates or a fragment thereof having the function of λ-N. In a further embodiment, the λ-N is bacteriophage-derived λ-N. In a further embodiment, the λ-N comprises the amino acid sequence of SEQ ID NO: 100 or an amino acid sequence having at least 85%, 90%, 91%, 92%, 93%, 94%, 95%, 96%, 97%, 98% or 99% sequence identity to SEQ ID NO: 100, or consists of said amino acid sequence.

In a particular embodiment, the STIF comprises or consists of a fusion protein comprising one eIFBP and one RBP, and optionally a further protein domain such as 2CaM-M13, or a tag such as FLAG. In one embodiment, the configuration of the fusion protein, from N-terminus to C-terminus, is eIFBP-RBP or RBP-eIFBP, and optionally with some further protein domains inserted or with a tag at N-terminus or C-terminus.

In an embodiment, the STIF comprises or consists of a fusion protein L7Ae-NSP3. In some embodiments, the L7Ae-NSP3 comprises the amino acid sequence of SEQ ID NO: 43 or an amino acid sequence having at least 85%, 90%, 91%, 92%, 93%, 94%, 95%, 96%, 97%, 98% or 99% sequence identity to SEQ ID NO: 43, or consists of said amino acid sequence. In some embodiment, the STIF comprises or consists of a fusion protein 3×FLAG-L7Ae-NSP3. In some embodiments, the L7Ae-NSP3 comprises the amino acid sequence of SEQ ID NO: 49 or an amino acid sequence having at least 85%, 90%, 91%, 92%, 93%, 94%, 95%, 96%, 97%, 98% or 99% sequence identity to SEQ ID NO: 49, or consists of said amino acid sequence. In an embodiment, the STIF comprises or consists of a fusion protein L7Ae-hNSP3. In some embodiments, the L7Ae-hNSP3 comprises the amino acid sequence of SEQ ID NO: 45 or an amino acid sequence having at least 85%, 90%, 91%, 92%, 93%, 94%, 95%, 96%, 97%, 98% or 99% sequence identity to SEQ ID NO: 45, or consists of said amino acid sequence.

In an embodiment, the STIF comprises or consists of a fusion protein L7Ae-eIF4E. In some embodiments, the L7Ae-eIF4E comprises the amino acid sequence of SEQ ID NO: 44 or an amino acid sequence having at least 85%, 90%, 91%, 92%, 93%, 94%, 95%, 96%, 97%, 98% or 99% sequence identity to SEQ ID NO: 44, or consists of said amino acid sequence.

In an embodiment, the STIF comprises or consists of a fusion protein MCP-NSP3. In some embodiments, the MCP-NSP3 comprises the amino acid sequence of SEQ ID NO: 59 or an amino acid sequence having at least 85%, 90%, 91%, 92%, 93%, 94%, 95%, 96%, 97%, 98% or 99% sequence identity to SEQ ID NO: 59, or consists of said amino acid sequence. In some embodiment, the STIF comprises or consists of a fusion protein 3×FLAG-MCP-NSP3. In some embodiments, the MCP-NSP3 comprises the amino acid sequence of SEQ ID NO: 260 or an amino acid sequence having at least 85%, 90%, 91%, 92%, 93%, 94%, 95%, 96%, 97%, 98% or 99% sequence identity to SEQ ID NO: 260, or consists of said amino acid sequence. In an embodiment, the STIF comprises or consists of a fusion protein MCP-hNSP3. In some embodiments, the MCP-hNSP3 comprises the amino acid sequence of SEQ ID NO: 60 or an amino acid sequence having at least 85%, 90%, 91%, 92%, 93%, 94%, 95%, 96%, 97%, 98% or 99% sequence identity to SEQ ID NO: 60, or consists of said amino acid sequence.

In an embodiment, the STIF comprises or consists of a fusion protein PABP-L7Ae. In some embodiments, the PABP-L7Ae comprises the amino acid sequence of SEQ ID NO: 65 or an amino acid sequence having at least 85%, 90%, 91%, 92%, 93%, 94%, 95%, 96%, 97%, 98% or 99% sequence identity to SEQ ID NO: 65, or consists of said amino acid sequence. In an embodiment, the STIF comprises or consists of a fusion protein PABP-L7Ae-3×FLAG. In some embodiments, the PABP-L7Ae-3×FLAG comprises the amino acid sequence of SEQ ID NO: 66 or an amino acid sequence having at least 85%, 90%, 91%, 92%, 93%, 94%, 95%, 96%, 97%, 98% or 99% sequence identity to SEQ ID NO: 66, or consists of said amino acid sequence.

In an embodiment, the STIF comprises or consists of a fusion protein eIF4G-2CaM-M13-L7Ae. In some embodiments, the eIF4G-2CaM-M13-L7Ae comprises the amino acid sequence of SEQ ID NO: 69 or an amino acid sequence having at least 85%, 90%, 91%, 92%, 93%, 94%, 95%, 96%, 97%, 98% or 99% sequence identity to SEQ ID NO: 69, or consists of said amino acid sequence.

In an embodiment, the STIF comprises or consists of a fusion protein PABP-MCP. In some embodiments, the PABP-MCP comprises the amino acid sequence of SEQ ID NO: 206 or an amino acid sequence having at least 85%, 90%, 91%, 92%, 93%, 94%, 95%, 96%, 97%, 98% or 99% sequence identity to SEQ ID NO: 206, or consists of said amino acid sequence.

In an embodiment, the STIF comprises or consists of a fusion protein MCP-eIF4E. In some embodiments, the MCP-eIF4E comprises the amino acid sequence of SEQ ID NO: 244 or an amino acid sequence having at least 85%, 90%, 91%, 92%, 93%, 94%, 95%, 96%, 97%, 98% or 99% sequence identity to SEQ ID NO: 244, or consists of said amino acid sequence.

In an embodiment, the STIF comprises or consists of a fusion protein eIF4G-MCP. In some embodiments, the eIF4G-MCP comprises the amino acid sequence of SEQ ID NO: 253 or an amino acid sequence having at least 85%, 90%, 91%, 92%, 93%, 94%, 95%, 96%, 97%, 98% or 99% sequence identity to SEQ ID NO: 253, or consists of said amino acid sequence.

In an embodiment, the STIF comprises or consists of a fusion protein MCP-VPg. In some embodiments, the MCP-VPg comprises the amino acid sequence of SEQ ID NO: 275 or an amino acid sequence having at least 85%, 90%, 91%, 92%, 93%, 94%, 95%, 96%, 97%, 98% or 99% sequence identity to SEQ ID NO: 275, or consists of said amino acid sequence. In some embodiment, the STIF comprises or consists of a fusion protein MCP^{V29I}-VPg. In some embodiments, the MCP^{V29I}-VPg comprises the amino acid sequence of SEQ ID NO: 276 or an amino acid sequence having at least 85%, 90%, 91%, 92%, 93%, 94%, 95%, 96%, 97%, 98% or 99% sequence identity to SEQ ID NO: 276, or consists of said amino acid sequence.

In an embodiment, the STIF comprises or consists of a fusion protein L7Ae-VPg. In some embodiments, the L7Ae-VPg comprises the amino acid sequence of SEQ ID NO: 281 or an amino acid sequence having at least 85%, 90%, 91%, 92%, 93%, 94%, 95%, 96%, 97%, 98% or 99% sequence identity to SEQ ID NO: 281, or consists of said amino acid sequence.

In a particular embodiment, the STIF comprises or consists of two recombinant fusion proteins A and B, wherein protein A can be RBP-Y or Y-RBP and protein B can be Y′-eIFBP or eIFBP-Y′, protein A can be eIFBP-Y or Y-eIFBP and protein B can be Y′-RBP or RBP-Y′.

In a preferable embodiment, the STIF comprises or consists of two recombinant fusion proteins A and B,
wherein protein A can be RBP-Y and protein B can be Y′-eIFBP, or
wherein protein A can be RBP-Y and protein B can be eIFBP-Y′, or
wherein protein A can be Y-RBP and protein B can be Y′-eIFBP, or
wherein protein A can be Y-RBP and protein B can be eIFBP-Y′, or
wherein protein A can be eIFBP-Y and protein B can be Y′-RBP, or
wherein protein A can be eIFBP-Y and protein B can be RBP-Y′, or
wherein protein A can be Y-eIFBP and protein B can be Y′-RBP, or
wherein protein A can be Y-eIFBP and protein B can be RBP-Y′, or
wherein protein A can be RBP-Y′ and protein B can be Y-eIFBP, or
wherein protein A can be RBP-Y′ and protein B can be eIFBP-Y, or
wherein protein A can be Y′-RBP and protein B can be Y-eIFBP, or
wherein protein A can be Y′-RBP and protein B can be eIFBP-Y, or
wherein protein A can be eIFBP-Y′ and protein B can be Y-RBP, or
wherein protein A can be eIFBP-Y′ and protein B can be RBP-Y, or
wherein protein A can be Y′-eIFBP and protein B can be Y-RBP, or
wherein protein A can be Y′-eIFBP and protein B can be RBP-Y.

In one embodiment, Y and Y′ can be bind with each other constitutively, or by trigger agent or signal or by a further protein Y″.

In some embodiments, protein A or B can comprise multiple tandem repeats of Y or

Y′, for example, 1-5 repeats, e.g., 1, 2, 3, 4, or 5 repeats.

In some embodiments, the Y and Y′ constitutively bind to each other.

In some particular embodiments, protein Y is dockerin and protein Y′ is cohesin; or protein Y is cohesin and protein Y′ is dockerin. In some embodiment, the Docs is Clostridium thermocellum-derived DocS. In a further embodiment, DocS comprises the amino acid sequence of SEQ ID NO: 80 or an amino acid sequence having at least 85%, 90%, 91%, 92%, 93%, 94%, 95%, 96%, 97%, 98% or 99% sequence identity to SEQ ID NO: 80, or consists of said amino acid sequence. In some embodiment, Coh2 is Clostridium thermocellum-derived Coh2. In a further embodiment, Coh2 comprises the amino acid sequence of SEQ ID NO: 77 or an amino acid sequence having at least 85%, 90%, 91%, 92%, 93%, 94%, 95%, 96%, 97%, 98% or 99% sequence identity to SEQ ID NO:77, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| 3xHA-(DocS)3-NSP3 | SEQ-ID NO: 274 |
| (DocS)1-NSP3 | SEQ-ID NO: 12 |
| (DocS)2-NSP3 | SEQ-ID NO: 13 |
| (DocS)3-NSP3 | SEQ-ID NO: 14 |
| DocS-VPg | SEQ-ID NO: 254 |
| DocS-eIF4E | SEQ-ID NO: 257 |
| DocS-eIF4G | SEQ-ID NO: 256 |
| MCP-DocS | SEQ-ID NO: 246 |
| PABP-DocS | SEQ-ID NO: 207 |
| Coh2-L7Ae | SEQ-ID NO: 8 |
| Coh2-NSP3 | SEQ-ID NO: 268 |
| (Coh2)₂-NSP3 | SEQ-ID NO: 269 |
| (Coh2)₃-NSP3 | SEQ-ID NO: 9 |
| L7Ae-Coh2 | SEQ-ID NO: 33 |
| L7Ae-(Coh2)₂ | SEQ-ID NO: 34 |
| L7Ae-(Coh2)₃ | SEQ-ID NO: 35 |
| L7Ae-(Coh2)₂-3xFLAG | SEQ-ID NO: 288 |
| MCP-Coh2 | SEQ-ID NO: 51 |
| MCP-(Coh2)₂ | SEQ-ID NO: 272 |
| MCP-(Coh2)₃ | SEQ-ID NO: 273 |
| λN-Coh2 | SEQ-ID NO: 205 |

In some particular embodiments, protein Y is hepatitis C virus protease NS3a or a mutant or fragment thereof and protein Y′ is apo NS3a reader ANR; or protein Y is ANR and protein Y′ is NS3a. In some embodiment, NS3a is catalytically active NS3a-variant NS3a (H1). In a further embodiment, the NS3a (H1) comprises the amino acid sequence of SEQ ID NO: 105 or an amino acid sequence having at least 85%, 90%, 91%, 92%, 93%, 94%, 95%, 96%, 97%, 98% or 99% sequence identity to SEQ ID NO: 105, or consists of said amino acid sequence. In some embodiment, ANR comprises the amino acid sequence of SEQ ID NO: 73 or an amino acid sequence having at least 85%, 90%, 91%, 92%, 93%, 94%, 95%, 96%, 97%, 98% or 99% sequence identity to SEQ ID NO: 73, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| L7Ae-NS3a(H1) | SEQ-ID NO: 42 |
| MCP-NS3a(H1) | SEQ-ID NO: 56 |
| MCP-(NS3a(H1))₂ | SEQ-ID NO: 57 |
| MCP-(NS3a(H1))₃ | SEQ-ID NO: 58 |
| ANR-NSP3 | SEQ-ID NO: 2 |
| (ANR)₂-NSP3 | SEQ-ID NO: 267 |
| (ANR)₄-NSP3 | SEQ-ID NO: 3 |
| (ANR)₆-NSP3 | SEQ-ID NO: 4 |
| (ANR)₈-NSP3 | SEQ-ID NO: 5 |

In some particular embodiments, protein Y is Bcl-XL and protein Y′ is LD1 or LD3; or protein Y is LD1 or LD3 and protein Y′ is Bcl-XL. In some embodiment, Bcl-XL comprises the amino acid sequence of SEQ ID NO: 74 or an amino acid sequence having at least 85%, 90%, 91%, 92%, 93%, 94%, 95%, 96%, 97%, 98% or 99% sequence identity to SEQ ID NO: 74, or consists of said amino acid sequence. In some embodiment, LD1 comprises the amino acid sequence of SEQ ID NO: 94 or an amino acid sequence having at least 85%, 90%, 91%, 92%, 93%, 94%, 95%, 96%, 97%, 98% or 99% sequence identity to SEQ ID NO: 94, or consists of said amino acid sequence. In some embodiment, LD3 comprises the amino acid sequence of SEQ ID NO: 95 or an amino acid sequence having at least 85%, 90%, 91%, 92%, 93%, 94%, 95%, 96%, 97%, 98% or 99% sequence identity to SEQ ID NO: 95, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| Bcl-XL-NSP3 | SEQ-ID NO: 7 |
| L7Ae-LD1 | SEQ-ID NO: 37 |
| L7Ae-LD3 | SEQ-ID NO: 38 |

In some particular embodiments, protein Y is EGFP and protein Y′ is LaG16; or protein Y is LaG16 and protein Y′ is EGFP. In some embodiment, EGFP comprises the amino acid sequence of SEQ ID NO: 82 or an amino acid sequence having at least 85%, 90%, 91%, 92%, 93%, 94%, 95%, 96%, 97%, 98% or 99% sequence identity to SEQ ID NO: 82, or consists of said amino acid sequence. In some embodiment, LaG16 comprises the amino acid sequence of SEQ ID NO: 93 or an amino acid sequence having at least 85%, 90%, 91%, 92%, 93%, 94%, 95%, 96%, 97%, 98% or 99% sequence identity to SEQ ID NO: 93, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| EGFP-NSP3 | SEQ-ID NO: 18 |
| L7Ae-EGFP | SEQ-ID NO: 251 |
| MCP-EGFP | SEQ-ID NO: 245 |
| MCP-LaG16 | SEQ-ID NO: 52 |
| MCP-(LaG16)₂ | SEQ-ID NO: 53 |

In some particular embodiments, protein Y is CCmut3 and protein Y′ is BCR; or protein Y is BCR and protein Y′ is CCmut3. In some embodiment, CCmut3 comprises the amino acid sequence of SEQ ID NO: 222 or an amino acid sequence having at least 85%, 90%, 91%, 92%, 93%, 94%, 95%, 96%, 97%, 98% or 99% sequence identity to SEQ ID NO: 222, or consists of said amino acid sequence. In some embodiment, BCR comprises the amino acid sequence of SEQ ID NO: 227 or an amino acid sequence having at least 85%, 90%, 91%, 92%, 93%, 94%, 95%, 96%, 97%, 98% or 99% sequence identity to SEQ ID NO: 227, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| 3xHA-CCmut3-NSP3 | SEQ-ID NO: 201 |
| CCmut3-NSP3 | SEQ-ID NO: 258 |

In some particular embodiments, protein Y is ABI (iDab) and protein Y′ is ABL1; or protein Y is ABL1 and protein Y′ is ABI (iDab). In some embodiment, ABI (iDab) comprises the amino acid sequence of SEQ ID NO: 223 or an amino acid sequence having at least 85%, 90%, 91%, 92%, 93%, 94%, 95%, 96%, 97%, 98% or 99% sequence identity to SEQ ID NO: 223, or consists of said amino acid sequence. In some embodiment, the ABL1 is human ABL1. In a further embodiment, the ABL1 comprises the amino acid sequence of SEQ ID NO: 227 or an amino acid sequence having at least 85%, 90%, 91%, 92%, 93%, 94%, 95%, 96%, 97%, 98% or 99% sequence identity to SEQ ID NO: 227, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| 3xFLAG-MCP-(ABI(iDab))₃ | SEQ-ID NO: 202 |
| MCP-ABI(iDab) | SEQ-ID NO: 181 |

In some particular embodiments, protein Y is antibody or antigen binding fragments (such as a nanobody, monobody, affibody, DARPin or scFv) that specifically binds to an antigen and protein Y′ is the antigen; or protein Y is the antigen and protein Y′ is the antibody or antigen binding fragments (such as a nanobody, monobody, affibody, DARPin or scFv). In some embodiments, the antigen is NS3 or its fragment (e.g., N-terminus of NS3), e.g., hepatitis C virus (HCV)-derived nNS3. In a further embodiment, the nNS3 comprises the amino acid sequence of SEQ ID NO: 303 or an amino acid sequence having at least 85%, 90%, 91%, 92%, 93%, 94%, 95%, 96%, 97%, 98% or 99% sequence identity to SEQ ID NO: 303, or consists of said amino acid sequence.

In some embodiments, the antigen binding fragment is an scFv that specifically binds to NS3 or its fragment (e.g., N-terminus of NS3), for example, said scFv comprises the amino acid sequence of SEQ ID NO: 112 or 113 or an amino acid sequence having at least 85%, 90%, 91%, 92%, 93%, 94%, 95%, 96%, 97%, 98% or 99% sequence identity to SEQ ID NO: 112 or 113, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| L7Ae-scFv35 | SEQ-ID NO: 47 |
| scFv162-NSP3 | SEQ-ID NO: 70 |
| (scFv162)₂-NSP3 | SEQ-ID NO: 261 |
| (scFv162)₃-NSP3 | SEQ-ID NO: 262 |

In some embodiments, Y and Y′ conditionally bind to each other in a trigger-inducible or trigger-repressible manner.

In some particular embodiments, protein Y is ABI and protein Y′ is PYL1; or protein Y is PYL1 and protein Y′ is ABI and the binding between ABI and PYL1 is triggered by abscisic acid. In some embodiment, ABI comprises the amino acid sequence of SEQ ID NO: 71 or an amino acid sequence having at least 85%, 90%, 91%, 92%, 93%, 94%, 95%, 96%, 97%, 98% or 99% sequence identity to SEQ ID NO: 71, or consists of said amino acid sequence. In some embodiment, PYL1 comprises the amino acid sequence of SEQ ID NO: 111 or an amino acid sequence having at least 85%, 90%, 91%, 92%, 93%, 94%, 95%, 96%, 97%, 98% or 99% sequence identity to SEQ ID NO: 111, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| L7Ae-(ABI)₃ | SEQ-ID NO: 31 |
| ABI-MCP | SEQ-ID NO: 259 |
| ABI-L7Ae | SEQ-ID NO: 265 |
| ABI-NSP3 | SEQ-ID NO: 266 |
| L7Ae-ABI | SEQ-ID NO: 284 |
| NSP3-ABI | SEQ-ID NO: 294 |
| NSP3-(PYL1)₃ | SEQ-ID NO: 64 |
| (PYL1)₃-NSP3 | SEQ-ID NO: 67 |
| L7Ae-PYL1 | SEQ-ID NO: 203 |
| PYL1-NSP3 | SEQ-ID NO: 278 |
| PYL1-L7Ae | SEQ-ID NO: 279 |
| NSP3-PYL1 | SEQ-ID NO: 291 |

In some particular embodiments, protein Y is a DrBPhP-specific affibody and protein Y′ is DrBPhP; or protein Y is DrBPhP and protein Y′ is a DrBPhP-specific affibody and the binding between the DrBPhP-specific affibody and DrBPhP is triggered by light. In some embodiments, the affibody is Aff6_{V18FΔN} and comprises the amino acid sequence of SEQ ID NO: 72 or an amino acid sequence having at least 85%, 90%, 91%, 92%, 93%, 94%, 95%, 96%, 97%, 98% or 99% sequence identity to SEQ ID NO: 72, or consists of said amino acid sequence. In some embodiment, DrBPhP comprises the amino acid sequence of SEQ ID NO: 81 or an amino acid sequence having at least 85%, 90%, 91%, 92%, 93%, 94%, 95%, 96%, 97%, 98% or 99% sequence identity to SEQ ID NO: 81, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| MCP-(Aff6_{V18FΔN})₄ | SEQ-ID NO: 50 |
| DrBPhP-NSP3 | SEQ-ID NO: 15 |

In some particular embodiments, protein Y is hepatitis C virus protease NS3a or a mutant or fragment thereof and protein Y′ is apo NS3a reader ANR; or protein Y is ANR and protein Y′ is NS3a and the binding between NS3a and ANR is inhibited by Grazoprevir. In some embodiment, NS3a comprises the amino acid sequence of SEQ ID NO: 105 or an amino acid sequence having at least 85%, 90%, 91%, 92%, 93%, 94%, 95%, 96%, 97%, 98% or 99% sequence identity to SEQ ID NO: 105, or consists of said amino acid sequence. In some embodiment, ANR comprises the amino acid sequence of SEQ ID NO: 73 or an amino acid sequence having at least 85%, 90%, 91%, 92%, 93%, 94%, 95%, 96%, 97%, 98% or 99% sequence identity to SEQ ID NO: 73, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| L7Ae-NS3a(H1) | SEQ-ID NO: 42 |
| MCP-NS3a(H1) | SEQ-ID NO: 56 |
| MCP-(NS3a(H1))₂ | SEQ-ID NO: 57 |
| MCP-(NS3a(H1))₃ | SEQ-ID NO: 58 |
| ANR-NSP3 | SEQ-ID NO: 2 |
| (ANR)₂-NSP3 | SEQ-ID NO: 267 |
| (ANR)₄-NSP3 | SEQ-ID NO: 3 |
| (ANR)₆-NSP3 | SEQ-ID NO: 4 |
| (ANR)₈-NSP3 | SEQ-ID NO: 5 |

In some particular embodiments, protein Y is CIB1 and protein Y′ is Cry2; or protein Y is Cry2 and protein Y′ is CIB1 and the binding between CIB1 and Cry2 is triggered by light. In some embodiment, CIB1 comprises the amino acid sequence of SEQ ID NO: 76 or an amino acid sequence having at least 85%, 90%, 91%, 92%, 93%, 94%, 95%, 96%, 97%, 98% or 99% sequence identity to SEQ ID NO: 76, or consists of said amino acid sequence. In some embodiment, Cry2 comprises the amino acid sequence of SEQ ID NO: 78 or an amino acid sequence having at least 85%, 90%, 91%, 92%, 93%, 94%, 95%, 96%, 97%, 98% or 99% sequence identity to SEQ ID NO: 78, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| L7Ac-CIB1 | SEQ-ID NO: 32 |
| MCP-CIB1 | SEQ-ID NO: 247 |
| Cry2-NSP3 | SEQ-ID NO: 10 |

In some particular embodiments, protein Y is DNCR and protein Y′ is hepatitis C virus protease NS3a or a mutant or fragment thereof; or protein Y is NS3a and protein Y′ is DNCR and the binding between DNCR and NS3a is triggered by Danoprevir. In some embodiment, DNCR comprises the amino acid sequence of SEQ ID NO: 79 or an amino acid sequence having at least 85%, 90%, 91%, 92%, 93%, 94%, 95%, 96%, 97%, 98% or 99% sequence identity to SEQ ID NO: 79, or consists of said amino acid sequence. In some embodiment, NS3a comprises the amino acid sequence of SEQ ID NO: 104 or an amino acid sequence having at least 85%, 90%, 91%, 92%, 93%, 94%, 95%, 96%, 97%, 98% or 99% sequence identity to SEQ ID NO: 104, or consists of said amino acid sequence. In some embodiment, the NS3a is catalytically active NS3a-variant NS3a (H1). In some embodiment, the NS3a (H1) NS3a comprises the amino acid sequence of SEQ ID NO: 105 or an amino acid sequence having at least 85%, 90%, 91%, 92%, 93%, 94%, 95%, 96%, 97%, 98% or 99% sequence identity to SEQ ID NO: 105, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| DNCR-NSP3 | SEQ-ID NO: 270 |
| (DNCR)₂-NSP3 | SEQ-ID NO: 271 |
| (DNCR)₃-NSP3 | SEQ-ID NO: 11 |
| L7Ae-NS3a | SEQ-ID NO: 39 |
| L7Ae-(NS3a)₂ | SEQ-ID NO: 40 |
| L7Ae-(NS3a)₃ | SEQ-ID NO: 41 |
| L7Ae-(NS3a)₃-3xFLAG | SEQ-ID NO: 48 |
| MCP-NS3a | SEQ-ID NO: 54 |
| MCP-(NS3a)₂ | SEQ-ID NO: 290 |
| MCP-(NS3a)₃ | SEQ-ID NO: 55 |
| NS3a-NSP3 | SEQ-ID NO: 62 |
| 3xFLAG-MCP-NS3a | SEQ-ID NO: 263 |
| L7Ae-NS3a(H1) | SEQ-ID NO: 42 |
| MCP-NS3a(H1) | SEQ-ID NO: 56 |
| MCP-(NS3a(H1))₂ | SEQ-ID NO: 57 |
| MCP-(NS3a(H1))₃ | SEQ-ID NO: 58 |

In some particular embodiments, protein Y is FKBP and protein Y′ is FRB; or protein Y is FRB and protein Y′ is FKBP and the binding between FKBP and FRB is triggered by Rapamycin. In some embodiment, FKBP comprises the amino acid sequence of SEQ ID NO: 86 or an amino acid sequence having at least 85%, 90%, 91%, 92%, 93%, 94%, 95%, 96%, 97%, 98% or 99% sequence identity to SEQ ID NO: 86, or consists of said amino acid sequence. In some embodiment, FRB comprises the amino acid sequence of SEQ ID NO: 87 or an amino acid sequence having at least 85%, 90%, 91%, 92%, 93%, 94%, 95%, 96%, 97%, 98% or 99% sequence identity to SEQ ID NO: 87, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| FKBP-L7Ae | SEQ-ID NO: 25 |
| FKBP-NSP3 | SEQ-ID NO: 252 |
| FRB-NSP3 | SEQ-ID NO: 26 |
| MCP-FRB | SEQ-ID NO: 243 |

In some particular embodiments, protein Y is GAI and protein Y′ is GID1; or protein Y is GID1 and protein Y′ is GAI and the binding between GAI and GID1 is triggered by Gibberellic acid. In some embodiment, GAI comprises the amino acid sequence of SEQ ID NO: 88 or an amino acid sequence having at least 85%, 90%, 91%, 92%, 93%, 94%, 95%, 96%, 97%, 98% or 99% sequence identity to SEQ ID NO: 88, or consists of said amino acid sequence. In some embodiment, GID1 comprises the amino acid sequence of SEQ ID NO: 89 or an amino acid sequence having at least 85%, 90%, 91%, 92%, 93%, 94%, 95%, 96%, 97%, 98% or 99% sequence identity to SEQ ID NO: 89, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| GAI-LZAe | SEQ-ID NO: 27 |
| L7Ae-GAI | SEQ-ID NO: 283 |
| (GAI)₃-L7Ae | SEQ-ID NO: 285 |
| GAI-NSP3 | SEQ-ID NO: 289 |
| NSP3-GAI | SEQ-ID NO: 293 |
| NSP3-GID1 | SEQ-ID NO: 63 |
| MCP-GID1 | SEQ-ID NO: 242 |
| L7Ae-GID1 | SEQ-ID NO: 282 |
| GID1-L7Ae | SEQ-ID NO: 286 |
| GID1-NSP3 | SEQ-ID NO: 287 |
| NSP3-(GID1)₃ | SEQ-ID NO: 292. |

In some particular embodiments, protein Y is GNCR and protein Y′ is hepatitis C virus protease NS3a or a mutant or fragment thereof; or protein Y is NS3a and protein Y′ is GNCR and the binding between GNCR and NS3a is triggered by Grazoprevir. In some embodiment, GNCR comprises the amino acid sequence of SEQ ID NO: 90 or an amino acid sequence having at least 85%, 90%, 91%, 92%, 93%, 94%, 95%, 96%, 97%, 98% or 99% sequence identity to SEQ ID NO: 90, or consists of said amino acid sequence. In some embodiment, NS3a comprises the amino acid sequence of SEQ ID NO: 104 or an amino acid sequence having at least 85%, 90%, 91%, 92%, 93%, 94%, 95%, 96%, 97%, 98% or 99% sequence identity to SEQ ID NO: 104, or consists of said amino acid sequence. In some embodiment, the NS3a is catalytically active NS3a-variant NS3a (H1). In a further embodiment, the NS3a (H1) comprises the amino acid sequence of SEQ ID NO: 105 or an amino acid sequence having at least 85%, 90%, 91%, 92%, 93%, 94%, 95%, 96%, 97%, 98% or 99% sequence identity to SEQ ID NO: 105, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| 3xHA-(GNCR)₃-NSP3 | SEQ-ID NO: 1 |
| GNCR-NSP3 | SEQ-ID NO: 28 |
| (GNCR)₂-NSP3 | SEQ-ID NO: 29 |
| (GNCR)₃-NSP3 | SEQ-ID NO: 30 |
| L7Ae-GNCR | SEQ-ID NO: 36 |
| L7Ae-NS3a | SEQ-ID NO: 39 |
| L7Ae-(NS3a)₂ | SEQ-ID NO: 40 |
| L7Ae-(NS3a)₃ | SEQ-ID NO: 41 |
| L7Ae-(NS3a)₃-3xFLAG | SEQ-ID NO: 48 |
| MCP-NS3a | SEQ-ID NO: 54 |
| MCP-(NS3a)₂ | SEQ-ID NO: 290 |
| MCP-(NS3a)₃ | SEQ-ID NO: 55 |
| NS3a-NSP3 | SEQ-ID NO: 62 |
| 3xFLAG-MCP-NS3a | SEQ-ID NO: 263 |
| L7Ae-NS3a(H1) | SEQ-ID NO: 42 |
| MCP-NS3a(H1) | SEQ-ID NO: 56 |
| MCP-(NS3a(H1))₂ | SEQ-ID NO: 57 |
| MCP-(NS3a(H1))₃ | SEQ-ID NO: 58 |

In some particular embodiments, protein Y is an mCherry-specific nanobody and protein Y′ is mCherry; or protein Y is mCherry and protein Y′ is an mCherry-specific nanobody and the binding between the nanobody and mCherry is triggered by light. In some embodiment, the nanobody is LaM8_{AK47} and comprises the amino acid sequence of SEQ ID NO: 220 or an amino acid sequence having at least 85%, 90%, 91%, 92%, 93%, 94%, 95%, 96%, 97%, 98% or 99% sequence identity to SEQ ID NO: 220, or consists of said amino acid sequence. In some embodiment, mCherry comprises the amino acid sequence of SEQ ID NO: 97 or an amino acid sequence having at least 85%, 90%, 91%, 92%, 93%, 94%, 95%, 96%, 97%, 98% or 99% sequence identity to SEQ ID NO: 97, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| MCP-LaM8_{AK47} | SEQ-ID NO: 241 |
| λN-mCherry | SEQ-ID NO; 204 |
| mCherry-NSP3 | SEQ-ID NO: 250 |

In some embodiments, binding between Y and Y′ is regulated by intracellular signaling dynamics, e.g., by agent that can activate the intracellular signaling.

In some particular embodiments, protein Y is ERK2 and protein Y′ is pE59; or protein Y is pE59 and protein Y′ is ERK2 and the binding between ERK2 and pE59 is triggered by activated MAPK signaling (e.g., epidermal growth factor EGF). In some embodiment, ERK2 comprises the amino acid sequence of SEQ ID NO: 85 or an amino acid sequence having at least 85%, 90%, 91%, 92%, 93%, 94%, 95%, 96%, 97%, 98% or 99% sequence identity to SEQ ID NO: 85, or consists of said amino acid sequence. In some embodiment, pE59 comprises the amino acid sequence of SEQ ID NO: 110 or an amino acid sequence having at least 85%, 90%, 91%, 92%, 93%, 94%, 95%, 96%, 97%, 98% or 99% sequence identity to SEQ ID NO: 110, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| ERK2-NSP3 | SEQ-ID NO: 277 |
| (ERK2)₂-NSP3 | SEQ-ID NO: 24 |
| L7Ae-pE59 | SEQ-ID NO: 280 |
| L7Ae-(pE59)₂ | SEQ-ID NO: 46 |
| MCP-(pE59)₂ | SEQ-ID NO: 249 |

In some embodiments, Y and Y′ can interact with each other via another protein Y″. That is, only when Y″ exists, Y and Y′ can associate with each other as target protein Y″ triggers a Y:Y′:Y″-interaction.

In some embodiment, Y″ can be any protein or agent as long as it can be bound by two different proteins, preferably in at different domains or different epitopes of Y″.

In some particular embodiments, protein Y″ is a fusion gene product, an oncoprotein, a virulence factor, an RNA-binding protein or any other intracellular or secreted protein containing one or multiple domains.

In some particular embodiments, protein Y and protein Y′ are two different scFvs that bind specifically to protein Y″, e.g., at different domains or different epitopes of Y″. In some particular embodiment, Y″ can be an antigen that can be specifically bound at different domains or different epitopes by two antibodies or antigen binding fragments.

In some particular embodiment, protein Y″ is selected from a disease-specific cellular signature, such as an oncoprotein, e.g., a fusion gene product or protein complex that is specifically expressed in a tumor cell or tumor tissue. For example, Y″ can be a the fusion protein BCR-ABL, or a virulence factor such as HCV or HCV specific proteins (e.g., NS3 protein).

In some particular embodiments, protein Y and protein Y′ are two different nanobodies that bind specifically to protein Y″, e.g., at different domains or different epitopes of Y″. In some particular embodiments, protein Y and protein Y′ are two different affibodies that bind specifically to protein Y″, e.g., at different domains or different epitopes of Y″. In some particular embodiments, protein Y and protein Y′ are two different monobodies that bind specifically to protein Y″, e.g., at different domains or different epitopes of Y″. In some particular embodiments, protein Y and protein Y′ are two different DARPins that bind specifically to protein Y″, e.g., at different domains or different epitopes of Y″.

In some particular embodiments, protein Y is any natural or synthetic protein that binds to protein Y″ with high affinity and protein Y′ is an scFv, a DARPin, a monobody, an affibody or a nanobody selected from antigen binding fragments that binds to protein Y″; or protein Y is an scFv, a DARPin, a monobody, an affibody or a nanobody selected from antigen binding fragments and protein Y′ is any natural or synthetic protein that binds to protein Y″ with high affinity; or both proteins Y and Y′ are any natural or synthetic proteins that bind to protein Y″ with high affinity.

In some embodiments, protein Y is CCmut3, and protein Y′ is ABI (iDab), or protein Y′ is CCmut3, and protein Y″ is ABI (iDab), and Y″ is a protein bound by both CCmut3 and ABI (iDab), e.g., BCR-ABL. In some embodiment, CCmut3 comprises the amino acid sequence of SEQ ID NO: 222 or an amino acid sequence having at least 85%, 90%, 91%, 92%, 93%, 94%, 95%, 96%, 97%, 98% or 99% sequence identity to SEQ ID NO: 222, or consists of said amino acid sequence. In some embodiment, ABI (iDab) comprises the amino acid sequence of SEQ ID NO: 223 or an amino acid sequence having at least 85%, 90%, 91%, 92%, 93%, 94%, 95%, 96%, 97%, 98% or 99% sequence identity to SEQ ID NO: 223, or consists of said amino acid sequence. In a further embodiment, the BCR-ABL comprises the amino acid sequence of SEQ ID NO: 301 or an amino acid sequence having at least 85%, 90%, 91%, 92%, 93%, 94%, 95%, 96%, 97%, 98% or 99% sequence identity to SEQ ID NO: 301, or consists of said amino acid sequence.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| 3xHA-CCmut3-NSP3 | SEQ-ID NO: 201 |
| CCmut3-NSP3 | SEQ-ID NO: 258 |
| 3xFLAG-MCP-ABI(iDab) | SEQ-ID NO: 202 |
| MCP-ABI(iDab) | SEQ-ID NO: 181 |

In some embodiments, protein Y is an antibody or antigen binding fragments (such as an scFv, a DARPin, amonobody, an affibody or a nanobody) that specifically binds to an antigen Y″, or protein Y′ is another antibody or antigen binding fragments (such as an scFv, a DARPin, a monobody, an affibody or a nanobody) that specifically binds to the same antigen Y″. In a specific embodiment, the antigen Y″ is NS3 or a mutant or fragment thereof (e.g., N-terminus of NS3; nNS3). In some embodiment, nNS3 comprises the amino acid sequence of SEQ ID NO: 303 or an amino acid sequence having at least 85%, 90%, 91%, 92%, 93%, 94%, 95%, 96%, 97%, 98% or 99% sequence identity to SEQ ID NO: 303, or consists of said amino acid sequence.

In some embodiments, the antigen binding fragment Y or Y′ is an scFv that specifically binds to NS3 or its fragment (e.g., N-terminus of NS3; nNS3), for example, said scFv comprises the amino acid sequence of SEQ ID NO: 112 (e.g., scFv162) or SEQ ID NO: 113 (e.g., scFv35) or an amino acid sequence having at least 85%, 90%, 91%, 92%, 93%, 94%, 95%, 96%, 97%, 98% or 99% sequence identity to SEQ ID NO: 112 or 113, or consists of said amino acid sequence. In some embodiments, Y is scFv162 and Y′ is scFv35. In some embodiment, Y′ is scFv162 and Y is scFv35.

In some embodiments, protein A or protein B is selected from the following fusion proteins:

| Protein A or protein B (Y-RBP, RBP-Y′, Y- | Comprising or consisting |
|---|---|
| eIFBP or eIFBP-Y′) is selected from | of the SEQ ID NO: |
| L7Ae-scFv35 | SEQ-ID NO: 47 |
| scFv162-NSP3 | SEQ-ID NO: 70 |
| (scFv162)₂-NSP3 | SEQ-ID NO: 261 |
| (scFv162)₃-NSP3 | SEQ-ID NO: 262 |

In an embodiment, the mRNA construct comprises (i) a coding region and (ii) an RNA segment specifically bound by STIFs.

In some embodiments, the RNA segment specifically bound by STIFs belongs to 5′-UTR and/or 3′-UTR of said mRNA. In some embodiments, the RNA segment specifically bound by STIFs comprises 5′-UTR and 3′-UTR.

In some embodiments, the coding region of said mRNA is flanked by the 5′-UTR and 3′-UTR. In some embodiments, the coding region encodes one or multiple target proteins or peptides, for example, the mRNA can be any nucleic acid segment encoding for any polypeptide of interest. In some embodiment, the coding region is any RNA sequence starting with nucleotides AUG and terminating with nucleotide sequences UAG, UAA or UGA). For example, the target mRNA can be the one which encodes target protein, preferably, the target protein can be selected from therapeutic proteins such as protein-based hormones e.g. insulin, pro-apoptotic proteins such as BAX, fluorescent protein such as EGFP or mCherry or any other secreted or intracellular protein that can be detected with its expression, e.g. reporter proteins such as SEAP or luciferase.

In some embodiment, the RNA segment specifically bound by STIFs is a poly-A signal or a poly (A)-surrogate, and more preferably, poly (A)-surrogate.

In some embodiment, the poly (A) surrogate can be any segment that contains or consists of one or more n aptamer repeats binding to a specific RBP. In some embodiments, the poly (A) surrogate is placed into the 3′-UTR or 5′-UTR of said mRNA.

In some embodiments, the aptamer is selected from C/D-box, MS2-box, or boxB.

In an embodiment, the poly (A) surrogate contains tandem repeats of the L7Ae-specific C/D-box aptamer e.g., (C/D-box)ₙ, or MCP-specific MS2-box aptamer (MS2-boxₙ, or λ-N-specific aptamer (boxB)ₙ, wherein n can be any number between 1 to 1000, e.g., 5-30, e.g., 8, 12, 16, or 24. The selection of the aptamer depends on the RBP, for example, if RBP is L7Ae, the aptamer is usually L7Ae-specific C/D-box aptamers, and if RBP is MCP, the aptamer is usually MCP-specific MS2-box aptamers.

In some embodiment, C/D-box comprises or consists of the nucleic acid sequence of SEQ ID NO: 123. In some embodiment, the MS2-box comprises or consists of the nucleic acid sequence of SEQ ID NO: 125 or 315. In some embodiment, the boxB comprises or consists of the nucleic acid sequence of SEQ ID NO: 121.

In a further embodiment, when the poly (A)-surrogate is placed into the 3′-UTR or 5′-UTR of said mRNA, the system further comprises a construct expressing RNase or CRISPR family of proteins.

In a further embodiment, when the poly (A)-surrogate is placed into the 3′-UTR or 5′-UTR of said mRNA, the mRNA construct further comprises an RNA cleavage site (e.g. a self-cleaving ribozyme signal such as HHR) enabling pre-programmed poly (A)-removal, which is located between the aptamer and the poly (A) and placed into the 3′-UTR.

In one embodiment, the cleavage is performed by RNA interference and the RNA cleavage site is a siRNA binding site or multiple copies thereof, a shRNA binding site or multiple copies or a miRNA binding site or multiple copies thereof. In a particular embodiment, when the RNA cleavage site is siRNA binding site, a construct expressing siRNA should be comprised in the system of the invention. In a particular embodiment, when the RNA cleavage site is shRNA binding site, a construct expressing shRNA should be comprised in the system of the invention. In a particular embodiment, when the RNA cleavage site is miRNA binding site, a construct expressing miRNA should be comprised in the system of the invention.

For example, the RNA cleavage site may comprise one or multiple repeats of (BS (shRNA-216))ₙ, wherein n can be any number between 1 and 100 and preferably n can be any number between 1 and 4. In some embodiment, the RNA cleavage site is BS (shRNA-216). In some embodiment, the BS (shRNA-216) comprises or consists of SEQ ID NO: 122. In some embodiment, when the RNA cleavage site is BS (shRNA-216), the system further comprises a construct that express shRNA-216 to cleave the polyA. In some embodiment, the shRNA-216 comprises or consists of SEQ ID NO: 126.

In another embodiment, the cleavage is performed by ribozyme and the cleavage site is a ribozyme. In some embodiment, the ribozyme is a self-cleaving ribozyme or multiple copies thereof or a fragment thereof. In some embodiment, the self-cleaving ribozyme is the hammerhead ribozyme (HHR)ₙ, wherein n can be any number between 1 and 100 and preferably n can be any number between 1 and 4. In some embodiment, the RNA cleavage site is HHR. In some embodiment, the HHR comprises or consists of SEQ ID NO: 124. In some embodiment, when the RNA cleavage site is HHR, the system does not need to comprise further construct to cleave the HHR since it triggers spontaneous self-excision of the poly (A) signal.

In some embodiment, the construct comprises, from 5′- to 3′-end, a 5′-UTR, the coding region, the poly (A)-surrogate, the cleavage site and other elements of the 3′ UTR.

In some embodiment, the construct comprises, from 5′- to 3′-end, a5′-UTR, the coding region and the following combination of poly (A)-surrogates cleavage sites in the 3′ UTR:

| Combination | Poly(A)-surrogate | Cleavage site |
|---|---|---|
| 1 | (C/D-box)₈ | (BS(shRNA-216))₂ |
| 2 | (C/D-box)₁₆ | (BS(shRNA-216))₂ |
| 3 | (C/D-box)₁₂ | (BS(shRNA-216))₂ |
| 4 | (C/D-box)₂₄ | (BS(shRNA-216))₂ |
| 5 | (C/D-box)₂₄ | (BS(shRNA-216))₂ |
| 6 | (C/D-box)₂₄ | HHR |
| 7 | (MS2-box)₂₄ | HHR |
| 8 | (MS2-box)₁₆ | HHR |
| 9 | (MS2-box)₂₄ | HHR |
| 10 | (MS2-box)₂₄ | HHR |
| 11 | none | (HHR)₂ |
| 12 | none | (HHR)₄ |
| 13 | (MS2-box)₂₄ | HHR |
| 14 | none | HHR |
| 15 | (MS2-box)₂₄ | HHR |
| 16 | (MS2-box)₂₄ | HHR |
| 17 | (MS2-box)₁₆ | HHR |
| 18 | (C/D-box)₂₄ | none |
| 19 | (C/D-box)₄ | none |
| 20 | (MS2-box)₂₄ | HHR |
| 21 | (MS2-box)₁₂ | HHR |
| 22 | (MS2-box)₈ | HHR |
| 23 | (MS2-box)₈ | (BS(shRNA-216)₂ |

wherein when the poly (A)-surrogate is (MS2-box)ₙ, the STIF comprises MCP; and when the poly (A)-surrogate is (C/D-box)ₙ, the STIF comprises L7Ae.

In some embodiment, the mRNA construct comprises RNA sequence as shown in anyone of SEQ ID NO: 131 to 149 or any RNA sequence in Table 2, or comprises RNA sequence having at least 85%, 90%, 91%, 92%, 93%, 94%, 95%, 96%, 97%, 98% or 99% sequence identity to anyone of SEQ ID NO: 131 to 149 or any RNA sequence or in Table 2, or consists of said RNA sequence.

In some embodiments, the mRNA construct can further comprise 5′-cap or 5′-cap surrogates. For example, the 5′-cap can be replaced by 5′-cap surrogates by cleaving the 5′-cap. In some embodiments, the poly (A) surrogate can also be used as 5′-cap surrogate. In some embodiments, the 5′-cap can by cleaved in the same way as that for poly (A). In some embodiments, the cleavage site for poly (A) can also be used as a cleavage site for 5′-cap.

In some embodiment, the construct comprises, from 5′- to 3′-end, a5′-UTR, a cleavage site, the 5′-cap surrogate, the coding region and 3′ UTR. In some embodiment, the construct comprises, from 5′- to 3′-end, a5′-UTR and the following combination of cleavage sites, 5′-cap surrogates, coding regions and poly(A)-surrogates in the 3′ UTR:

In some embodiment, the construct, from N-terminus to C-terminus, the following elements:

| 5′-cleavage | 5′cap-surrogate | Coding region | poly(A)-surrogate | 3′-cleavage |
|---|---|---|---|---|
| (HHR)ₙ | (C/D-box)n | Coding region | (C/D-box)ₙ | (HHR)ₙ |
| (HHR)ₙ | (MS2-box)ₙ | encoding one or | (MS2-box)ₙ | (HHR)ₙ |
| (HHR)ₙ | (C/D-box)ₙ | multiple target | none | none |
| (HHR)ₙ | (MS2-box)ₙ | proteins or | none | none |
| (HHR)ₙ | (C/D-box)ₙ | peptides, for | (MS2-box)ₙ | (HHR)ₙ |
| (HHR)ₙ | (MS2-box)ₙ | example, the | (C/D-box)ₙ | (HHR)ₙ |
| (HHR)ₙ | (C/D-box)ₙ | mRNA can be any | (C/D-box)ₙ | BS(shRNA-216)ₙ |
| (HHR)ₙ | (MS2-box)ₙ | nucleic acid | (MS2-box)ₙ | BS(shRNA-216)ₙ |
| (HHR)ₙ | (C/D-box)ₙ | segment encoding | none | none |
| (HHR)ₙ | (MS2-box)ₙ | for any | none | none |
| (HHR)ₙ | (C/D-box)ₙ | polypeptide of | (MS2-box)ₙ | BS(shRNA-216)ₙ |
| (HHR)ₙ | (MS2-box)ₙ | interest. | (C/D-box)ₙ | BS(shRNA-216)ₙ |
| BS(shRNA-216)ₙ | (C/D-box)ₙ | For example, any | (C/D-box)ₙ | (HHR)ₙ |
| BS(shRNA-216)ₙ | (MS2-box)ₙ | RNA sequence | (MS2-box)ₙ | (HHR)ₙ |
| BS(shRNA-216)ₙ | (C/D-box)ₙ | starting with | none | none |
| BS(shRNA-216)ₙ | (MS2-box)ₙ | nucleotides AUG | none | none |
| BS(shRNA-216)ₙ | (C/D-box)ₙ | and terminating | (MS2-box)ₙ | (HHR)ₙ |
| BS(shRNA-216)ₙ | (MS2-box)ₙ | with nucleotide | (C/D-box)ₙ | (HHR)ₙ |
| BS(shRNA-216)ₙ | (C/D-box)ₙ | sequences UAG, | (C/D-box)ₙ | BS(shRNA-216)ₙ |
| BS(shRNA-216)ₙ | (MS2-box)ₙ | UAA or UGA). | (MS2-box)ₙ | BS(shRNA-216)ₙ |
| BS(shRNA-216)ₙ | (C/D-box)ₙ | | none | none |
| BS(shRNA-216)ₙ | (MS2-box)ₙ | | none | none |
| BS(shRNA-216)ₙ | (C/D-box)ₙ | | (MS2-box)ₙ | BS(shRNA-216)ₙ |
| BS(shRNA-216)ₙ | (MS2-box)ₙ | | (C/D-box)ₙ | BS(shRNA-216)ₙ |

wherein in BS (shRNA-216), or (HHR)ₙ, wherein n can be any number between 1 and 100 and preferably n can be any number between 1 and 4; in (C/D-box)ₙ or (MS2-box)ₙ, n can be any number between 1 to 1000, e.g., 5-30, e.g., 8, 12, 16, or 24;
when the poly (A)-surrogate is (MS2-box)ₙ, the STIF comprises MCP; and when the poly (A)-surrogate is (C/D-box)ₙ, the STIF comprises L7Ae.

In a specific embodiments, the a gene regulation system comprises a construct expressing Synthetic translation initiation Factor (STIF) (STIF construct) and an mRNA construct comprising the mRNA encoding the target protein, wherein the STIF and the expression construct respectively comprises the following elements:

| | mRNA construct |
|---|---|
| STIF | 5′- to 3′-end (GOI (Gene of Interest) |
| from N-terminus to C-terminus | refers to coding region as described herein) |
| Y-L7Ae or L7Ae-Y′ or Y′-L7Ae or L7Ae-Y & | 5′-UTR-GOI-(C/D-Box)₈ |
| Y-NSP3 or NSP3-Y′ or Y′-NSP3 or NSP3-Y | |
| Y-L7Ae or L7Ae-Y′ or Y′-L7Ae or L7Ae-Y & | 5′-UTR-GOI-(C/D-Box)₁₂ |
| Y-NSP3 or NSP3-Y′ or Y′-NSP3 or NSP3-Y | |
| Y-L7Ae or L7Ae-Y′ or Y′-L7Ae or L7Ae-Y & | 5′-UTR-GOI-(C/D-Box)₁₆ |
| Y-NSP3 or NSP3-Y′ or Y′-NSP3 or NSP3-Y | |
| Y-L7Ae or L7Ae-Y′ or Y′-L7Ae or L7Ae-Y & | 5′-UTR-GOI-(C/D-Box)₂₄ |
| Y-NSP3 or NSP3-Y′ or Y′-NSP3 or NSP3-Y | |
| Y-MCP or MCP-Y′ or Y′-MCP or MCP-Y & Y- | 5′-UTR-GOI-(MS2-Box)₈ |
| NSP3 or NSP3-Y′ or Y′-NSP3 or NSP3-Y | |
| Y-MCP or MCP-Y′ or Y′-MCP or MCP-Y & Y- | 5′-UTR-GOI-(MS2-Box)₁₂ |
| NSP3 or NSP3-Y′ or Y′-NSP3 or NSP3-Y | |
| Y-MCP or MCP-Y′ or Y′-MCP or MCP-Y & Y- | 5′-UTR-GOI-(MS2-Box)₁₆ |
| NSP3 or NSP3-Y′ or Y′-NSP3 or NSP3-Y | |
| Y-MCP or MCP-Y′ or Y′-MCP or MCP-Y & Y- | 5′-UTR-GOI-(MS2-Box)₂₄ |
| NSP3 or NSP3-Y′ or Y′-NSP3 or NSP3-Y | |

wherein the mRNA construct can be either genetically encoded by DNA-based expression vectors or delivered as in vitro transcribed RNA, and/or wherein
the STIF can be either genetically encoded by DNA-based expression vectors, delivered as in vitro transcribed RNA or directly in form of purified proteins, and/or wherein
Y and Y′ are two different proteins that bind to each other in a constitutive, trigger-inducible or protein Y″-dependent manner as described in the present invention.

In an aspect, the present invention relates to a gene regulation system triggered by Grazoprevir.

In an embodiment, the Grazoprevir-triggered system comprises
(i) Synthetic translation initiation Factors (STIFs) and mRNA construct that comprises the mRNA encoding the target protein; or
(ii) A nucleic acid that encodes STIF and a nucleic acid that encodes the mRNA construct that comprises the mRNA encoding the target protein;
wherein the STIFs comprise or consist of two recombinant fusion proteins A and B,
wherein protein A can be RBP-Y or Y-RBP and protein B can be Y′-eIFBP or eIFBP-Y′, or
wherein protein A can be eIFBP-Y or Y-eIFBP and protein B can be Y′-RBP or RBP-Y′
wherein Y is NS3a and Y′ is GNCR.

In one embodiment, the RBP is L7Ae or MCP.

In some embodiments, protein A or B can comprise multiple tandem repeats of GNCR or NS3a, for example, 1-5 repeats, e.g., 1, 2, 3, 4, or 5 repeats.

In one embodiment, protein A has a L7Ae-(NS3a), configuration and protein B has a (GNCR)ₙ-NSP3 configuration, or protein A has a L7Ae-(GNCR), configuration and protein B has a (NS3a)ₙ-NSP3 configuration, wherein n is an integer from 1 to 10, e.g., 1, 2 or 3. In a further embodiment, the mRNA construct comprises a coding region encoding the target protein and an RNA segment specifically bound by STIF, wherein the RNA segment specifically bound by STIF comprises tandem repeats of the L7Ae-specific C/D-box aptamers e.g., (C/D-box)ₙ, optionally placed into the 3′ UTR or 5′ UTR of the mRNA construct. Preferably, the mRNA construct comprises, from the 5′- to 3′-end, a5′ UTR, the coding region, (C/D-box)ₙ (wherein n=1-30, e.g., 24), (BS (shRNA-216))ₙ (wherein n=1, 2 or 3, e.g, 2) or (HHR)ₙ (wherein n=1, 2, 3 or 4, e.g, 1) and 3′ UTR.

In one embodiment, protein A has a MCP-(NS3a)ₙ configuration and protein B has a (GNCR)ₙ-NSP3 configuration or protein A has a MCP-(GNCR)ₙ configuration and protein B has a (NS3a)ₙ-NSP3 configuration, wherein n is an integer from 1 to 10, e.g., 1, 2 or 3. In a further embodiment, the mRNA construct comprises a coding region encoding the target protein and an RNA segment specifically bound by STIF, wherein the RNA segment specifically bound by STIF comprises tandem repeats of the MCP-specific MS2-box aptamers e.g., (MS2-box)ₙ, optionally placed into the 3′ UTR or 5′ UTR of the mRNA construct. Preferably, the mRNA construct comprises, from the 5′- to 3′-end, a5′ UTR, the coding region, (MS2-box), (wherein n=1-30, e.g., 24), (BS (shRNA-216))ₙ (wherein n=1, 2 or 3, e.g, 2) or (HHR)ₙ (wherein n=1, 2, 3 or 4, e.g, 1) and 3′ UTR.

In a further embodiment, the system further comprises a module that expresses shRNA-216.

In one embodiment, the target protein is selected from therapeutic proteins such as protein-based hormones e.g. insulin, fluorescent protein such as EGFP or mCherry or any other secreted or intracellular protein that can be detected with its expression, e.g. reporter proteins such as SEAP or luciferase.

### (III) Methods to Produce the Gene Regulation System

In one embodiment, the present invention relates to a nucleic acid encoding the STIF protein(s).

In another embodiment, the present invention relates to a nucleic acid encoding the mRNA construct.

It is well known to those skilled in the art, because of codon degeneracy, each amino acid sequence can be encoded by multiple nucleic acid sequences. Nucleic acid sequences encoding the molecules of the present invention may be produced by methods well known in the art, such as by de novo solid DNA synthesis, or by PCR amplification.

In other embodiments, the present invention relates to vectors comprising said nucleic acid. In one embodiment, the vector is an expression vector, such as a eukaryotic expression vector. Vectors include, but are not limited to viruses, plasmids, mucoids, phages or yeast artificial chromosomes (YAC). In one embodiment, the expression vector is an episomal vector, e.g. derived from pcDNA3.1 (+) (Invitrogen, CA; cat-no. V79020). In other embodiments, the expression vector is an AAV vector or lenti virus.

AAV can be any AAV vector known in the art ((Li and Samulski, 2020)), e.g., AAV1, AAV2, AAV2.7m8, AAV3a, AAV3b, AAV4, AAV5, AAV6, AAV7, AAV8, AAV9 or AAV10.

In some embodiments the AAV vector comprises anyone or more or all of the following elements:
(i) ITR, e.g., an ITR comprising the nucleic acid sequence of SEQ ID NO: 212 or SEQ ID NO: 316, or the nucleic acid sequence having at least 90%, 91%, 92%, 93%, 94%, 95%, 96%, 97%, 98% or 99% sequence identity to SEQ ID NO: 212 or 316, or consists of said amino acid sequence;
(ii) nucleic acid sequences flanked by ITR and encoding the STIF protein(s) or said mRNA constructs.

In an embodiment, the lenti virus comprises 5′-LTR, e.g., a5′-LTR comprising the nucleic acid sequence of SEQ ID NO: 215, or the nucleic acid sequence having at least 90%, 91%, 92%, 93%, 94%, 95%, 96%, 97%, 98% or 99% sequence identity to SEQ ID NO: 215, or consists of said nucleic acid sequence.

In some embodiment, the vector comprises a promoter, e.g., CMV promoter, U6 promoter, phosphoglycerate kinase gene promoter, elongation factor 1αpromoter, or mammalian CREB1-specific promoter. In some embodiment, the promoter comprises or consists of the nuclei acid sequence of anyone of SEQ ID NO: 127, SEQ ID NO: 129, SEQ ID NO: 210, SEQ ID NO: 214, SEQ ID NO: 233-240, or a nucleic acid having at least 90%, 91%, 92%, 93%, 94%, 95%, 96%, 97%, 98% or 99% sequence identity to said nuclei acid.

The vector can also comprises IRES for expressing two or more STIF protein and/or mRNA construct.

In an embodiment, the mRNA construct of the present invention can be synthesized directly. For example, the genetically-encoded expression construct comprising the mRNA encoding the target protein can be synthesized directly. There are several methods in the art to directly synthesize the mRNA constructs of the system, such as those listed in “(Parr et al., 2020; Yu et al., 2020)”.

In another embodiment, the STIF can be produced by culturing a host cell comprising the nucleic acid encoding the STIF under a condition suitable for expressing said STIF, and thus producing the STIF protein(s). When STIF comprises two proteins, the nucleic acids encoding each protein may be in the same vectors or in different vectors. In a further embodiment, nucleic acids encoding each proteins of STIF of the invention may be introduced into the same or different host cells for expression.

In one embodiment, the host cell is of eukaryotic origin. In another embodiment, the host cells are selected from yeast cells, mammalian cells such as CHO cells (such as CHO-S, such as ExpiCHO-S) or 293 cells (such as 293F or HEK293 cells), or other cells suitable for the preparation of STIF. In one embodiment, the host cell is prokaryotic, such as a bacterium, such as E. coli.

### (IV). Pharmaceutical Composition and Pharmaceutical Formulations

In some embodiments, the present invention provides a composition comprising the system described herein, preferably the composition is a pharmaceutical composition. In one embodiment, the composition further comprises pharmaceutically acceptable supplementary material. In one embodiment, the composition, for example, the pharmaceutical composition, comprises a combination of the system of invention, and one or more other therapeutic agents, or agents that can trigger the expression of the system.

The invention further includes a composition comprising the system. In some embodiments, the system comprises STIF and the mRNA construct. In some further embodiments, the system comprises nucleic acid encoding STIF and nucleic acid encoding mRNA construct. In some further embodiment, the composition comprises STIF fusion protein, and mRNA construct. In some further embodiments, the composition comprises a DNA encoding STIF and a DNA encoding mRNA construct. In some embodiments, the DNA is in a vector, such as expression vector, such as AAV vector, e.g., pAAV2/8, or pcDNA, e.g., pcDNA3.1. In some embodiments, the RNA or DNA can be in formulated in protein particles, e.g. viruses or virus-like particles, polymer participles, e.g. PEI or polymersomes, or lipid particles, e.g. liposomes or lipid nanoparticles (LNP). In some further embodiments, the composition comprises an RNA encoding STIF and an RNA encoding mRNA construct. In a specific embodiment, the RNA is mRNA. In some embodiments, the RNA is an in vitro transcribed mRNA produced from the vectors comprising the DNA encoding the system, or directly synthesized according to techniques described in “(Parr et al., 2020; Yu et al., 2020)”.

These compositions can further comprise suitable pharmaceutically acceptable supplement, such as pharmaceutically acceptable carriers and pharmaceutically acceptable excipients, including buffers known in the art.

As used herein, “pharmaceutically acceptable carrier” includes any physiologically compatible solvents, dispersion media, isotonic agents and absorption retardants that is suitable in the composition.

For the use of pharmaceutically acceptable supplementary material, see also “Handbook of Pharmaceutical Excipients”, 8th edition, R.C. Rowe, P. J. Seskey and S. C. Owen, Pharmaceutical Press, London, Chicago.

The composition of the present invention can be in various forms. These forms include, for example, liquid, semi-solid and solid dosage forms, such as liquid solution (for example, injectable solution and infusion solution), powder or suspension, liposome and suppository. The preferred form depends on the intended mode of administration and therapeutic use.

A pharmaceutical formulation comprising the system described herein can be prepared by mixing each component of the system of the invention with the required purity with one or more optional pharmaceutically acceptable supplementary material, preferably in the form of lyophilized preparation or aqueous solution.

The pharmaceutical composition or formulation of the invention can further comprise more than one active ingredients, e.g., the ingredient that is necessary for trigger the system, or the ingredients which are required for the specific indication to be treated, preferably those active ingredients with complementary activities that will not adversely affect each other. The said active ingredients are appropriately combined in an effective amount for the intended use.

### (V). Pharmaceutical Combination and Kit

In some embodiments, the invention further provides a pharmaceutical combination or a pharmaceutical combination product, which include the system of the invention, and one or more other agents.

In one embodiment, the other agent is the agent that can be used to trigger or induce or repress STIF-specific translation. In a particular embodiment, the other agent can be protein Y″, Abscisic acid, Grazoprevir, Danoprevir, MAPK, Rapamycin or Gibberellic acid. In a further embodiment, the other agent is other therapeutic agents.

Another object of the invention is to provide a kit comprising the pharmaceutical combination of the invention, preferably in the form of drug dose unit. Therefore, the dose unit can be provided according to the regimen or interval of the administration.

In one embodiment, the kit of the invention comprises:
one or more containers comprising each components of the system, for example, the different components can be comprised in one contain together, or comprised in separate containers;
another container comprising the other agents or a pharmaceutical composition containing the other agents.

### (VI). Use and Method

In one aspects, the present invention relates to a cell-based expression system for one or several mRNA constructs whose translation is regulated in a trigger-inducible manner in a living cell, including

delivering the mRNA construct(s) and STIF directly in the form of RNA and proteins into the living cell, and expressing the mRNA construct in said living cell; or

delivering the nucleic acid encoding the mRNA construct(s) or expression vector comprising said nucleic acid or the nucleic acid encoding STIF or expression vector comprising said nucleic acid into the living cell, e.g., by transfection with expression vector comprises said nucleic acids, and express the target protein.

In a preferred embodiment, said living cell is of mammalian origin. In a more preferred embodiment, said living cell is of human origin. In another preferred embodiment, said living cell is part of an organism's live tissue.

In a further aspect, the present invention relates to the use or implementation of the gene regulation system of the present invention of said mRNA in a cell-free system. Possible applications of said implementation include but are not limited to point-of-care testing involving sensing of disease metabolites, virulence factors or environmental pollutants in vitro.

### (VI-I) for Therapy

In a further aspect, the present invention relates to the gene regulation system of the present invention, for use as a medicament, e.g., for gene therapy.

In some embodiments, the therapy with the system of the present invention can achieve long-term treatment efficacy in vivo.

In one embodiment, the present invention relates to a gene therapy, comprising administering the gene regulation system or the pharmaceutical composition or pharmaceutical combination or kit of the present invention to a subject need thereof.

In another embodiment, the present invention provides a method for preventing or treating a disease, comprising administering the gene regulation system or the pharmaceutical composition or pharmaceutical combination or kit of the present invention to a subject need thereof.

In another embodiment, the present invention relates to a use of the gene regulation system or the pharmaceutical composition or pharmaceutical combination or kit in the manufacture of a medicament, e.g., a gene therapy approach for diagnosis, treatment or prevention of a disease.

In some specific embodiments, the disease is selected from cancer or immune diseases or metabolic disease or infectious disease. In one embodiment, the cancer is solid tumor or blood tumor. In one embodiment, the immune disease is auto-immune diseases. In one embodiment, the metabolic disease is diabetes. In one embodiments, the infectious disease is viral (e.g., HCV) infection.

The subject can be a mammal, such as a primate, preferably a higher primate, such as a human (for example, an individual suffering from the disease described herein or at risk of suffering from the disease described herein). In one embodiment, the subject suffers from the disease described herein (for example, cancer) or is at risk of suffering from the disease described herein.

In some embodiments, the system or pharmaceutical composition or pharmaceutical combination or kit of the invention may delay, attenuate or cure the onset of the disease and/or symptoms related to the disease.

In some embodiments, the system or pharmaceutical composition or pharmaceutical combination or kit of the invention can also be used in combination with one or more other therapies, such as therapeutic modes and/or other therapeutic agents, for the uses described herein, such as for the diagnosis and/or prevention and/or treatment of related diseases or disorders mentioned herein.

Such combination therapy covers combination administration (for example, two or more therapeutic agents are included in the same or separate formulation), and separate administration. In this case, the administration of the system of the invention can occur before, at the same time, and/or after the administration of other therapeutic agents and/or therapeutic modes.

The route of administration of the system is based on known methods for gene therapy, and depending on the type of the system. For example, for AAV therapy, injection may be applicable to introduce the system to the subject. The trigger agent can be administered based on known methods, such as oral, intravenous injection, intraperitoneal, intracerebral (parenchymal), intraventricular, intramuscular, ophthalmic, intra-arterial, intra-portal or intrafocal route; by continuous release system or by implantable device. In some embodiments, the system or the trigger agent may be administered by bolus injection or by continuous infusion or by an implant device.

In one embodiment, the target protein is insulin, e.g., human insulin, and the trigger agent is Grazoprevir. The disease to be treated by the system is Diabetes, e.g., Type I Diabetes.

In a further aspect, the system of the present invention can be used to act as a translation-based protein sensors, and used as next-generation therapeutic gene circuits providing programmable, broadly adjustable and self-sufficient gene therapies for treatment of many human diseases.

It is known that whereas fusion proteins (e.g. native BCR-ABL) are prominent cases where a single intracellular target protein can unambiguously identify pathologic cell states, some diseases however typically lack such unique biomarkers. In these cases, a “true” disease-specific cellular signature must be resolved through a combined detection of various subordinate checkpoint signals.

The system of the present invention thus can be used in a scenario requiring such multiplexed cell-state detection.

In a specific embodiment, the system can treat tumor or cancer, especially those involving the kind of complexity and specificity issues that would be relevant in a clinical context. Specifically, the system can be flexibly interconnected with other genetically encoded sensors to eventually achieve any desired custom combination of tissue- and target-specificity in vivo. The system of the present invention is not limited to systematic and empirical design of highly specific fusion gene sensors for treatment of hitherto intractable cancers, but is amenable to the detection of any intracellular target signal of interest for which suitable sets of proteinaceous binder moieties (e.g. nanobodies) can be found.

In one embodiment, the present invention provides method to treat tumor, e.g., specifically kill the tumor cells or tumor tissue.

Particularly, the system comprises
(i) Synthetic translation initiation Factors (STIFs) and mRNA construct that comprises the mRNA encoding the target protein; or
(ii) A nucleic acid that encodes STIF and a nucleic acid that encodes the mRNA construct that comprises the mRNA encoding the target protein;
wherein the STIFs comprise or consist of two recombinant fusion proteins A and B, wherein protein A can be RBP-Y or Y-RBP and protein B can be Y′-eIFBP or eIFBP-Y′, or wherein protein A can be eIFBP-Y or Y-eIFBP and protein B can be Y′-RBP or RBP-Y′, wherein Y and Y′ can interact with each other via another protein Y″. That is, only when Y″ exists, Y and Y′ can associate with each other as target protein Y″ triggers a Y:Y′:Y″-interaction.

In one embodiment, the RBP is L7Ae or MCP.

In one embodiment, Y or Y′ is any protein or fragment that can bind to the Y″.

In some embodiment, Y″ can be any protein or agent as long as it can be bound by two different proteins, preferably in at different domains or different epitopes of Y″. In some particular embodiment, protein Y″ is selected from a disease-specific cellular signature, such as an oncoprotein, e.g., a fusion gene product or protein complex that is specifically expressed in a tumor cell or tumor tissue. For example, Y″ can be a the fusion protein BCR-ABL, or a virulence factor such as HCV or HCV specific proteins (e.g., NS3 protein).

In some embodiments, protein A or B can comprise multiple tandem repeats of Y or Y′, for example, 1-5 repeats, e.g., 1, 2, 3, 4, or 5 repeats. In some particular embodiments, Y and Y′ are two different nanobodies that bind specifically to protein Y″, e.g., at different domains or different epitopes of Y″. In some particular embodiments, protein Y and protein Y′ are two different affibodies that bind specifically to protein Y″, e.g., at different domains or different epitopes of Y″. In some particular embodiments, protein Y and protein Y′ are two different monobodies that bind specifically to protein Y″, e.g., at different domains or different epitopes of Y″. In some particular embodiments, protein Y and protein Y′ are two different DARPins that bind specifically to protein Y″, e.g., at different domains or different epitopes of Y″.

In some embodiment, Y″ is selected from a disease-specific cellular signature, such as a fusion gene product, an oncoprotein, a virulence factor, an RNA-binding protein or any other intracellular or secreted protein containing one or multiple domains, and the target protein is a protein that can kill the tumor cells (e.g., pro-apoptotic protein, e.g., Bax protein).

### (VI-II) for Complex Biocomputation in Mammalian Cells or In Vivo

In one aspect of the present invention, the system of the present invention can be used for biocomputation in vitro or in vivo.

In one embodiment, genetic tristate buffers can be created through regulated expression of trigger-inducible gene switches programmed with either “buffered” (BUF) and “inverted” (NOT) signal processing logics in mammalian cells or in vivo.

In one specific embodiment, the present invention provides a transcription-translation based gene circuit, wherein control input B operating at an upstream layer (e.g. transcription) monitors the expression of a synthetic translation initiation factor (STIF) that modulates target protein expression from a downstream layer (e.g. translation). Tristate buffers can be engineered to contain up to 4 types of gene switches: B activates STIF expression (IF1), B terminates STIF expression (IF0), A activates target protein expression (BUF) and A terminates target protein expression (NOT). For example, STIFs use rotavirus-derived nonstructural protein 3 domains (NSP3) to bind the preinitiation complex eIF4F, resulting in translation of MCP-specific mRNA upon inducible association of NSP3- and MCP-containing factors.

In one embodiment, the BUF- and NOT-switches can be grazoprevir-inducible system comprising a STIF comprising the grazoprevir-controlled triad NS3a/GNCR/ANR.

In one embodiment, for initiation of translation, a circularized mRNA configuration must be established with the 5′- and 3′-ends brought into close proximity by a synthetic translational initiation factor (STIF) comprising a NSP3 domain and synthetic tethers (MCP-NS3a (H1)) consisting of an RNA-binding MCP domain and a STIF-binding NS3a (H1) domain. Since grazoprevir concomitantly triggers dissociation of ANR from NS3a (H1) and association of GNCR to NS3a (H1), STIFs can be engineered to contain GNCR and NSP3 enable grazoprevir-inducible translation (BUF switch). Likewise, fusion of ANR to NSP3 results in grazoprevir-repressible translation (NOT switch).

In some embodiments of the tristate circuits, BUF and NOT switches are governed by an “Active-HIGH” (IF1) or an “Active-LOW” (IF0) control signal producing “normal” or “inverted” output, respectively. Biological implementations of “Active-HIGH” and “Active-LOW” switches should be orthogonal to each other to enable interference-free operation in mammalian cells. For example, a vanillic acid-inducible gene switch based on an PKA/CREB1-responsive promoter activated by olfactory receptor MOR9-1-regulated cAMP-signaling (Saxena et al., 2016) could be a potential IF1 switch, whereas the IF0 switch could be completed by a VanR-dependent mammalian transactivator (VanR-VP64) modulating gene expression from cognate VanO-containing promoters (Gitzinger et al., 2012).

In a further embodiment, the present invention provides two additional sets of grazoprevir-responsive gene switches that can be combined with (vanillic acid-regulated) IF1/IF0-switches (as illustrated in FIG. 25A).
(i) GEMS-based systems, grazoprevir-inducible gene expression from synthetic STAT3-specific promoters (BUF2) occurs upon dimerization of a synthetic cell membrane receptor containing IL6RB-derived intracellular domains and extracellular GNCR- and NS3a (H1)-domains. Exchange of the GNCR domain by ANR results in grazoprevir-repressible gene expression (NOT2).
(ii) For StaPLd-based systems, grazoprevir inhibits autoproteolysis of a synthetic mammalian trans-activator PcaV-StaPLd-VP64 or trans-silencer PcaV-StaPLd-KRAB, resulting in trigger-inducible activation (BUF3) or inhibition (NOT3) of gene expression from synthetic PcaV-specific promoters.

In one embodiment, the present invention provides tristate buffers in four types, based on combination of grazoprevir-controlled (BUF and NOT) with vanillic acid-controlled gene switches (Active-HIGH and Active-LOW):Active-High Buffer (IF1 regulates BUF:BUFIF1), Active-High Inverted Buffer (IF1 regulates NOT:NOTIF1), Active-Low Buffer (IF0 regulates BUF:BUFIF0) and Active-Low Inverted Buffer (IF0 regulates NOT:NOTIF0). In a biological context, BUFIF1 shows logic similarity with a conventional AND gate, while NOTIF0 is logically similar to a conventional NOR gate. Likewise, NOTIF1 and BUFIF0 show typical gene expression signatures of both variants of NIMPLY (AND NOT) gates, e.g., as illustrated in FIG. 27B.

In one embodiment, the present invention provides a half-adder and half-subtractor gene circuit. A half-adder returns the digits sum S (representative for the 2⁰ digit) and carry Y (representative for the 2¹ digit) through binary addition of the two inputs A and B. Likewise, a half-subtractor performs binary subtraction of B from A using two different output signals for borrow W (representative for the −1×2¹ digit) and difference D (representative for the 2⁰ digit). In one embodiment, a half-adder is produced through addition of the grazoprevir- and vanillic acid-regulated BUF1IF0, NOT1IF1 and BUF2IF1 tristate buffers, e.g., as illustrated in FIG. 30A. In another embodiment, a half-subtractor is assembled through the three tristate buffers BUF1IF0, NOT1IF1 and NOT2IF1 (e.g., as illustrated in FIG. 30B).

In a further embodiment, the present invention can provide different biocomputational calculation modules, for example, as listed in the following table:

| Module Name | Related COMPONENT (SEQ ID NO)* |
|---|---|
| BUF1-switch | MCP-(NS3a(H1))₃ (SEQ-ID No. 58), and |
| | (GNCR)₃-NSP3 (SEQ-ID No. 30), and |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| NOT1-switch | MCP-(NS3a(H1))₃ (SEQ-ID No. 58), and |
| | (ANR)₈-NSP3 (SEQ-ID No. 5) |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| BUF2-switch | GEMS_{NS3aH1}(IL6RB) (SEQ-ID No. 192), and. |
| | GEMS_{GNCR}(IL6RB) (SEQ-ID No. 191), and |
| | (O_{STAT3})₈-P_{hCMVmin}-SEAP (SEQ-ID No. 169), or |
| | (O_{STAT3})₈-P_{hCMVmin}-NanoLuc (SEQ-ID No. 164) |
| NOT2-switch | GEMS_{NS3aH1}(IL6RB) (SEQ-ID No. 192), and |
| | GEMS_{8xANR}(IL6RB) (SEQ-ID No. 188), and |
| | (O_{STAT3})₈-P_{hCMVmin}-SEAP (SEQ-ID No. 169), or |
| | (O_{STAT3})₈-P_{hCMVmin}-NanoLuc (SEQ-ID No. 164) |
| BUF3-switch | NLS-PcaV-StaPL-VP64 (SEQ-ID No. 198), and |
| | (O)_{PcaV})₅-P_{hCMVmin}-SEAP (SEQ-ID No. 165) |
| NOT3-switch | NLS-PcaV-StaPL-KRAB (SEQ-ID No. 197), and |
| | (O_{PcaV})₅-P_{SV40}-SEAP (SEQ-ID No. 166) |
| IF0-switch | VanR-VP64 (SEQ-ID No. 296), and |
| | (VanO)₅-P_{hCMVmin}-SEAP (SEQ-ID No. 171), or |
| | (VanO)₅-P_{hCMVmin}-NanoLuc (SEQ-ID No. 170) |
| IF1-switch | MOR9-1 (SEQ-ID No. 208), and |
| | P_{CRE}-SEAP (SEQ-ID No. 172) |
| BUF₁IF1 (AND-gate) | MOR9-1 (SEQ-ID No. 208), and |
| | MCP-(NS3a(H1))₃ (SEQ-ID No. 58), and |
| | P_{CRE}-(GNER)₃-NSP3 (SEQ-ID No. 162), and |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| NOT₁IF1 | MOR9-1 (SEQ-ID No. 208), and |
| (VA AND NOT Gra) | MCP-(NS3a(H1))₃ (SEQ-ID No. 58), and |
| | P_{CRE}-(ANR)₄-NSP3 (SEQ-ID No. 160), and |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| BUF₁IF0 | VanR-VP64 (SEQ-ID No. 296), and |
| (Gra AND NOT VA) | (VanO)₅-P_{hCMVmin}-(GNCR)₃-NSP3 (SEQ-ID No. 159), and |
| | MCP-(NS3a(H1))₃ (SEQ-ID No. 58), and |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| NOT₁IF0 (NOR-gate) | VanR-VP64 (SEQ-ID No. 296), and |
| | (VanO)₅-Pₘᵢₙ-(ANR)₄-NSP3 (SEQ-ID No. 161), and |
| | MCP-(NS3a(H1))₃ (SEQ-ID No. 58), and |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| BUF₂IF1 (AND-gate) | MOR9-1 (SEQ-ID No. 208), and |
| | P_{CRE}-GEMS_{GNCR} (SEQ-ID No. 158), and |
| | P_{CRE}-GEMS_{NS3a(H1)} (SEQ-ID No. 157), and |
| | (O_{STAT3})₈-P_{hCMVmin}-SEAP (SEQ-ID No. 169), or |
| | (O_{STAT3})₈-P_{hCMVmin}-NanoLuc (SEQ-ID No. 164) |
| NOT₂IF1 | MOR9-1 (SEQ-ID No. 208), and |
| (VA AND NOT Gra) | P_{CRE}-GEMS_{ANR} (SEQ-ID No. 154), and |
| | P_{CRE}-GEMS_{NS3a(H1)} (SEQ-ID No. 157), and |
| | (O_{STAT3})₈-P_{hCMVmin}-SEAP (SEQ-ID No. 169), or |
| | (O_{STAT3})₈-P_{hCMVmin}-NanoLuc (SEQ-ID No. 164) |
| BUF₂IF0 | VanR-VP64 (SEQ-ID No. 296), and |
| (Gra AND NOT VA) | (VanO)₅-P_{hCMVmin}-GEMS_{NS3a(H1)} (SEQ-ID No. 156), and |
| | (VanO)₅-P_{hCMVmin}-GEMS_{GNCR} (SEQ-ID No. 313), and |
| | (O_{STAT3})₈-P_{hCMVmin}-SEAP (SEQ-ID No. 169), or |
| | (O_{STAT3})₈-P_{hCMVmin}-NanoLuc (SEQ-ID No. 164) |
| NOT₂IF0 (NOR-gate) | VanR-VP64 (SEQ-ID No. 296), and |
| | (VanO)₅-P_{hCMVmin}-GEMS_{NS3a(H1)} (SEQ-ID No. 156), and |
| | (VanO)₅-P_{hCMVmin}-GEMS_{ANR} (SEQ-ID No. 155), and |
| | (O_{STAT3})₈-P_{hCMVmin}-SEAP (SEQ-ID No. 169), or |
| | (O_{STAT3})₈-P_{hCMVmin}-NanoLuc (SEQ-ID No. 164) |
| OR-Gate | MOR9-1 (SEQ-ID No. 208), and |
| | P_{CRE}-(ANR)₈-NSP3 (SEQ-ID No. 153), and |
| | (GNCR)₃-NSP3 (SEQ-ID No. 30), and. |
| | MCP-(NS3a(H1))₃ (SEQ-ID No. 58), and |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| XOR-Gate | MOR9-1 (SEQ-ID No. 208), and |
| | P_{CRE}-(ANR)₄-NSP3 (SEQ-ID No. 160), and |
| | VanR-VP64 (SEQ-ID No. 296), and |
| | (VanO)₅-P_{hCMVmin}-(GNCR)₃-NSP3 (SEQ-ID No. 159), and |
| | MCP-(NS3a(H1))₃ (SEQ-ID No. 58), and |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| XNOR-Gate | MOR9-1 (SEQ-ID No. 208), and |
| | P_{CRE}-(GNCR)₃-NSP3 (SEQ-ID No. 162), and |
| | VanR-VP64 (SEQ-ID No. 296), and |
| | (VanO)₅-Pₘᵢₙ-(ANR)₈-NSP3 (SEQ-ID No. 151), and |
| | MCP-(NS3a(H1))₃ (SEQ-ID No. 58), and |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| NAND-Gate | VanR-VP64 (SEQ-ID No. 296), and |
| | (VanO)₅-Pₘᵢₙ-(GNCR)₃-NSP3 (SEQ-ID No. 152), and. |
| | (ANR)₈-NSP3 (SEQ-ID No. 5), |
| | MCP-(NS3a(H1))₃ (SEQ-ID No. 58), and |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| VA IMPLY Gra Gate | VanR-VP64 (SEQ-ID No. 296), and |
| | (VanO)₅-Pₘᵢₙ-(ANR)₈-NSP3 (SEQ-ID No. 151), and |
| | (GNCR)₃-NSP3 (SEQ-ID No. 30), and |
| | MCP-(NS3a(H1))₃ (SEQ-ID No. 58), and |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| Gra IMPLY VA Gate | MOR9-1 (SEQ-ID No. 208), and |
| | P_{CR}_{E}-(GNCR)₃-NSP3 (SEQ-ID No. 162), and |
| | (ANR)₈-NSP3 (SEQ-ID No. 5), |
| | MCP-(NS3a(H1))₃ (SEQ-ID No. 58), and |
| | 5′-UTR-SEAP-(MS2-Box)₂₄-HHR (SEQ-ID No. 137), or |
| | 5′-UTR-NanoLuc-(MS2-Box)₂₄-HHR (SEQ-ID No. 163), or |
| | 5′-UTR-EGFP-(MS2-Box)₂₄-HHR (SEQ-ID No. 149), or |
| | 5′-UTR-NanoLuc-P2A-mCherry-(MS2-Box)₂₄-HHR (SEQ-ID No. 139) |
| Half-Adder | MOR9-1 (SEQ-ID No. 208), and |
| (BUF1&BUF3) | P_{CRE}-(ANR)₄-NSP3 (SEQ-ID No. 160), and |
| | VanR-VP64 (SEQ-ID No. 296), and |
| | (VanO)₅-P_{hCMVmin}-(GNCR)₃-NSP3 (SEQ-ID No. 159), and |
| | MCP-(NS3a(H1))₃ (SEQ-ID No. 58), and |
| | 5′-UTR-EGFP-(MS2-Box)₂₄-HHR (SEQ-ID No. 149), and. |
| | P_{CRE}-NLS-PcaV-StaPL-VP64 (SEQ-ID No. 130), and |
| | (O_{PcaV})₅-P_{hCMVmin}-mCherry (SEQ-ID No. 141) |
| Half-Adder | MOR9-1 (SEQ-ID No. 208), and |
| (BUF1&BUF2) | P_{CRE}-(ANR)₄-NSP3 (SEQ-ID No. 160), and |
| | VanR-VP64 (SEQ-ID No. 296), and |
| | (VanO)₅-Pₘᵢₙ-(GNCR)₃-NSP3 (SEQ-ID No. 152), and |
| | MCP-(NS3a(H1))₃ (SEQ-ID No. 58), and |
| | 5′-UTR-EGFP-(MS2-Box)₂₄-HHR (SEQ-ID No. 149), and |
| | P_{CRE}-GEMS_{GNCR} (SEQ-ID No. 158), and |
| | P_{CRE}-GEMS_{NS3a(H1)} (SEQ-ID No. 157), and |
| | (O_{STAT3})₈-P_{hCMVmin}-mCherry (SEQ-ID No. 150) |
| Half-Subtractor | MOR9-1 (SEQ-ID No. 208, and |
| [symbol] | [symbol] |

[symbol]

indicates data missing or illegible when filed

“Related COMPONENT (SEQ ID NO)” means that the component comprises or consists of the SEQ ID NO, or comprises or consists of the amino acid sequence having at least 90%, 91%, 92%, 93%, 94%, 95%, 96%, 97%, 98% or 99% identity with SEQ ID NO. Distinctive components of each module is shown in bold letters.

In each module, the target protein (the mRNA construct comprises its mRNA encoding the target protein), such as SEAP, or NanoLuc, EGFP, or NanoLuc-P2A-mCherry can be substituted by other target protein, such as those illustrated in the present invention.

### (VI-III) for Diagnosis

In a further aspect, the present invention relates to the gene regulation system of the present invention, for use as a diagnosing kit, e.g., for medical diagnosis ex vivo or real-time monitoring of cellular process in vivo. In one embodiment, the present invention relates to a medical diagnostics or a real-time monitoring of cellular process, comprising diagnosis or monitoring with the gene regulation system. In another embodiment, the present invention relates to a use of the gene regulation system in the manufacture of a diagnosing kit, e.g., medical diagnosis or real-time monitoring of cellular process. In particular embodiments, the detection can be performed in vitro or in vivo.

In some embodiment, the system can be used for detecting disease metabolites, virulence factors or environmental pollutants in vitro or in vivo.

The STIF-based translational regulation strategy and the system of the present invention can also be repurposed to engineer intracellular protein sensors. For example, when each split-STIF component is fused to a different member of a protein heterotrimer system, STIF-dependent gene expression from poly (A)-deficient mRNA would strictly depend on the presence of the remaining member(s) of the full protein complex, as illustrated by FIG. 34.

In one embodiment, the STIF comprises or consists of two recombinant fusion proteins A and B, wherein protein A can be RBP-Y or Y-RBP and protein B can be Y′-eIFBP or eIFBP-Y′, or wherein protein A can be eIFBP-Y or Y-eIFBP and protein B can be Y′-RBP or RBP-Y′, wherein Y and Y′ can interact with each other via another protein Y″. That is, only when Y″ exists, Y and Y′ can associate with each other as target protein Y″ triggers a Y:Y′:Y″-interaction.

Specifically, the Y and Y′ in the system can be fused either to the N-terminus or C-terminus of RBP or eIFBP domains of STIF regulators, allowing Y″ to initiate translational initiation upon triggering the circularized configuration of mRNA that contain RBP-specific poly (A)-surrogate, e.g., as illustrated in FIG. 34. To sense a specific target Y″, a pair of proteins Y and Y′ both binding to different epitopes of Y″ can be fused either to the N-terminus or C-terminus of RBP- or eIFBP-domains of STIF regulators, rendering the formation of a circularized mRNA configuration and translational initiation exclusively dependent on the presence of intracellular Y″. Alternatively, RBP-specific 5′-cap and/or poly (A)-surrogates could be replaced by aptamer motifs directly binding to Y″ to form a circularized mRNA configuration in combination with Y or Y′ fused to eIFBPs.

In some embodiment, Y″ can be any disease signature, such as disease metabolites, virulence factors, or any other substance that need to be detected, such as environmental pollutants.

In one embodiment, the STIF-based system of the present invention can be used as a sensor for real-time detection and treatment of cells harboring gene fusions in vivo.

In one embodiment, the present invention provides method to detect any pathogens such as virus Hepatitis C virus (HCV) (e.g., by detecting the virulence factor specific to the virus) either in cells, or in cell-free context or in a subject, by a system of the present invention.

In one embodiment, Y or Y′ is any protein or fragment that can bind to the Y″.

In some embodiment, Y″ can be any protein or agent as long as it can be bound by two different proteins, preferably in at different domains or different epitopes of Y″. In some particular embodiment, protein Y″ is selected from a disease-specific cellular signature, such as an oncoprotein, e.g., a fusion gene product or protein complex that is specifically expressed in a tumor cell or tumor tissue, a virulence factor, e.g. HCV or HCV specific proteins (e.g., NS3 protein) or any other substance that need to be detected, such as environmental pollutants.

Particularly, the system comprises
(i) Synthetic translation initiation Factors (STIFs) and mRNA construct that comprises the mRNA encoding the target protein; or
(ii) A nucleic acid that encodes STIF and a nucleic acid that encodes the mRNA

construct that comprises the mRNA encoding the target protein;

wherein the STIFs comprise or consist of two recombinant fusion proteins A and B, wherein protein A can be RBP-Y or Y-RBP and protein B can be Y′-eIFBP or eIFBP-Y′, or wherein protein A can be eIFBP-Y or Y-eIFBP and protein B can be Y′-RBP or RBP-Y′, wherein Y and Y′ can interact with each other via another protein Y″. That is, only when Y″ exists, Y and Y′ can associate with each other as target protein Y″ triggers a Y:Y′:Y″-interaction, wherein Y″ is selected from a disease-specific cellular signature, such as a fusion gene product, an oncoprotein, a virulence factor, an RNA-binding protein, or any other substance that need to be detected, such as environmental pollutants or any other intracellular or secreted protein containing one or multiple domains, and wherein the target protein contains at least a reporter proteins such as a fluorescent protein e.g. EGFP or mCherry or any secreted or intracellular protein that can be detected with its expression, e.g. SEAP or luciferase.

In one embodiment, the RBP is L7Ae or MCP.

In some embodiments, protein A or B can comprise multiple tandem repeats of Y or Y′, for example, 1-5 repeats, e.g., 1, 2, 3, 4, or 5 repeats. In some particular embodiments, Y and Y′ are two different nanobodies that bind specifically to protein Y″, e.g., at different domains or different epitopes of Y″. In some particular embodiments, protein Y and protein Y′ are two different affibodies that bind specifically to protein Y″, e.g., at different domains or different epitopes of Y″. In some particular embodiments, protein Y and protein Y′ are two different monobodies that bind specifically to protein Y″, e.g., at different domains or different epitopes of Y″. In some particular embodiments, protein Y and protein Y′ are two different DARPins that bind specifically to protein Y″, e.g., at different domains or different epitopes of Y″.

In one embodiment, Y or Y′ is an antibody or antigen binding fragment (e.g., scFv) specifically binding to NS3 as protein Y″, e.g., scF35 or scFv162.

In a specific embodiment, protein A comprises amino acid of SEQ ID NO: 147, or comprising amino acid sequence having at least 90%, 91%, 92%, 93%, 94%, 95%, 96%, 97%, 98%, or 99% identity with SEQ ID NO: 47, or consists of said amino acid sequence. In a specific embodiment, protein B comprises amino acid of SEQ ID NO: 70, 261 or 262, or comprising amino acid sequence having at least 90%, 91%, 92%, 93%, 94%, 95%, 96%, 97%, 98%, or 99% identity with SEQ ID NO: 70, 261 or 262, or consists of said amino acid sequence.

In another embodiment, the present invention provides method to diagnose the chronic myelogenous leukemia (CML), especially at early stage, by a system of the present invention, comprising detecting the BCR-ABL, a representative cytosolic biomarker of chronic myelogenous leukemia (CML).

Particularly, the system comprises
(i) Synthetic translation initiation Factors (STIFs) and mRNA construct that comprises the mRNA encoding the target protein; or
(ii) A nucleic acid that encodes STIF and a nucleic acid that encodes the mRNA construct that comprises the mRNA encoding the target protein;
wherein the STIFs comprise or consist of two recombinant fusion proteins A and B, wherein protein A can be RBP-Y or Y-RBP and protein B can be Y′-eIFBP or eIFBP-Y′, or wherein protein A can be eIFBP-Y or Y-eIFBP and protein B can be Y′-RBP or RBP-Y′, wherein Y and Y′ can interact with each other via another protein Y″. That is, only when Y″ exists, Y and Y′ can associate with each other as target protein Y″ triggers a Y:Y′:Y″-interaction, wherein Y″ is selected from a disease-specific cellular signature, such as a fusion gene product, an oncoprotein, a virulence factor, an RNA-binding protein, or any other substance that need to be detected, such as environmental pollutants or any other intracellular or secreted protein containing one or multiple domains, wherein the target protein contains at least a reporter proteins such as a fluorescent protein e.g. EGFP or mCherry or any secreted or intracellular protein that can be detected with its expression, e.g. SEAP or luciferase.

In one embodiment, the RBP is L7Ae or MCP.

In some embodiments, protein A or B can comprise multiple tandem repeats of Y or Y′, for example, 1-5 repeats, e.g., 1, 2, 3, 4, or 5 repeats. In some particular embodiments, Y and Y′ are two different nanobodies that bind specifically to protein Y″, e.g., at different domains or different epitopes of Y″. In some particular embodiments, protein Y and protein Y′ are two different affibodies that bind specifically to protein Y″, e.g., at different domains or different epitopes of Y″. In some particular embodiments, protein Y and protein Y′ are two different monobodies that bind specifically to protein Y″, e.g., at different domains or different epitopes of Y″. In some particular embodiments, protein Y and protein Y′ are two different DARPins that bind specifically to protein Y″, e.g., at different domains or different epitopes of Y″.

In one embodiment, Y or Y′ are proteins specifically binding to BCR or ABL, respectively. Thus, proteins A and B can specifically bind to the fusion gene product BCR-ABL as protein Y″. For example, the protein specifically binding to ABL is an ABL-specific intrabody, e.g., ABI (iDab), which comprises amino acid of SEQ ID NO: 223, or comprising amino acid sequence having at least 90%, 91%, 92%, 93%, 94%, 95%, 96%, 97%, 98%, or 99% identity with SEQ ID NO: 223, or consists of said amino acid sequence. For example, the protein specifically binding to BCR is a BCR-specific coiled-coil domain, e.g., CCmut3, which comprises amino acid of SEQ ID NO: 222, or comprising amino acid sequence having at least 90%, 91%, 92%, 93%, 94%, 95%, 96%, 97%, 98%, or 99% identity with SEQ ID NO: 222, or consists of said amino acid sequence.

In a specific embodiment, protein A comprises amino acid of SEQ ID NO: 181 or 202, or comprising amino acid sequence having at least 90%, 91%, 92%, 93%, 94%, 95%, 96%, 97%, 98%, or 99% identity with SEQ ID NO: 181 or 202, or consists of said amino acid sequence. In a specific embodiment, protein B comprises amino acid of SEQ ID NO: 201 or 258, or comprising amino acid sequence having at least 90%, 91%, 92%, 93%, 94%, 95%, 96%, 97%, 98%, or 99% identity with SEQ ID NO: 201 or 258, or consists of said amino acid sequence.

In one embodiment, the present invention relates to a kit comprising the system of the present invention, which can be use for diagnosis or detecting protein, e.g., protein bound by Y and Y′ of the STIF.

In a particular embodiment, the system can be applied on abiotic materials such as paper discs, for detection or diagnosis in vitro.

In a further embodiment, the present invention relates to a method of treating disease of the present invention, including first diagnosing the disease in vivo, e.g., by the system of the present invention, and then treating the disease by the system of the invention, such as those described in the present invention.

The following examples further illustrate the invention. However, it is to be understood that the examples are described by way of illustration and not limitation, and various modifications may be made by those skilled in the art.

All embodiments of any aspect of the invention can be used in combination, unless the context clearly dictates otherwise.

### DESCRIPTION OF THE DRAWINGS

FIG. 1. Working scheme of synthetic translation initiation factor (STIF)-dependent gene expression control. Translation of reporter mRNA with genetically modified 3′-UTR containing an RNA-binding protein (RBP)-specific aptamer region and an shRNA- or hammerhead ribozyme (HHR)-mediated cleavage site for preprogrammed poly (A) removal depends on the presence of STIFs consisting of different RBPs fused to different eIF4F-binding proteins (eIFBP). Because STIFs are designed to mimic the role of natural poly (A)-binding protein (PABP) in simultaneously binding target mRNA and one member of the eIF4F complex (i.e., eIF4G, eIF4E, eIF4A, eIF4B, etc.) to form a “closed-loop” mRNA configuration and activate translation, poly (A) removal is important to avoid putative crosstalk with endogenous PABP-driven processes.

FIG. 2. Design and validation of different STIF constructs consisting of RNA-binding protein (RBPs) fused to different eIF4F-binding moieties (eIFBP). (A) Synthetic translation initiation factors (STIFs)-mediated translation of L7Ae-specific SEAP-mRNA with shRNA-216-cleavable poly (A). HEK-293 cells were co-transfected with a SEAP expression vector containing 8 tandem L7Ae-specific C/D-box repeats (pSL31), an shRNA-216 expression vector (pSL4) and expression vectors for different STIF variants (PABP-L7Ae, pLZ16; eIF4G-L7Ae, pWH127; L7Ae-eIF4E, pDJ55; L7Ae-NSP3, pLZ27; L7Ae-VPg, pLZ248) or an L7Ae-Coh2 protein incapable of translational initiation (pSL44, negative control). SEAP levels in culture supernatants were quantified at 48 h post transfection. Data are shown as the mean±SD of n=4 independent experiments. (B) STIF-mediated translation of MCP-specific SEAP-mRNA with shRNA-216-cleavable poly (A). HEK-293 cells were co-transfected with a SEAP expression vector containing 8 tandem MCP-specific MS2-box repeats (pSL1331), an shRNA-216 expression vector (pSL4) and expression vectors for different STIF variants (PABP-MCP, pSL1315; eIF4G-MCP, pSL154; MCP-eIF4E, pSL1316; MCP-NSP3, pSL95; MCP-VPg, pLYL47) or an MCP-Coh2 protein incapable of translational initiation (pSL674, negative control). SEAP levels in culture supernatants were quantified at 48 h post-transfection. Data is the mean±SD of n=4 independent experiments. (C) Correlation between C/D-box-based aptamer region size and STIF-mediated SEAP expression. HEK-293 cells were co-transfected with SEAP expression vectors containing different tandem repeats of the L7Ae-specific C/D-box aptamer ((C/D-box)₈, pSL31; (C/D-box)₁₂, pSL81; (C/D-box)₁₆, pSL80; (C/D-box)₂₄, pSL88), an shRNA-216 expression vector (pSL4) and an expression vector for either PABP-L7Ae (ON; pLZ16) or a Coh2-L7Ae protein incapable of binding eIF4F (OFF; pSL44). SEAP expression in culture supernatants was scored at 48 h post transfection. Data presented are mean±SD, n=4 individual experiments. (D) Correlation between MS2-box-based aptamer region size and STIF-mediated SEAP expression. HEK-293 cells were transfected with SEAP expression vectors containing different tandem repeats of the MCP-specific MS2-box aptamer ((MS2-box)₈, pSL515; (MS2-box)₁₂, pSL1284; (MS2-box)₁₆, pSL516; (MS2-box)₂₄, pSL468) and constitutive expression vectors for either MCP-NSP3 (ON; pSL95) or an MCP-Coh2 protein incapable of binding eIF4F (OFF; pSL674). SEAP expression in culture supernatants was scored at 48 h post-transfection. Data presented are mean±SD, n=4 individual experiments.

FIG. 3. Characterization of stability and non-specific binding of STIF-specific target mRNA. (A) Quantification of the binding capability of PABP to poly (A)-containing RNA by RIP-qPCR. HEK-293 cells were transfected with a constitutive expression vector for 3×FLAG-tagged PABP-L7Ae (pSL763) reflecting the RNA binding capacity of endogenous PABP. After 24 h, 10 μg of in vitro-transcribed EGFP mRNA with (+) or without (−) a poly (A) tail was added. At 3 h after transfection, RNA was extracted and co-immunoprecipitated using anti-Flag affinity gel. Data show the results of qRT-PCR analysis as the ratio (%) of EGFP-mRNA in samples before (input) and after immunoprecipitation (IP) (±SD, n=3). (B) Impact of engineered L7Ae-specific poly (A) surrogates on mRNA stability. SEAP-mRNA in vitro-transcribed to contain poly (A) (from pSL1091), no poly (A) (from pSL517) or poly (A)-surrogates consisting of 8 (from pSL31) or 24 tandem C/D-box repeats in the 3′-UTR (from pSL355) was transfected into HEK-293 cells expressing Coh2-L7Ae (24 h after transfection of pSL44) or not expressing Coh2-L7Ae (24 h after transfection of pcDNA3.1 (+)). SEAP-mRNA levels were analyzed by qRT-PCR at 4 (*arbitrarily set as 100%), 8, 12 and 24 hours after mRNA transfection. Data presented are mean±SD, n=4 independent experiments. (C) Impact of engineered MCP-specific poly (A) surrogates on mRNA stability. (Left) SEAP-mRNA in vitro-transcribed to contain poly (A) (from pSL1091), no poly (A) (from pSL517), or poly (A)-surrogates consisting of 8 (from pSL515) or 24 tandem MS2-box repeats in the 3′-UTR (from pSL468) were transfected into HEK-293 cells expressing MCP-Coh2 (24 h after transfection of pSL674) or not expressing MCP-Coh2 (24 h after transfection of pcDNA3.1 (+)). SEAP-mRNA levels were analyzed with RT-PCR at 4 (*arbitrarily set as 100%), 8, 12, and 24 hours after mRNA transfection. Data presented are mean±SD, n=4 independent experiments. (Right) HEK-293 cells were co-transfected with a SEAP expression vector containing 24 MS2-box repeats in the 3′-UTR (reporter; pSL468) and an MCP-Coh2 protein incapable of translational initiation (pSL674). SEAP levels in culture supernatants and relative mRNA expression levels in cells were quantified at 48 h post-transfection. Transcript levels of SEAP were normalized to glyceraldehyde 3-phosphate dehydrogenase (GAPDH) expression by setting undetermined values to a maximum Ct of 40 cycles. Data presented are mean±SD, n=3. (D) mRNA stabilization through ABP/aptamer binding. HEK-293 cells were co-transfected with a SEAP expression vector (reporter; pSL88) and constitutive expression vectors for shRNA-216 (pSL4) and a Coh2-L7Ae protein incapable of translational initiation (pSL44). SEAP levels in culture supernatants (left) and mRNA expression levels in cells (right) were quantified at 48 h post-transfection. Transcript levels of SEAP were normalized to glyceraldehyde 3-phosphate dehydrogenase (GAPDH) expression by setting undetermined values to a maximum Ct of 40 cycles. Data presented are mean±SD, n=3.

FIG. 4. cis- and trans-removal strategies for pre-programmed poly (A)-cleavage. (A) Validation of mRNA knockdown efficiency of shRNA-216. HEK-293 cells were co-transfected with an expression vector for shRNA-216-repressible SEAP-mRNA (pSL31) and different amounts of a Pₕᵤ₆-driven shRNA-216 expression vector (pSL4). SEAP expression in the culture supernatant was scored at 48 h after transfection. Data presented are mean±SD of n=4 individual experiments. (B) Quantification of HHR-mediated mRNA self-cleavage. HEK-293 cells were co-transfected with SEAP expression vectors containing zero (pSL517), one (pSLM97), two (pSL767) or four HHR tandem repeats (pSL768) before SEAP levels in culture supernatants were scored at 48 h post transfection. Data presented are mean±SD of n=4 individual experiments.

FIG. 5. Translational regulation of SEAP-, Firefly Luciferase and EGFP-mRNA with self-cleavable poly (A). (A) STIF-mediated translation of L7Ae-specific SEAP-mRNA with HHR-cleavable poly (A). HEK-293 cells were co-transfected with a SEAP expression vector containing 24 tandem L7Ae-specific C/D-box repeats (pSL355) and expression vectors for different STIF variants (PABP-L7Ae, pLZ16; eIF4G-L7Ae, pWH127; L7Ae-eIF4E, pDJ55; L7Ae-NSP3, pLZ27; L7Ae-VPg, pLZ248) or an L7Ae-Coh2 protein incapable of translational initiation (pSL44, negative control). SEAP levels in culture supernatants were quantified at 48 h post transfection. Data are shown as the mean±SD of n=4 independent experiments. (B) STIF-mediated translation of MCP-specific SEAP-mRNA with HHR-cleavable poly (A). HEK-293 cells were co-transfected with a SEAP expression vector containing 24 tandem MCP-specific MS2-box repeats (pSL468) and expression vectors for different STIF variants (PABP-MCP, pSL1315; eIF4G-MCP, pSL154; MCP-eIF4E, pSL1316; MCP-NSP3, pSL95; MCP-VPg, pLYL47) or an MCP-Coh2 protein incapable of translational initiation (pSL674, negative control). SEAP levels in culture supernatants were quantified at 48 h post transfection. Data are shown as the mean±SD of n=4 independent experiments. (C, D) STIF-mediated regulation at the protein level of gene expression. HEK-293 cells were co-transfected with an expression vector for FLAG-tagged Firefly Luciferase (FLuc) containing MCP-specific poly (A) surrogates in the 3′-UTR (pSL781) and constitutive expression vectors for MCP-Coh2 (pSL674) or MCP-NSP3 (pSL95). At 48 h post-transfection, (C) Luciferase activity and (D) absolute protein levels of FLuc were detected using Western Blot. Data in (C) are shown as the mean±SD, n=4 independent experiments. (E, F) STIF-mediated translational regulation of EGFP-mRNA. HEK-293 cells were co-transfected with an expression vector for EGFP-mRNA containing 24 tandem MS2-box repeats in the 3′-UTR (pSL1308) and constitutive expression vectors for MCP-Coh2 (pSL674) or MCP-NSP3 (pSL95). At 48 h post-transfection, EGFP expression was scored by (E) fluorescence microscopy (scale bar: 10 μm) or (F) flow cytometry. Data are mean±SD, n=3.

FIG. 6. Characterization of endogenous RNA-binding and eIF4F-association of overexpressed STIFs. (A) STIFs associate with the endogenous eIF4F complex. HEK-293 cells were transfected with expression vectors for 3×FLAG-tagged MCP (−, pSL1084) or MCP-NSP3 (+, pSL1083) before one lysate fraction was immunoprecipitated at 48 h post transfection. Target proteins in lysate fractions before (input) and after immunoprecipitation (Flag-IP) were detected with anti-FLAG, anti-eIF4G and anti-eIF4E antibodies. Numbers on the left axis of Western blots represent molecular weights (MW) of target proteins. (B) Quantification of endogenous RNA binding capacity of PABP- and NSP3-fusion proteins by RIP-qPCR. At 48 h after transfection of HEK-293 cells with expression vectors for 3×FLAG-tagged L7Ae-NSP3 (pSL762) or PABP-L7Ae (pSL763), RNA was extracted and co-immunoprecipitated using anti-Flag affinity gel. Data show the results of qRT-PCR analysis as the ratio (%) of GAPDH RNA in samples before (input) and after immunoprecipitation (IP), taken as a measure of the amount of RNA bound on PABP- and NSP3-containing constructs (±SD, n=3). Bars represent the mean and SD, and filled circles show individual results. ***, p<0.001.

FIG. 7. Translational regulation by protein-protein interaction (PPI)-mediated STIF reconstitution. Bipartite STIF systems with NSP3 and RBP domains split into two independent proteins allow flexible incorporation of different PPI systems to regulate conditional STIF assembly and translation of RBP-specific mRNA.

FIG. 8. Translational regulation by spontaneous assembly of bipartite STIFs. (A) Translational regulation through constitutive STIF reconstitution. HEK-293 cells were co-transfected with plasmids encoding SEAP mRNA containing an L7Ae-specific poly (A)-surrogate (pSL355) and constitutive expression vectors for different combinations of L7Ae- and NSP3-fusion proteins (L7Ae-NS3a (H1) &ANR₄-NSP3: pSL703/pSL549; L7Ae-NS3a&ANR₄-NSP3: pYF5/pSL549; L7Ae-LD1&Bcl-XL-NSP3: pSL667/pSL615; L7Ae-LD3&Bcl-XL-NSP3: pSL661/pSL615; L7Ae-Coh2&DocS-NSP3: pSL65/pSL66). Transfection of pcDNA3.1 (+) instead of NSP3-fusion proteins was used as a negative control. SEAP levels in culture supernatants were quantified at 48 h post-transfection. Data are shown as the mean±SD of n=4 independent experiments. (B) Compatibility of STIF-mediated translational regulation with split-complementation approaches. (left) HEK-293 cells were co-transfected with a SEAP expression vector containing 24 tandem MCP-specific aptamer repeats (pSL468) and different combinations of constitutive expression vectors for MCP-NSP3 (pSL674) and (DocS)₃-NSP3 (pSL86). (right) HEK-293 cells were co-transfected with a SEAP expression vector containing bacteriophage N-Peptide (λ-N)-repressible HHR placed upstream of poly (A) (pMX116) and constitutive expression vectors for λN-Coh2 (pSL334) and DocS-mCherry (pSL1099). For (−) conditions, pcDNA3.1 (+) was transfected instead of expression vectors. SEAP levels in culture supernatants were scored at 48 h after transfection. Data are shown as the mean±SD, n=4 independent experiments.

FIG. 9. Translational regulation through constitutive Coh2/DocS-mediated STIF reconstitution. (A) Site-specific translational activation by eIFBP overexpression. HEK-293 cells were (co-) transfected with an expression vector for SEAP-mRNA containing 24 tandem C/D-box-(left panel, pSL355) or MS2-box repeats in the 3′-UTR (right panel, pSL468), constitutive expression vectors for L7Ae (left) or MCP (right) fused to Coh2 (L7Ae-Coh2, pSL83; MCP-Coh2, pSL674) or EGFP (L7Ae-EGFP, pSL1078; MCP-EGFP, pSL435) and constitutive expression vectors for various chimeric Coh2-specific DocS-containing eIFBP fusions (PABP-DocS, pSL47; DocS-eIF4G, pSL87; DocS-eIF4E, pLZ312; DocS-NSP3, pSL66; DocS-VPg, pLZ311). Transfection of pcDNA3.1 (+) instead of DocS- or MCP-expressing vectors was used as a negative control. SEAP levels in culture supernatants were quantified at 48 h post-transfection. Data are shown as the mean±SD, n=4 independent experiments. (B, C) Natural 5′-cap-independent translation by site-specific eIFBP recruitment to the (B) 5′-UTR or (C) intergenic regions of target gene mRNA. (B) HEK-293 cells were (co-) transfected with an expression vector for SEAP mRNA containing 4 tandem C/D-box repeats in the 5′-UTR (pQZ8), a constitutive L7Ae-(Coh2)₃ expression vector (pSL83) and constitutive expression vectors for various DocS-based fusion constructs (PABP-DocS, pSL47; DocS-eIF4G, pSL87; DocS-eIF4E, pLZ312; DocS-NSP3, pSL66; DocS-VPg, pLZ311). Transfection of pcDNA3.1 (+) instead of DocS-expressing vectors was used as a negative control. SEAP levels in culture supernatants were quantified at 48 h post-transfection. Data are shown as the mean±SD, n=4 independent experiments. (C) HEK-293 cells were (co-) transfected with reporter mRNA containing 24 tandem C/D-box repeats placed downstream of SEAP- and upstream of NanoLuc-coding regions (pPW21), a constitutive L7Ae-(Coh2)₃ expression vector (pSL83) and constitutive expression vectors for various DocS-based fusion constructs (PABP-DocS, pSL47; DocS-eIF4G, pSL87; DocS-eIF4E, pLZ312; DocS-NSP3, pSL66; DocS-VPg, pLZ311). Transfection of pcDNA3.1 (+) instead of DocS-expressing vectors was used as a negative control. SEAP (left) and NanoLuc levels in culture supernatants (right) were quantified at 48 h post-transfection. Data are shown as the mean±SD, n=4 independent experiments.

FIG. 10. Engineering of trigger-inducible gene switches using bipartite STIFs. (A) Translational regulation of L7Ae-specific mRNA through trigger-inducible STIF reconstitution. For danoprevir-inducible SEAP translation, HEK-293 cells were co-transfected with pSL355 and constitutive expression vectors for L7Ae-(NS3a)₃ (pLZ76) and (DNCR)₃-NSP3 (pLZ72). For abscisic acid-inducible SEAP translation, HEK-293 cells were co-transfected with pSL355 and constitutive expression vectors for L7Ae-(ABI)₃ (pPW3) and (PYL1)₃-NSP3 (pPW4). For gibberellic acid-inducible SEAP translation, HEK-293 cells were co-transfected with pSL355 and constitutive expression vectors for GAI-L7Ae (pPW14) and NSP3-GID1 (pPW17). For grazoprevir-inducible SEAP translation, HEK-293 cells were co-transfected with pSL355 and constitutive expression vectors for L7Ae-(NS3a)₃ (pLZ76) and (GNCR)₃-NSP3 (pLZ74). For rapamycin-inducible SEAP translation, HEK-293 cells were co-transfected with pSL355 and constitutive expression vectors for FKBP-L7Ae (pMX331) and FRB-NSP3 (pLZ42). For blue light-inducible SEAP translation, HEK-293 cells were co-transfected with pSL355 and constitutive expression vectors for L7Ae-CIB1 (pLZ55) and Cry2-NSP3 (PLZ68). SEAP levels in culture supernatants were scored at 48 h after addition of corresponding inducers (danoprevir, 1 μM; abscisic acid, 100 μM; gibberellic acid, 100 μM; grazoprevir, 0.5 μM; rapamycin, 0.01 μM) or at 24 h after exposure to blue light (450 nm; ON, 30 s at 5 mW/cm²; OFF, 30 s) or red light (660 nm, constantly 1 W/m²). Data are shown as the mean±SD, n=3. (B) Regulation of MCP-specific mRNA through trigger-inducible STIF reconstitution. For danoprevir-inducible SEAP translation, HEK-293 cells were co-transfected with a SEAP expression vector containing poly (A)-surrogates with 24 tandem MCP-specific MS2-box repeats (pSL468) and constitutive expression vectors for MCP-NS3a (pSL497) and DNCR-NSP3 (pLZ72). For abscisic acid-inducible SEAP translation, HEK-293 cells were co-transfected with pSL468 and constitutive expression vectors for ABI-MCP (pPW22) and (PYL1)₃-NSP3 (pPW4). For gibberellic acid-inducible SEAP translation, HEK-293 cells were co-transfected with pSL468 and constitutive expression vectors for MCP-GID (pPW23) and GAI-NSP3 (pPW2). For grazoprevir-inducible SEAP translation, HEK-293 cells were co-transfected with pSL468 and constitutive expression vectors for MCP-NS3a (pSL497) and GNCR-NSP3 (pYF3). For rapamycin-inducible SEAP translation, HEK-293 cells were co-transfected with a SEAP expression vector containing poly (A)-surrogates with 16 tandem MCP-specific MS2-box repeats (pSL516) and constitutive expression vectors for MCP-FRB (pSL1097) and FKBP-NSP3 (pSL1098). For blue light-inducible SEAP translation, HEK-293 cells were co-transfected with pSL516 and constitutive expression vectors for MCP-CIB1 (pSL1096) and Cry2-NSP3 (pSL71). For red light-inducible SEAP translation, HEK-293 cells were co-transfected with pSL516 and constitutive expression vectors for MCP-(Aff6V18FAN)₄ (pSL917) and DrBPhP-NSP3 (pSL901). SEAP levels in culture supernatants were scored at 48 h after addition of corresponding inducers (danoprevir, 0.5 μM; abscisic acid, 100 μM; gibberellic acid, 100 μM; grazoprevir, 0.5 μM; rapalog, 0.1 μM) or at 48 h after exposure to blue light (450 nm; ON, 30 s at 5 mW/cm²; OFF, 30 s) or at 24 h after exposure to red light (660 nm, constantly 1 W/m²). Data are shown as the mean±SD, n=4 independent experiments. (C) Light-inducible translational regulation by a blue light-activated mCherry nanobody LaM8-AK47. HEK-293 cells were transfected with an expression vector for SEAP-mRNA containing 16 tandem MS2-box repeats in the 3′-UTR (pSL516) and constitutive expression vectors for MCP-LaM8_AK47 (pSL875) and mCherry-NSP3 (pSL876). At 6 h post transfection, cells were illuminated for 48 h with blue light (450 nm, 2 mW/cm²) before SEAP levels in culture supernatants were scored. Data are mean±SD, n=4.

FIG. 11. Genetically encoded signalling-specific sensors employing phosphorylation-dependent STIF reconstitution. (A) L7Ae-based STIFs. HEK-293 cells were co-transfected with a dual reporter vector containing a constitutive FLuc expression unit and an expression unit for NanoLuc-mRNA containing L7Ae-specific poly (A)-surrogate (pSL274), an shRNA-216 expression vector (pSL4) and different combinations of constitutive expression vectors for L7Ae-(pE59)₂ (pSL169) and (ERK2)₂-NSP3 (pSL189). Luciferase levels in culture supernatants were quantified at 48 h after the addition of 100 ng/mL recombinant human EGF. For (−) conditions, pcDNA3.1 (+) was transfected instead of expression vectors. Data presented are mean±SD of relative luciferase activity (NanoLuc/FLuc), n=3 individual experiments. (B) MCP-based STIFs. HEK-293 cells were co-transfected with a constitutive FLuc expression vector (pYW99), an expression vector for NanoLuc-mRNA containing MCP-specific poly (A)-surrogates (pSL683) and different combinations of constitutive expression vectors for MCP-(pE59)₂ (pSL637) and (ERK2)₂-NSP3 (pSL189) before cultivation in cell culture medium containing 2% FBS (v/v). Luciferase levels in culture supernatants were quantified at 48 h after the addition of 0 or 100 ng/mL recombinant human EGF. For (−) conditions, pcDNA3.1 (+) was transfected instead of expression vectors. Data presented are mean±SD of relative luciferase activity (NanoLuc/FLuc), n=3 individual experiments.

FIG. 12. STIFs-mediated translational regulation by engineered 5′-cap surrogates. RNA cleavage sites can also be placed between the guanine-rich 5′-cap and the RBP-specific aptamer region to allow pre-programmed cap-removal, rendering the aptamer region a 5′-cap surrogate for recruitment of various STIF constructs described throughout this invention.

FIG. 13. Regulation of therapeutic transgene expression using the FDA-approved drug grazoprevir. Delivery of the genetic components for GNCR: NS3a-dependent STIF assembly and STIF-specific target mRNA in vivo is compatible with various administration routes of non-integrative gene therapy (e.g., DNA-encoded vectors or formulated mRNA drugs), allowing oral uptake of grazoprevir to trigger in situ production of various therapeutic proteins of interest (e.g., insulin).

FIG. 14. Optimization of an L7Ae-based grazoprevir-inducible gene switch. (A) Translational regulation by grazoprevir-dependent STIF association. HEK-293 cells were transfected with plasmids encoding SEAP-mRNA containing L7Ae-specific poly (A)-surrogate (produced by transfection of pSL88&pSL4) and different grazoprevir-regulated L7Ae- and NSP3-fusion proteins (L7Ae-GNCR&NS3a-NSP3: pYF6 and pYF1; L7Ae-NS3a&GNCR-NSP3: pYF5 and pYF3). SEAP levels in culture supernatants were scored at 48 h after addition of 0.1 μM grazoprevir dissolved in DMSO. Data are shown as the mean±SD, n=4 independent experiments. (B) Translational regulation by different grazoprevir-dependent NSP3-fusion proteins. HEK-293 cells were co-transfected with plasmids encoding SEAP-mRNA containing L7Ae-specific poly (A)-surrogate (pSL88&pSL4), L7Ae-NS3a (pYF5) and different NSP3-fusion proteins containing one (pYF3), two (pLZ73) or three N-terminal GNCR repeats (pLZ74). SEAP levels in culture supernatants were scored at 48 h after addition of 0.1 μM grazoprevir dissolved in DMSO (vehicle control). Data are shown as the mean±SD of n=4 independent experiments. (C) Translational regulation by different grazoprevir-dependent L7Ae-fusion proteins. HEK-293 cells were co-transfected with plasmids encoding SEAP-mRNA containing L7Ae-specific poly (A)-surrogate (pSL88&pSL4), GNCR-NSP3 (pYF3) and different L7Ae-fusion proteins consisting of one (pYF5), two (pLZ75) or three C-terminal NS3a repeats (pLZ76). SEAP levels in culture supernatants were scored at 48 h after addition of 0.1 μM grazoprevir dissolved in DMSO (vehicle control). Data are shown as the mean±SD of n=4 independent experiments.

FIG. 15. Optimized L7Ae- and MCP-based grazoprevir-inducible gene switch in mammalian cells. (A) Optimized grazoprevir-inducible regulation of SEAP translation. For L7Ae-based systems, HEK-293 cells were co-transfected with plasmids encoding SEAP-mRNA containing C/D-box-based poly (A)-surrogate (pSL88 & pSL4), (GNCR)₃-NSP3 (pLZ74) and L7Ae-(NS3a)₃ (pLZ76). For MCP-based systems, HEK-293 cells were co-transfected with plasmids encoding SEAP-mRNA containing an MS2-box-based poly (A)-surrogate (pSL468), (GNCR)₃-NSP3 (pSL1032) and MCP-(NS3a)₃ (pSL1042). SEAP levels in culture supernatants were scored at 48 h after addition of 0.1 μM grazoprevir dissolved in DMSO (vehicle control). Data are shown as the mean±SD of n=4 independent experiments. (B, C) Dose-dependent grazoprevir-inducible SEAP expression. (B) HEK-293 cells were co-transfected with plasmids encoding SEAP-mRNA containing L7Ae-specific poly (A)-surrogate (pSL88&pSL4) and constitutive expression vectors for (GNCR)₃-NSP3 (pLZ74) and L7Ae-(NS3a)₃ (PLZ76). SEAP levels in culture supernatants were scored at 24 h after addition of different concentrations of grazoprevir. Data are shown as the mean±SD of n=4 independent experiments. (C) HEK-293 cells were co-transfected with plasmids encoding (GNCR)₃-NSP3 (pSL1032), MCP-(NS3a)₃ (pSL1042) and SEAP mRNA containing MCP-specific poly (A)-surrogate (pSL468) before SEAP levels in culture supernatants were scored at 24 h after addition of different concentrations of grazoprevir. Data are shown as the mean±SD of n=4 independent experiments.

FIG. 16. Comparative analysis of gene switches based on STIF-based translation and state-of-the-art transcription-centered designs. (A) Kinetics of grazoprevir-inducible SEAP expression. For grazoprevir-inducible translation, HEK-293 cells were co-transfected with plasmids encoding MCP-(NS3a)₃ (pSL503) and (GNCR)₃-NSP3 (pLZ74) and SEAP-mRNA containing MCP-specific poly (A)-surrogate (pSL468). For grazoprevir-inducible transcription, HEK-293 cells were co-transfected with a TetR-specific SEAP expression vector (pLZ79) and constitutive expression vectors for TetR-NS3a (pLZ88) and (GNCR)₃-VP64 (pLZ85). 24 h after transfection, 0 or 0.5 μM grazoprevir was added to culture supernatants and SEAP levels were followed for another 24 h. Data show the ±SD of fold-changes calculated by dividing SEAP levels of Grazoprevir-treated samples by SEAP levels of samples not treated with Grazoprevir (n=3 individual experiments). (B) Endpoint performance of grazoprevir-induced translational and transcriptional gene switch switches. For grazoprevir-inducible translation, HEK-293 cells were co-transfected with a vector encoding SEAP-mRNA containing MCP-specific poly (A)-surrogate (pSL468) and constitutive expression vectors for MCP-(NS3a)₃ (pSL503) and (GNCR)₃-NSP3 (pLZ74) before cultivation in cell culture medium containing 0 or 0.5 μM grazoprevir. For grazoprevir-inducible transcription, HEK-293 cells were co-transfected with a TetR-specific SEAP expression vector (pLZ79) and constitutive expression vectors for TetR-NS3a (pLZ88) and (GNCR)₃-VP64 (pLZ85). SEAP levels in culture supernatants were scored at 48 h after grazoprevir addition. Data show the mean±SD of fold-changes calculated by dividing SEAP levels of grazoprevir-treated samples by SEAP levels of samples not treated with grazoprevir (n=4 individual experiments).

FIG. 17. Grazoprevir-inducible STIF-association with the endogenous eIF4F complex. HEK-293 cells were co-transfected with expression vectors for 3×HA-tagged (GNCR)₃-NSP3 (pSL476) and FLAG-tagged MCP-NS3a (left, pSL1093) or L7Ae-(NS3a)₃ (right, pSL475) and treated with (+) or without (−) grazoprevir before immunoprecipitation. Target proteins in each lysate fraction before (input) and after immunoprecipitation (Flag-IP) were detected with anti-FLAG, anti-HA, anti-eIF4G and anti-eIF4E antibodies. Numbers on the right axis of Western blots represent molecular weights (MW) of target proteins.

FIG. 18. Characterization of grazoprevir-inducible gene switches in terms of long-term performance and compatibility with RNA-only delivery. (A) Grazoprevir-inducible SEAP translation by mRNA delivery. HEK-293 cells were (co-) transfected with in vitro-transcribed mRNA encoding for MCP-(NS3a)₃ (from pSL1085), (GNCR)₃-NSP3 (from pYW361) and SEAP-(MS2-box)₂₄ (from pSL468). SEAP levels in culture supernatants were quantified at 48 h after addition of grazoprevir. Data are shown as the mean±SD, n=4 independent experiments. (B) Reversibility of grazoprevir-inducible protein secretion in mammalian cells. HEK-293_{LSCCS1} (stably expressing MCP-(NS3a)₃, (GNCR)₃-NSP3 and NanoLuc-mRNA containing 24 tandem MS2-box repeats) were cultivated for 7 days while grazoprevir levels in the culture medium were successively switched between 0 and 500 nM. NanoLuc levels were measured every 12 h. The cell density was readjusted to 1×10⁵ cells per ml every 2-3 days.

FIG. 19. Design and optimization of a grazoprevir-inducible gene switch for therapeutic transgene expression in vivo. (A) Grazoprevir-inducible SEAP production in mice. For grazoprevir-inducible SEAP production mediated by a poly (A)-surrogate created through cis-acting mRNA cleavage, corresponding encoding plasmid DNA consisting of pLZ76/pLZ74/pSL355 was hydrodynamically injected into the tail vein of C57BL/6 mice. For grazoprevir-inducible SEAP production mediated by a poly (A)-surrogate created through trans-acting mRNA cleavage, corresponding encoding plasmid DNA consisting of pLZ76/pLZ74/pSL4/pSL88 was hydrodynamically injected into the tail vein of C57BL/6 mice. After 6 h, mice received the first of 3 daily intraperitoneal injections of grazoprevir (1 mg/kg dissolved in PBS). SEAP levels in the bloodstream of mice were measured at 24 h after the first grazoprevir injection. Data are presented as the mean±SEM of n=5 mice per group. (B) Dose-dependent SEAP production induced by oral grazoprevir in mice. pLZ76 (P_{hCMV}-L7Ae-(NS3a)₃-pA), pLZ74 (P_{hCMV}-(GNCR)₃-NSP3-pA) and pSL355 (P_{hCMV}-SEAP-(C/D-box)₂₄-HHR-pA) were hydrodynamically injected into the tail vein of C57BL/6 mice. At 6 h post injection, mice received the first of 3 daily oral grazoprevir administrations at different doses. SEAP levels in the bloodstream of mice were measured at 24 h after the first grazoprevir administration. Data are presented as the mean±SEM; n=5 mice per group. (C) Optimized grazoprevir-inducible SEAP production in mice. Plasmid DNA consisting of pSL468/pLZ74/pSL548 was hydrodynamically injected into the tail vein of C57BL/6 mice. After 6 h, mice received the first of 3 daily intraperitoneal injections of grazoprevir (3 mg/kg dissolved in PBS). SEAP levels in the bloodstream of mice were measured at 24 h after the first grazoprevir injection. Data are presented as the mean±SEM of n=5 mice per group. (D) Grazoprevir-inducible insulin expression in vitro. HEK-293 cells were co-transfected with 200 ng of pSL1042 (P_{hCMV}-MCP-(NS3a)₃-PA), 200 ng of pSL1032 (P_{hCMV}-(GNCR)₃-NSP3-pA) and different amounts of pSL1003 (P_{hCMV}-NanoLuc-P2A-mINS-(MS2-box)₁₆-HHR-pA). NanoLuc and mINS levels in culture supernatants were scored at 48 h after addition of 0.5 μM grazoprevir. Data are mean±SD, n=3 independent experiments. The therapeutic efficacy window represented by physiological blood insulin levels>0.4 μg/L is marked with a blue shaded box. ns, not significant; *, p<0.1; ***, p<0.001.

FIG. 20. Therapeutic efficacy and long-term performance of a grazoprevir-inducible gene switch in mice. (A-C) Treatment of type-1 diabetes (T1D) by oral grazoprevir-inducible insulin expression. Plasmids encoding MCP-(NS3a)₃, (GNCR)₃-NSP3 and insulin-mRNA containing MCP-specific poly (A)-surrogate (pSL548/pLZ74pSL685) were hydrodynamically injected into the tail vein of TID mice. At 6 h post injection, mice were fed with the first of 3 daily administrations of 3 mg/kg grazoprevir. (A) Blood insulin levels and (B) fasting glycemia of mice were measured at 20 h after the first grazoprevir administration. (C) Intraperitoneal glucose tolerance tests (GTT) were performed at 24 h after the first grazoprevir administration (4 h after quantification of blood insulin). Data are presented as the mean±SEM; n=6 mice per group. (D) Long-term control of grazoprevir-inducible SEAP production in mice. C57BL/6 mice received intravenous injection of 7×10¹¹ AAV2/8 particles for constitutive expression of (GNCR)₃-NSP3, MCP-(NS3a)₃ and SEAP-mRNA containing MCP-specific poly (A)-surrogate before SEAP production in the bloodstream was monitored over 10 weeks. At 24 h before each measurement, mice were fed the first of 3 daily administrations of 3 mg/kg grazoprevir. Data are presented as the mean±SEM; n=4 mice per group.

FIG. 21. Molecular architecture and design principles of genetic tristate buffers. (A) Principle of tristate buffers. In both electronics and biology, tristate buffers comprise an upstream switch (regulated by a control input B) that directly controls a downstream switch in its ON/OFF-state (regulated by a data input A). Thus, control input B allows data input A to determine overall activity Y of the circuit unless B is inactivated or “unplugged” by the upstream switch. In such inactivated state (NOT B), overall activity of the circuit would fall into a third “high impedance” state Z that no longer depends on the status (0 or 1 value) of A. (B) Example of a genetic tristate buffer. In mammalian cells, genetic tristate buffers can be created through regulated expression of trigger-inducible gene switches programmed with either “buffered” (BUF) and “inverted” (NOT) signal processing logics. In the present example illustrating a transcription-translation based gene circuit, control input B operating at an upstream layer (e.g. transcription) monitors the expression of a synthetic translation initiation factor (STIF) that modulates target protein expression from a downstream layer (e.g. translation). Tristate buffers can be engineered to contain up to 4 types of gene switches: B activates STIF expression (IF1), B terminates STIF expression (IF0), A activates target protein expression (BUF) and A terminates target protein expression (NOT). STIFs use rotavirus-derived nonstructural protein 3 domains (NSP3) to bind the preinitiation complex eIF4F, resulting in translation of MCP-specific mRNA upon inducible association of NSP3- and MCP-containing factors.

FIG. 22. Design and validation of BUF- and NOT-switches controlled by Grazoprevir. (A) Comparative analysis of different MCP-NS3a variants for grazoprevir-inducible SEAP translation. HEK-293 cells were co-transfected with plasmids encoding MCP-specific SEAP mRNA (pSL468), (GNCR)₃-NSP3 (pLZ74) and fusion proteins of MCP with either NS3a (pSL497) or NS3a (H1) (pSL546). SEAP levels in culture supernatants were scored at 48 h after addition of 0.5 μM Grazoprevir dissolved in DMSO (vehicle control). Data are mean±SD, n=4. (B) Comparative analysis of different MCP-NS3a variants for grazoprevir-repressible SEAP translation. HEK-293 cells were co-transfected with plasmids encoding MCP-specific SEAP mRNA (pSL468), (ANR)₄-NSP3 (pSL549) and fusion proteins of MCP with either NS3a (pSL497) or NS3a (H1) (pSL546). SEAP levels in culture supernatants were scored at 48 h after addition of 0.5 μM Grazoprevir dissolved in DMSO (vehicle control). Data are mean±SD, n=4.

FIG. 23. Optimization of grazoprevir-regulated BUF- and NOT-switches. (A) Grazoprevir-dependent translation in mammalian cells. For initiation of translation, a circularized mRNA configuration must be established with the 5′- and 3′-ends brought into close proximity by a synthetic translational initiation factor (STIF) comprising a NSP3 domain and synthetic tethers (MCP-NS3a (H1)) consisting of an RNA-binding MCP domain and a STIF-binding NS3a (H1) domain. Since grazoprevir concomitantly triggers dissociation of ANR from NS3a (H1) and association of GNCR to NS3a (H1), STIFs engineered to contain GNCR and NSP3 enable grazoprevir-inducible translation (BUF switch). Likewise, fusion of ANR to NSP3 results in grazoprevir-repressible translation (NOT switch). (B) BUF-switch by grazoprevir-inducible translation. HEK-293 cells were co-transfected with plasmids encoding MCP-specific SEAP mRNA (pSL468), (GNCR)₃-NSP3 (pLZ74) and different MCP-fusion proteins consisting of one (pSL546), two (pSL547) or three C-terminal NS3a (H1)-repeats (pSL548). SEAP levels in culture supernatants were scored at 48 h after addition of 0.5 μM Grazoprevir dissolved in DMSO (vehicle control). (C) NOT-inverter by grazoprevir-repressible translation. HEK-293 cells were co-transfected with plasmids encoding MCP-specific SEAP mRNA (pSL468), (ANR)₄-NSP3 (pSL549) and different MCP-fusion proteins consisting of one (pSL546), two (pSL547) or three C-terminal NS3a (H1)-repeats (pSL548). SEAP levels in culture supernatants were scored at 48 h after addition of 0.5 μM Grazoprevir dissolved in DMSO (vehicle control).

FIG. 24. Design and validation of IF0- and IF1-switches controlled by vanillic acid. (A) Upstream gene switches in biological tristate circuits. In tristate circuits, BUF and NOT switches are governed by an “Active-HIGH” (IF1) or an “Active-LOW” (IF0) control signal producing “normal” or “inverted” output, respectively. Biological implementations of “Active-HIGH” and “Active-LOW” switches should be orthogonal to each other to enable interference-free operation in mammalian cells. One example for a set of mutually orthogonal gene switches responding to a same control input are the vanillic acid-inducible MOR9-1 system triggering cAMP-dependent transcription combined with the vanillic acid-repressible mammalian trans-activator VanR-VP64 driving gene expression from inducible VanR-specific promoters. (B) Vanillic acid (VA)-inducible SEAP expression in mammalian cells. HEK-293 cells were transfected with a constitutive MOR9-1 expression vector (pLYL76) and a cAMP-responsive SEAP expression vector (pCK53). After cultivation in medium containing 0 or 400 μM Vanillic Acid (dissolved in DMSO), SEAP levels in the culture supernatants were scored at 48 h post transfection. Data presented are mean±SD, n=4 individual experiments. (C) Vanillic acid (VA)-repressible SEAP expression in mammalian cells. HEK-293 cells were transfected with a constitutive VanR-VP64 expression vector (pSL175) and a vanillic acid-inducible SEAP expression vector (pSL173). After cultivation in medium containing 0 or 400 μM Vanillic Acid (dissolved in DMSO), SEAP levels in the culture supernatants were scored at 48 h post transfection. Data presented are mean±SD, n=4 individual experiments. (D) Parallel operation of vanillic acid-dependent Active-HIGH (IF1) and Active-LOW (IF0) gene switches in mammalian cells. HEK-293 cells were transfected with a constitutive MOR9-1 expression vector (pLYL76), acAMP-responsive SEAP expression vector (pCK53), a constitutive VanR-VP64 expression vector (pSL175) and a VanR-specific NanoLuc expression vector (pLZ345). SEAP levels in the culture supernatants were scored at 48 h after cultivation in cell culture medium containing different concentrations of Vanillic Acid. Data presented are mean±SD, n=4 individual experiments. Numbers represent fold-changes calculated by division of SEAP levels in grazoprevir-containing samples by SEAP levels in grazoprevir-free samples.

FIG. 25. Engineering of different sets of mutually orthogonal grazoprevir-regulated BUF/NOT-switches. (A) Independent sets of grazoprevir-controlled BUF- and NOT-switches. Principles of STIF-based systems (BUF₁ and NOT₁) were described in FIG. 23A. To create GEMS-based systems, grazoprevir-inducible gene expression from synthetic STAT3-specific promoters (BUF2) occurs upon dimerization of a synthetic cell membrane receptor containing IL6RB-derived intracellular domains and extracellular GNCR- and NS3a (H1)-domains. Exchange of the GNCR domain by ANR results in grazoprevir-repressible gene expression (NOT₂). For StaPLd-based systems, grazoprevir inhibits autoproteolysis of a synthetic mammalian trans-activator PcaV-StaPLd-VP64 or trans-silencer PcaV-StaPLd-KRAB, resulting in trigger-inducible activation (BUF3) or inhibition (NOT₃) of gene expression from synthetic PcaV-specific promoters. (B) Design and validation of grazoprevir-inducible and grazoprevir-repressible GEMS variants (BUF₂ and NOT₂). GEMS receptors comprise an extracellular ligand binding domain, a transmembrane domain GEMS™ (yellow) and an intracellular domain derived from IL6RB-, FGFR- or VEGFR-derived receptor scaffolds (grey). Co-expression of GEMS receptors containing NS3a (H1) and GNCR as extracellular domains (GEMS_{NS3a (H1)} &GEMS_{GNCR}) could account for grazoprevir-inducible target gene expression. GEMS receptors containing NS3a (H1) and ANR as extracellular domains (GEMS_{NS3a (H1)} &GEMS_{ANR}) could account for grazoprevir-repressible target gene expression. (left) For grazoprevir-inducible target gene expression by GEMS variants containing IL6RB-derived intracellular domains, HEK-293 cells were co-transfected with a STAT3-specific SEAP expression vector (pLZ284) and constitutive expression vectors for corresponding GEMS_{NS3a (H1)} and GEMS_{GNCR} constructs (pSL890/pSL889). For grazoprevir-inducible target gene expression by GEMS variants containing FGFR1-derived intracellular domains, cells were co-transfected with a TetR-specific SEAP expression vector (pMF111) and constitutive expression vectors for TetR-Elk1 and corresponding GEMS_{NS3a (H1)} and GEMS_{GNCR} constructs (pSL892/pSL891). For grazoprevir-inducible target gene expression by GEMS variants containing VEGFR-derived intracellular domains, cells were co-transfected with a calcium-inducible SEAP expression vector (pMX57) and constitutive expression vectors for corresponding GEMS_{NS3a (H1)} and GEMS_{GNCR} constructs (pSL894/pSL893). (right) For grazoprevir-repressible target gene expression by GEMS variants containing IL6RB-derived intracellular domains, cells were co-transfected with a STAT3-specific SEAP expression vector (pLZ284) and constitutive expression vectors for corresponding GEMS_{NS3a (H1)} and GEMS_{ANR} constructs (pSL890/pLZ267). For grazoprevir-repressible target gene expression by GEMS variants containing FGFR1-derived intracellular domains, cells were co-transfected with a TetR-specific SEAP expression vector (pMF111) and constitutive expression vectors for TetR-Elk1 and corresponding GEMS_{NS3a (H1)} and GEMS_{ANR} constructs (pSL892/pLZ269). For grazoprevir-repressible target gene expression by GEMS variants containing VEGFR-derived intracellular domains, cells were co-transfected with a calcium-inducible SEAP expression vector (pMX57) and constitutive expression vectors for corresponding GEMS_{NS3a (H1)} and GEMS_{ANR} constructs (pSL894/pLZ271). SEAP levels in culture supernatants were scored at 48 h after addition of 10 μM Grazoprevir dissolved in DMSO (vehicle control). Data are mean±SD, n=3. (C) Design and validation of StaPLd-based grazoprevir-inducible trans-activators and trans-silencers (BUF3 and NOT3). (Left) For grazoprevir-repressible gene expression, HEK-293 were co-transfected with a constitutive PcaV-StaPLd-KRAB expression vector (pLZ417) and a PcaV-repressible SEAP expression vector (pLZ419). (Right) For grazoprevir-inducible gene expression, HEK-293 were co-transfected with a constitutive PcaV-StaPLd-VP64 expression vector (pLZ418) and a PcaV-specific SEAP expression vector (pSL172). SEAP levels in culture supernatants were quantified at 48 h after addition of 10 μM grazoprevir. Data are shown as the mean±SD, n=3 independent experiments.

FIG. 26. Orthogonality requirements for gene switches in tristate-based gene circuits. (A) Selection criteria for downstream modules. Each individual set of parallel BUFₙ/NOTₙ switches monitoring a different output signal n must be orthogonal to each other for interference-free biocomputation. (B, C) Parallel operation of grazoprevir-dependent sets of BUF/NOT-switches. (B) A grazoprevir-regulated GEMS-based BUF switch driving NanoLuc expression (BUF2; GNCR-GEMS_{IL6RB} &NS3a (H1)-GEMS_{IL6RB} &P_{STAT3}-NanoLuc; pSL889/pSL890/pLZ368) was co-administered with a grazoprevir-regulated STIF-based NOT switch driving SEAP expression (NOT₁; (ANR)₈-NSP3 &MCP-(NS3a (H1))₃ &P_{hCMV}-SEAP-(MS2-box) 24-HHR-pA; pSL582/pSL548/pSL468), while a grazoprevir-regulated GEMS-based NOT switch driving NanoLuc expression (NOT₂; ANR-GEMS_{IL6RB}&NS3a (H1)-GEMS_{IL6RB}&P_{STAT3}-NanoLuc; pLZ268/pSL890/pLZ368) was co-administered with a grazoprevir-regulated STIF-based BUF switch driving SEAP expression (BUF1; (GNCR)₃-NSP3&MCP-(NS3a (H1))₃&P_{hCMV}-SEAP-(MS2-box)₂₄-HHR-pA; pSL1032/pSL548/pSL468) into HEK-293 cells. SEAP and NanoLuc levels in the culture supernatants were scored at 48 h after cultivation in cell culture medium containing 0 or 10 μM Grazoprevir. Data presented are mean±SD, n=3 individual experiments. (C) Agrazoprevir-regulated StaPLd-based BUF switch driving NanoLuc expression (BUF3; PcaV-StaPLd-VP64; pLZ418&pSL172) was co-administered with a grazoprevir-regulated STIF-based NOT switch driving NanoLuc expression (NOT₁; ANR-NSP3 &NS3a (H1)-MCP &P_{hCMV}-NanoLuc-(MS2-box)₂₄-HHR-pA; pQZ111&pSL548&pSL582), while a grazoprevir-regulated StaPLd-based NOT switch driving SEAP expression (NOT₃; PcaV-StaPLd-KRAB; pLZ417&pLZ419) was co-administered with a grazoprevir-regulated STIF-based BUF switch driving NanoLuc expression (BUF1; GNCR-NSP3&NS3a (H1)-MCP&P_{hCMV}-NanoLuc-(MS2-box)₂₄-HHR-pA; pSL1032&pSL548&pQZ111) into HEK-293 cells. SEAP and NanoLuc levels in the culture supernatants were scored at 48 h after cultivation in cell culture medium containing 0 or 10 μM Grazoprevir. Data presented are mean±SD, n=3 individual experiments. (D) Orthogonality between vanillic Acid (VA)-inducible CAMP-dependent SEAP expression and grazoprevir-inducible STAT3-dependent NanoLuc expression in mammalian cells. A vanillic acid-inducible gene switch controlling SEAP expression (IF1; pCK53) was co-administered with grazoprevir-regulated GEMS-based BUF switch driving NanoLuc expression (BUF₂; pSL889/pSL890/pLZ368) into HEK-MOR9 (C0) cells and cultivated in cell culture medium containing Vanillic Acid (VA, 400 μM) and/or Grazoprevir (Gra, 10 μM). SEAP levels in the culture supernatants were scored at 48 h post transfection. Data presented are mean±SD, n=3 individual experiments.

FIG. 27. (Bio) computational logic similarities of genetically engineered tristate buffers. (A) Logical characteristics of tristate buffers. Combination of grazoprevir-controlled (BUF and NOT) with vanillic acid-controlled gene switches (Active-HIGH and Active-LOW) produces the 4 different types of tristate buffers: Active-High Buffer (IF1 regulates BUF:BUFIF1), Active-High Inverted Buffer (IF1 regulates NOT:NOTIF1), Active-Low Buffer (IF0 regulates BUF:BUFIF0) and Active-Low Inverted Buffer (IF0 regulates NOT:NOTIF0). (B) Grazoprevir and vanillic acid-controlled tristate buffers in mammalian cells. In a biological context, Hi-Z (electrically disconnected) and 0 states (electrically switched off) of tristate buffers are considered functionally similar from the viewpoint of inactive gene expression (OFF). Thus, BUFIF1 shows logic similarity to a Boolean AND gate, NOTIF0 shows logic similarity to an NOR gate, while NOTIF1 and BUFIF0 are logically similar to conventional NIMPLY (AND NOT) gates. For BUFIF1, cells were co-transfected with plasmids encoding MCP-specific EGFP mRNA (pQZ112), constitutive expression vectors for MCP-(NS3a (H1))₃ (PSL548), MOR9-1 (pLYL76) and VanR-VP64 (pSL175) and a cAMP-responsive (GNCR)₃-NSP3 expression vector (pLYL62). For NOTIF0, cells were co-transfected with pQZ112, pSL548, pSL175 and a vanillic acid-responsive (ANR)₄-NSP3 expression vector (pLYL85). For NOTIF1, cells were co-transfected with pQZ112, pSL548, pLYL76 and a cAMP-responsive (ANR)₄-NSP3 expression vector (pLYL67). For BUFIF0, cells were co-transfected with pQZ112, pSL548, pSL175 and a vanillic acid-responsive (GNCR)₃-NSP3 expression vector (pLYL63). Vanillic Acid (VA, 400 μM) and Grazoprevir (Gra, 0.5 μM) were added at 6 h post transfection. At 36 hours post transfection, fluorescent images showing EGFP signals (scale bar: 100 μm) were acquired and flow-cytometry analysis was performed with 10000 cells per group (FIG. 28). Data show weighted fluorescence units as mean±SD, n=3 individual experiments. (C) Tristate buffers formed by vanillic acid-regulated IF0/IF1 and grazoprevir-regulated BUF2/NOT₂ switches. For BUF2IF1, HEK-293 cells stably expressing MOR9-1 (HEK-MOR9 (C0)) were co-transfected with a STAT3-specific SEAP expression vector (pLZ284) and cAMP-responsive expression vectors for GEMS_{GNCR} (pLZ285) and GEMS_{NS3a (H1)} (pLZ286). For NOT₂IF0, HEK-293 cells were co-transfected with pLZ284, a constitutive VanR-VP64 expression vector (pSL175) and vanillic acid-responsive expression vectors for GEMS_{NS3a (H1)} (pLZ412) and GEMS_{ANR} (pLZ413). For NOT₂IF1, HEK-MOR9 (C0) cells were co-transfected with pLZ284 and cAMP-responsive expression vectors for GEMS_{ANR} (pLZ310) and GEMS_{NS3a (H1)} (pLZ286). For BUF2IF0, HEK-293 cells were co-transfected with pLZ284, a constitutive VanR-VP64 expression vector (pSL175) and vanillic acid-responsive expression vectors for GEMS_{NS3a (H1)} (PLZ412) and GEMS GNCR (pLZ411). After cultivation in medium containing Vanillic Acid (VA, 400 μM) and/or Grazoprevir (Gra, 10 μM), SEAP levels in the culture supernatants were scored at 48 h post transfection. Data presented are mean±SD, n=3 individual experiments.

FIG. 28. Flow cytometric data related to FIG. 27B.

FIG. 29. Modular assembly of Boolean logic circuits using tristate buffers and BUF/NOT switches. For tristate-based circuits showing OR-logics, cells were transfected with a constitutive expression vector for (GNCR)₃-NSP3 (pLZ74; module 6) and a cAMP-responsive (ANR) g-NSP3 expression vector (pSL580; module 2). For tristate-based circuits showing XOR-logics, cells were transfected with a vanillic acid-responsive (GNCR)₃-NSP3 expression vector (pLYL63; module 3) and a cAMP-responsive (ANR)₄-NSP3 expression vector (pLYL67; module 2). For tristate-based circuits showing NAND-logics, cells were transfected with a constitutive expression vector for (ANR) g-NSP3 (pSL582; module 5) and a vanillic acid-responsive (GNCR) 3-NSP3 expression vector (pLYL87; module 3). For tristate-based circuits showing XNOR-logics, cells were transfected with a cAMP-responsive (GNCR)₃-NSP3 expression vector (pLYL62; module 1) and a vanillic acid-responsive (ANR)₈-NSP3 expression vector (pLYL95; module 4). For tristate-based circuits showing Gra IMPLY VA logics, cells were transfected with a constitutive expression vector for (ANR)₈-NSP3 (pSL582; module 5) and a cAMP-responsive (GNCR)₃-NSP3 expression vector (pLYL62; module 1). For tristate-based circuits showing VA IMPLY Gra logics, cells were transfected with a constitutive expression vector for (GNCR)₃-NSP3 (pLZ74; module 6) and a vanillic acid-responsive (ANR) g-NSP3 expression vector (pLYL95; module 4). For circuits involving modules 1 and 2, cells were further co-transfected with a constitutive expression vector for MOR9-1 (pLYL76). For circuits involving modules 3 and 4, cells were further co-transfected with a constitutive expression vector for VanR-VP64 (pSL175). For all circuits, cells were co-transfected with a reporter plasmid encoding MCP-specific EGFP mRNA (pQZ112) and constitutive expression vectors for MCP-(NS3a (H1))₃ (pSL548) forming the computational core unit. At 6 h post transfection, different combinations of Vanillic Acid (V, 400 μM) and Grazoprevir (G, 0.5 μM) were added. At 36 hours post transfection, fluorescent images showing EGFP signals (scale bar: 100 μm) were acquired. Representative data for 3 independent experiments are shown.

FIG. 30. Design and validation of a half-adder and half-subtractor gene circuit in mammalian cells. (A) Half-adder. A half-adder calculates the sum of two Boolean integers (e.g. vanillic acid and grazoprevir) by returning a two-output result consisting of Boolean numbers for CARRY (C: representing 2¹ digits) and SUM (S: representing 2⁰ digits). For biological implementation, HEK-MOR9 (C0) cells were co-transfected with plasmids encoding genetic components for the computational core unit driving EGFP expression [a constitutive expression vector for MCP-(NS3a (H1))₃ and MCP-specific EGFP mRNA (pSL548/pQZ112)], module 3 [a constitutive expression vector for VanR-VP64 and a vanillic acid-responsive (GNCR)₃-NSP3 expression vector (pSL175/pLYL63)], module 2 [a constitutive expression vector for MOR9-1 and a cAMP-responsive (ANR)₄-NSP3 expression vector (pLYL76/pLYL67)] and module 7 [cAMP-responsive expression vectors for GEMS_{GNCR} and GEMS_{NS3a (H1)} (pLZ285/pLZ286) and a STAT3-specific mCherry expression vector (pLZ287)]. Flow cytometric analysis of EGFP- and mCherry signals (FIG. 31A) was performed at 48 h after cultivation in medium containing different combinations of Vanillic Acid (V, 400 μM) and Grazoprevir (G, 10 μM). Data show weighted fluorescence units as mean±SD representative for 3 individual experiments. (B) Half-subtractor. A half-subtractor calculates the difference between two Boolean integers (e.g. vanillic acid and grazoprevir) by returning a two-output result consisting of Boolean numbers for BORROW (B_{O}: representing-1*2¹ digits) and DIFFERENCE (D: representing 2⁰ digits). For biological implementation, HEK-MOR9 (C0) cells were co-transfected with plasmids encoding genetic components for modules no. 8 [cAMP-responsive expression vectors for GEMS_{ANR} and GEMS_{NS3a (H1)} and a STAT3-specific mCherry expression vector (pLZ310/pLZ286/pLZ287)], no. 3 (pSL175/pLYL63), no. 2 (pLYL76/pLYL67) and the computational core unit driving EGFP expression (pSL548/pQZ112). Flow cytometric analysis of EGFP- and mCherry signals (FIG. 31B) was performed at 48 h after cultivation in medium containing different combinations of Vanillic Acid (V, 400 μM) and Grazoprevir (G, 10 μM). Data show weighted fluorescence units as mean±SD representative for 3 individual experiments.

FIG. 31. Flow cytometric data related to FIG. 30. (A) Flow cytometric data related to FIG. 30A. (B) Flow cytometric data related to FIG. 30B.

FIG. 32: Grazoprevir and Vanillic Acid responsive Half-adder using BUF3. HEK-293 cells were co-transfected with plasmids encoding EGFP-mRNA with MCP-specific poly (A)-surrogate (pQZ112), constitutive expression vectors for MCP-(NS3a (H1))₃ (pSL548), VanR-VP64 (pSL175) and MOR9-1 (pLYL76), cAMP-responsive (ANR)₄-NSP3 (pLYL67) and NLS-PcaV-StaPLd-VP64 expression vectors (pSL754), a vanillic acid-responsive (GNCR)₃-NSP3 expression vector (pLYL63) and a PcaV-specific grazoprevir-inducible mCherry expression vector (pSL648). At 6 h post transfection, different combinations of Vanillic Acid (V, 400 μM) and Grazoprevir (G, 0.5 μM) were added. At 36 hours post transfection, fluorescent images showing EGFP signals (scale bar: 100 μm) were acquired. Representative data for 3 independent experiments are shown.

FIG. 33. Vanillic acid and grazoprevir-responsive logic gates in vivo. (A) Vanillic acid AND NOT Grazoprevir gate. 420 μg of plasmid DNA (pSL683/pSL548/pLYL76/pLYL67 in a 2:6:16:3 (w/w/w/w) ratio) were hydrodynamically injected into the tail vein of WT C57BL/6 mice. At 6 h post injection, different combinations of Vanillic Acid (500 mg/kg/day) and Grazoprevir (9 mg/kg/day) were administered by intraperitoneal injection (3 times per day). NanoLuc levels in the bloodstream of mice were measured at 24 h after first stimulation. Data are presented as the mean±SEM; n=3 mice. (B) Vanillic Acid (VA)-repressible SEAP expression (IF0). 25 μg of plasmid DNA (pSL175/pSL173 in a 3:2 (w/w) ratio) were hydrodynamically injected into the tail vein of WT C57BL/6 mice. At 6 h post injection, Vanillic Acid (500 mg/kg/day) dissolved in PBS was administered by intraperitoneal injection (3 times per day). SEAP levels in the bloodstream of mice were measured at 24 h after the first Vanillic Acid injection. (C) Grazoprevir-repressible SEAP production (NOT₁). 450 μg of plasmid DNA (pSL468 (50 μg)/pSL549 (200 μg)/pSL548 (200 μg) in a 1:4:4 (w/w/w) ratio) were hydrodynamically injected into the tail vein of WT C57BL/6 mice. At 6 h post injection, Grazoprevir (3 mg/kg) dissolved in PBS was administered by intraperitoneal injection 3 times per day. SEAP levels in the bloodstream of mice were measured at 24 h after the first Grazoprevir injection. Data are presented as the mean±SEM; n=5 mice. (D) Vanillic Acid (VA)-inducible SEAP expression (IF1). 430 μg of plasmid DNA (pLYL76/pCK53 in a 40:3 (w/w) ratio) were hydrodynamically injected into the tail vein of WT C57BL/6 mice. At 6 h post injection, Vanillic Acid (500 mg/kg/day) dissolved in PBS was administered by intraperitoneal injection (3 times per day). SEAP levels in the bloodstream of mice were measured at 24 h after the first Vanillic Acid injection. Data are presented as the mean±SEM; n=5 mice.

FIG. 34. A general design strategy for STIF-based “molecular pincers” to sense various intracellular targets of interest. To sense a specific target Y″, a pair of proteins Y and Y′ both binding to different epitopes of Y″ can be fused either to the N-terminus or C-terminus of RBP- or eIFBP-domains of STIF regulators, rendering the formation of a circularized mRNA configuration and translational initiation exclusively dependent on the presence of intracellular Y″. Alternatively, RBP-specific 5′-cap and/or poly (A)-surrogates could be replaced by aptamer motifs directly binding to Y″ to form a circularized mRNA configuration in combination with Y or Y′ fused to eIFBPs.

FIG. 35. Preliminary experiments for development of STIF-based protein sensors (Part 1). (A, B) Optimization of NSP3-fusion constructs. (A) HEK-293 cells were co-transfected with plasmids encoding SEAP-mRNA containing an MCP-specific poly (A)-surrogate (pSL468), MCP-Coh2 (pSL674) and NSP3-fusion proteins consisting of one (pSL66), two (pSL85) or three N-terminal DocS-repeats (pSL86). SEAP expression in culture supernatants were scored at 48 h post transfection. Data are mean±SD, n=4. (B) HEK-293 cells were co-transfected with plasmids encoding SEAP-mRNA containing MCP-specific poly (A)-surrogate (pSL468), MCP-DocS (pSL1311), and NSP3-fusion proteins consisting of one (pSL241), two (pSL242) or three N-terminal Coh2-repeats (pSL243). SEAP expression in culture supernatants was scored at 48 h post-transfection. Data are mean±SD, n=4. (C) Translational regulation by different C/D-box-specific DocS-tethers. HEK-293 cells were co-transfected with plasmids encoding SEAP mRNA containing an L7Ae-specific poly (A)-surrogate (pSL88 & pSL4), DocS-NSP3 (pSL66 or pcDNA3.1 (+) as negative control) and different L7Ae-fusion proteins with one (pSL65), two (pSL82) or three C-terminal Coh2-repeats (pSL83). SEAP expression in culture supernatants were scored at 48 h post transfection. Data are mean±SD, n=4. (D) Translational regulation by different MS2-box-specific DocS-tethers. HEK-293 cells were co-transfected with plasmids encoding SEAP-mRNA containing an MCP-specific poly (A)-surrogate (pSL468), (DocS)₃-NSP3 (pSL86) and different MCP-fusion proteins consisting of one (pSL674), two (pSL1079) or three N-terminal Coh2-repeats (pSL1080). SEAP expression in culture supernatants were scored at 48 h post transfection. Data are mean±SD, n=4.

FIG. 36. Preliminary experiments for development of STIF-based protein sensors (Part 2). (A) Translational regulation of C/D-box-containing mRNA by different Coh2-specific eIFBPs. HEK-293 cells were co-transfected with plasmids encoding SEAP-mRNA containing an L7Ae-specific poly (A)-surrogate (pSL88&pSL4), L7Ae-(Coh2)₃ (pSL83) and NSP3-fusion proteins consisting of one (pSL66), two (pSL85) or three N-terminal DocS-repeats (pSL86). SEAP expression in culture supernatants were scored at 48 h post transfection. Data are mean±SD, n=4. (B) Translational regulation of MS2-box-containing mRNA by different Coh2-specific eIFBPs. HEK-293 cells were co-transfected with plasmids encoding SEAP-mRNA containing MCP-specific poly (A)-surrogate (pSL468), MCP-(Coh2)₃ (pSL1080), and NSP3-fusion proteins consisting of one (pSL66), two (pSL85) or three N-terminal DocS-repeats (pSL86). SEAP expression in culture supernatants was scored at 48 h post-transfection. Data are mean±SD, n=4. (C, D) DocS-dependent NSP3-mediated activation of STIF-specific mRNA. (C) For MCP-based systems, HEK-293 cells were co-transfected with plasmids encoding SEAP-mRNA containing an MCP-specific poly (A)-surrogate (pSL468), MCP-(Coh2)₃ (pSL1080) and different amounts of pSL86. (D) For L7Ae-based systems, HEK-293 cells were co-transfected with plasmids encoding SEAP-mRNA containing an L7Ae-specific poly (A)-surrogate (pSL88&pSL4), L7Ae-(Coh2)₃ (pSL83) and different amounts of (DocS)₃-NSP3 expression vectors (pSL86). SEAP expression in culture supernatants were scored at 48 h post transfection. Data are mean±SD, n=4.

FIG. 37. Engineering of STIF-based protein sensors for in vitro and in vivo diagnostics. (A) Genetically encoded protein sensors employing protein association-dependent STIF reconstitution. HEK-293 cells were co-transfected with pSL468 and constitutive expression vectors for MCP-(Coh2)₃ (PSL1080), (Coh2)₃-NSP3 (pSL243) and (DocS)₃ (different amounts of pSL244). SEAP expression in culture supernatants were scored at 48 h post transfection. Data are shown as the mean±SD of n=4 independent experiments. Bars represent the mean SD, and filled circles show individual results. (B) Construction of a hepatitis C virus (HCV)-specific protein sensor in mammalian cells. HEK-293 were co-transfected with plasmids encoding SEAP-mRNA containing L7Ae-specific poly (A)-surrogate (pSL88&pSL4), L7Ae-scFv35 (pSL136), (scFv162)₃-NSP3 (pSL139) and different amounts of a synthetic trimeric target protein (nNS3)₃ (pSL107). SEAP expression in culture supernatants were scored at 48 h post transfection. Data are mean±SD, n=3. (C) Implementation of synthetic gene circuits in cell-free systems. STIF-based protein sensors designed according to the principles shown in FIG. 34 can also be applied on abiotic materials such as paper discs. In such cases, the genetic componentry of the STIF-based sensors (either in form of plasmid DNA, mRNA or purified proteins) can be freeze-dried alongside cell lysates onto abiotic materials to enable long-term storage. Detection of target compounds (such as virulence factors of bacteria or viruses) in humidified air will trigger STIF reconstitution, translational initiation and production of reporter proteins within the paper discs that is eventually visible to the naked eye.

FIG. 38. Genetically encoded sensors for intracellular target proteins of different subcellular localization. (A) Engineering of transcription- and translation-based sensors for detection of differentially localized intracellular proteins. The synthetic target protein EGFP-NS3a (H1) was targeted to different intracellular compartments through fusion with different localization signals (1, NLS (nuclear localization signal); 2, NES (nuclear export signal); 3, CAAX (prenylation motif); 4, transmembrane localization signal; 5, secretory signal peptide) to allow detection with co-expressed genetic sensors engineered on the basis of LaG16 (an EGFP nanobody) and ANR (a peptide motif binding NS3a (H1)). For transcription-based sensing, LaG16 was fused to TetR and ANR was fused to VP64 to allow EGFP-NS3a (H1)-dependent activation of TetR-specific promoters. For translation-based sensing, LaG16 was fused to MCP and ANR was fused to NSP3 to allow EGFP-NS3a (H1)-dependent STIF reconstitution and translation of MCP-specific mRNA. (B) Dose-dependent detection of differentially translocated EGFP-NS3a (H1). HEK-293 cells were co-transfected with plasmids encoding the translation-based EGFP-NS3a (H1) sensor (pSL776/pSL582/pSL468: for expression of MCP-LaG16, (ANR)₈-NSP3 and SEAP-mRNA with MCP-specific poly (A)-surrogate) or the transcription-based EGFP-NS3a (H1) sensor (pSL834/pSL836/pMF111: for expression of TetR-LaG16, (ANR)₈-VP64 and a TetR-specific promoter controlling SEAP transcription) and different amounts of constitutive expression vectors for different target proteins (0, native EGFP-NS3a (H1), pSL775; 1, nuclear NLS-EGFP-NS3a (H1), pSL797; 2, cytosolic NES-EGFP-NS3a (H1), pSL824; 3, prenylated EGFP-NS3a (H1)-CAAX, pSL799; 4, membrane-localized TM-EGFP-NS3a (H1), pSL798; 5, secretory SP-EGFP-NS3a (H1), pSL796). At 48 h after transfection, fluorescence images of cellular EGFP signals were acquired (scale bar: 10 μm) and SEAP levels in the culture supernatant were profiled. Data are shown as the ±SD fold change of SEAP activity relative to basal SEAP levels detected for no EGFP-NS3a (H1) expression (Ong-columns).

FIG. 39. Engineering of a STIF-based protein sensor for an artificial fusion protein EGFP-NS3a (H1). (A) Translational regulation by different EGFP-specific MCP-fusion proteins. HEK-293 cells were co-transfected with plasmids encoding SEAP-mRNA containing MCP-specific poly (A)-surrogate (pSL468), a constitutive EGFP-NSP3 expression vector (pSL942) and expression vectors for different MCP-LaG16 variants containing one (pSL776) or two (pSL777) tandem LaG16 repeats. Transfection of pcDNA3.1 (+) instead of pSL942 was used as a negative control. SEAP expression in the culture supernatant were profiled at 48 h after transfection. Data presented are mean±SD, n=4. (B) Translational regulation by different NS3a (H1)-specific NSP3-fusion proteins. HEK-293 cells were co-transfected with plasmids encoding SEAP-mRNA containing MCP-specific poly (A)-surrogate (pSL468), a constitutive MCP-(NS3a (H1))₃ expression vector (pSL548) and expression vectors for different ANR-NSP3 variants containing different tandem ANR repeats (ANR-NSP3, pSL704; (ANR)₂-NSP3, pSL549; (ANR)₆-NSP3, pSL581; (ANR)₈-NSP3, pSL582). SEAP expression in the culture supernatant were profiled at 48 h after transfection. Data presented are mean±SD, n=4. (C) Construction of an EGFP-NS3a (H1) sensor in mammalian cells. HEK-293 cells were co-transfected with plasmids encoding reporter SEAP-mRNA containing MCP-specific poly (A)-surrogate (pSL468), a constitutive EGFP-NS3a (H1) expression vector (pSL775) and different combinations of MCP-LaG16 (pSL776) and (ANR)₈-NSP3 expression vectors (pSL582). Transfection of pcDNA3.1 (+) instead of pSL776 and/or pSL582 was used as a negative control (−). SEAP expression in the culture supernatant were profiled at 48 h after transfection. Data presented are mean±SD, n=4.

FIG. 40. Engineering of a STIF-based sensor for cancer-related fusion genes. (A) Engineering of a STIF-based sensor for the BCR-ABL fusion protein. In chronic myelogenous leukemia (CML), chromosomal translocation genetically fuses the BCR gene at 22q11 with the ABL1 tyrosine kinase-encoding gene at 9q34, forming a hybrid oncoprotein BCR-ABL with increased kinase activity. Bipartite STIFs where each split-component is engineered to contain an intrabody that specifically binds each individual BCR or ABL1 protein in its native form can only be reconstituted to activate STIF-dependent translation in cells containing the fusion protein configuration. (B) Selectivity of the EGFP-NS3a (H1) fusion protein sensor. HEK-293 cells were co-transfected with the EGFP-NS3a (H1) sensor (pSL468/pSL776/pSL582; FIG. 39C) and expression vectors for either EGFP (pWS164), NS3a (H1) (pSL818) or EGFP-NS3a (H1) (pSL775). SEAP expression in the culture supernatant were profiled at 48 h after transfection. Data presented are mean±SD of n=3 individual experiments. (C) Selectivity of the BCR-ABL fusion protein sensor. HEK-293 cells were co-transfected with a SEAP-producing BCR-ABL sensor (constitutive expression of MCP-ABI (iDab) (pSL860), CCmut3-NSP3 (pSL863) and MCP-specific SEAP-mRNA (pSL468)) and expression vectors for either BCR (pSL1045), ABL (pSL1046) or BCR-ABL (pSL1014). SEAP expression in the culture supernatant were profiled at 48 h after transfection. Data presented are mean±SD, n=4 individual experiments. (D) BCR-ABL-mediated association of MCP-ABI (iDab) and CCmut3-NSP3. HEK-293 cells were co-transfected with expression vectors for 3×FLAG-tagged MCP-ABI (iDab) (pSL1101), 3×HA-tagged CCmut3-NSP3 and BCR-ABL (+, pSL1014) or pcDNA3.1 (+) (−, negative control) at 48 h before immunoprecipitation. Target proteins in each lysate fraction before (input) and after immunoprecipitation (Flag-IP) were detected with anti-FLAG and anti-HA antibodies. Numbers on the right axis of Western blots represent molecular weights (MW) of target proteins.

FIG. 41. Target-specificity of a genetically encoded EGFP-NS3a (H1) protein sensor. HEK-293 cells were co-transfected with plasmids encoding mCherry-mRNA containing MCP-specific poly (A)-surrogate (pSL683, P_{hCMV}-NanoLuc-P2A-mCherry-(MS2-box)₂₄-HHR-pA) and constitutive expression vectors for MCP-LaG16 (pSL776), (ANR)₈-NSP3 (pSL582) and EGFP-NS3a (H1) (pSL775). Transfection of pcDNA3.1 (+) instead of pSL775 was used as a negative control. At 48 h post transfection, (A) fluorescent images showing co-localized EGFP/mCherry signals were acquired (scale bar: 50 μm) before (B) flow-cytometric analysis was performed (10,000 cells per group). Representative data from 3 independent experiments are shown.

FIG. 42. Validation of therapeutic efficacy using xenograft mouse models. Cell lines with different molecular signatures hypothetically distinguishable by a cytoplasmic target protein were subcutaneously implanted into lower back of mice to allow tumor growth over 17 days. On day 7, gene circuits engineered for cancer-selective activation of apoptosis were injected into tumors every 2 days. The impact on tumor size was constantly monitored over the entire experimental timespan.

FIG. 43. Engineering of target-specific protein sensors driving self-sufficient cancer gene therapies. (A) Validation of therapeutic efficacy using xenograft mouse models. Plasmid DNA encoding for a STIF-based protein sensor for EGFP-NS3a (H1)-triggered expression of a pro-apoptotic Bax protein were injected into tumors of mice described in FIG. 42. In healthy cells, Bax expression remains OFF due to the absence of the EGFP-NS3a (H1) target protein. Overexpression of EGFP-NS3a (H1) in malignant cells triggers self-sufficient STIF assembly, Bax translation and apoptosis. (B-D) EGFP-NS3a (H1)-specific activation of apoptosis in mice. Mice harboring subcutaneous B16-F10_{EGFP-NS3a (H1)}-derived tumors received local injections of pcDNA3.1 (+) (negative control, n=5 mice per group) or plasmid DNA mixes comprising pSL831 (P_{hCMV}-mBax-(MS2-box)₂₄-HHR-pA), pSL776 (P_{hCMV}-MCP-LaG16-pA) and pSL582 (P_{hCMV}-(ANR)₈-NSP3-pA) (treatment group, n=5 mice per group). (B) Daily changes of tumor size were assessed by calculating V=(length*width)²/2. (C) Tumors were harvested at the final experimental day for image analysis and (D) measurement of Bax protein levels by Western Blot. (E-G) No activation of apoptosis by a P_{hCMV}-driven EGFP-NS3a (H1) sensor in EGFP-NS3a (H1)-deficient tissues. Mice harboring subcutaneous B16-F10-derived tumors received local injections of pcDNA3.1 (+) (negative control, n=5 mice per group) or plasmid DNA mixes comprising pSL831/pSL776/pSL582 (treatment group, n=5 mice per group). (E) Daily changes of tumor size were assessed by calculating V=(length*width)²/2. (F) Tumors were harvested at the final experimental day for image analysis and (G) measurement of Bax protein levels by Western Blot. Numbers on the left axis of Western blots represent molecular weights (MW) of target proteins.

FIG. 44. Therapeutic biocomputer coupling tissue-specific detection and STIF-based protein sensing. To precisely distinguish complex cell signatures within heterogenous tissues (e.g., tumors), STIF-based protein sensors with programmable target-specificity could be coupled with tissue-specific promoters (TSP) driving the production of STIF-specific mRNA for suicide gene expression (e.g., Bax). Upon delivery of such gene circuits into living tissues in vivo, self-sufficient apoptosis is exclusively triggered in cells that meet the criteria of both tissue-(TSP-triggered transcription of poly (A)-deficient STIF-dependent Bax mRNA) AND target-specificity (translation of Bax mRNA only upon STIF-mediated detection of intracellular protein markers).

FIG. 45. Characterization of tissue- and target-specificity in mammalian cells and mice. (A) Quantification of AFP-levels using (left panel) TSP-driven reporter gene expression and (right panel) qRT-PCR. (left panel) N2A, Hepa1-6 and B16-F10 cells were co-transfected with a P_{MusAFP}-driven NanoLuc expression vector (pSLM54) and a constitutive FLuc expression vector (pYW99). Luciferase levels were quantified at 48 h after transfection. Data presented are mean±SD of relative luciferase activity (NanoLuc/FLuc) for n=3 individual experiments. (right panel) Relative AFP expression levels in N2A, Hepa1-6 and B16-F10 cells were profiled by qRT-PCR using murine AFP-specific primers listed in Table S1 and normalized to the gene encoding murine ribosomal protein (Rplp0). Data presented are mean±SD, n=3. Bars represent the mean and SD, and filled circles show individual results. (B) Tissue-specific sensing of EGFP-NS3a (H1) in vitro. Native (WT) or stably EGFP-NS3a (H1)-transgenic N2A and Hepa1-6 cells were co-transfected with P_{MusAFP}-driven (pSL813) or constitutive expression vectors for NanoLuc-P2A-mCherry mRNA containing MCP-specific poly (A)-surrogate (pSL683) and constitutive expression vectors for MCP-LaG16 (pSL776), (ANR)₈-NSP3 (pSL582) and FLuc (pYW99). Luciferase levels were quantified at 48 h after transfection. Data presented are mean±SD of relative luciferase activity (NanoLuc/FLuc), n=3 individual experiments. (C) Validation of tissue-specific EGFP-NS3a (H1) sensing in mice. Plasmids encoding P_{hCMV}-driven (pSL468/pSL582/pSL776) and P_{MusAFP}-driven (pSL857/pSL582/pSL776) EGFP-NS3a (H1) sensors and EGFP-NS3a (H1) expression vectors (pSL775) were hydrodynamically injected into the tail vein of C57BL/6 mice before SEAP levels in the bloodstream were measured after 24 h. Mice receiving pcDNA3.1 (+) instead of pSL775 were used as negative controls with no hepatic EGFP-NS3a (H1) expression (WT). Data are presented as the mean±SEM; n=5 mice per group.

FIG. 46. Demonstration of self-sufficient elimination of malignant cells in mice. (A-D) P_{MusAFP}-AND EGFP-NS3a (H1)-specific activation of apoptosis in mice. Mice harboring subcutaneous Hepa1-6_{EGFP-NS3a (H1)}-derived tumors received local injections of pcDNA3.1 (+) (negative control, n=5 mice per group) or plasmid DNA mixes comprising pSL886, pSL776 and pSL582 (treatment group, n=5 mice per group). (A) Daily changes of tumor size were assessed by calculating V=(length*width)²/2. (B) Tumors were weighed and harvested at the final experimental day for (C) image analysis and (D) measurement of Bax protein levels by Western Blot. Numbers on the left axis of Western blots represent molecular weights (MW) of target proteins.

### EXAMPLES

### Materials and Methods

Vector Design. References and molecular architectures for all expression vectors are provided in Table S2. Some expression vectors were constructed by in-fusion cloning using the Seamless Cloning Kit (Beyotime Biotechnology, Shanghai, China; cat. no. D7010M). PCR-amplification reactions were performed using KOD One PCR Master Mix (Toyobo Inc., Osaka, Japan; cat. no. KMM-201). Ligation reactions were performed using T4 DNA ligase (New England Biolabs, Beverly, MA; cat. no. M0202L). Restriction endonucleases were purchased from New England Biolabs.

Chemicals and recombinant proteins. Abscisic acid (cat. no. A8060; stock solution 100 mM in DMSO) and dimethyl sulfoxide (DMSO; cat. no. D8371) were purchased from Solarbio Life Sciences (Beijing, China). Danoprevir (cat. no. HY-10238; stock solution 10 mM in DMSO) and gibberellic acid (cat. no. HY-N1964; stock solution 100 mM in DMSO) were purchased from MedChemExpress (Monmouth Junction, NJ). Animal-free recombinant human EGF (cat. no. AF-100-15; 1000×stock solution in ddH₂O) was purchased from PeproTech EC Ltd. (London, United Kingdom). Grazoprevir (cat. no. S3728; stock solution 10 mM in DMSO (for in vitro experiments) or 100 g/L in DMSO (for in vivo experiments)) was purchased from Selleck Chemicals (Houston, TX). Rapamycin (cat. no. MC0181; stock solution 10 mM in DMSO) was purchased from ImmunoWay Biotechnology (Plano, TX). Rapamycin analogue (Rapalog; cat. no. 535057; stock solution 100 μM in EtOH) was purchased from Takara Bio Inc. (Kusatsu, Japan). 4-Nitrophenyl phosphate disodium salt hexahydrate (pNPP; cat. no. 333338-18-4) was purchased from Aladdin Biochemical Technology (Shanghai, China). Phenylmethylsulfonyl fluoride 100 mM solution (PMSF; cat. no. ST506-2) and poly-D-lysine 5 mg/ml solution (cat. no. C0312) were purchased from Beyotime Biotechnology (Shanghai, China). Diethanolamine (DEA; cat. no. D807525) and ethanol anhydrous (EtOH; cat. no. E809056) were purchased from Macklin Inc. (Shanghai, China). Polyethyleneimine MAX (PEI; cat. no. 24765; stock solution 1 mg/ml in ddH₂O) was purchased from Polysciences (Eppelheim, Germany). Vanillic acid (cat. no. R017640; stock solution 165 mM in DMSO) and isopropanol (cat. no. R018247) were purchased from Rhawn Chemicals (Shanghai, China). D-Glucose anhydrous (dextrose; cat. no. A610219), glycerol (cat. no. A501745; stock solution 10% w/w in ddH₂O), L-homoarginine hydrochloride (cat. no. A602842), magnesium chloride hexahydrate (MgCl₂; cat. no. A610328) and Tween-20 (cat. no. A100777) were purchased from Sangon Biotech (Shanghai, China). 4% PFA solution (cat. no. R20497) was purchased from Shanghai Yuanye Bio-Technology (Shanghai, China). Streptozotocin (STZ; cat. no. s0130) was purchased from Sigma-Aldrich (MilliporeSigma; Burlington, MA). Calcium chloride anhydrous (CaCl₂); cat. no. 10005861), chloroform (cat. no. 10006818), sodium chloride (NaCl; cat. no. 10019318; stock solution 5 M in ddH₂O), potassium chloride (KCl; cat. no. 10016308), sodium acetate (cat. no. 10018818; stock solution 3 M in ddH₂O) and trisodium citrate dihydrate (cat. no. 10019418) were purchased from Sinopharm Chemical Reagent (Shanghai, China). Puromycin dihydrochloride (cat. no. A1113803) and Blasticidin S HCl (cat. no. R21001) were purchased from Thermo Fisher Scientific (Waltham, MA). Triton X-100 (cat. no. X11206) was purchased from Xinyu Biological Technology (Shanghai, China). Murine RNase inhibitor (cat. no. R301) was purchased from Vazyme Biotech (Nanjing, China). Home-made stock solutions of 1 M Tris-HCl (pH 7.5) and 0.5 M EDTA were provided by Westlake University Core Facility.

Cell culture and transfection. Cell lines derived from human embryonic kidney cells (HEK-293T, ATCC: CRL-3216), murine hepatoma cells (Hepa1-6, ATCC: CRL-1830) and murine neuroblasts (N2A, ATCC: CRL-131) were cultivated in Dulbecco's modified Eagle's medium (DMEM; Thermo Fisher Scientific, Waltham, MA; cat. no. 12100046) supplemented with 10% (v/v) fetal bovine serum (Gibco FBS, Australia; Thermo Fisher Scientific, Waltham, MA; cat. no. 10099141, lot no. 2177370) and 1% (v/v) penicillin/streptomycin solution (PenStrep; Beyotime Biotechnology, Shanghai, China; cat. no. ST488). Cells derived from murine melanoma (B16-F10, ATCC: CRL-6475) were cultivated in Roswell Park Memorial Institute 1640 Medium (RPMI 1640; Sartorius AG, Göttingen, Germany; cat. no. 01-100-1A) supplemented with 10% (v/v) FBS and 1% PenStrep. All cells were cultured at 37° C. in a humidified atmosphere of 5% CO₂ in air. For passaging, cells of pre-confluent cultures were detached by incubation in 0.05% trypsin-EDTA (Sangon Biotech, Shanghai, China; cat no. A610629-0050; lot no. F319BA0030) for 3 min at 37° C., collected in 10 ml of cell culture medium, centrifuged for 2 min at 1000 rpm, and resuspended in fresh culture medium at 1.5×10⁵ cells/mL, and then seeded into new tissue culture plates. Cell number and viability were quantified using an Invitrogen Countess II AMQAX1000 Cell Counter (Thermo Fisher Scientific; cat no. AMQAX1000).

Unless indicated otherwise, transfection was performed at 12 h after seeding 50000 mammalian cells into each well of a 24-well plate. The cell culture medium was replaced with fresh medium (not containing transfection reagents) at 6 h after transfection. HEK-293T were transfected using a PEI-based protocol at a PEI: DNA ratio of 5:1 (w/w) and in a transfection volume of 50 μL native serum-free DMEM per well. N2A, Hepa1-6 and B16-F10 cells were transfected using Lipofectamine 3000 (ThermoFisher Scientific, cat. no. L3000015) reagent according to the manufacturer's instructions. In vitro-transcribed mRNA was transfected by mixing 0.75 μL Lipofectamine 3000 (without the P3000 reagent) with 500 ng nucleic acids in a transfection volume of 50 μL native serum-free Opti-MEM medium (Thermo Fisher Scientific, cat. no. 31985062) per well. Transfection agents and nucleic acids were incubated for 15 min at 25° C. before dropwise addition to cells. For RNA transfection, cell culture medium was replaced with Lipofectamine 3000-free medium at 4 h post transfection.

Lentivirus Production. Recombinant replication-deficient lentivirus particles were generated by transfecting 5×106 native HEK-293T cells (cultured in a 10 cm dish) with 5 μg pMD2. G (Addgene plasmid no. 12259), 10 μg psPAX2 (Addgene plasmid no. 12260) and 10 μg transfer plasmid carrying the desired gene expression cassette. At 48 h after transfection, culture supernatants containing lentiviruses were collected and cells were cultivated for another 48 h following medium exchange. Both harvested stocks were mixed and purified with a 0.45 mm filter for experimental use or storage at −80° C.

Generation of stable cell lines. Polyclonal HEK_{EGFP-NS3a (H1)}, N2A_{EGFP-NS3a (H1)}, Hepa1-6_{EGFP-NS3a (H1)}, and B16-F10_{EGFP-NS3a (H1)} populations, transgenic for stable expression of EGFP-NS3a (H1), were constructed by co-transfecting 500 ng pSL816 (Table S2) and 5 ng pCMV-T7-SB100 (Addgene plasmid no. 34879) into 5×10⁴ native HEK-293T, N2A, Hepa1-6, and B16-F10 cells, respectively. After selection with 100 μg/ml puromycin dihydrochloride, 10% of the surviving population with highest EGFP expression was subjected to FACS-mediated cell sorting for using the MA900 Multi-Application Cell Sorter (Sony Biotechnology; San Jose, CA). Monoclonal HEK-MOR9 (C0) cells stably transgenic for constitutive MOR9-1 expression were constructed by transduction of 5×10⁴ HEK-293T cells with supernatants containing lentiviral particles created with pLZ276 (Table S2) as transfer plasmid. Following selection with 100 μg/ml puromycin, single cell clones showing highest MOR9-1 expression were be picked and harvested.

In vitro transcription. Template DNA fragments containing a T7 promoter were isolated from corresponding plasmids by restriction endonuclease treatment and transcribed with a T7 High Yield RNA Transcription Kit (Vazyme Biotech, Nanjing, China; cat. no. TR-101) after the addition of 40 mM 3′-O-Me-m1G (5′) ppp (5′) G RNA Cap Structure Analog (New England Biolabs, Beverly, MA; cat. no. S1411L). After the removal of template DNA using 1 U RNase-free DNaseI (Vazyme Biotech; cat. no. EN401-01), mRNA was purified by precipitation with 0.3 M sodium acetate at −20° C., followed by centrifugation at 4° C. and 15000 rpm for 30 min and washing with 70% EtOH. Purified mRNA was re-suspended in RNA-free ddH₂O and RNA was quantified by measuring UV absorption with a UVP Crosslinker CL-3000 (Analytik Jena GmbH, Jena, Germany).

RNA extraction and cDNA synthesis. Total RNA of cells was isolated using the Trizol RNA extraction method. In brief, 3000 cells were mixed with 1 ml TRIzol™ Reagent (ThermoFisher Scientific, cat. no. 15596018) and vortexed until no precipitate could be observed. The cell lysate was then mixed with 200 μl chloroform and centrifuged at 12,000 g at 4° C. for 15 min. The aqueous phase was collected, mixed with 500 μl isopropanol and incubated at −20° C. for 30 min to precipitate RNA. The RNA pellet was harvested by centrifugation at 4° C. and 15000 g for 30 min. washed with 70% EtOH, air-dried and resuspended in 50 μl RNase-free water. For cDNA synthesis, 1 μg RNA was taken for cDNA synthesis using the HiScript II Q RT SuperMix (Vazyme Biotech; cat. no. R223-01) for qPCR.

Reverse transcription-polymerase chain reaction (RT-PCR). For quantitative analysis, PCR reaction was carried out with an initial step of 95° C. for 30 s followed by 40 cycles of 95° C. for 10 s and 60° C. for 30 s on the Applied Biosystems QuantStudio 1 Real-Time PCR System (ThermoFisher Scientific) using the ChamQ Universal SYBR qPCR Master Mix (Vazyme Biotech; cat. no. Q711-02) and the primers listed in Table S1. The relative cycle threshold (CT) was determined and normalized against the expression level of endogenous human glyceraldehyde 3-phosphate dehydrogenase (GAPDH) or murine ribosomal protein (Rplp0) genes.

RNA immunoprecipitation (RIP)-qPCR. At 48 h after transfection, cells were harvested with ice-cold lab-made PBS (Westlake University Core Facility), resuspended in RNA-free NETN300 cell lysis buffer (50 mM Tris-HCl, 300 mM NaCl, 2 mM EDTA, 0.05% Triton X-100, 1 mM PMSF and 50 U/ml murine RNase inhibitor) and incubated for 15 min on ice. Following centrifugation at 16,000 g and 4° C. for 15 min, 4% of the supernatant was collected as the “input sample” and the remaining supernatant was diluted with RNA-free NETN0 Buffer (NETN300 cell lysis buffer without NaCl) to reach a final NaCl concentration of 100 mM before samples were immunoprecipitated through incubation with anti-Flag affinity gel (Beyotime Biotechnology; cat. no. P2271) for 90 min at 4° C. The anti-Flag affinity gel was then centrifuged at 6000 g for 30 s and washed 3 times with ice-cold NETN3000 wash buffer (50 mM Tris-HCl, 300 mM NaCl, 0.05% Triton X-100 and 1 mM PMSF). RNA in both the “input sample” and affinity gel was extracted for RT-PCR analysis. The method was adapted from that described elsewhere (Kim and Dekker, 2018).

Co-immunoprecipitation (co-IP). At 48 h after transfection, cells were harvested and lysed for 30 min at 4° C. with cell lysis buffer (Beyotime Biotechnology; cat. no. P0013) supplemented with 1 mM PMSF. Following centrifugation at 14,000 g for 10 min, 100 μl of supernatant was collected as the “input sample” and the remaining supernatant was immunoprecipitated by incubation for 3 h at 4° C. with anti-Flag affinity gel (Beyotime Biotechnology; cat. no. P2271). The anti-Flag affinity gel was then centrifuged at 6000 g for 30 s and washed 3 times with IP wash buffer (20 mM Tris-HCl, 0.2 mM EDTA, 100 mM KCl, 2 mM MgCl₂, 0.1% Tween 20 and 10% glycerol). Proteins in both the “input sample” and affinity gel were mixed with 5×SDS page loading buffer (Beyotime Biotechnology; cat. no. P0015), boiled at 98° C. for 10 min and prepared for Western blotting. The method was adapted from that described elsewhere (DeCaprio and Kohl, 2020).

Western blotting. After treatment with 5×SDS page loading buffer (Beyotime Biotechnology; cat. no. P0015) at 98° C. for 10 min, samples were resolved on 8%, 10% or 12% SDS polyacrylamide gels from the SDS-PAGE preparation kit (Sangon Biotech, Shanghai, China; cat. no. C631100) and electroblotted onto polyvinylidene fluoride (PVDF) Western blotting membranes (Merck Millipore KGaA, Darmstadt, Germany; cat. no. 03010040001). Membranes were incubated in BeyoECL Moon detection reagent (Beyotime Biotechnology; cat. no. P0018FS) and visualized using the Amersham Imager 600 (AI600 RGB; GE Healthcare, Uppsala, Sweden) after application of primary rabbit anti-Flag antibody (Sigma-Aldrich; cat. no. F7425), rabbit anti-HA antibody (Cell Signaling Technology, Danvers, MA; cat. no. 3724; lot. no. 10), rabbit anti-eIF4G antibody (Cell Signaling Technology; cat. no. 2498; lot. no. 4), rabbit anti-eIF4E antibody (Cell Signaling Technology; cat. no. 2067; lot. no. 8), rabbit anti-GAPDH (6C5) antibody (Santa Cruz Biotechnology; cat. no. sc-32233; lot. no. L2019) or rabbit anti-Bax antibody (Cell Signaling Technology Cat. no. 14796; Lot. No. 800) and secondary HRP-labeled goat anti-rabbit IgG (Beyotime Biotechnology; cat. no. A0208; lot. no. 110219200406).

Quantification of target gene expression. SEAP Assay. Expression levels of human placental secreted alkaline phosphatase (SEAP) in culture supernatants were quantified based on p-nitrophenyl phosphate-derived light absorbance at 415 nm (Wang et al., 2015). SEAP levels in mouse serum were profiled using a SEAP chemiluminescence assay kit (Roche Diagnostics GmbH, Mannheim, Germany; cat. no. 11 779 842 001). NanoLuc Assay. NanoLuc levels were profiled using the Nano-Gio® Luciferase Assay System (Promega, Madison, WI; cat. no. N1120). FLuc Assay. Firefly luciferase levels were profiled using the Luciferase Reporter Gene Assay Kit (Yeasen Biotechnology, Shanghai, China; cat. no. 11401ES60) after lysis of the cells for 15 min at 4° C., followed by centrifugation at 12000 g for 5 min. Insulin ELISA. Modified rodent insulin levels (mINS) in culture supernatants and mouse serum were quantified with a mouse insulin ELISA kit (Mercodia Inc., Uppsala, Sweden; cat. no. 10-1247-01).

Flow cytometry. Cell populations were analyzed with a CytoFLEX LX flow cytometer (Beckman Coulter, Indianapolis, IN) equipped for detection of EGFP (488 nm laser, 525/40 emission filter) and mCherry (561 nm laser, 610/20 emission filter) and set to exclude dead cells and cell doublets. 10,000 cells were recorded per data set and analyzed with FlowJo™ software (v10; BD Biosciences). Weighted EGFP or mCherry expression levels were determined by setting an arbitrary threshold of 10⁵ fluorescence units and multiplying the percentage of gated cells by their median fluorescence.

Fluorescence imaging. Fluorescence microscopy was performed with a Nikon ECLIPSE Ts2-FL fluorescence microscope (Nikon Instruments Inc., Melville, NY) equipped with a C-mount camera, F-mount camera, a20× objective, an excitation and emission filter set (EGFP: 488/509 nm; mCherry: 587/610 nm) and OPLENIC software (version x64, 10.1.14643.20190511).

Confocal microscopy. At 24 h after transfecting lug plasmid DNA into 1×10⁵ cells seeded on a 20 mm glass-bottomed cell culture dish (Wuxi NEST Biotechnology, Wuxi, China; cat. no. 801001) coated with 5 mg/ml poly-D-lysine, the cell culture medium was removed and cells were washed with 1 mL PBS and fixed with 1 mL 4% PFA solution. After 10 min, cells were washed with PBS and stained with DAPI staining solution (Beyotime Biotechnology; cat. no. C1005) for 15 min in the dark. Finally, the cells were washed three times with PBS and imaged with a A1R HD25 confocal microscope (Nikon Instruments Inc., Melville, NY).

AAV production. AAV2/8-(GNCR)₃-NSP3, AAV2/8-MCP-(NS3a)₃ and AAV2/8-SEAP-(MS2)₂₄-HHR-pA were produced by PackGene Biotech (Guangzhou, China) using the transfer plasmids pSL511 (Table S2), pSL512 (Table S2) or pSL446 (Table S2), respectively.

Animal experiments. Animal experiments were performed according to the protocol (Protocol ID: 20-001-XMQ) approved by the Institutional Animal Care and Use Committee (IACUC) of Westlake University and in accordance with the Animal Care Guidelines of the Ministry of Science and Technology of the People's Republic of China. Hydrodynamic tail vein injection. Endotoxin-free plasmids were diluted in Ringer's solution (147 mM NaCl, 4 mM KCl, 1.13 mM CaCl₂)) to reach final injection volume of 100 μL per gram body-weight and injected into tail veins of >6-week-old mice using 5 mL syringes. STZ TID mouse model. Fasted 6-week-old male WT C57BL/6 mice were injected daily with freshly diluted STZ (50 mg/kg in 200 μl ice-cold sodium citrate buffer) for five consecutive days. Chronic fasting hyperglycemia (>15 mM) developed after 3 weeks. Xenograft tumor model. 1×10⁶ B16-F10- or 2×10⁶ Hepa1-6-derived cell lines in 0.1 mL sterile PBS were subcutaneously injected into the right lower back of 4-week-old male WT C57BL/6 mice. After 7 days, each animal received intratumoral injection of 60 μL Lipofectamine 3000 solution containing 20 μg of plasmid DNA on different days after cell implantation under anesthesia. Drug administration. Grazoprevir 100 μg/μl was administered by intraperitoneal (i.p.) injection or oral gavage. Blood sampling. Whole blood was collected from the submandibular vein of mice and clotted by incubation at 4° C. for 2 h, and then serum was isolated by centrifugation for 8 min at 8000 g. Glycemia measurement. Glycemia of mice was measured with a commercial glucometer (Sinocare plus Code Glucometer; detection range: 1.1-33.3 mM) purchased at a local pharmacy. Glucose tolerance tests (GTT). D-Glucose was freshly prepared and intraperitoneally injected in mice at a 0.75 g/kg dose before time zero.

Data analysis. Two-tailed unpaired Student t-tests were used to evaluate the statistical significance of differences between two groups. For tumor volume studies, two-way ANOVA was used for statistical analysis. P values less than 0.05 were considered statistically significant. Statistical parameters and corresponding P values are included in the figure legends. All analyses were performed using GraphPad Prism 9 (Graph Pad Software, San Diego, CA).

### Example 1: Gene Regulation by Trigger-Inducible mRNA Circularization in Mammalian Cells

Following the “closed-loop” model (Gray et al., 2000; Jackson et al., 2010), we considered that manipulating the mRNA circularization process would be an attractive accession point to achieve trigger-inducible regulation of translational initiation. In the basal state (OFF), the absence of a circularized configuration would compromise scanning efficiency and promote mRNA degradation by endogenous deadenylases (Tang et al., 2019). Thus, initiation of translation (ON) should depend on how efficiently the closed-loop configuration is established-atask that is naturally mediated by endogenous PABP binding to the poly (A) region (Jackson et al., 2010; Passmore and Coller, 2021).

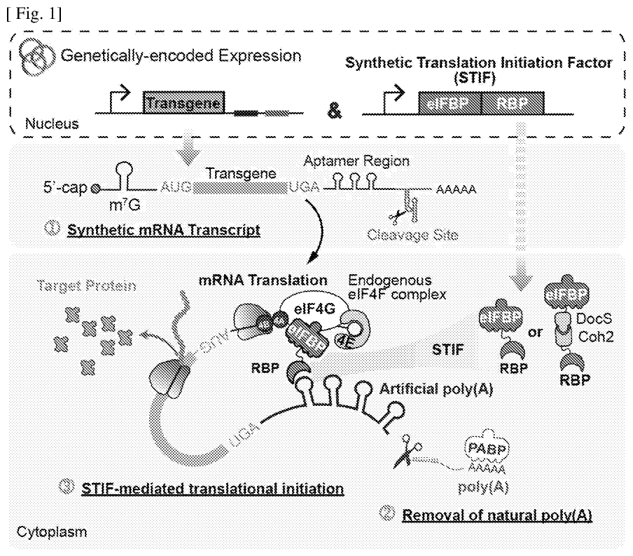

To engineer an analogous regulation framework for user-defined (trans) gene control (FIG. 1), we designed an mRNA transcript containing a control region comprising RBP-specific aptamers in the 3′-UTR (e.g. those listed in Table 2). Thus, circularization and translation of the synthetic mRNA transcript would depend on the presence of a synthetic translation initiation factor (STIF; e.g. those listed in Table 1) that mimics PABP function by simultaneously binding the aptamer-based control region and any one member of the preinitiation complex (FIG. 1).

For proof of concept, we engineered various STIF constructs by fusing different RNA-binding protein (RBPs) (e.g. the archaeal ribosomal protein L7Ae (SEQ-ID NO: 92) or bacteriophage-derived MS2 coat protein (MCP, SEQ-ID NO: 98)) to different eIF4F-binding proteins (eIFBP), such as human PABP (SEQ-ID NO: 108), eIF4G (SEQ-ID NO: 84) and eIF4E (SEQ-ID NO: 83), as well as rotaviral non-structural protein 3 (NSP3, SEQ-ID NOs: 91&106) and caliciviral VPg (SEQ-ID NO: 120), resulting in the following specific STIF constructs:

TABLE 1
STIF constructs designed in this invention.

| STIF construct | RBP-domain | eIFBP-domain |
|---|---|---|
| L7Ae-NSP3 (SEQ-ID NO: 43) | L7Ae (SEQ-ID NO: 92) | NSP3 (SEQ-ID NO: 106) |
| L7Ae-eIF4E (SEQ-ID NO: 44) | L7Ae (SEQ-ID NO: 92) | eIF4E(K119A) (SEQ-ID NO: 83) |
| L7Ae-hNSP3 (SEQ-ID NO: 45) | L7Ae (SEQ-ID NO: 92) | hNSP3 (SEQ-ID NO: 91) |
| 3xFLAG-L7Ae-NSP3 (SEQ-ID NO: 49) | L7Ae (SEQ-ID NO: 92) | NSP3 (SEQ-ID NO: 106) |
| MCP-NSP3 (SEQ-ID NO: 59) | MCP (SEQ-ID NO: 98) | NSP3 (SEQ-ID NO: 106) |
| MCP-hNSP3 (SEQ-ID NO: 60) | MCP (SEQ-ID NO: 98) | hNSP3 (SEQ-ID NO: 91) |
| PABP-L7Ae (SEQ-ID NO: 65) | L7Ae (SEQ-ID NO: 92) | PABP (SEQ-ID NO: 108) |
| PABP-L7Ae-3xFLAG (SEQ-ID NO: 66) | L7Ae (SEQ-ID NO: 92) | PABP (SEQ-ID NO: 108) |
| eIF4G-2CaM-M13-L7Ae (SEQ-ID NO: 69) | L7Ae (SEQ-ID NO: 92) | eIF4G (SEQ-ID NO: 84) |
| PABP-MCP (SEQ-ID NO: 206) | MCP (SEQ-ID NO: 98) | PABP (SEQ-ID NO: 108) |
| MCP-eIF4E (SEQ-ID NO: 244) | MCP (SEQ-ID NO: 98) | eIF4E(K119A) (SEQ-ID NO: 83) |
| eIF4G-MCP (SEQ-ID NO: 253) | MCP (SEQ-ID NO: 98) | eIF4G (SEQ-ID NO: 84) |
| 3xFLAG-MCP-NSP3 (SEQ-ID NO: 260) | MCP (SEQ-ID NO: 98) | NSP3 (SEQ-ID NO: 106) |
| MCP-VPg (SEQ-ID NO: 275) | MCP (SEQ-ID NO: 98) | VPg (SEQ-ID NO: 120) |
| MCP^{V29I}-VPg (SEQ-ID NO: 276) | MCP^{V29I} (SEQ-ID NO: 200) | VPg (SEQ-ID NO: 120) |
| L7Ae-VPg (SEQ-ID NO: 281) | L7Ae (SEQ-ID NO: 92) | VPg (SEQ-ID NO: 120) |

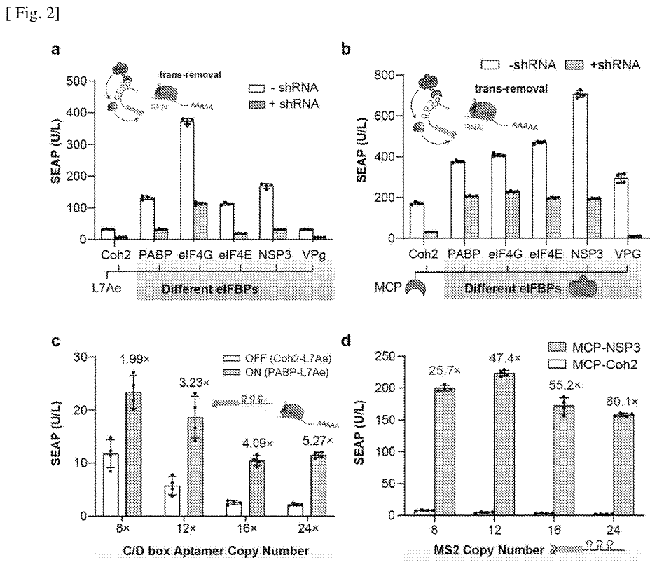

To validate their functions, HEK-293 cells (ATCC: CRL-3216; see Methods section above) were co-transfected with a SEAP expression vector containing 8 tandem L7Ae-specific C/D-box repeats (SEQ-ID NO: 131), an shRNA-216 expression vector (SEQ-ID NO: 126) and expression vectors for different STIF variants as described in Table 1 above or an L7Ae-Coh2 protein incapable of translational initiation (SEQ-ID NO: 33, negative control). SEAP levels in culture supernatants were quantified at 48 h post transfection according to the Quantification of target gene expression in the Materials and Methods (FIG. 2A). Similarly, HEK-293 cells were co-transfected with a SEAP expression vector containing 8 tandem MCP-specific MS2-box repeats (SEQ-ID NO: 180), an shRNA-216 expression vector (SEQ-ID NO: 126) and expression vectors for different STIF variants or an MCP-Coh2 protein incapable of translational initiation (SEQ-ID NO: 51, negative control). SEAP levels in culture supernatants were quantified at 48 h post-transfection (FIG. 2B). For STIF overexpression, any method that result in production of protein sequences listed in Table 1 in living cells can be used.

From the results in FIG. 2A and FIG. 2B we can see that
(i) poly (A)-removal through shRNA-216 overexpression (whenever SEQ-ID NO: 126 was present) was essential to reduce basal expression levels that correspond to conditions where Coh2 (SEQ-ID NO: 77) was fused to L7Ae (SEQ-ID NO: 92) (FIG. 2A) or MCP (SEQ-ID NO: 98) (FIG. 2B),
(ii) that fusion of MCP (SEQ-ID NO: 98) or L7Ae (SEQ-ID NO: 92) to eIF4G (SEQ-ID NO: 84) or NSP3 (SEQ-ID NOs: 91&106) allowed for the best performance in RBP-dependent translational (up) regulation,
(iii) that RBP and eIFBP domains can be flexibly swapped to produce functional STIF regulators (i.e., in eIF4G-MCP (SEQ-ID NO: 253), the RBP-domain MCP is on the C-terminus; in MCP-eIF4E (SEQ-ID NO: 244), this RBP-domain can also be on the N-terminus), and
(iv) that STIFs can also contain other arbitrary protein domains inserted between RBP and eIFBP domains (such as in the case of eIF4G-2CaM-M13-L7Ae (SEQ-ID NO: 69), where a calmodulin-like motif 2CaM-M13 (SEQ-ID NO: 312) was inserted between eIF4G and L7Ae).

Based on these experimental findings, it suggested that STIF variants containing any reversed configuration that are not listed in Table 1 (e.g. MCP-PABP instead of PABP-MCP; L7Ae-PABP instead of PABP-L7Ae; etc.), as well as changing MCP or L7Ae to any or their mutants (e.g. in the case of MCP (V29I)-VPg, SEQ-ID NO: 276) or to any other RBPs (e.g. Bacteriophage λ-derived N-Peptide (AN, SEQ-ID NO: 100)), would also result in functional STIF construction showing similar results as in FIG. 2A and FIG. 2B.

Next, we showed that the relative fold-changes between basal STIF-independent expression and STIF-mediated upregulation could be further fine-tuned by increasing the number of aptamer repeats placed downstream of the protein-coding region (FIG. 2C, 2D). Experimentally, HEK-293 cells were co-transfected with SEAP expression vectors containing different tandem repeats of the L7Ae-specific C/D-box aptamer ((C/D-box)₈, SEQ-ID NO: 131; (C/D-box)₁₂, SEQ-ID NO: 133; (C/D-box)₁₆, SEQ-ID NO: 132; (C/D-box)₂₄, SEQ-ID NO: 134), an shRNA-216 expression vector (SEQ-ID NO: 126) and any expression vector producing either PABP-L7Ae (ON; SEQ-ID NO: 65) or a Coh2-L7Ae protein incapable of binding eIF4F (OFF; SEQ-ID NO: 8). SEAP expression in culture supernatants was scored at 48 h post transfection. For MCP-based STIF systems, HEK-293 cells were transfected with SEAP expression vectors containing different tandem repeats of the MCP-specific MS2-box aptamer ((MS2-box)₈, SEQ-ID NO: 179; (MS2-box)₁₂, SEQ-ID NO: 178; (MS2-box)₁₆, SEQ-ID NO: 138; (MS2-box)₂₄, SEQ-ID NO: 137) and any expression vector producing either MCP-NSP3 (ON; SEQ-ID NO: 59) or an MCP-Coh2 protein incapable of binding eIF4F (OFF; SEQ-ID NO: 51). SEAP expression in culture supernatants was scored at 48 h post-transfection.

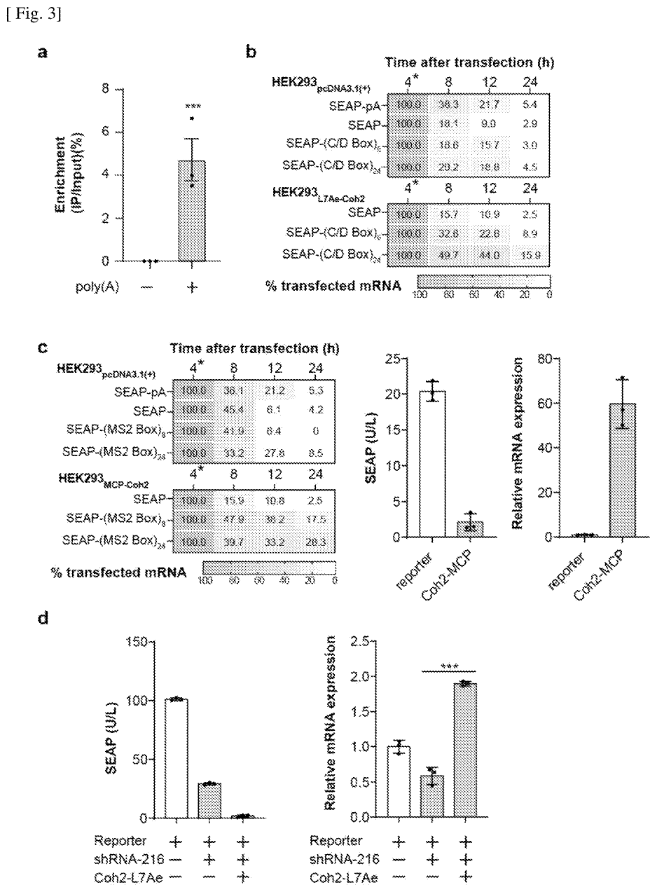

Thus, this aptamer region (e.g. (C/D-box), or (MS2-box) n) can be regarded as an artificial poly (A) signal or a “poly (A)-surrogate” mediating strict STIF-dependent mRNA translation, with genetically encoded poly (A)-excision emerging as seminal for effective (trans) gene control by enabling “escape” from endogenous PABP-mediated processes (FIG. 3A).

In fact, using RNA Immunoprecipitation-qPCR (detailed experimental procedure is described in the Methods section above), we quantified the binding capability of PABP to poly (A)-containing RNA. For this purpose, HEK-293 cells were transfected with a constitutive expression vector for 3×FLAG-tagged PABP-L7Ae (SEQ-ID NO: 66) reflecting the RNA binding capacity of endogenous PABP. After 24 h, 10 μg of in vitro-transcribed EGFP-mRNA with (+) or without (−) a poly (A) tail was added (produced from pWS164, Table S2) according to the description in the “in vitro transcription” part of Methods section above). At 3 h after transfection, RNA was extracted and co-immunoprecipitated using anti-Flag affinity gel (Beyotime Biotechnology; cat. no. P2271). Results of qRT-PCR analysis (detailed experimental procedure is described in the Methods section above) showing the ratio (%) of EGFP-mRNA in samples before (input) and after immunoprecipitation (IP) confirm that mRNAs containing “poly (A)-surrogates” instead of native poly (A) are no longer bound by endogenous PABP (FIG. 3A). Furthermore, the size of engineered poly (A)-surrogates (reflected the number n of tandem (C/D-box), or (MS2-box), repeats placed in the 3′-UTR) appears to have a positive impact to delay mRNA decay in mammalian cells (FIG. 3B, 3C). Also, any aptamer-specific protein binding to such region may confer increased stability on the target mRNA (FIGS. 3C, 3D).

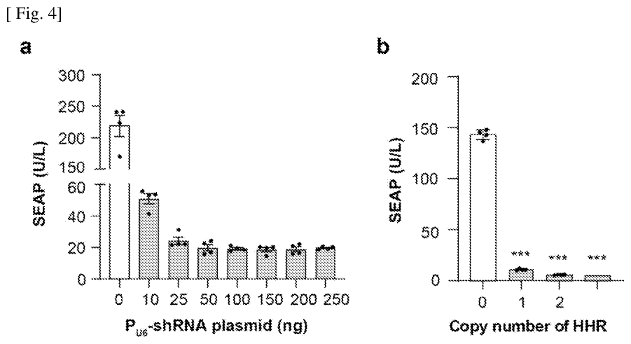

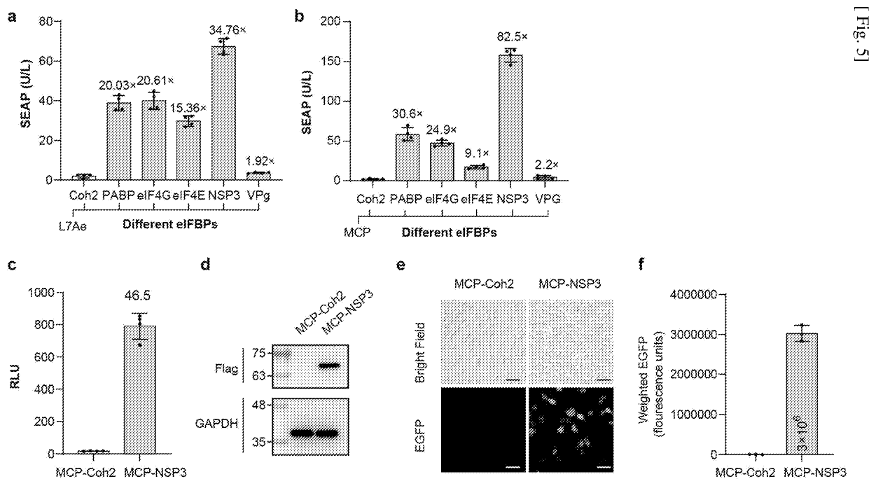

Next, we engineered one-component expression vectors for poly (A)-deficient mRNA for strict STIF-specific target gene expression. For this purpose, we replaced trans-acting shRNA-binding sites in SEQ-ID NOs: 131-134 (FIG. 4A) with cis-acting hammerhead ribozyme HHR motifs (SEQ-ID NO: 124) (FIG. 4B), which should trigger spontaneous self-excision of the natural poly (A) signal either before or immediately after nuclear mRNA export. Using this strategy, fusion of different eIFBPs to L7Ae (FIG. 5A) or MCP (FIG. 5B) could also result in activation of gene expression from poly (A)-deficient mRNA, with L7Ae-NSP3 (SEQ-ID NO: 43) and MCP-NSP3 (SEQ-ID NO: 59) showing highest induction folds between basal and activated states following HHR-mediated poly (A)-removal (FIGS. 5A, 5B). Essentially, these experiments are analogous to those described in FIG. 2A and FIG. 2B, only with the difference that SEAP expression vectors containing 24 instead of 8 tandem L7Ae-specific C/D-box repeats (FIG. 5A) or MCP-specific MS2-box repeats (FIG. 5B) is now used as reporter vectors, and that shRNA-216-specific binding sites (SEQ-ID NO: 122) are exchanged into HHR elements (SEQ-ID NO: 124). When we replaced SEAP expression vectors by isogenic target mRNA encoding for Firefly Luciferase (FLuc; SEQ-ID NO: 305) (FIGS. 5C, 5D) or enhanced green fluorescence protein (EGFP; SEQ-ID NO: 82) (FIGS. 5E, 5F), we showed that MCP-NSP3 (SEQ-ID NO: 59) could also regulate a variety of other reporter genes.

Therefore, any mRNA with generic descriptions that include, but are not limited to “5′-UTR-GOI-(MS2-box)ₙ-BS (shRNA)ₙ-PA-3′”, “5′-UTR-GOI-(C/D-box)ₙ-BS (shRNA)ₙ-pA-3′”, “5′-UTR-GOI-(MS2-box)ₙ-HHRₙ-pA-3′” and “5′-UTR-GOI-(C/D-box)ₙ-HHRₙ-pA-3′”, whose 3′-UTR of a particular gene of interest (GOI) consist of a poly (A)-surrogate capable of binding a specific target protein (RBP: including but not limited to L7Ae (SEQ-ID NO: 92), MCP (SEQ-ID NO: 98) and λ-N (SEQ-ID NO: 100)) and an RNA cleavage site enabling pre-programmed poly (A)-removal (including but not limited to BS (shRNA-216) (SEQ-ID NO: 122) and HHR (SEQ-ID NO: 124)) are considered “STIF-specific target gene mRNA” created by the present invention (FIG. 1, green box). In this study, the following constructs were created and validated (Table 2):

TABLE 2
Synthetic mRNA transcripts containing RBP-specific poly (A) - surrogates
enabling STIF-dependent translation and expression of different GOIs.

| SEQ- | | Poly(A)- | | Related | Specific |
|---|---|---|---|---|---|
| ID | GOIs | surrogate | Cleavage site | Plasmids | to RBP |
| 131 | SEAP | (C/D-box)₈ | (BS(shRNA-216))₂ | pSL31 | L7Ae |
| 132 | SEAP | (C/D-box)₁₆ | (BS(shRNA-216))₂ | pSL80 | L7Ae |
| 133 | SEAP | (C/D-box)₁₂ | (BS(shRNA-216))₂ | pSL81 | L7Ae |
| 134 | SEAP | (C/D-box)₂₄ | (BS(shRNA-216))₂ | pSL88 | L7Ae |
| 135 | NanoLuc | (C/D-box)₂₄ | (BS(shRNA-216))₂ | pSL274 | L7Ae |
| 136 | SEAP | (C/D-box)₂₄ | HHR | pSL355 | L7Ae |
| 137 | SEAP | (MS2-box)₂₄ | HHR | pSL468, pSL511 | MCP |
| 138 | SEAP | (MS2-box)₁₆ | HHR | pSL516 | MCP |
| 139 | NanoLuc & mCherry | (MS2-box)₂₄ | HHR | PSL683 | MCP |
| 140 | NanoLuc & mINS | (MS2-box)₂₄ | HHR | pSL685, pSL688 | MCP |
| 142 | SEAP | none | (HHR)₂ | pSL767 | none |
| 143 | SEAP | none | (HHR)₄ | pSL768 | none |
| 145 | mBax | (MS2-box)₂₄ | HHR | pSL831 | MCP |
| 148 | SEAP | none | HHR | PSLM97 | none |
| 149 | EGFP | (MS2-box)₂₄ | HHR | pQZ112, pSL1308 | MCP |
| 163 | NanoLuc | (MS2-box)₂₄ | HHR | pQZ111 | MCP |
| 174 | NanoLuc & mCherry | (MS2-box)₁₆ | HHR | pSL1003 | MCP |
| 175 | SEAP & NanoLuc | (C/D-box)₂₄ | none | pPW21 | L7Ae |
| 176 | SEAP | (C/D-box)₄ | none | pQZ8 | L7Ae |
| 177 | 3xFLAG-FLuc | (MS2-box)₂₄ | HHR | pSL781 | MCP |
| 178 | SEAP | (MS2-box)₁₂ | HHR | pSL1284 | MCP |
| 179 | SEAP | (MS2-box)₈ | HHR | pSL515 | MCP |
| 180 | SEAP | (MS2-box)₈ | (BS(shRNA-216))₂ | pSL1331 | MCP |

Based on the results and findings in the present application, it can be known:
(i) the gene on interest (GOI) can be exchanged to any nucleic acid segment encoding for any polypeptide of interest (i.e. any RNA sequence starting with nucleotides AUG and terminating with nucleotide sequences UAG, UAA or UGA);
(ii) the poly (A)-surrogate can be exchanged to any segment that contains one or multiple n aptamer repeats binding to a specific RNA-binding protein (RBP). According to our experimental results, n can be any number between 1 and 1000 and most preferably n shall be 8, 16 or 24;
(iii) For genetically encoded expression in mammalian cells, the cleavage site can comprise one or multiple n repeats of any ribozyme or target site for nucleases. According to our experimental results, n can be any number between 1 and 100 and most preferably n shall be any number between 1 and 4.

Full sequences of all key elements are shown in Sequence List. To create such sequences, any molecular cloning techniques or nucleic acids synthesis strategies capable of producing corresponding plasmid DNA or synthetic mRNA can be used.

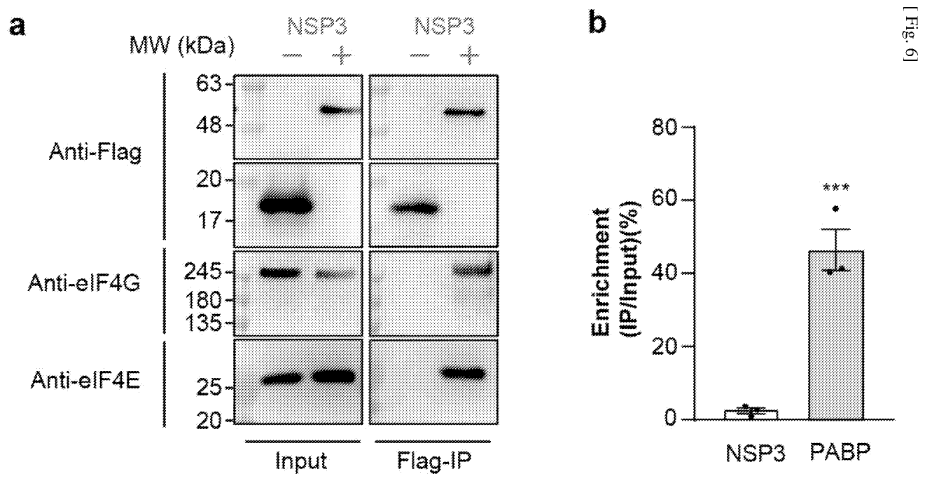

Because STIFs were designed to mimic endogenous PABP in binding both mRNA and the eIF4F complex, we used co-immunoprecipitation and Western Blot to experimentally confirm that NSP3-containing STIFs do associate with the eIF4F complex underlying the closed-loop model (FIG. 6A) and that NSP3 showed much less non-specific binding to endogenous RNA than PABP (FIG. 6B). Specifically, HEK-293 cells were transfected with expression vectors for 3×FLAG-tagged MCP (−, SEQ-ID NO: 264) or MCP-NSP3 (+, SEQ-ID NO: 260) before one lysate fraction was immunoprecipitated at 48 h post transfection. Target proteins in lysate fractions before (input) and after immunoprecipitation (Flag-IP) were detected with anti-FLAG (Sigma-Aldrich; cat. no. F7425), anti-eIF4G (Cell Signaling Technology; cat. no. 2498; lot. no. 4) and anti-eIF4E antibodies (Cell Signaling Technology; cat. no. 2067; lot. no. 8). Detailed information on experimental procedures and involved consumables are described in the Methods section above. Western Blot results in FIG. 6A reveal that colocalization of FLAG-tagged proteins with endogenous eIF4G and eIF4E depends on NSP3, where the input sample serves as a control showing that eIF4G/4E are all present in the cells. Similarly, to quantify binding capacity of PABP- and NSP3-fusion proteins to endogenous RNA, we used RNA Immunoprecipitation-qPCR using a similar experimental setup as described above (for FIG. 3A). At 48 h after transfection of HEK-293 cells with expression vectors for 3×FLAG-tagged L7Ae-NSP3 (SEQ-ID NO: 49) or PABP-L7Ae (SEQ-ID NO: 66), RNA was extracted and co-immunoprecipitated using anti-Flag affinity gel (Beyotime Biotechnology; cat. no. P2271). The ratio (%) of endogenous GAPDH expression levels in samples before (input) and after immunoprecipitation (IP) revealed by qRT-PCR analysis (detailed experimental procedure is described in the Methods section above) was finally taken as a measure for the amount of RNA bound on PABP- and NSP3-containing constructs (FIG. 6B). These results confirm that STIF-dependent regulation of translation indeed occurs through the closed-loop model of mRNA circularization. This stage of gene expression was hitherto unexplored during various cell engineering strategies.

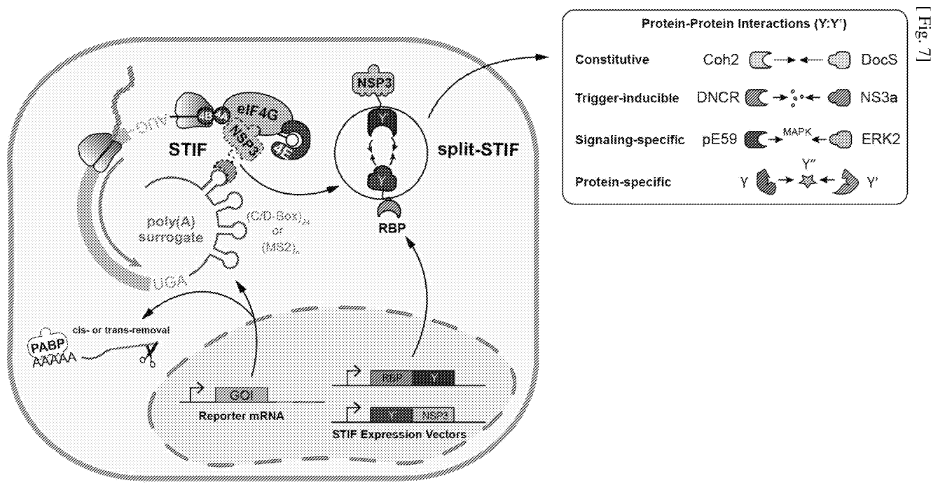

To use the STIF-based translational regulation strategy to engineer various “sense-and-response” features such as gene switches and genetic sensors, we split the conventional RBP-eIFBP or eIFBP-RBP architecture described in FIG. 1 and Table 1 into two independent proteins RBP-Y and Y′-eIFBP (FIG. 7). In such bipartite STIF systems, assembly of a functional STIF capable of activating RBP-specific mRNA translation would depend on the nature of protein-protein interactions (PPI) between any protein Y and Y′. It is worth noting (i) that the protein moieties Y and Y′ are flexibly interchangeable, (ii) that each domain of each split STIF component RBP-Y and Y′-eIFBP can be flexibly swapped to produce Y-RBP, RBP-Y′, Y-eIFBP and eIFBP-Y′ architectures and (iii) that multiple tandem repeats of Y and Y′ can be used to fine tune regulation performance. Based on these principles, following bipartite STIF systems were created for proof-of-concept:

TABLE 3
Bipartite STIF systems containing constitutive Y: Y′ protein-protein interactions.

| | STIF regulators (Y-RBP, RBP-Y′, | |
|---|---|---|
| Protein Y domain | Y-eIFBP or eIFBP-Y′) | Interacts with protein Y′ |
| DocS (SEQ-ID NO: 80) | 3xHA-(DocS)3-NSP3 (SEQ-ID NO: 274) | Coh2 (SEQ-ID NO: 77) |
| | (DocS)₁-NSP3 (SEQ-ID NO: 12) | |
| | (DocS)₂-NSP3 (SEQ-ID NO: 13) | |
| | (DocS)₃-NSP3 (SEQ-ID NO: 14) | |
| | DocS-VPg (SEQ-ID NO: 254) | |
| | DocS-eIF4E (SEQ-ID NO: 257) | |
| | DocS-eIF4G (SEQ-ID NO: 256) | |
| | MCP-DocS (SEQ-ID NO: 246) | |
| | PABP-DocS (SEQ-ID NO: 207) | |
| Coh2 (SEQ-ID NO: 77) | Coh2-L7Ae (SEQ-ID NO: 8) | DocS (SEQ-ID NO: 80) |
| | Coh2-NSP3 (SEQ-ID NO: 268) | |
| | (Coh2)₂-NSP3 (SEQ-ID NO: 269) | |
| | (Coh2)₃-NSP3 (SEQ-ID NO: 9) | |
| | L7Ae-Coh2 (SEQ-ID NO: 33) | |
| | L7Ae-(Coh2)₂ (SEQ-ID NO: 34) | |
| | L7Ae-(Coh2)₃ (SEQ-ID NO: 35) | |
| | L7Ae-(Coh2)₂-3xFLAG (SEQ-ID NO: 288) | |
| | MCP-Coh2 (SEQ-ID NO: 51) | |
| | MCP-(Coh2)₂ (SEQ-ID NO: 272) | |
| | MCP-(Coh2)₃ (SEQ-ID NO: 273) | |
| | λN-Coh2 (SEQ-ID NO: 205) | |
| NS3a(H1) (SEQ-ID NO: 105) | L7Ae-NS3a(H1) (SEQ-ID NO: 42) | ANR (SEQ-ID NO: 73) |
| | MCP-NS3a(H1) (SEQ-ID NO: 56) | |
| | MCP-(NS3a(H1))₂ (SEQ-ID NO: 57) | |
| | MCP-(NS3a(H1))₃ (SEQ-ID NO: 58) | |
| Bcl-XL (SEQ-ID NO: 74) | Bcl-XL-NSP3 (SEQ-ID NO: 7) | LD1 (SEQ-ID NO: 94), |
| | | LD3 (SEQ-ID NO: 95) |
| LD1 (SEQ-ID NO: 94) | L7Ae-LD1 (SEQ-ID NO: 37) | Bcl-XL (SEQ-ID NO: 74) |
| LD3 (SEQ-ID NO: 95) | L7Ae-LD3 (SEQ-ID NO: 38) | Bcl-XL (SEQ-ID NO: 74) |
| ANR (SEQ-ID NO: 73) | ANR-NSP3 (SEQ-ID NO: 2) | NS3a(H1) (SEQ-ID NO: 105) |
| | (ANR)₂-NSP3 (SEQ-ID NO: 267) | |
| | (ANR)₄-NSP3 (SEQ-ID NO: 3) | |
| | (ANR)₆-NSP3 (SEQ-ID NO: 4) | |
| | (ANR)₈-NSP3 (SEQ-ID NO: 5) | |
| EGFP (SEQ-ID NO: 82) | EGFP-NSP3 (SEQ-ID NO: 18) | LaG16 (SEQ-ID NO: 93) |
| | L7Ae-EGFP (SEQ-ID NO: 251) | |
| | MCP-EGFP (SEQ-ID NO: 245) | |
| LaG16 (SEQ-ID NO: 93) | MCP-LaG16 (SEQ-ID NO: 52) | EGFP (SEQ-ID NO: 82) |
| | MCP-(LaG16)₂ (SEQ-ID NO: 53) | |
| CCmut3 | 3xHA-CCmut3-NSP3 (SEQ-ID NO: 201) | BCR (SEQ-ID NO: 227) |
| | CCmut3-NSP3 (SEQ-ID NO: 258) | |
| ABI(iDab) (SEQ-ID NO: 223) | 3xFLAG-MCP-ABI(iDab) (SEQ-ID NO: 202) | ABL1 (SEQ-ID NO: 226) |
| | MCP-ABI(iDab) (SEQ-ID NO: 181) | |
| scFv35 (SEQ-ID NO: 113) | L7Ae-scFv35 (SEQ-ID NO: 47) | nNS3 (SEQ-ID NO: 303) |
| scFv162 (SEQ-ID NO: 112) | scFv162-NSP3 (SEQ-ID NO: 70) | nNS3 (SEQ-ID NO: 303) |
| | (scFv162)₂-NSP3 (SEQ-ID NO: 261) | |
| | (scFv162)₃-NSP3 (SEQ-ID NO: 262) | |

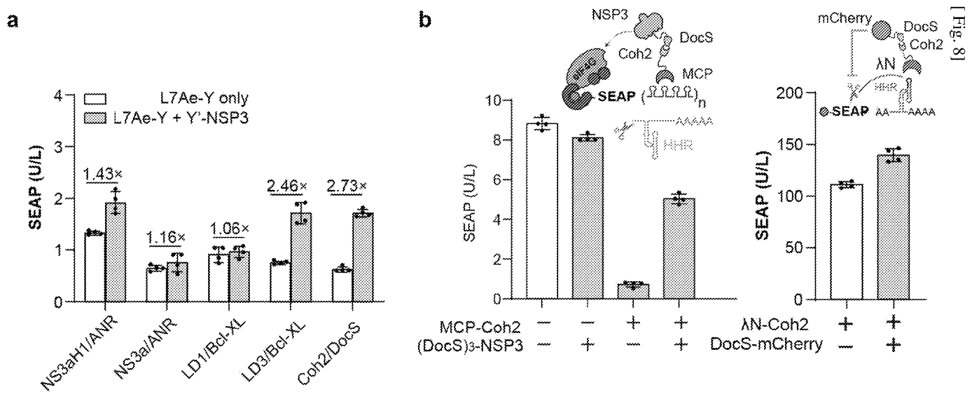

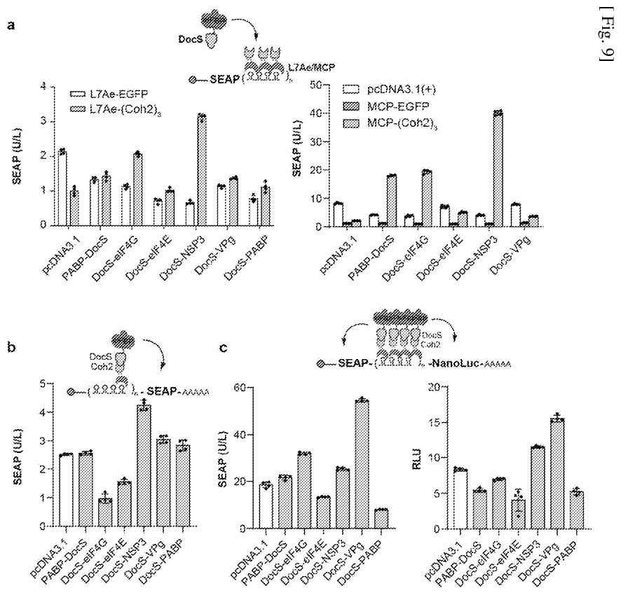

Full sequences of all key elements are shown in Sequence List. To create such sequences, any molecular cloning techniques or nucleic acids synthesis strategies capable of producing corresponding plasmid DNA or synthetic mRNA can be used. In this study, constructs were validated by transfection of encoding plasmids into HEK-293 whose cell culture and transfection methods are described in the Methods section above. To examine the ability of different constitutive protein dimerization pairs to induce spontaneous STIF assembly (FIG. 8A), HEK-293 cells were co-transfected with plasmids encoding SEAP mRNA containing an L7Ae-specific poly (A)-surrogate (SEQ-ID NO: 136) and constitutive expression vectors for different combinations of L7Ae- and NSP3-fusion proteins (e.g. STIF regulators listed in Table 3). Transfection of pcDNA3.1 (+) (Invitrogen, CA; cat-no. V79020) instead of NSP3-fusion proteins was used as a negative control. SEAP levels in culture supernatants were quantified at 48 h post-transfection. Results show that the high affinity pair Coh2/DocS (Barak et al., 2005) was effective in mediating L7Ae- and MCP-specific target gene translation (FIGS. 8A, 8B). In line with this, HEK-293 co-transfected with a SEAP expression vector containing 24 tandem MCP-specific aptamer repeats (SEQ-ID NO: 137) and different combinations of constitutive expression vectors for MCP-Coh2 (SEQ-ID NO: 51) and (DocS)₃-NSP3 (SEQ-ID NO: 14) also demonstrated modular and effective translational regulation (FIG. 8B). Furthermore, we were able to use MCP-Coh2 (SEQ-ID NO: 51) or L7Ae-Coh2 (SEQ-ID NO: 33) as mRNA-specific tethers to recruit various DocS-containing eIFBP constructs to different mRNA sites, such as 3′-UTR regions (FIG. 9A), 5′-UTR regions (FIG. 9B) or intergenic regions (FIG. 9C). In FIG. 9A, HEK-293 cells were transfected with an expression vector for SEAP-mRNA containing 24 tandem C/D-box-(left panel: SEQ-ID NO: 136) or MS2-box repeats in the 3′-UTR (right panel: SEQ-ID NO: 137), constitutive expression vectors for L7Ae or MCP fused to Coh2 (producing SEQ-ID NO: 33 or SEQ-ID NO: 51) or EGFP (producing SEQ-ID NO: 251 or SEQ-ID NO: 245), and constitutive expression vectors for various chimeric Coh2-specific DocS-containing eIFBP fusions (PABP-DocS, SEQ-ID NO: 207; DocS-eIF4G, SEQ-ID NO: 256; DocS-eIF4E, SEQ-ID NO: 257; DocS-NSP3, SEQ-ID NO: 12; DocS-VPg, SEQ-ID NO: 254). In FIG. 9B, HEK-293 cells were (co-) transfected with an expression vector for SEAP-mRNA containing 4 tandem C/D-box repeats in the 5′-UTR (SEQ-ID NO: 176), a constitutive L7Ae-(Coh2)₃ expression vector (SEQ-ID NO: 35) and constitutive expression vectors for various DocS-based fusion constructs such as PABP-DocS (PABP-DocS, SEQ-ID NO: 207; DocS-eIF4G, SEQ-ID NO: 256; DocS-eIF4E, SEQ-ID NO: 257; DocS-NSP3, SEQ-ID NO: 12; DocS-VPg, SEQ-ID NO: 254). In FIG. 9C, the reporter vector was exchanged into a mRNA construct containing 24 tandem C/D-box repeats placed downstream of SEAP- and upstream of NanoLuc-coding regions (SEQ-ID NO: 175). In all cases, transfection of pcDNA3.1 (+) (Invitrogen, CA; cat-no. V79020) instead of DocS- or MCP-expressing vectors was used as a negative control and SEAP levels in culture supernatants were quantified at 48 h post-transfection.

Next, we replaced constitutive Y:Y′-association pairs by various trigger-inducible PPI systems into the bipartite STIF framework, thus producing the following regulation systems:

TABLE 4
Bipartite STIF systems containing trigger-inducible Y: Y′ protein-protein interactions.

| | STIF regulators (Y-RBP, RBP-Y′, | Conditional interaction with |
|---|---|---|
| Protein Y domain | Y-eIFBP or eIFBP-Y′) | protein Y′ |
| ABI (SEQ-ID NO: 71) | L7Ae-(ABI)₃ (SEQ-ID NO: 31) | Abscisic acid-inducible binding to |
| | ABI-MCP (SEQ-ID NO: 259) | PYL1 (SEQ-ID NO: 111) |
| | ABI-L7Ae (SEQ-ID NO: 265) | |
| | ABI-NSP3 (SEQ-ID NO: 266) | |
| | L7Ae-ABI (SEQ-ID NO: 284) | |
| | NSP3-ABI (SEQ-ID NO: 294) | |
| PYL1 (SEQ-ID NO: 111) | NSP3-(PYL1)₃ (SEQ-ID NO: 64) | Abscisic acid-inducible binding to |
| | (PYL1)₃-NSP3 (SEQ-ID NO: 67) | ABI (SEQ-ID NO: 71) |
| | L7Ae-PYL1 (SEQ-ID NO: 203) | |
| | PYL1-NSP3 (SEQ-ID NO: 278) | |
| | PYL1-L7Ae (SEQ-ID NO: 279) | |
| | NSP3-PYL1 (SEQ-ID NO: 291) | |
| Aff6_{VI8FΔN} (SEQ-ID NO: 72) | MCP-(Aff6_{VI8FΔN})4 (SEQ-ID NO: 50) | Red light-inducible binding to |
| | | DrBPhP (SEQ-ID NO: 81) |
| ANR (SEQ-ID NO: 73) | ANR-NSP3 (SEQ-ID NO: 2) | Grazoprevir-repressible binding |
| | (ANR)₂-NSP3 (SEQ-ID NO: 267) | from NS3a(H1) (SEQ-ID NO: |
| | (ANR)₄-NSP3 (SEQ-ID NO: 3) | 105) |
| | (ANR)₆-NSP3 (SEQ-ID NO: 4) | |
| | (ANR)₈-NSP3 (SEQ-ID NO: 5) | |
| CIB1 (SEQ-ID NO: 76) | L7Ae-CIB1 (SEQ-ID NO: 32) | Blue light-inducible binding to |
| | MCP-CIB1 (SEQ-ID NO: 247) | Cry2 (SEQ-ID NO: 78) |
| Cry2 (SEQ-ID NO: 78) | Cry2-NSP3 (SEQ-ID NO: 10) | Blue light-inducible binding to |
| | | CIB1 (SEQ-ID NO: 76) |
| DNCR (SEQ-ID NO: 79) | DNCR-NSP3 (SEQ-ID NO: 270) | Danoprevir-inducible binding to |
| | (DNCR)₂-NSP3 (SEQ-ID NO: 271) | NS3a (SEQ-ID NO: 104) and |
| | (DNCR)₃-NSP3 (SEQ-ID NO: 11) | NS3a(H1) (SEQ-ID NO: 105) |
| DrBPhP (SEQ-ID NO: 81) | DrBPhP-NSP3 (SEQ-ID NO: 15) | Red light-inducible binding to |
| | | Aff6_{VI8FΔN} (SEQ-ID NO: 72) |
| ERK2 (SEQ-ID NO: 85) | ERK2-NSP3 (SEQ-ID NO: 277) | MAPK-inducible binding to pE59 |
| | (ERK2)₂-NSP3 (SEQ-ID NO: 24) | (SEQ-ID NO: 110) |
| FKBP (SEQ-ID NO: 86) | FKBP-L7Ae (SEQ-ID NO: 25) | Rapamycin-inducible binding to |
| | FKBP-NSP3 (SEQ-ID NO: 252) | FRB (SEQ-ID NO: 87) |
| FRB (SEQ-ID NO: 87) | FRB-NSP3 (SEQ-ID NO: 26) | Rapamycin-inducible binding to |
| | MCP-FRB (SEQ-ID NO: 243) | FKBP (SEQ-ID NO: 86) |
| GAI (SEQ-ID NO: 88) | GAI-L7Ae (SEQ-ID NO: 27) | Gibberellic acid-inducible binding |
| | L7Ae-GAI (SEQ-ID NO: 283) | to GID1 (SEQ-ID NO: 89) |
| | (GAI)₃-L7Ae (SEQ-ID NO: 285) | |
| | GAI-NSP3 (SEQ-ID NO: 289) | |
| | NSP3-GAI (SEQ-ID NO: 293) | |
| GID1 (SEQ-ID NO: 89) | NSP3-GID1 (SEQ-ID NO: 63) | Gibberellic acid-inducible binding |
| | MCP-GID1 (SEQ-ID NO: 242) | to GAI (SEQ-ID NO: 88) |
| | L7Ae-GID1 (SEQ-ID NO: 282) | |
| | GID1-L7Ae (SEQ-ID NO: 286) | |
| | GID1-NSP3 (SEQ-ID NO: 287) | |
| | NSP3-(GID1)₃ (SEQ-ID NO: 292) | |
| GNCR (SEQ-ID NO: 90) | 3xHA-(GNCR)₃-NSP3 (SEQ-ID NO: 1) | Grazoprevir-inducible binding to |
| | GNCR-NSP3 (SEQ-ID NO: 28) | NS3a (SEQ-ID NO: 104) and |
| | (GNCR)₂-NSP.3 (SEQ-ID NO: 29) | NS3a(H1) (SEQ-ID NO: 105) |
| | (GNCR)₃-NSP3 (SEQ-ID NO: 30) | |
| | L7Ae-GNCR (SEQ-ID NO: 36) | |
| NS3a (SEQ-ID NO: 104) | L7Ae-NS3a (SEQ-ID NO: 39) | Grazoprevir-inducible binding to |
| | L7Ae-(NS3a)₂ (SEQ-ID NO: 40) | GNCR (SEQ-ID NO: 90); |
| | L7Ae-(NS3a)₃ (SEQ-ID NO: 41) | Danoprevir-inducible binding to |
| | L7Ae-(NS3a)₃-3xFLAG (SEQ-ID NO: 48) | DNCR (SEQ-ID NO: 79) |
| | MCP-NS3a (SEQ-ID NO: 54) | |
| | MCP-(NS3a)₂ (SEQ-ID NO: 290) | |
| [symbol] | [symbol] | [symbol] |

[symbol]

indicates data missing or illegible when filed

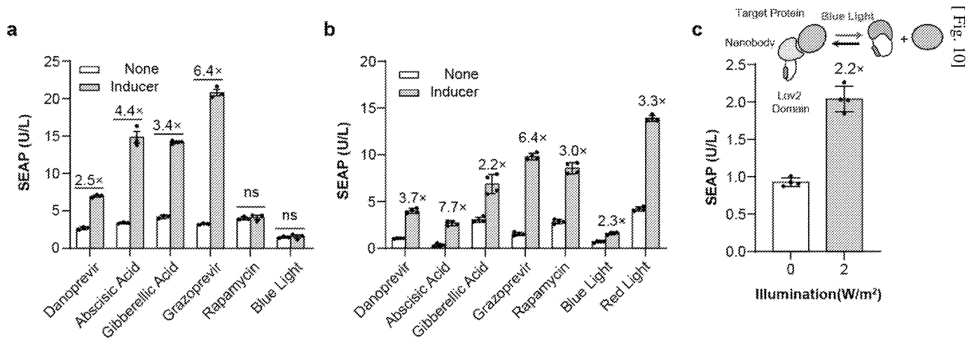

For L7Ae-based systems (FIG. 10A), SEAP-mRNA containing L7Ae-specific poly (A) surrogate with 24 C/D-box repeats (SEQ-ID NO: 136) was used as the reporter construct. For danoprevir-inducible SEAP translation, HEK-293 cells were further co-transfected with constitutive expression vectors for L7Ae-(NS3a)₃ (SEQ-ID NO: 41) and (DNCR) 3-NSP3 (SEQ-ID NO: 271). For abscisic acid-inducible SEAP translation, HEK-293 cells were further co-transfected with constitutive expression vectors for L7Ae-(ABI) 3 (SEQ-ID NO: 31) and (PYL1)₃-NSP3 (SEQ-ID NO: 67). For gibberellic acid-inducible SEAP translation, HEK-293 cells were further co-transfected with constitutive expression vectors for GAI-L7Ae (SEQ-ID NO: 27) and NSP3-GID1 (SEQ-ID NO: 63). For grazoprevir-inducible SEAP translation, HEK-293 cells were further co-transfected with constitutive expression vectors for L7Ae-(NS3a)₃ (SEQ-ID NO: 41) and (GNCR)₃-NSP3 (SEQ-ID NO: 30). For rapamycin-inducible SEAP translation, HEK-293 cells were further co-transfected with constitutive expression vectors for FKBP-L7Ae (SEQ-ID NO: 25) and FRB-NSP3 (SEQ-ID NO: 26). For blue light-inducible SEAP translation, HEK-293 cells were further co-transfected with constitutive expression vectors for L7Ae-CIB1 (SEQ-ID NO: 32) and Cry2-NSP3 (SEQ-ID NO: 10). SEAP levels in culture supernatants were scored at 48 h after addition of corresponding inducers (danoprevir, 1 μM; abscisic acid, 100 μM; gibberellic acid, 100 μM; grazoprevir, 0.5 μM; rapamycin, 0.01 μM) or at 24 h after exposure to blue light (450 nm; ON, 30 s at 5 mW/cm²; OFF, 30 s). Commercial information on the chemical reagents used are listed in Chemicals and Recombinant proteins in the Methods section above. For MCP-based systems (FIG. 10B), SEAP-mRNA containing MCP-specific poly (A)-surrogate with either 16 (SEQ-ID NO: 138) or 24 MS2-box repeats (SEQ-ID NO: 137) were used as reporter constructs. For danoprevir-inducible SEAP translation, HEK-293 cells were co-transfected with SEQ-ID NO: 137 and constitutive expression vectors for MCP-NS3a (SEQ-ID NO: 54) and DNCR-NSP3 (SEQ-ID NO: 270). For abscisic acid-inducible SEAP translation, HEK-293 cells were co-transfected with SEQ-ID NO: 137 and constitutive expression vectors for ABI-MCP (SEQ-ID NO: 259) and (PYL1)₃-NSP3 (SEQ-ID NO: 67). For gibberellic acid-inducible SEAP translation, HEK-293 cells were co-transfected with SEQ-ID NO: 137 and constitutive expression vectors for MCP-GID1 (SEQ-ID NO: 242) and GAI-NSP3 (SEQ-ID NO: 289). For grazoprevir-inducible SEAP translation, HEK-293 cells were co-transfected with SEQ-ID NO: 137 and constitutive expression vectors for MCP-NS3a (SEQ-ID NO: 54) and GNCR-NSP3 (SEQ-ID NO: 28). For rapamycin-inducible SEAP translation, HEK-293 cells were co-transfected with SEQ-ID NO: 138 and constitutive expression vectors for MCP-FRB (SEQ-ID NO: 243) and FKBP-NSP3 (SEQ-ID NO: 252). For blue light-inducible SEAP translation, HEK-293 cells were co-transfected with SEQ-ID NO: 138 and constitutive expression vectors for MCP-CIB1 (SEQ-ID NO: 247) and Cry2-NSP3 (SEQ-ID NO: 10). For red light-inducible SEAP translation, HEK-293 cells were co-transfected with SEQ-ID NO: 138 and constitutive expression vectors for MCP-(Aff6v18FAN)₄ (SEQ-ID NO: 50) and DrBPhP-NSP3 (SEQ-ID NO: 15). SEAP levels in culture supernatants were scored at 48 h after addition of corresponding inducers (danoprevir, 0.5 μM; abscisic acid, 100 μM; gibberellic acid, 100 μM; grazoprevir, 0.5 μM; rapalog, 0.1 μM) or at 48 h after exposure to blue light (450 nm; ON, 30 s at 5 mW/cm²; OFF, 30 s) or red light (660 nm, constantly 1 W/m²). Commercial information on the chemical reagents used are listed in Chemicals and Recombinant proteins in the Methods section above.

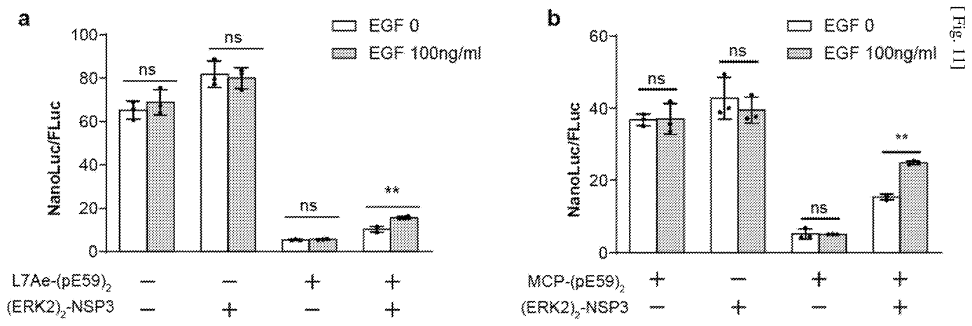

Translational regulation by trigger-inducible STIF systems can also be engineered in various other ways. For example, we incorporated the blue light-dependent LaM8AK47 nanobody (SEQ-ID NO: 220) into the STIF framework, thereby demonstrating versatility and design flexibility for translational regulation (FIG. 10C). In this configuration, HEK-293 cells were transfected with an expression vector for SEAP-mRNA containing 16 tandem MS2-box repeats in the 3′-UTR (SEQ-ID NO: 138) and constitutive expression vectors for MCP-LaM8AK47 (SEQ-ID NO: 241) and mCherry-NSP3 (SEQ-ID NO: 250). At 6 h post transfection, cells were illuminated for 48 h with blue light (450 nm, 2 mW/cm²) before SEAP levels in culture supernatants were scored to assess the blue light-triggered gene switch. To demonstrate the ability to also engineer genetically encoded sensors responding to intracellular signaling dynamics, we integrated the ERK2-specific pE59 DARPin system (SEQ-ID NO: 110) into the bipartite STIF framework. As pE59 was developed to specifically bind phosphorylated ERK2 during MAPK signaling, the individual STIF components RBP-(pE59)ₙ (SEQ-ID NOs: 46, 249&280) and (ERK2)ₙ-NSP3 (SEQ-ID NOs: 24&277) only assembled and activated RBP-specific reporter gene expression in cells of high MAPK activity (FIG. 11). For L7Ae-based RBP systems (FIG. 11A), HEK-293 cells were co-transfected with a dual reporter vector containing a constitutive FLuc expression unit and an expression unit for NanoLuc-mRNA containing L7Ae-specific poly (A)-surrogate (SEQ-ID NO: 135), an shRNA-216 expression vector (SEQ-ID NO: 126) and different combinations of constitutive expression vectors for L7Ae-(pE59)₂ (SEQ-ID NO: 46) and (ERK2)₂-NSP3 (SEQ-ID NO: 24). For MCP-based RBP systems (FIG. 11B), HEK-293 cells were co-transfected with a constitutive FLuc expression vector (SEQ-ID NO: 305), an expression vector for NanoLuc-mRNA containing MCP-specific poly (A)-surrogate (SEQ-ID NO: 139) and different combinations of constitutive expression vectors for MCP-(pE59)₂ (SEQ-ID NO: 249) and (ERK2)₂-NSP3 (SEQ-ID NO: 24) before cultivation in cell culture medium containing 2% FBS (v/v) (Gibco FBS, Australia; Thermo Fisher Scientific, Waltham, MA; cat. no. 10099141, lot no. 2177370). Luciferase levels in culture supernatants were quantified at 48 h after the addition of 100 ng/mL recombinant human EGF (PeproTech EC Ltd., cat. no. AF-100-15) according to the descriptions in the Methods section above. For (−) conditions, pcDNA3.1 (+) (Invitrogen, CA; cat-no. V79020) was transfected instead of expression vectors. Results show that increases in reporter levels correlate with increased MAPK activity triggered by EGF (FIG. 11). In the future, engineering of similar sensors for other signalling pathways can be readily achieved by exchanging the pE59 (SEQ-ID NO: 110) and ERK2 (SEQ-ID NO: 85) domains by other signalling specific PPI systems. This will be useful for cell-based applications involving real-time monitoring of signaling pathway dynamics, for which current technologies such as genetically encoded fluorescent reporters and/or Western Blot analysis are either limited to visualization-centered studies and end-point measurements, respectively.

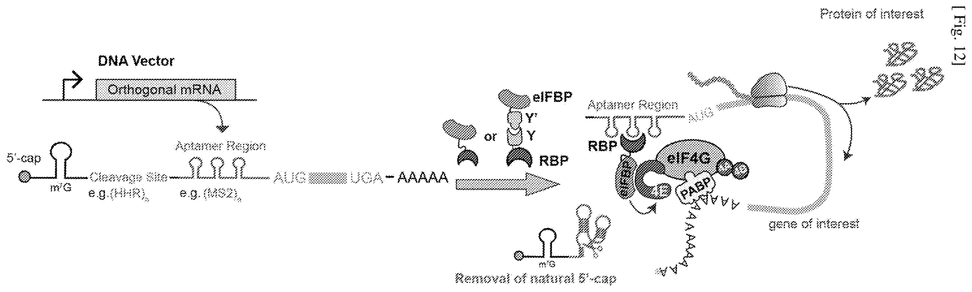

Based on these results, we believe that mRNA circularization can also be triggered through engineered 5′-cap surrogates at the 5′-UTR akin to the working principles for poly (A)-surrogates in the 3′-UTR. In such cases, shRNA- or HHR-based RNA cleavage sites would be placed immediately downstream of the guanine-rich 5′-cap and upstream of an RBP-specific aptamer region to allow pre-programmed cap-removal (FIG. 12). Thus, the aptamer region would serve as a 5′-cap surrogate to recruit the same STIF constructs described in Tables 1, 3 and 4 for either constitutive or trigger-inducible initiation of mRNA circularization and target gene translation (FIG. 12). In such cases, corresponding target gene mRNA would comprise the following architecture:

TABLE 5
Synthetic mRNA transcripts containing RBP-specific 5′ -cap-surrogates enabling
STIF-dependent translation and expression of different genes of interest (GOIs).

| | | Coding | | |
|---|---|---|---|---|
| 5′-cleavage | 5′cap-surrogate | region | poly(A)-surrogate | 3′-cleavage |
| (HHR)ₙ | (C/D-box)ₙ | GOI | (C/D-box)ₙ | (HHR)ₙ |
| (HHR)ₙ | (MS2-box)ₙ | GOI | (MS2-box)ₙ | (HHR)ₙ |
| (HHR)ₙ | (C/D-box)ₙ | GOI | none | none |
| (HHR)ₙ | (MS2-box)ₙ | GOI | none | none |
| (HHR)ₙ | (C/D-box)ₙ | GOI | (MS2-box)ₙ | (HHR)ₙ |
| (HHR)ₙ | (MS2-box)ₙ | GOI | (C/D-box)ₙ | (HHR)ₙ |
| (HHR)ₙ | (C/D-box)ₙ | GOI | (C/D-box)ₙ | BS(shRNA-216)ₙ |
| (HHR)ₙ | (MS2-box)ₙ | GOI | (MS2-box)ₙ | BS(shRNA-216)ₙ |
| (HHR)ₙ | (C/D-box)ₙ | GOI | none | none |
| (HHR)ₙ | (MS2-box)ₙ | GOI | none | none |
| (HHR)ₙ | (C/D-box)ₙ | GOI | (MS2-box)ₙ | BS(shRNA-216)ₙ |
| (HHR)ₙ | (MS2-box)ₙ | GOI | (C/D-box)ₙ | BS(shRNA-216)ₙ |
| BS(shRNA-216)ₙ | (C/D-box)ₙ | GOI | (C/D-box)ₙ | (HHR)ₙ |
| BS(shRNA-216)ₙ | (MS2-box)ₙ | GOI | (MS2-box)ₙ | (HHR)ₙ |
| BS(shRNA-216)ₙ | (C/D-box)ₙ | GOI | none | none |
| BS(shRNA-216)ₙ | (MS2-box)ₙ | GOI | none | none |
| BS(shRNA-216)ₙ | (C/D-box)ₙ | GOI | (MS2-box)ₙ | (HHR)ₙ |
| BS(shRNA-216)ₙ | (MS2-box)ₙ | GOI | (C/D-box)ₙ | (HHR)ₙ |
| BS(shRNA-216)ₙ | (C/D-box)ₙ | GOI | (C/D-box)ₙ | BS(shRNA-216)ₙ |
| BS(shRNA-216)ₙ | (MS2-box)ₙ | GOI | (MS2-box)ₙ | BS(shRNA-216)ₙ |
| BS(shRNA-216)ₙ | (C/D-box)ₙ | GOI | none | none |
| BS(shRNA-216)ₙ | (MS2-box)ₙ | GOI | none | none |
| BS(shRNA-216)ₙ | (C/D-box)ₙ | GOI | (MS2-box)ₙ | BS(shRNA-216)ₙ |
| BS(shRNA-216)ₙ | (MS2-box)ₙ | GOI | (C/D-box)ₙ | BS(shRNA-216)ₙ |

### Example 2: Grazoprevir-Controlled Gene Expression in Mammalian Cells

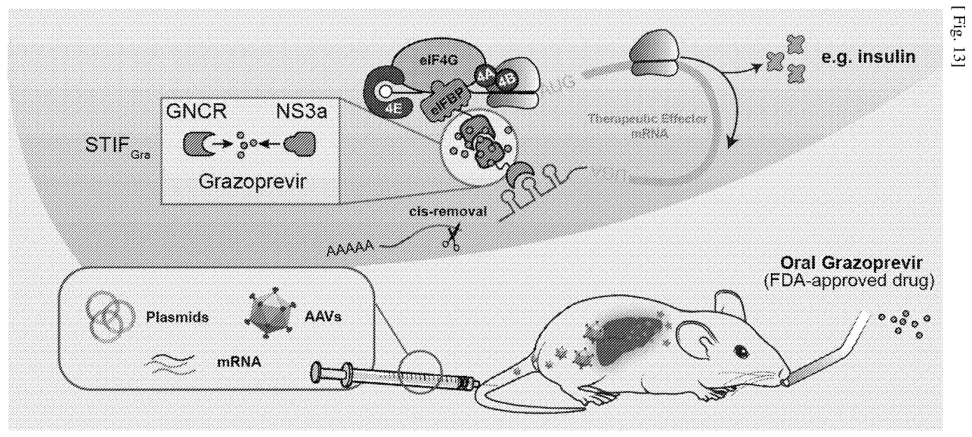

Trigger-inducible translational regulation systems (FIG. 7 and Table 4) engineered on the basis of STIF-mediated mRNA circularization enables a variety of cell-based applications. For example, based on the grazoprevir-inducible GNCR: NS3a system (FIG. 10) we demonstrate how a clinically relevant gene switch can be created either for regulation of a variety of mammalian cell activities or for long-term therapeutic transgene delivery in vivo. Grazoprevir-inducible gene switches have two major advantages. First, grazoprevir is an FDA-approved drug for hepatitis C treatment and is therefore bioavailable, non-toxic and metabolically inert, and should be applicable to regulation of a wide variety of protein therapeutics without interfering with their efficacy in vivo. At the same time, translation-based gene switches would be compatible with various clinically approved gene therapy products by design; they could be administered to patients either using AAV vectors for DNA-encoded treatments, or directly formulated as an in vitro-manufactured mRNA drug (FIG. 13).

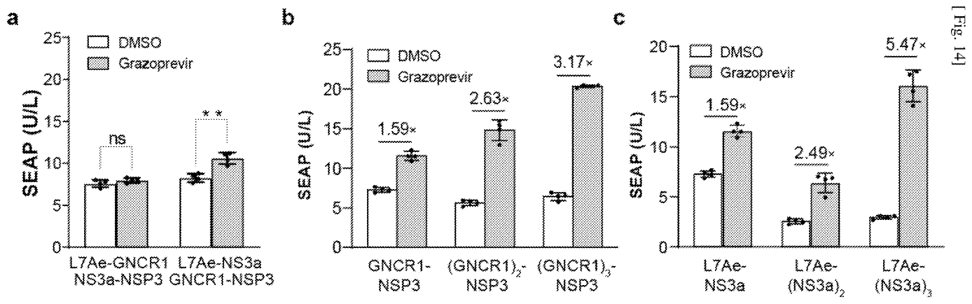

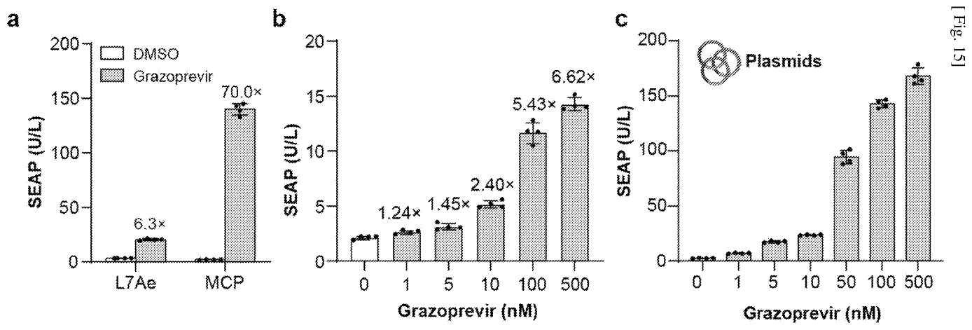

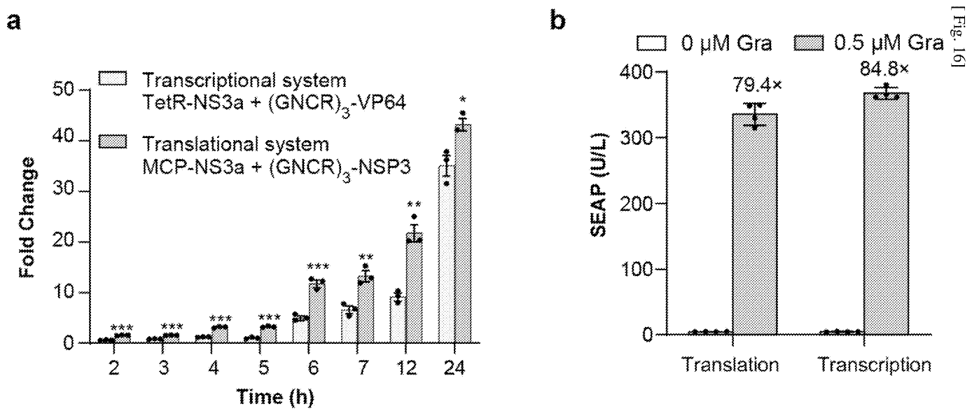

To fine-tune a grazoprevir-inducible gene switch using the design strategy and building blocks described in FIG. 7 and Table 4, we show that toggling L7Ae-NS3a/GNCR-NSP3 and L7Ae-GNCR/NS3a-NSP3 configuration (FIG. 14A) as well as increasing the number of tandem GNCR (SEQ-ID NO: 90) (FIG. 14B) and NS3a repeats (SEQ-ID NO: 104) (FIG. 14C) were all considerable options to increase fold-changes of grazoprevir-triggered gene expression. Experimentally, HEK-293 cells were transfected with plasmids encoding SEAP-mRNA containing L7Ae-specific poly (A)-surrogate (SEQ-ID NOs: 126&134) and different grazoprevir-regulated L7Ae- and NSP3-fusion proteins (SEQ-ID NOs: 36&62 or 39&28), different NSP3-fusion proteins containing one (SEQ-ID NO: 28), two (SEQ-ID NO: 29) or three N-terminal GNCR repeats (SEQ-ID NO: 30) or different L7Ae-fusion proteins consisting of one (SEQ-ID NO: 39), two (SEQ-ID NO: 40) or three C-terminal NS3a repeats (SEQ-ID NO: 41). Specifically, these constructs were validated by transfection of encoding plasmids into HEK-293 whose cell culture and transfection methods are described in the Methods section above. SEAP levels in culture supernatants were scored at 48 h after addition of 0.1 μM grazoprevir dissolved in DMSO. Eventually, split-STIF constructs each containing three tandem GNCR and NS3a repeats ((GNCR)₃-NSP3, SEQ-ID NO: 30) combined with either L7Ae-(NS3a)₃ (SEQ-ID NO: 41, specific for C/D-box-containing target mRNA) or MCP-(NS3a)₃ (SEQ-ID NO: 55, specific for MS2-box-containing target mRNA) showed the best regulation performance in terms of fold-change (FIG. 15A), dose-dependence (FIGS. 15B, 15C) and activation kinetics (FIG. 16).

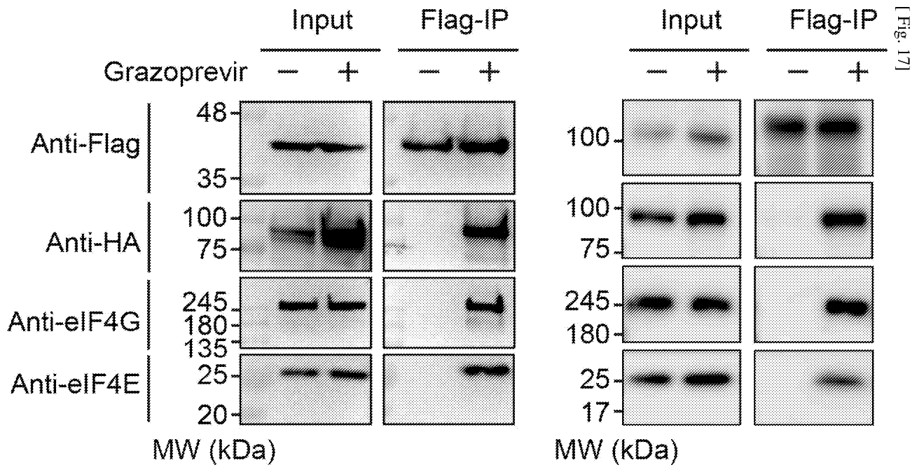

For L7Ae-based systems, HEK-293 cells were co-transfected with plasmids encoding SEAP-mRNA containing C/D-box-based poly (A)-surrogate (SEQ-ID NOs: 126&134), (GNCR)₃-NSP3 (SEQ-ID NO: 30) and L7Ae-(NS3a)₃ (SEQ-ID NO: 41). For MCP-based systems, HEK-293 cells were co-transfected with plasmids encoding SEAP-mRNA containing MS2-box-based poly (A)-surrogate (SEQ-ID NO: 137), (GNCR)₃-NSP3 (SEQ-ID NO: 30) and MCP-(NS3a)₃ (SEQ-ID NO: 55). For fold-change analysis (FIGS. 15A, 16B), SEAP levels in culture supernatants were scored at 48 h after addition of 0.1 μM grazoprevir dissolved in DMSO (vehicle control). For dose-dependence analysis (FIGS. 15B, 15C), SEAP levels in culture supernatants were scored at 48 h after addition of different concentrations of grazoprevir dissolved in DMSO (vehicle control). For analysis of activation speed (FIG. 16A), SEAP levels in culture supernatants were scored at 24 h after addition of different concentrations of grazoprevir. Hence, we could show that our STIF-based grazoprevir-inducible translational gene switch was significantly faster than a counterpart state-of-the art gene switch operating at the transcriptional level (through expression of TetR-NS3a (SEQ-ID NO: 299), (GNCR)₃-VP64 (SEQ-ID NO: 300) and a TetR-inducible promoter driving SEAP transcription (SEQ-ID NO: 168), not only generating higher induction-folds over the entire 24 h experimental timespan (FIG. 16A) but also producing similar absolute expression strengths over longer timespans (FIG. 16B). Importantly, we used co-immunoprecipitation and Western Blot technologies (as described in FIG. 6A and in the Methods section above) to confirm that endogenous (GNCR)₃-NSP3-specific eIF4G and eIF4E only colocalized with RBP-(NS3a)₃ (SEQ-ID NOs: 48&263) in the presence of grazoprevir (FIG. 17), which is the mechanistic basis for trigger-inducible formation circularized mRNA configurations. This experiment is also critical to identify gene regulation systems that are developed on the basis of the present invention; eIF4G (detected by anti-eIF4G antibodies, e.g. product no. 2498, lot. no. 4 of Cell Signaling Technology), eIF4E (detected by anti-eIF4E antibodies, e.g. product no. 2067, lot. no. 8 of Cell Signaling Technology) and HA-tagged NSP3 (detected by anti-HA antibodies, e.g. product no. 3724, lot. no. 10 of Cell Signaling Technology) would not co-localize with FLAG-tagged L7Ae or MCP constructs if there is no formation of a circularized mRNA configuration triggered by grazoprevir. Therefore, demonstration that such co-localization strictly depends on the presence of a user-defined trigger signal (in this case, grazoprevir) is critical to show that future regulation systems use the concept of STIF-based strategy developed in this work (Example 1).

### Example 3: Grazoprevir-Regulated Gene Therapy In Vivo

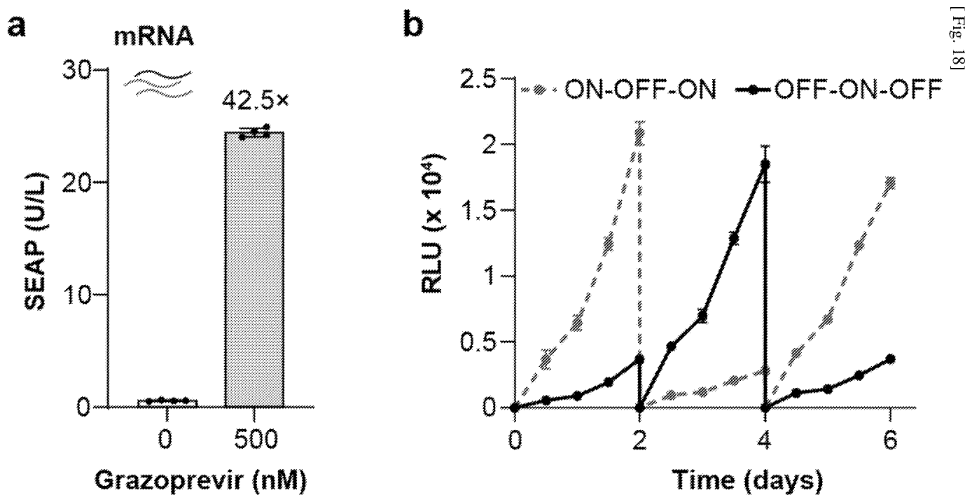

In this example, we use the grazoprevir-inducible translational regulation system (described in Example 2) to show how STIF-mediated gene switches can be developed into clinically eligible gene therapy products for in vivo applications. As already introduced in Example 2 (FIG. 13), translational regulation systems can be administered to patients either using AAV vectors for DNA-encoded treatments, or directly formulated as an in vitro-manufactured mRNA drug. Thus, we show that our grazoprevir-inducible translational gene switch is indeed compatible with either DNA-(FIGS. 15C, 16A) and RNA-centered delivery strategies (FIG. 18A). To show grazoprevir-inducible SEAP translation by mRNA delivery, HEK-293 cells were (co-) transfected with in vitro-transcribed mRNA encoding for MCP-(NS3a)₃ (SEQ-ID NO: 55 from pSL1085), (GNCR)₃-NSP3 (SEQ-ID NO: 30 from pYW361) a SEAP expression vector containing MCP-specific poly (A)-surrogate (SEQ-ID NO: 137 from pSL468). Here, in vitro transcription was performed using a T7 High Yield RNA Transcription Kit (Vazyme Biotech, Nanjing, China; cat. no. TR-101) with detailed settings described in the Methods section above. To show grazoprevir-inducible SEAP translation by DNA delivery, HEK-293 cells were transfected with plasmids encoding corresponding constructs (e.g. (GNCR)₃-NSP3, MCP-(NS3a)₃ and SEAP-mRNA containing MS2-box-based poly (A)-surrogate; SEQ-IDs NO: 30, 55 and 137). SEAP levels in culture supernatants were quantified at 48 h after addition of grazoprevir. In direct comparison, DNA-based delivery can be advantageous in enabling various durable applications in mammalian cells, such as selection of stable cell lines allowing for reversible sense-and-response dynamics over extended time periods (FIG. 18B) and/or integration of genetic components into AAV-based vectors for various therapeutic purposes in vivo. For reversibility studies, we first created a HEK-293_{LSCCS1} cell line stably transgenic for grazoprevir-triggered co-expression of NanoLuc (SEQ-ID NO: 101) and murine insulin (mINS; SEQ-ID NO: 99). Specifically, pSL721 (Sleeping Beauty (SB)-specific transposon expressing (MCP-(NS3a)₃, SEQ-ID NO: 55), pSL722 (SB-specific transposon expressing (GNCR)₃-NSP3, SEQ-ID NO: 30), pSL688 (SB-specific transposon expressing NanoLuc and mINS mRNA with MCP-specific poly (A)-surrogate, SEQ-ID NO: 140) and pCMV-T7-SB100 (Addgene plasmid no. 34879 for constitutive expression of SB-transposase) were transfected into HEK-293 cells before selection with 1 μg/ml puromycin (Thermo Fisher Scientific; cat. no. A1113803), 10 μg/ml Blasticidin (Thermo Fisher Scientific; cat. no. R21001) and 100 μg/ml zeocin (Thermo Fisher Scientific; cat. no. R25005) for 15 days. 21 monoclonal cell lines were harvested and selected by quantifying NanoLuc expression upon treatment with 500 nM Grazoprevir for 24 hours. The monoclonal cell line HEK-293_{LSCCS1} was then cultivated for 7 days with grazoprevir levels in the culture medium successively switched between 0 and 500 nM by medium exchange and washing for 3 times with grazoprevir-free medium. NanoLuc levels were measured every 12 h. The cell density was readjusted to 1×10⁵ cells per ml every 2-3 days. For AAV production, transfer plasmids pSL511 (expressing SEAP-mRNA with MCP-specific poly (A)-surrogate, SEQ-ID NO: 137), pSL512 (expressing MCP-(NS3a)₃ SEQ-ID NO: 55) and pSL446 (expressing (GNCR)₃-NSP3, SEQ-ID NO: 30) were constructed according to descriptions in the Methods section above, producing the corresponding AAV2/8-(GNCR)₃-NSP3, AAV2/8-MCP-(NS3a)₃ and AAV2/8-SEAP-(MS2-box)₂₄-HHR-pA particles.

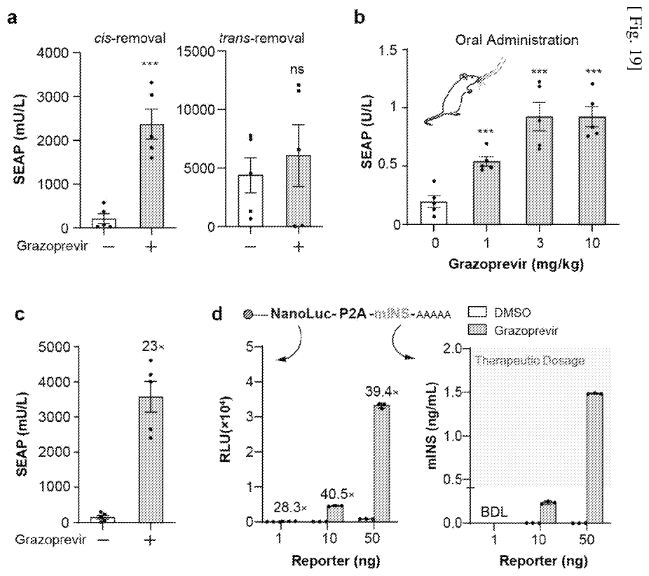

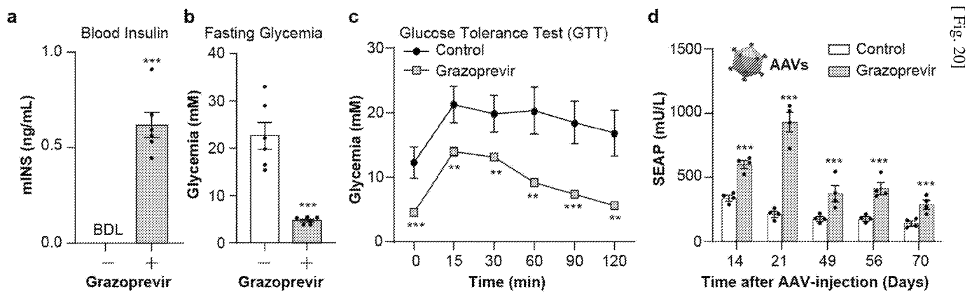

To evaluate grazoprevir-inducible transgene regulation in vivo, we hydrodynamically injected STIF-encoding vectors and L7Ae-specific SEAP expression vectors into the tail vein of mice (FIG. 19A). Specifically, 300 μg of plasmids comprising of pLZ74 (encoding for (GNCR)₃-NSP3, SEQ-ID NO: 30), pLZ76 (encoding for L7Ae-(NS3a) 3, SEQ-ID NO: 41) and either pSL355 (SEQ-ID NO: 136: for HHR-mediated cis-removal of poly (A)) or pSL88&pSL4 (SEQ-IDs NO: 126 and 134: for shRNA-216-mediated trans-removal of poly (A)) were administered into C57BL/6 mice by hydrodynamic tail vein injection as described in the Methods section above. After 6 h, mice received the first of 3 daily intraperitoneal injections of grazoprevir (1 mg/kg dissolved in PBS). SEAP levels in the bloodstream of mice were measured at 24 h after the first grazoprevir injection. According to our experimental results (FIG. 19A), the HHR-dependent reporter (SEQ-ID NO: 136) was established as the class of STIF-specific target mRNA for in vivo applications (FIG. 19A), with an oral dose of 3 mg/kg grazoprevir being sufficient to fully activate the system (FIGS. 19B, 19C). Next, using insulin as an exemplary therapeutic output, we tested the treatment potential of the grazoprevir-inducible gene switch (FIG. 19D). First, the therapeutic efficacy window of insulin expression was determined by co-transfection of HEK-293 cells with 200 ng of pSL1042 (expressing MCP-(NS3a)₃, SEQ-ID NO: 55), 200 ng of pSL1032 (expressing (GNCR)₃-NSP3, SEQ-ID NO: 30) and different amounts of pSL1003 (expressing NanoLuc and mINS-mRNA containing MCP-specific poly (A)-surrogate, SEQ-ID NO: 174). NanoLuc and mINS levels in culture supernatants were scored at 48 h after addition of 0.5 μM grazoprevir. Thus, the gene switch configuration where the grazoprevir-triggered ON state reaches physiological insulin levels (blue shaded box in FIG. 19D), while the basal OFF state falling below this threshold (50 ng of pSL1003), was selected for subsequent gene therapy applications related to diabetes treatment. In line with this, plasmids encoding MCP-(NS3a)₃, (GNCR)₃-NSP3 and insulin-mRNA containing MCP-specific poly (A)-surrogate (SEQ-ID NOs: 30, 55 and 140) were hydrodynamically injected into the tail vein of type-1 diabetic (T1D) mice. At 6 h post injection, mice were fed with the first of 3 daily administrations of 3 mg/kg grazoprevir before blood insulin levels (FIG. 20A) and fasting glycemia (FIG. 20B) of mice were measured at 20 h after the first grazoprevir administration. Intraperitoneal glucose tolerance tests (GTT) were also performed at 24 h after the first grazoprevir administration (4 h after quantification of blood insulin, according to the experimental procedures described in the Methods section above). Overall, our results show that oral grazoprevir administration accounted for an almost complete correction of insulin deficiency (FIG. 20A), hyperglycemia (FIG. 20B) and glucose intolerance (FIG. 20C) of type-1 diabetic mice treated with a gene therapy consisting of the genetic components for grazoprevir-inducible insulin translation (i.e., MCP-(NS3a)₃, SEQ-ID NO: 55; (GNCR)₃-NSP3, SEQ-ID NO: 30; and mINS-mRNA containing MCP-specific poly (A)-surrogates, SEQ-ID NOs: 140&174). To test long-term control of grazoprevir-inducible SEAP production in vivo, C57BL/6 mice received intravenous injection of 7×1011 AAV2/8-(GNCR)₃-NSP3, AAV2/8-MCP-(NS3a)₃ and AAV2/8-SEAP-(MS2-box)₂₄-HHR-pA particles for constitutive expression of (GNCR)₃-NSP3 (SEQ-ID NO: 30), MCP-(NS3a)₃ (SEQ-ID NO: 55) and SEAP-mRNA containing MCP-specific poly (A)-surrogate (SEQ-ID NO: 137), before SEAP production in the bloodstream was monitored over 10 weeks. At 24 h before each measurement, mice were fed the first of 3 daily administrations of 3 mg/kg grazoprevir. Results show that mice injected with AAV2/8 particles carrying the grazoprevir-inducible gene switch maintained the expected profile of regulated protein secretion for at least 10 weeks, indicating the potential for long-term treatment efficacy in vivo (FIG. 20D). Thus, we were able to engineer a grazoprevir-inducible gene switch based on the high-affinity grazoprevir: NS3a interaction (K; 140 μM (Foight et al., 2019)), demonstrating rapid, tightly controlled activation kinetics in vitro, as well as compatibility with therapeutic transgene delivery in vivo. Furthermore, we show that STIF-dependent gene switches are compatible with state-of-the-art gene therapy delivery strategies.

### Example 4: Grazoprevir-Controlled Gene Expression for Complex Biocomputation in Mammalian Cells and Mice

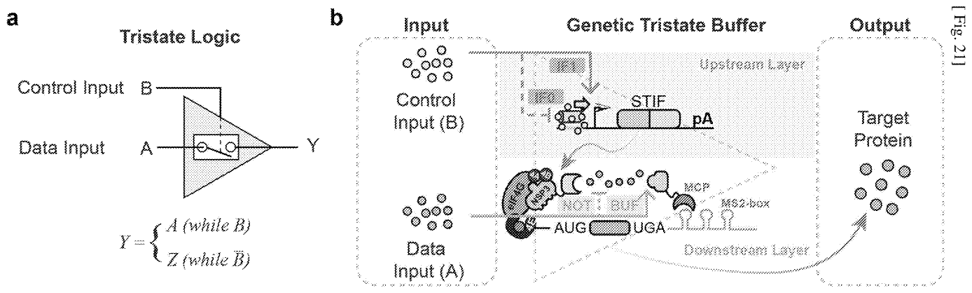

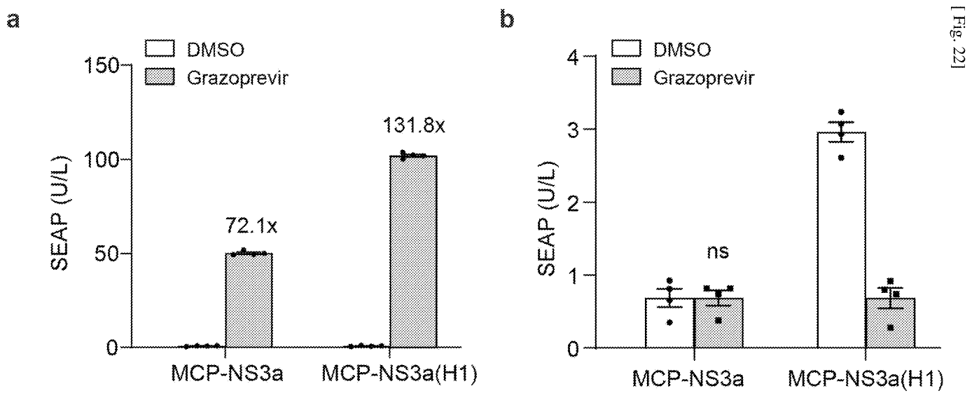

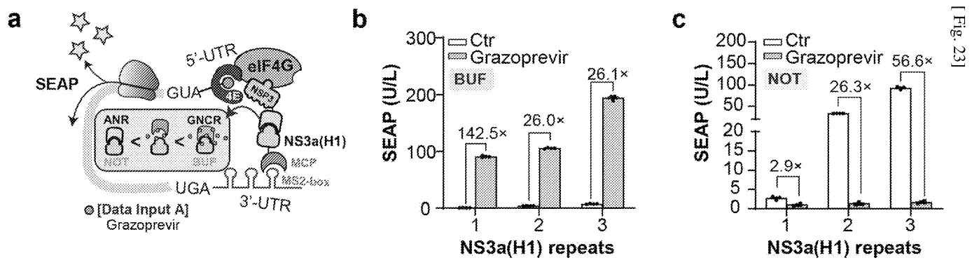

Engineering of genetic circuits consisting of interconnected tristate buffers has long remained an elusive goal to achieve complex biocomputation in mammalian cells. However, genetic implementation of tristate buffers has been prohibited by the lack of robust and mutually compatible gene switches that can be integrated in complex gene networks. In tristate buffers, the connectivity of a binary switch regulated by an input A must be strictly governed by an upstream switch through another signal B (FIG. 21A). Thus, control input B allows data input A to determine overall activity Y of the circuit unless B is inactivated or “unplugged” by the upstream switch. In such inactivated state (NOT B), overall activity of the circuit would fall into a third “high impedance” state Z that no longer depends on the status (0 or 1 value) of A (FIG. 21A). For implementation of such tristate buffers in mammalian cells, regulated expression of trigger-inducible gene switches programmed with either “buffered” (BUF) and “inverted” (NOT) signal processing logics are therefore required. Thus, integration of the grazoprevir-controlled triad NS3a/GNCR/ANR (SEQ-ID NOs: 73, 90&104: introduced in Table 3&4; FIGS. 8-10) into the STIF-based framework of translational regulation would be ideal candidates to create potential BUF and NOT switches (FIG. 21B). Grazoprevir-inducible translational gene switches designed in Examples 2 and 3 inherently follow BUF logics, with SEAP-mRNA containing MCP-specific poly (A)-surrogate (SEQ-ID NO: 137) and (GNCR)₃-NSP3 (SEQ-ID NO: 30) combined with either MCP-NS3a (SEQ-ID NO: 54) or MCP-NS3a (H1) (SEQ-ID NO: 56) constitute the related genetic componentry (FIG. 22A). To engineer a grazoprevir-repressible translational gene switch with NOT logics, HEK-293 cells were co-transfected with plasmids encoding SEAP-mRNA containing MCP-specific poly (A)-surrogate (SEQ-ID NO: 137), (ANR)₄-NSP3 (SEQ-ID NO: 3) and fusion proteins between and either NS3a (producing MCP-NS3a, SEQ-ID NO: 54) or NS3a (H1) (producing MCP-NS3a (H1), SEQ-ID NO: 56). SEAP levels in culture supernatants were scored at 48 h after addition of 0.5 μM Grazoprevir dissolved in DMSO (vehicle control) (FIG. 22B). Thus, STIFs engineered to contain GNCR (SEQ-ID NO: 90) and NSP3 (SEQ-ID NO: 106) enable grazoprevir-inducible translation (BUF switch), while fusion of ANR motifs (SEQ-ID NO: 73) to NSP3 (SEQ-ID NO: 106) results in grazoprevir-repressible translation (NOT switch) (FIG. 23A). Furthermore, the signal-to-noise ratio of corresponding BUF- (FIG. 23B) and NOT-switches (FIG. 23C) could be systematically fine-tuned by increasing the number of tandem NS3a (H1)-repeats (SEQ-ID NO: 105) added to MCP (SEQ-ID NO: 98). Specifically, HEK-293 cells were co-transfected with plasmids encoding MCP-specific SEAP mRNA (SEQ-ID NO: 137), (GNCR)₃-NSP3 (SEQ-ID NO: 30) and different MCP-fusion proteins consisting of one (SEQ-ID NO: 56), two (SEQ-ID NO: 57) or three C-terminal NS3a (H1)-repeats (SEQ-ID NO: 58) (FIG. 23B) or co-transfected with plasmids encoding MCP-specific SEAP mRNA (SEQ-ID NO: 137), (ANR)₄-NSP3 (SEQ-ID NO: 3) and different MCP-fusion proteins consisting of one (SEQ-ID NO: 56), two (SEQ-ID NO: 57) or three C-terminal NS3a (H1)-repeats (SEQ-ID NO: 58) (FIG. 23C). SEAP levels in culture supernatants were scored at 48 h after addition of 0.5 μM Grazoprevir dissolved in DMSO (vehicle control).

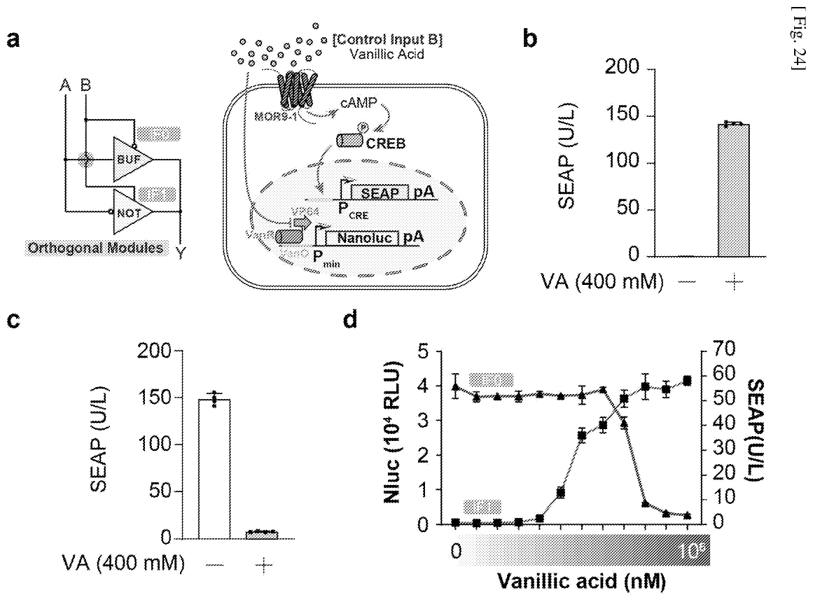

To form tristate buffers (FIG. 21A), expression of these grazoprevir-controlled BUF and NOT switches must be regulated by an upstream gene switch, which in turn is governed by a further control input B (FIG. 21B). Such upstream gene switch can either produce an inverted (known as “Active-LOW” control signal resulting from an “IF0” switch) or non-inverted output signal (known as “Active-HIGH” control signal from an “IF1” switch) by default. Thus, tristate buffers can contain up to 4 types of gene switches: B activates STIF expression (IF1), B terminates STIF expression (IF0), A activates target protein expression (BUF) and A terminates target protein expression (NOT). For implementation in mammalian cells, it is essential that the mutually antagonistic upstream switches IF0 and IF1 are orthogonal to each other and at the same time governed by a same trigger signal (FIG. 24A). For example, a vanillic acid-inducible gene switch based on an PKA/CREB1-responsive promoter activated by olfactory receptor MOR9-1-regulated cAMP-signaling (Saxena et al., 2016) could be a potential IF1 switch, whereas the IF0 switch could be completed by a VanR-dependent mammalian transactivator (VanR-VP64) modulating gene expression from cognate VanO-containing promoters (Gitzinger et al., 2012). For IF1 (FIG. 24B), HEK-293 cells were transfected with a constitutive MOR9-1 expression vector (SEQ-ID NO: 208) and a cAMP-responsive SEAP expression vector (SEQ-ID NO: 172). For IF0 (FIG. 24C), HEK-293 cells were transfected with a constitutive VanR-VP64 expression vector (SEQ-ID NO: 296) and a vanillic acid-inducible SEAP expression vector (SEQ-ID NO: 171). After cultivation in medium containing 0 or 400 μM Vanillic Acid (dissolved in DMSO), SEAP levels in the culture supernatants were scored at 48 h post transfection. To demonstrate parallel and orthogonal operation of IF1 and IF0 in mammalian cells, HEK-293 cells were further transfected with constitutive expression vectors for MOR9-1 (SEQ-ID NO: 208) and VanR-VP64 (SEQ-ID NO: 296), a cAMP-responsive SEAP expression vector (SEQ-ID NO: 172) and an VanR-specific NanoLuc expression vector (SEQ-ID NO: 170). SEAP levels in the culture supernatants were scored at 48 h after cultivation in cell culture medium containing different concentrations of Vanillic Acid. Results showed that vanillic acid-triggered IF0 and IF1 switches showed no crosstalk to each other when introduced into the same cells (FIG. 24D), thus fulfilling the eligibility requirements for upstream gene switches in tristate-based gene circuits (FIG. 24A).

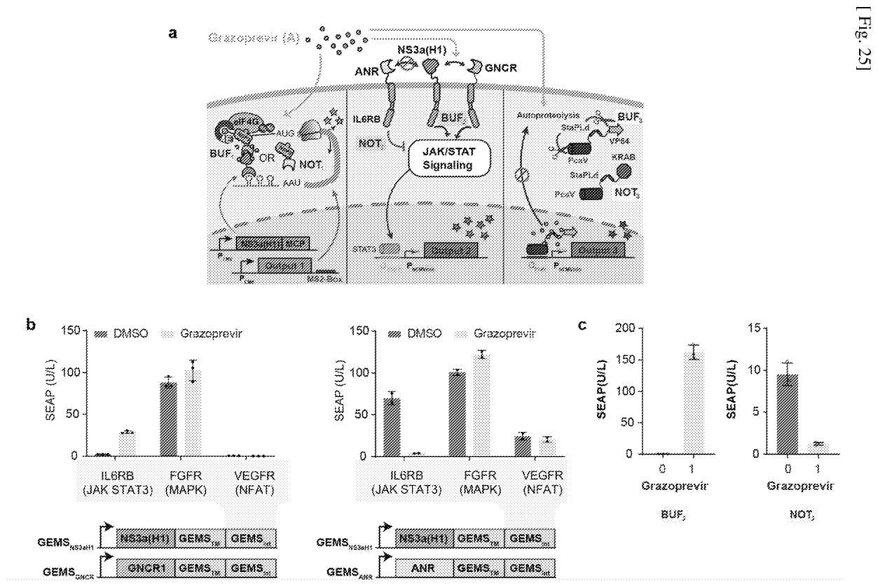

Using tristate buffers, a same set of upstream IF1/IF0 switches can be flexibly combined with multiple sets of downstream BUF/NOT switches in parallel, thus enabling resource-efficient data transfer without sacrificing switching speed. Apart from the “first” set of grazoprevir-regulated switches based on STIF-dependent translation (FIG. 23A; designated BUF₁/NOT₁), we therefore created two additional sets of grazoprevir-responsive gene switches that can be combined with (vanillic acid-regulated) IF1/IF0-switches (FIG. 25A). In one set (designated BUF2/NOT₂), the mutually exclusive triad NS3a (H1)/GNCR/ANR (SEQ-ID NOs: 73, 90&105) was incorporated into the framework of synthetic GEMS receptors (Scheller et al., 2018). GEMS receptors typically comprise an antibody-derived extracellular ligand binding domain, an EpoR-derived transmembrane domain (GEMS™, SEQ-ID NO: 182) and an intracellular signal transduction domain that mediates activation of different signaling pathways in human cells upon dimerization of the cell surface receptor. To create GEMS-based BUF/NOT switches regulated by grazoprevir, we replaced the antibody-domain of conventional GEMS constructs by either NS3a (H1) (SEQ-ID NO: 105), GNCR (SEQ-ID NO: 90) or ANR (SEQ-ID NO: 73) (FIG. 25B). Each GEMS variant was subsequently tested for different intracellular signaling domains, such as an IL-6RBₘ (triggering JAK/STAT3-signaling, SEQ-ID NO: 184), FGFR1int (triggering MAPK-signaling, SEQ-ID NO: 186) or VEGFR2ᵢₙₜ (triggering NFAT-signaling, SEQ-ID NO: 185). For grazoprevir-inducible target gene expression by GEMS variants containing IL6RB-derived intracellular domains, HEK-293 cells were co-transfected with a STAT3-specific SEAP expression vector (SEQ-ID NO: 169) and constitutive expression vectors for corresponding GEMS_{NS3a (H1)} and GEMS_{GNCR} constructs (SEQ-ID NOs: 191&192). For grazoprevir-inducible target gene expression by GEMS variants containing FGFR1-derived intracellular domains, cells were co-transfected with a TetR-specific SEAP expression vector (SEQ-ID NO: 168) and constitutive expression vectors for TetR-Elk1 (SEQ-ID NO: 199) and corresponding GEMS_{NS3a (H1)} and GEMS_{GNCR} Constructs (SEQ-ID NOs: 193 & 194). For grazoprevir-inducible target gene expression by GEMS variants containing VEGFR-derived intracellular domains, cells were co-transfected with a calcium-inducible SEAP expression vector (SEQ-ID NO: 167) and constitutive expression vectors for corresponding GEMS_{NS3a (H1)} and GEMS_{GNCR} constructs (SEQ-ID NOs: 195&196). For grazoprevir-repressible target gene expression by GEMS variants containing IL6RB-derived intracellular domains, cells were co-transfected with a STAT3-specific SEAP expression vector (SEQ-ID NO: 169) and constitutive expression vectors for corresponding GEMS_{NS3a (H1)} and GEMS_{ANR} constructs (SEQ-ID NOs: 187&192). For grazoprevir-repressible target gene expression by GEMS variants containing FGFR1-derived intracellular domains, cells were co-transfected with a TetR-specific SEAP expression vector (SEQ-ID NO: 168) and constitutive expression vectors for TetR-Elk1 (SEQ-ID NO: 199) and corresponding GEMS_{NS3a (H1)} and GEMS_{ANR} constructs (SEQ-ID NOs: 189&194). For grazoprevir-repressible target gene expression by GEMS variants containing VEGFR-derived intracellular domains, cells were co-transfected with a calcium-inducible SEAP expression vector (SEQ-ID NO: 167) and constitutive expression vectors for corresponding GEMS_{NS3a (H1)} and GEMS_{ANR} constructs (SEQ-ID NOs: 190&196). SEAP levels in culture supernatants were scored at 48 h after addition of 10 μM Grazoprevir dissolved in DMSO (vehicle control). Based on experimental results shown in (FIG. 25B), the new grazoprevir-inducible BUF2 switch was established by co-expression of NS3a (H1)-GEMS_{TM}-IL-6RBₘ (designated GEMS_{NS3a (H1)}, SEQ-ID NO: 192), GNCR-GEMS_{TM}-IL-6RBₘ (designated GEMS_{GNCR}, SEQ-ID NO: 191) and a reporter gene expression vector driven by synthetic STAT3-specific promoters (SEQ-ID NOs: 164&169). Likewise, the new grazoprevir-repressible NOT₂ switch comprises (ANR)ₙ-GEMS_{TM}-IL-6RBₘ instead of GEMS_{GNCR} (designated GEMS_{ANR}, SEQ-ID NOs: 187&188; n can be any number between 1 and 1000, and most preferably 4 or 8) (FIG. 25). To build a third set of grazoprevir-regulated BUF/NOT switches, we capitalized on a NS3a-containing self-cleaving degron StaPLd (SEQ-ID NO: 116). Because StaPLd triggers autoproteolysis of each polypeptide construct it is residing in, engineered transcription factors PcaV-StaPL-VP64 (SEQ-ID NO: 198) and PcaV-StaPL-KRAB (SEQ-ID NO: 197) will undergo spontaneous degradation unless the presence of grazoprevir inhibits self-cleavage activity of StaPLd by binding to its NS3a-domain (FIG. 25). Thus, grazoprevir enables PcaV-StaPL-VP64 to trans-activate minimal PcaV-specific promoters in a typical BUF3 manner, whereas NOT₃ results from grazoprevir-dependent silencing of constitutive promoters harboring binding sites for PcaV-StaPL-KRAB (FIG. 25C). For grazoprevir-repressible gene expression (NOT₃), HEK-293 were co-transfected with a constitutive PcaV-StaPL-KRAB expression vector (SEQ-ID NO: 197) and a PcaV-repressible SEAP expression vector (SEQ-ID NO: 166). For grazoprevir-inducible gene expression (BUF3), HEK-293 were co-transfected with a constitutive PcaV-StaPL-VP64 expression vector (SEQ-ID NO: 198) and a PcaV-specific SEAP expression vector (SEQ-ID NO: 165). SEAP levels in culture supernatants were quantified at 48 h after addition of 10 μM Grazoprevir.

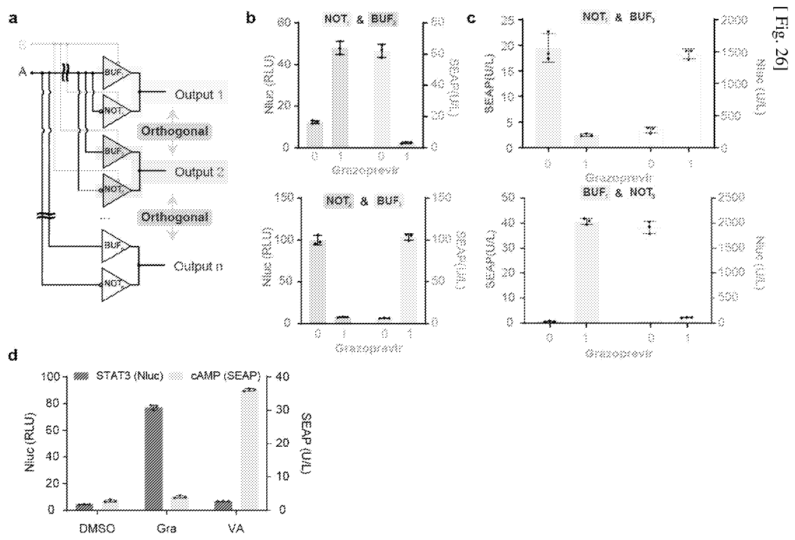

To integrate different sets of BUFₙ/NOTₙ switches into a same tristate-based gene circuit, it is essential that there is no signal crosstalk between each individual set of gene switches when regulating different output modules in parallel (FIG. 26A). Therefore, we co-expressed an NOT switch controlling secreted alkaline phosphatase (SEAP, SEQ-ID NO: 114) as a first reporter gene with a BUF2 switch controlling secreted Nano luciferase (NLuc, SEQ-ID NO: 101) as a second reporter gene. We also co-expressed the NOT₂ switch controlling NLuc with a BUF switch controlling SEAP in the same cells (FIG. 26B). Specifically, HEK-293 cells were co-transfected with plasmids encoding for a grazoprevir-regulated BUF2 switch driving NanoLuc expression (GNCR-GEMS_{IL6RB}&NS3a (H1)-GEMS_{IL6RB}&P_{STAT3}-NanoLuc; SEQ-ID NOs: 191, 192&164) and a grazoprevir-regulated NOT₁ switch driving SEAP expression ((ANR)₈-NSP3&MCP-(NS3a (H1))₃&SEAP-mRNA with MCP-specific poly (A)-surrogate; SEQ-ID NOs: 5, 58&137). Similarly, HEK-293 cells were co-transfected with plasmids encoding for a grazoprevir-regulated NOT₂ switch driving NanoLuc expression (ANR-GEMS_{IL6RB}&NS3a (H1)-GEMS_{IL6RB}&a STAT3-specific NanoLuc expression vector; SEQ-ID NOs: 188, 192&164) and a grazoprevir-regulated BUF1 switch driving SEAP expression ((GNCR)₃-NSP3&MCP-(NS3a (H1))₃&SEAP-mRNA with MCP-specific poly (A)-surrogate; SEQ-ID NOs: 30, 58&137). SEAP and NanoLuc levels in the culture supernatants were scored at 48 h after cultivation in cell culture medium containing 0 or 10 μM Grazoprevir. Similar experiments were also performed to demonstrate interference-free operation between NOT₁&BUF3 and NOT₃&BUF₁ (FIG. 26C). For example, a grazoprevir-regulated BUF3 switch driving SEAP expression (SEQ-ID NOs: 198&165) was co-administered with a grazoprevir-regulated NOT₁ switch driving NanoLuc expression (SEQ-ID NOs: 5, 58&163) into HEK-293 cells. Also, a grazoprevir-regulated NOT₃ switch driving SEAP expression (SEQ-ID NOs: 197&166) was co-administered with a grazoprevir-regulated BUF1 switch driving NanoLuc expression (SEQ-ID NOs: 30, 58&163) into HEK-293 cells. SEAP and NanoLuc levels in the culture supernatants were scored at 48 h after cultivation in cell culture medium containing 0 or 10 μM Grazoprevir. Results showed that each individual switch operated in a highly autonomous manner when triggered by grazoprevir, demonstrating robust and interference-free performance in mammalian cells.

Because both IF1 and BUF2/NOT₂ utilize different intracellular signaling pathways in mammalian cells, we need to test whether there is potential signal crosstalk between respective key components before plugging new sets of grazoprevir-regulated BUF/NOT switches onto the output wire of the vanillic acid-controlled upstream module. For this purpose, a vanillic acid-inducible gene switch controlling SEAP expression (IF1; SEQ-ID NO: 172) was co-administered with grazoprevir-regulated GEMS-based BUF switch driving NanoLuc expression (SEQ-ID NOs: 191, 192&164) into HEK-293 cells stably transgenic for MOR9-1 expression (HEK-MOR9 (C0)), before cultivation in cell culture medium containing Vanillic Acid (VA, 400 μM) and/or Grazoprevir (Gra, 10 μM). SEAP levels in the culture supernatants were scored at 48 h post transfection. Experiments show that such an IF1-switch controlling SEAP expression could operate in parallel to a BUF2-switch driving NLuc expression in same cells. This not only supports feasibility to create tristate buffers, but also indicates potential orthogonality between intracellular cAMP and STAT3-signaling from a cell biology perspective in general (FIG. 26D).

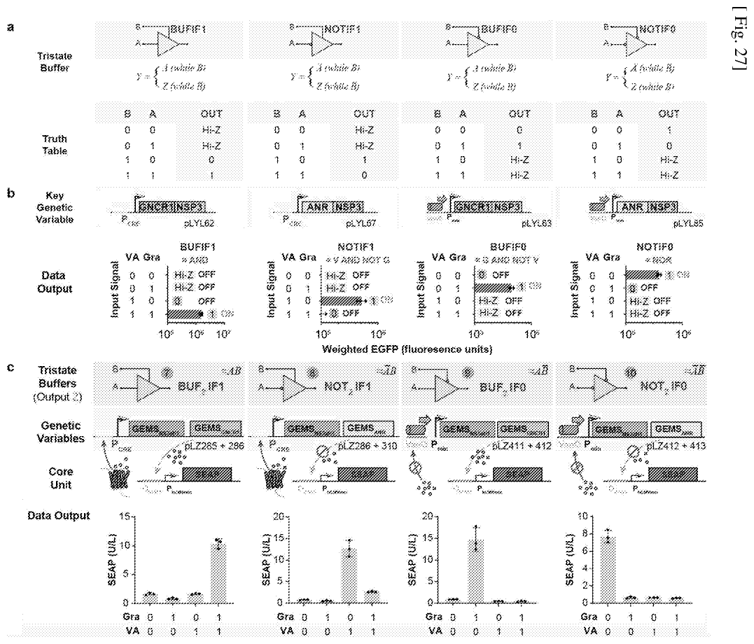

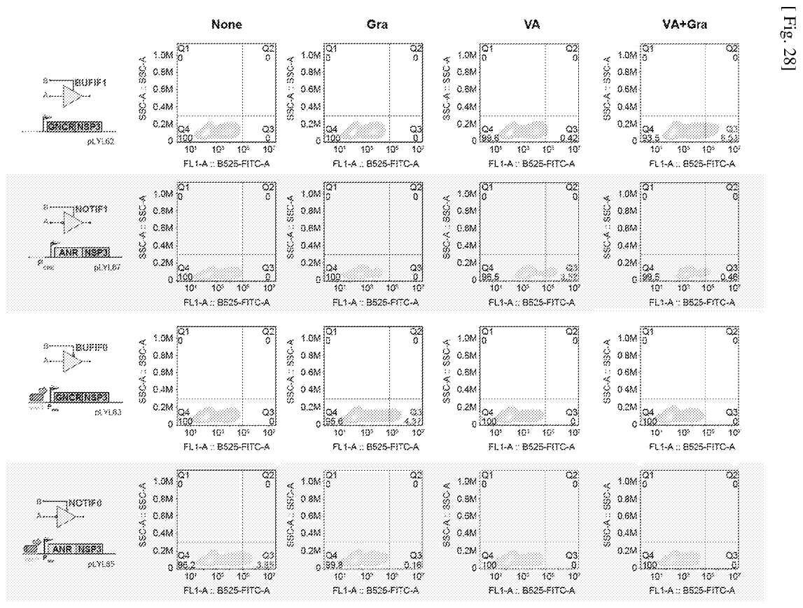

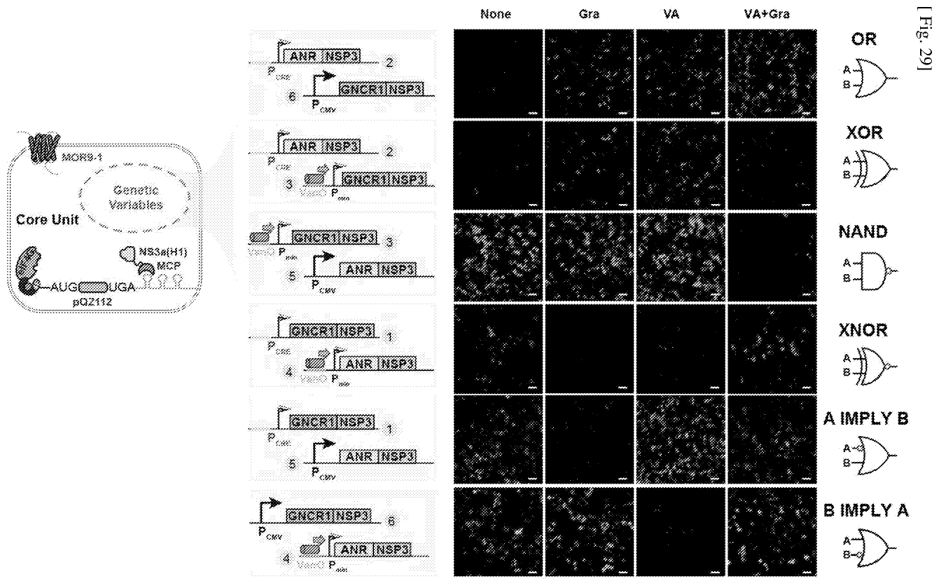

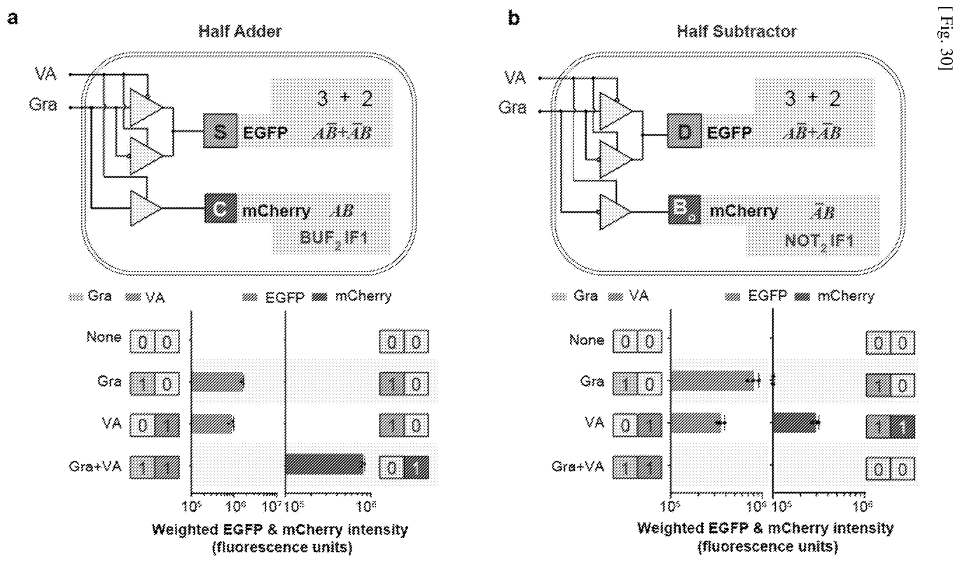

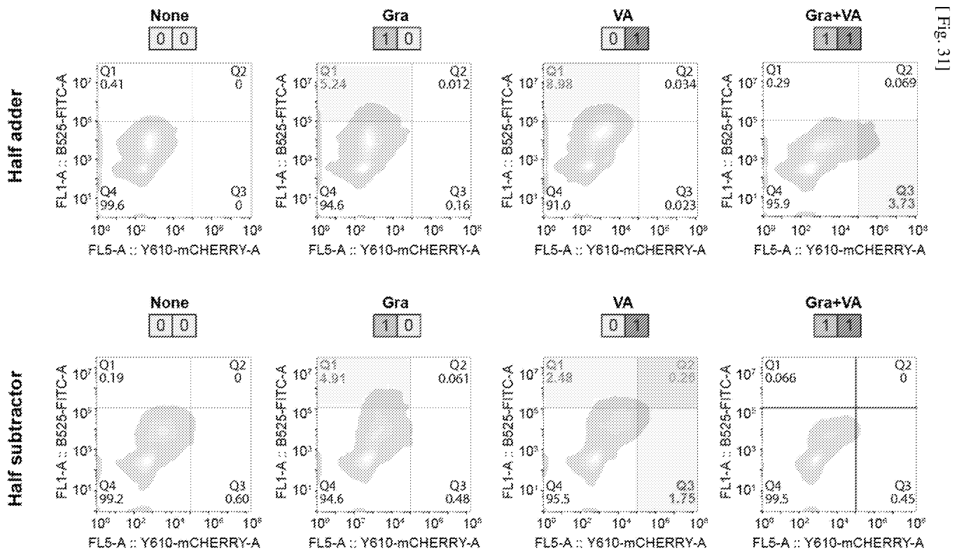

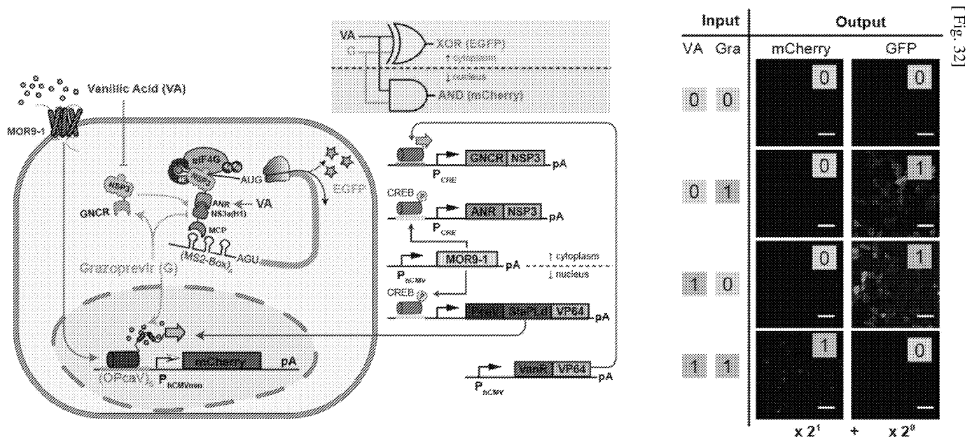

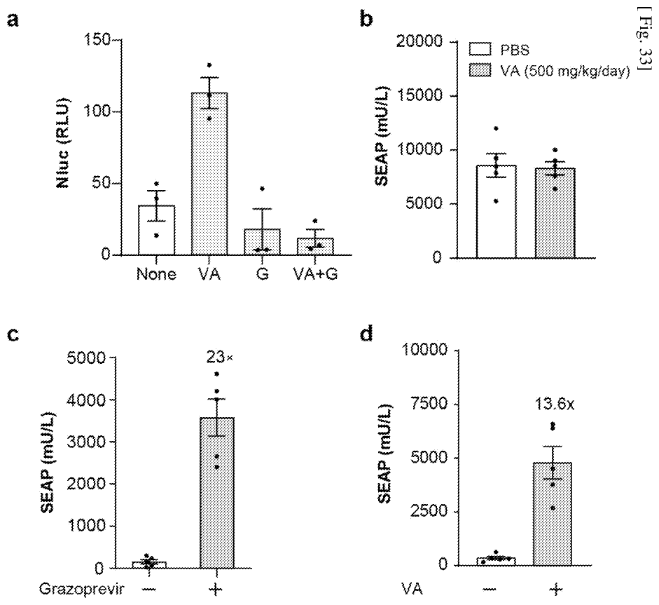

Finally, we connected vanillic acid (VA)-regulated IF0 and IF1 switches (upstream module) and grazoprevir (Gra)-regulated BUF and NOT switches (downstream module), thus yielding the four different types of tristate buffers: BUFIF1 (Active-High Buffer where IF1 regulates BUF), NOTIF1 (Active-High Inverted Buffer where IF1 regulates NOT), BUFIF0 (Active-Low Buffer where IF0 regulates BUF) and NOTIF0 (Active-Low Inverted Buffer where IF0 regulates NOT) (FIG. 27A). In a biological context, BUFIF1 shows logic similarity with a conventional AND gate, while NOTIF0 is logically similar to a conventional NOR gate. Likewise, NOTIF1 and BUFIF0 show typical gene expression signatures of both variants of NIMPLY (AND NOT) gates (FIG. 27B). For BUFIF1, HEK-293 cells were co-transfected with plasmids encoding MCP-specific EGFP mRNA (SEQ-ID NO: 149), constitutive expression vectors for MCP-(NS3a (H1))₃ (SEQ-ID NO: 58) and MOR9-1 (SEQ-ID NO: 208) and a cAMP-responsive (GNCR)₃-NSP3 expression vector (SEQ-ID NO: 162). For NOTIF0, cells were co-transfected with SEQ-ID NO: 149, constitutive expression vectors for MCP-(NS3a (H1))₃ (SEQ-ID NO: 58) and VanR-VP64 (SEQ-ID NO: 296) and a vanillic acid-responsive (ANR)₄-NSP3 expression vector (SEQ-ID NO: 161). For NOTIF1, cells were co-transfected with SEQ-ID NO: 149, constitutive expression vectors for MCP-(NS3a (H1))₃ (SEQ-ID NO: 58) and MOR9-1 (SEQ-ID NO: 208) and a cAMP-responsive (ANR)₄-NSP3 expression vector (SEQ-ID NO: 160). For BUFIF0, cells were co-transfected with SEQ-ID NO: 149, constitutive expression vectors for MCP-(NS3a (H1))₃ (SEQ-ID NO: 58) and VanR-VP64 (SEQ-ID NO: 296) and a vanillic acid-responsive (GNCR)₃-NSP3 expression vector (SEQ-ID NO: 159). Vanillic Acid (VA, 400 μM) and Grazoprevir (Gra, 0.5 μM) were added at 6 h post transfection. At 36 hours post transfection, fluorescent images showing EGFP signals (scale bar: 100 μm) were acquired and flow-cytometry analysis was performed with 10000 cells per group (FIG. 28). The same procedure can be performed using the second set of grazoprevir (Gra)-regulated BUF and NOT switches (BUF2/NOT₂) (FIG. 27C). For BUF2IF1, HEK-293 cells stably expressing MOR9-1 (HEK-MOR9 (C0)) were co-transfected with a STAT3-specific SEAP expression vector (SEQ-ID NO: 169) and cAMP-responsive expression vectors for GEMS_{GNCR} (SEQ-ID NO: 158) and GEMS_{NS3a (H1)} (SEQ-ID NO: 157). For NOT₂IF0, HEK-293 cells were co-transfected with SEQ-ID NO: 169, a constitutive VanR-VP64 (SEQ-ID NO: 296) expression vector and vanillic acid-responsive expression vectors for GEMS_{NS3a (H1)} (SEQ-ID NO: 156) and GEMS_{ANR} (SEQ-ID NO: 155). For NOT₂IF1, HEK-MOR9 (C0) cells were co-transfected with SEQ-ID NO: 169 and cAMP-responsive expression vectors for GEMS_{ANR} (SEQ-ID NO: 154) and GEMS_{NS3a (H1)} (SEQ-ID NO: 157). For BUF2IF0, HEK-293 cells were co-transfected with SEQ-ID NO: 169, a constitutive VanR-VP64 (SEQ-ID NO: 296) expression vector and vanillic acid-responsive expression vectors for GEMS_{NS3a (H1)} (SEQ-ID NO: 156) and GEMS_{GNCR} (SEQ-ID NO: 313). After cultivation in medium containing Vanillic Acid (VA, 400 μM) and/or Grazoprevir (Gra, 10 μM), SEAP levels in the culture supernatants were scored at 48 h post transfection. Thus, combination of vanillic acid-regulated IF1/IF0 with either set of grazoprevir-regulated BUFₙ/NOTₙ switches can produce the four tristate buffers BUFIF1, NOTIF1, BUFIF0 and NOTIF0 with logic similarity to AND-, NOR- and IMPLY-gates in mammalian cells. This would set the stage to engineer various sophisticated mammalian cell functions of interest resembling the computational logics of any 2-input 1-output Boolean logic gate or 2-input 2-output Boolean calculus. For example, combination of NOTIF1 with another BUF switch produces a gene circuit that shows the expression profile of a conventional OR gate (FIG. 29). Experimentally, HEK-293 cells were transfected with a constitutive expression vector for (GNCR)₃-NSP3 (SEQ-ID NO: 30) and a cAMP-responsive (ANR) g-NSP3 expression vector (SEQ-ID NO: 153). Similarly, NAND gate-like logics are achieved through addition of NOT to BUFIF0 (transfection of HEK-293 cells with constitutive expression vectors for (ANR)₈-NSP3 (SEQ-ID NO: 5) and VanR-VP64 (SEQ-ID NO: 296) and a vanillic acid-responsive (GNCR)₃-NSP3 expression vector (SEQ-ID NO: 152)), while combinations of NOT with BUFIF1 or addition of BUF to NOTIF0 produce the two variants of IMPLY gates (FIG. 29). For Gra IMPLY VA logics, cells were transfected with a constitutive expression vector for (ANR)₈-NSP3 (SEQ-ID NO: 5) and a cAMP-responsive (GNCR)₃-NSP3 expression vector (SEQ-ID NO: 162). For VA IMPLY Gra logics, cells were transfected with constitutive expression vectors for (GNCR)₃-NSP3 (SEQ-ID NO: 30) and VanR-VP64 (SEQ-ID NO: 296) and a vanillic acid-responsive (ANR)₈-NSP3 expression vector (SEQ-ID NO: 151). XOR logics is achieved by combining BUFIF0 with NOTIF1 (transfection of HEK-293 cells with a constitutive VanR-VP64 (SEQ-ID NO: 296) expression vector, a vanillic acid-responsive (GNCR)₃-NSP3 expression vector (SEQ-ID NO: 159) and a cAMP-responsive (ANR)₄-NSP3 expression vector (SEQ-ID NO: 160)), while XNOR was produced through superimposition of NOTIF0 to BUFIF1 (transfection of HEK-293 cells with a constitutive VanR-VP64 (SEQ-ID NO: 296) expression vector, a cAMP-responsive (GNCR)₃-NSP3 expression vector (SEQ-ID NO: 162) and a vanillic acid-responsive (ANR)₈-NSP3 expression vector (SEQ-ID NO: 151)) (FIG. 29). For all circuits, cells were co-transfected with a reporter plasmid encoding MCP-specific EGFP mRNA (SEQ-ID NO: 149) and a constitutive expression vector for MCP-(NS3a (H1))₃ (SEQ-ID NO: 58), forming the computational basis to run all the different circuits. All experiments can be either performed in native HEK-293 cells (through co-transfection of a constitutive MOR9-1 (SEQ-ID NO: 208) expression vector or in HEK-293 cells stably expressing MOR9-1 (HEK-MOR9 (C0)). Methods for generation of stable cell lines are described above. At 6 h post transfection, different combinations of Vanillic Acid (V, 400 μM) and Grazoprevir (G, 0.5 μM) were added. At 36 hours post transfection, fluorescent images showing EGFP signals (scale bar: 100 μm) were acquired.

Whereas Boolean logic gates convert multiple input signals into a single output signal according to pre-programmed algorithms³⁹, calculators typically produce multiple output signals. For example, adders and subtractors perform Boolean algebra between two or more inputs in a way where every bit is displayed as a different output signal representing a different 2ⁿ digit. For example, a half-adder returns the digits sum S (representative for the 2⁰ digit) and carry Y (representative for the 21 digit) through binary addition of the two inputs A and B. Likewise, a half-subtractor performs binary subtraction of B from A using two different output signals for borrow W (representative for the −1×2¹ digit) and difference D (representative for the 2⁰ digit). To this end, tristate buffers also allow modular and systematic assembly of various Boolean calculators in mammalian cells. For example, a half-adder is produced through addition of the grazoprevir- and vanillic acid-regulated BUF₁IF0, NOT₁IF1 and BUF₂IF1 tristate buffers (FIG. 30A). Experimentally, HEK-MOR9 (C0) cells were co-transfected with constitutive expression vectors for VanR-VP64 (SEQ-ID NO: 296), MOR9-1 (SEQ-ID NO: 208), MCP-(NS3a (H1))₃ (SEQ-ID NO: 58) and MCP-specific EGFP mRNA (SEQ-ID NO: 149), a vanillic acid-responsive (GNCR)₃-NSP3 expression vector (SEQ-ID NO: 152), cAMP-responsive expression vectors for (ANR)₄-NSP3, GEMS_{GNCR} and GEMS_{NS3a (H1)} (SEQ-ID NOs: 157, 158&160) and a STAT3-specific mCherry expression vector (SEQ-ID NO: 150). Flow cytometric analysis of EGFP- and mCherry signals (FIG. 31A) was performed at 48 h after cultivation in medium containing different combinations of Vanillic Acid (V, 400 μM) and Grazoprevir (G, 10 μM). Data show weighted fluorescence units as mean±SD representative for 3 individual experiments. A half-adder was also created on the basis of BUF₁IF0, NOT₁IF1 and BUF₃IF1 (FIG. 32). Specifically, HEK-293 cells were co-transfected with plasmids encoding EGFP-mRNA containing MCP-specific poly (A)-surrogate (SEQ-ID NO: 149), constitutive expression vectors for MCP-(NS3a (H1))₃ (SEQ-ID NO: 58), VanR-VP64 (SEQ-ID NO: 296) and MOR9-1 (SEQ-ID NO: 208), cAMP-responsive expression vectors for (ANR)₄-NSP3 and NLS-PcaV-StaPLd-VP64 (SEQ-ID NOs: 160&130), a vanillic acid-responsive (GNCR)₃-NSP3 expression vector (SEQ-ID NO: 159) and a PcaV-specific mCherry expression vector (SEQ-ID NO: 141). At 6 h post transfection, different combinations of Vanillic Acid (V, 400 μM) and Grazoprevir (G, 0.5 μM) were added. At 36 hours post transfection, fluorescent images showing EGFP signals (scale bar: 100 μm) were acquired and flow-cytometry analysis was performed (10000 cells per group). Furthermore, a half-subtractor is assembled through the three tristate buffers BUF₁IF0, NOT₁IF1 and NOT₂ IF1 (FIG. 30B). Specifically, HEK-MOR9 (C0) cells were co-transfected with cAMP-responsive expression vectors for (ANR)₄-NSP3, GEMS_{ANR} and GEMS_{NS3a (H1)} (SEQ-ID NOs: 160, 154&157) and a STAT3-specific mCherry expression vector (SEQ-ID NO: 150), constitutive expression vectors for MCP-(NS3a (H1))₃ (SEQ-ID NO: 58), VanR-VP64 (SEQ-ID NO: 296) and MOR9-1 (SEQ-ID NO: 208), a vanillic acid-responsive (GNCR)₃-NSP3 expression vector (SEQ-ID NO: 159) and EGFP-mRNA containing MCP-specific poly (A)-surrogate (SEQ-ID NO: 149). Flow cytometric analysis of EGFP- and mCherry signals (FIG. 31B) was performed at 48 h after cultivation in medium containing different combinations of Vanillic Acid (V, 400 μM) and Grazoprevir (G, 10 μM). In vivo, only the vanillic acid AND NOT grazoprevir gate was functional (FIG. 33A), probably due to the inability of the transcription-based IF0 switch to properly operate in mice (FIG. 33B). However, all other modules operating at the translational (NOT₁; FIG. 33C) and cell signaling-level (IF1; FIG. 33D) were fully functional in mice, implying a possible limitation of currently used transcription-based gene switches for in vivo applications. To test vanillic acid AND NOT grazoprevir logics (NOTIF1) in vivo, 420 μg of plasmid DNA (pSL683/pSL548/pLYL76/pLYL67 in a 2:6:16:3 (w/w/w/w) ratio were hydrodynamically injected into the tail vein of WT C57BL/6 mice according to the experimental details described in the Methods section above (producing SEQ-ID NOs: 208, 58, 160&139). At 6 h post injection, different combinations of Vanillic Acid (500 mg/kg/day) and Grazoprevir (9 mg/kg/day) were administered by intraperitoneal injection (3 times per day). NanoLuc levels in the bloodstream of mice were measured at 24 h after first stimulation. To test the vanillic Acid (VA)-repressible IF0 switch in vivo, 25 μg of plasmid DNA (pSL175/pSL173 in a 3:2 (w/w) ratio) were hydrodynamically injected into the tail vein of WT C57BL/6 mice (producing SEQ-ID NOs: 171&296). At 6 h post injection, Vanillic Acid (500 mg/kg/day) dissolved in PBS was administered by intraperitoneal injection (3 times per day). SEAP levels in the bloodstream of mice were measured at 24 h after the first Vanillic Acid injection. To test the grazoprevir-repressible NOT₁ switch in vivo, 450 μg of plasmid DNA (pSL468/pSL549/pSL548 in a 1:4:4 (w/w/w) ratio) were hydrodynamically injected into the tail vein of WT C57BL/6 mice (producing SEQ-ID NOs: 3, 58&137). At 6 h post injection, Grazoprevir (3 mg/kg) dissolved in PBS was administered by intraperitoneal injection 3 times per day. SEAP levels in the bloodstream of mice were measured at 24 h after the first Grazoprevir injection. To test the vanillic Acid (VA)-inducible IF1 switch in vivo, 430 μg of plasmid DNA (pLYL76/pCK53 in a 40:3 (w/w) ratio) were hydrodynamically injected into the tail vein of WT C57BL/6 mice (producing SEQ-ID NOs: 172&208). At 6 h post injection, Vanillic Acid (500 mg/kg/day) dissolved in PBS was administered by intraperitoneal injection (3 times per day). SEAP levels in the bloodstream of mice were measured at 24 h after the first Vanillic Acid injection.

### Example 5: Engineering of Intracellular Protein Sensors for In Vitro Diagnostics in Cell-Based and Cell-Free Contexts

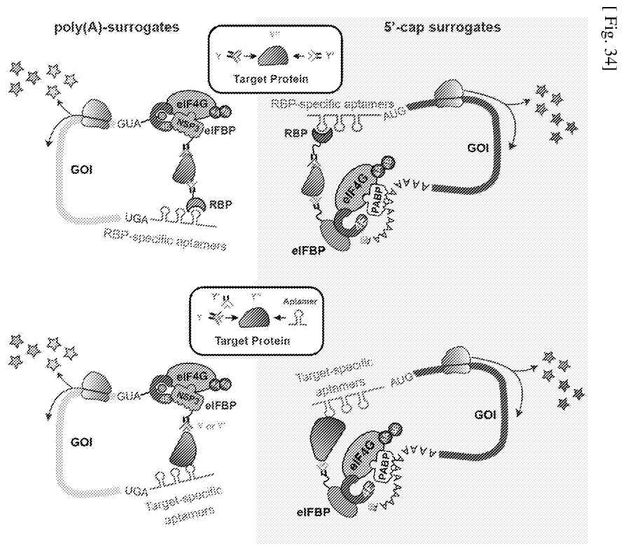

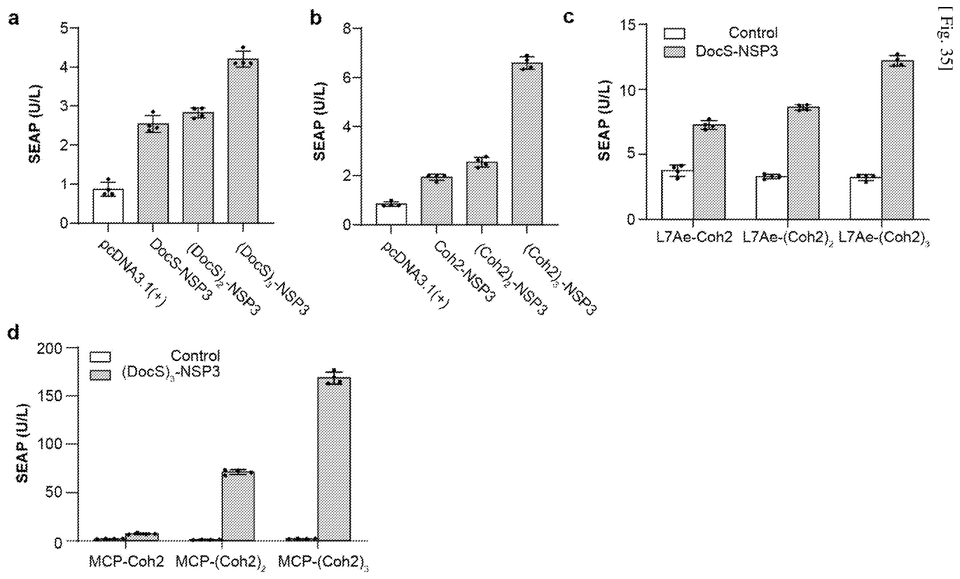

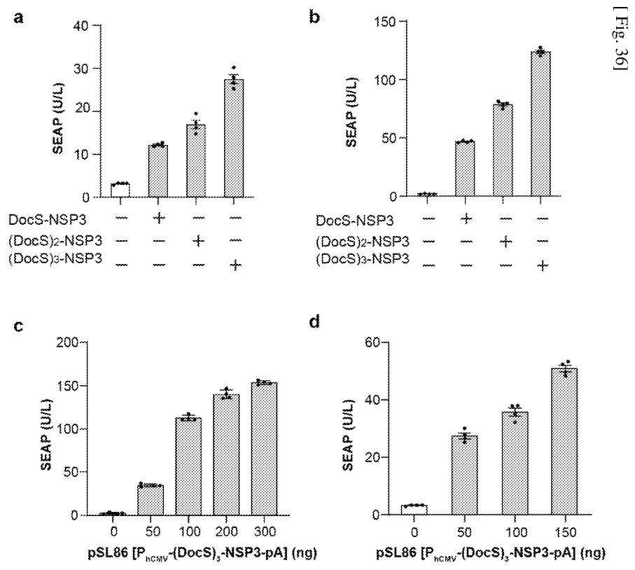

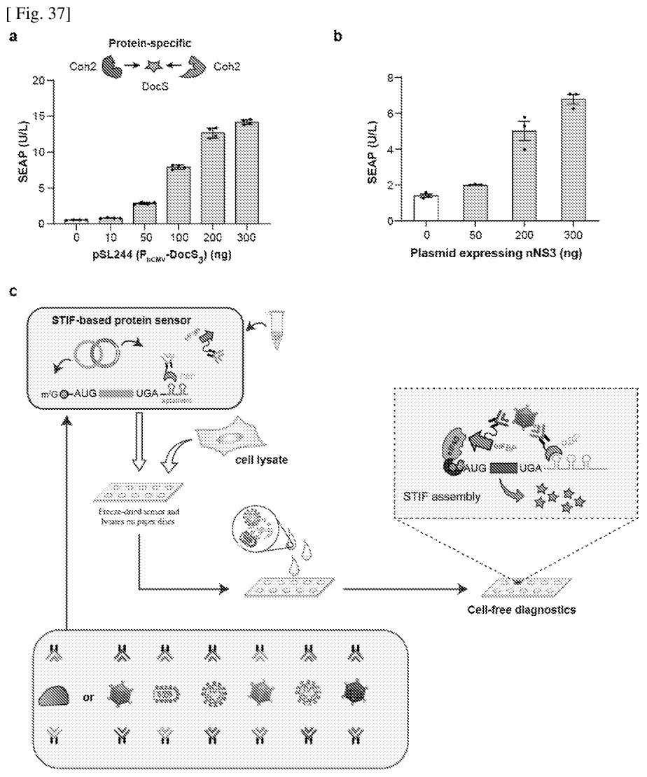

The STIF-based translational regulation strategy can also be repurposed to engineer intracellular protein sensors. For example, when each split-STIF component is fused to a different member of a protein heterotrimer system, STIF-dependent gene expression from poly (A)-deficient mRNA would strictly depend on the presence of the remaining member(s) of the full protein complex (FIG. 34). For this purpose, we first show that bipartite STIF constructs can be engineered on the basis of both MCP-Coh2/DocS-NSP3 (FIG. 35A) as well as MCP-DocS/Coh2-NSP3 combinations of bipartite STIF constructs (FIG. 35B). Then, we established that STIF components containing three tandem repeats of Coh2 (SEQ-ID NO: 77) (FIGS. 35C, 35D) and DocS (SEQ-ID NO: 80) (FIGS. 36A, 36B) showed optimal efficiency for dose-dependent translational initiation of poly (A)-deficient SEAP mRNA (FIGS. 36C, 36D). Hence, we fused three tandem Coh2 repeats to both the RBP-domain (L7Ae or MCP) and the NSP3 domain and used both STIF components MCP-(Coh2)₃ (SEQ-ID NO: 273) and (Coh2)₃-NSP3 (SEQ-ID NO: 9) as a highly specific “molecular pincer” to detect cytosolic (DocS)₃ (SEQ-ID NO: 302) in a dose-dependent manner (FIG. 37A). For example (FIG. 35A), HEK-293 cells were co-transfected with plasmids encoding SEAP-mRNA containing MCP-specific poly (A)-surrogate (SEQ-ID NO: 137), MCP-Coh2 (SEQ-ID NO: 51) and NSP3-fusion proteins consisting of one (SEQ-ID NO: 12), two (SEQ-ID NO: 13) or three N-terminal DocS-repeats (SEQ-ID NO: 14). Alternatively (FIG. 35B), HEK-293 cells were co-transfected with plasmids encoding SEAP-mRNA containing MCP-specific poly (A)-surrogate (SEQ-ID NO: 137), MCP-DocS (SEQ-ID NO: 246), and NSP3-fusion proteins consisting of one (SEQ-ID NO: 268), two (SEQ-ID NO: 269) or three N-terminal Coh2-repeats (SEQ-ID NO: 9). For bipartite STIFs based on L7Ae-Coh2 (FIG. 35C), HEK-293 cells were co-transfected with plasmids encoding SEAP-mRNA containing L7Ae-specific poly (A)-surrogate (SEQ-ID NOs: 126&134), DocS-NSP3 (SEQ-ID NO: 12) and different L7Ae-fusion proteins with one (SEQ-ID NO: 33), two (SEQ-ID NO: 34) or three C-terminal Coh2-repeats (SEQ-ID NO: 35). For bipartite STIFs based on MCP-Coh2 (FIG. 35D), HEK-293 cells were co-transfected with plasmids encoding SEAP-mRNA containing MCP-specific poly (A)-surrogate (SEQ-ID NO: 137), (DocS)₃-NSP3 (SEQ-ID NO: 14) and different MCP-fusion proteins consisting of one (SEQ-ID NO: 51), two (SEQ-ID NO: 272) or three C-terminal Coh2-repeats (SEQ-ID NO: 273). For bipartite STIFs based on DocS-NSP3 (FIGS. 36A, 36B), HEK-293 cells were co-transfected with plasmids encoding NSP3-fusion proteins consisting of one (SEQ-ID NO: 12), two (SEQ-ID NO: 13) or three N-terminal DocS-repeats (SEQ-ID NO: 14) and SEAP-mRNA containing either L7Ae-specific poly (A)-surrogate (SEQ-ID NOs: 126&134) and L7Ae-(Coh2)₃ (SEQ-ID NO: 35) (FIG. 36A) or MCP-specific poly (A)-surrogate (SEQ-ID NO: 137) and MCP-(Coh2)₃ (SEQ-ID NO: 273) (FIG. 36B). For dose-dependent translational activation of MCP-(Coh2)₃-specific mRNA (FIG. 36C), HEK-293 cells were co-transfected with plasmids encoding SEAP-mRNA containing an MCP-specific poly (A)-surrogate (SEQ-ID NO: 137), MCP-(Coh2)₃ (SEQ-ID NO: 273) and different amounts of (DocS)₃-NSP3 expression vectors (SEQ-ID NO: 14). For dose-dependent translational activation of L7Ae-(Coh2)₃-specific mRNA (FIG. 36D) HEK-293 cells were co-transfected with plasmids encoding SEAP-mRNA containing L7Ae-specific poly (A)-surrogate (SEQ-ID NOs: 126&134), L7Ae-(Coh2)₃ (SEQ-ID NO: 35) and different amounts of (DocS)₃-NSP3 expression vectors (SEQ-ID NO: 14). To validate STIF-based protein sensors (FIG. 37A), HEK-293 cells were co-transfected with plasmids encoding SEAP-mRNA containing an MCP-specific poly (A)-surrogate (SEQ-ID NO: 137) and constitutive expression vectors for MCP-(Coh2)₃ (SEQ-ID NO: 273), (Coh2)₃-NSP3 (SEQ-ID NO: 9) and (DocS)₃ (SEQ-ID NO: 302, by different amounts of pSL244). SEAP expression in culture supernatants were scored at 48 h post transfection.

Such molecular pincers can be used to detect various proteins of interest in living cells, as soon as pairs of highly specific binders for a same target is available. As a general design blueprint to engineer STIF-based sensors for a specific target Y″, a pair of proteins Y and Y′ both binding to different epitopes of Y″ are therefore required. Then, Y and Y′ can be fused either to the N-terminus or C-terminus of RBP or eIFBP domains of STIF regulators, allowing Y″ to initiate translational initiation upon triggering the circularized configuration of mRNA that contain RBP-specific poly (A)-surrogate (FIG. 34). For example, we engineered a sensor for the Hepatitis C virus (HCV)-specific nNS3-protein (SEQ-ID NO: 303) by fusing two different nNS3-specific scFvs to either L7Ae and NSP3, yielding L7Ae-scFv35 (SEQ-ID NO: 47) and (scFv162)₃-NSP3 (SEQ-ID NO: 262). HEK-293 cells were then co-transfected with plasmids encoding SEAP-mRNA containing L7Ae-specific poly (A)-surrogate (SEQ-ID NOs: 126&134), L7Ae-scFv35 (SEQ-ID NO: 47), (scFv162)₃-NSP3 (SEQ-ID NO: 262) and different amounts of overexpressed nNS3 (SEQ-ID NO: 303). SEAP expression in culture supernatants were scored at 48 h post transfection. Results show that co-expression of L7Ae-scFv35 and (scFv162)₃-NSP3 in mammalian cells activated translation of target mRNA containing L7Ae-specific poly (A)-surrogate in a strict nNS3-dependent manner (FIG. 37B), demonstrating the application potential of such STIF-based protein sensors for molecular diagnostics either through gene delivery into living cells or by developing point-of-care testing devices based on synthetic gene circuits operating in cell-free systems (Pardee et al., 2014) (FIG. 37C).

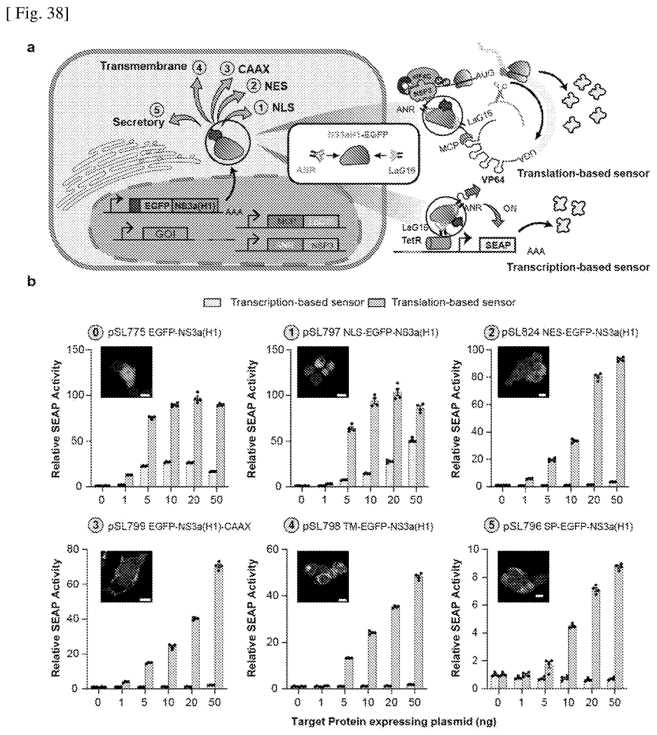

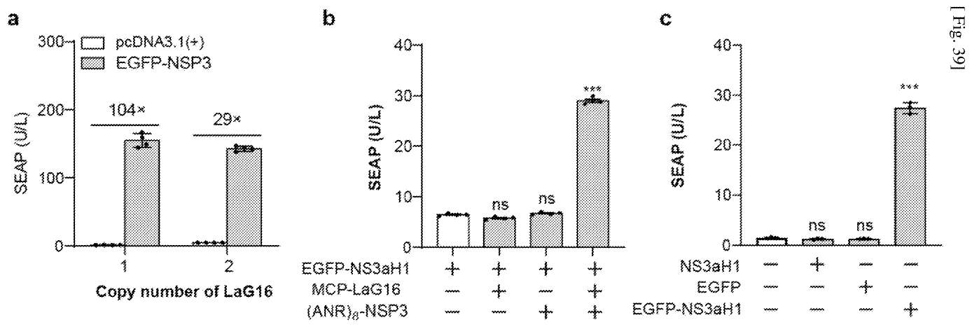

To demonstrate the advantage of translation-based sensors over state-of-the art transcription-based sensors, we created a synthetic EGFP-NS3a (H1) protein (SEQ-ID NO: 17) as a model target signal for detection (FIG. 38A). This synthetic target protein EGFP-NS3a (H1) was then targeted to different intracellular compartments through fusion with different localization signals (NLS (nuclear localization signal, SEQ-ID NO: 103), producing SEQ-ID NO: 21; NES (nuclear export signal, SEQ-ID NO: 102), producing SEQ-ID NO: 20; CAAX (prenylation motif, SEQ-ID NO: 75), producing SEQ-ID NO: 19; TM (transmembrane localization signal, SEQ-ID NO: 118), producing SEQ-ID NO: 23; SP (secretory signal peptide, SEQ-ID NO: 115), producing SEQ-ID NO: 22), allowing detection of each differentially localized proteins with co-expressed genetic sensors consisting of LaG16 (an EGFP nanobody, SEQ-ID NO: 93) and ANR (a peptide motif binding to NS3a (H1), SEQ-ID NO: 105). For transcription-based sensing, LaG16 (SEQ-ID NO: 93) was fused to TetR (SEQ-ID NO: 117) while different repeats of ANR (SEQ-ID NO: 105) were fused to VP64 (SEQ-ID NO: 119), resulting in EGFP-NS3a (H1)-dependent transcriptional activation of TetR-specific promoters. For translation-based sensing, LaG16 (SEQ-ID NO: 93) was fused to MCP (SEQ-ID NO: 98) while different repeats of ANR (SEQ-ID NO: 105) were fused to NSP3 (SEQ-ID NO: 106), resulting in EGFP-NS3a (H1)-dependent STIF reconstitution and translation of MCP-specific mRNA. To establish the STIF-based EGFP-NS3a (H1) sensor, we found that one LaG16 repeat was sufficient to associate with EGFP (FIG. 39A), whereas NS3a (H1) detection required multiple tandem ANR-peptide motifs fused to the N-terminus of NSP3 (FIG. 39B). For example, HEK-293 cells were co-transfected with plasmids encoding SEAP-mRNA containing MCP-specific poly (A)-surrogate (SEQ-ID NO: 137), a constitutive EGFP-NSP3 (SEQ-ID NO: 18) expression vector and expression vectors for different MCP-LaG16 variants containing one (SEQ-ID NO: 52) or two tandem LaG16 repeats (SEQ-ID NO: 53). Transfection of pcDNA3.1 (+) (Invitrogen, CA; cat-no. V79020) instead of EGFP-NSP3 expression vectors was used as a negative control. Likewise, HEK-293 cells were co-transfected with plasmids encoding SEAP-mRNA containing MCP-specific poly (A)-surrogate (SEQ-ID NO: 137), a constitutive MCP-(NS3a (H1))₃ (SEQ-ID NO: 58) expression vector and expression vectors for different ANR-NSP3 variants containing different numbers tandem ANR repeats (SEQ-ID NOs: 2-5&267). SEAP expression in the culture supernatant were profiled at 48 h after transfection. Thus, the combination of MCP-LaG16 (SEQ-ID NO: 52), (ANR) 8-NSP3 (SEQ-ID NO: 5) and reporter mRNAs containing MCP-specific poly (A)-surrogate (Table 2) was established as a highly accurate genetic sensor for intracellular EGFP-NS3a (H1) (FIG. 39C). To demonstrate sensing accuracy, HEK-293 cells were eventually co-transfected with plasmids encoding reporter SEAP-mRNA containing MCP-specific poly (A)-surrogate (SEQ-ID NO: 137), a constitutive EGFP-NS3a (H1) (SEQ-ID NO: 17) expression vector and different combinations of MCP-LaG16 (SEQ-ID NO: 52) and (ANR) g-NSP3 expression vectors (SEQ-ID NO: 5). Transfection of pcDNA3.1 (+) (Invitrogen, CA; cat-no. V79020) instead of MCP-LaG16 and/or (ANR)₈-NSP3 expression vectors was used as a negative control. SEAP expression in the culture supernatant were profiled at 48 h after transfection.

When directly comparing the sensing capacity of the translation-based sensor with that of an analogous transcriptional sensor (based on constitutive expression of TetR-LaG16 (SEQ-ID NO: 68), (ANR)₈-VP64 (SEQ-ID NO: 6) and a TetR-specific promoter tetO₇-P_{hCMVmin} (SEQ-ID NO: 238)) in terms of spatial resolution and signal-to-noise ratio, we showed that the transcription-based sensor was limited to detection of untagged and nuclear-localized target proteins (FIG. 38B). Instead, the STIF-based translational sensor could quantitatively detect all the differentially localized proteins (FIG. 38B). Specifically, HEK-293 cells were co-transfected with plasmids encoding the translation-based EGFP-NS3a (H1) sensor (SEQ-ID NOs: 52, 5&137: for MCP-LaG16, (ANR)₈-NSP3 and SEAP-mRNA with MCP-specific poly (A)-surrogate) or the transcription-based EGFP-NS3a (H1) sensor (SEQ-ID NOs: 68, 6&168: for TetR-LaG16, (ANR)₈-VP64 and a TetR-specific promoter controlling SEAP transcription) and different amounts of constitutive expression vectors for different target proteins (native EGFP-NS3a (H1), SEQ-ID NO: 17; NLS-EGFP-NS3a (H1), SEQ-ID NO: 21; NES-EGFP-NS3a (H1), SEQ-ID NO: 20; prenylated EGFP-NS3a (H1)-CAAX, SEQ-ID NO: 19; membrane-localized TM-EGFP-NS3a (H1), SEQ-ID NO: 23; secretory SP-EGFP-NS3a (H1), SEQ-ID NO: 22). At 48 h after transfection, fluorescence images of cellular EGFP signals were acquired (scale bar: 10 μm) and SEAP levels in the culture supernatant were profiled.

### Example 6: Fusion Protein Sensor for Programmable and Self-Sufficient Cancer Gene Therapy

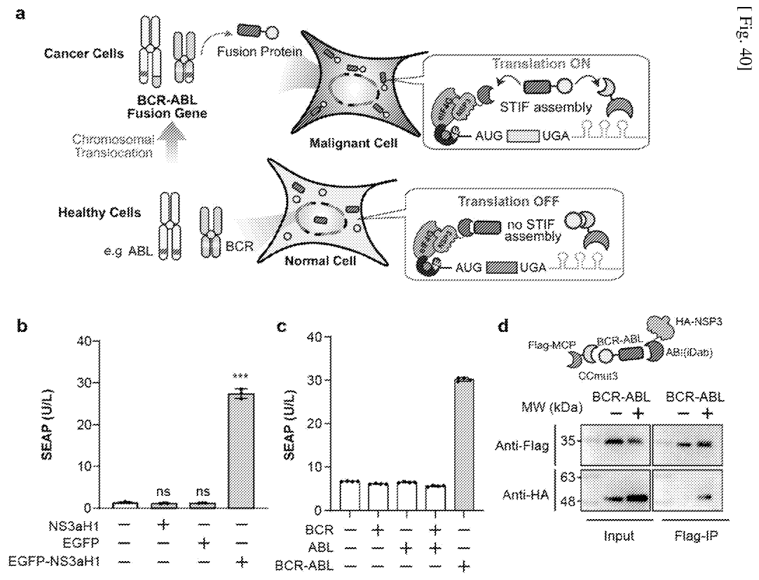

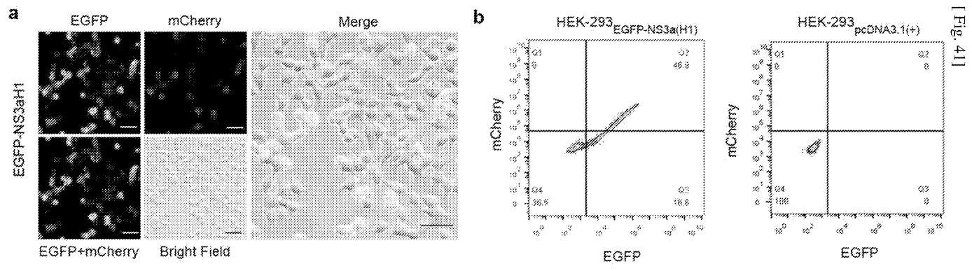

Chromosomal translocations and gene fusions are hallmarks of neoplastic transformation during the early stages of cancer (Mitelman et al., 2007). In many cases, the chromosomal aberration produces a characteristic fusion protein, which disrupts essential function(s) of each individual gene product, and consequently triggers various malignant cellular processes (FIG. 40A). While these gene rearrangements represent important and early steps of carcinogenesis, there is currently no technology that can detect such gene fusions at an early stage in living tissues, despite their prognostic importance for cancer diagnosis (Mitelman et al., 2007). We have found that the molecular architecture of STIF-based protein sensors were effective for real-time detection and treatment of cells harboring gene fusions; we have shown that the MCP-LaG16/(ANR)₈-NSP3-based translational sensor can quantitatively detect intracellular target proteins independently of their subcellular localization (FIG. 38), and furthermore our synthetic target EGFP-NS3a (H1) (SEQ-ID NO: 17) exemplifies a fusion protein arbitrarily formed from “normal” EGFP (SEQ-ID NO: 82) and NS3a (H1) genes (SEQ-ID NO: 105). Indeed, the MCP-LaG16/(ANR)₈-NSP3-based sensor is highly selective for the fusion gene configuration, as overexpression of native EGFP (SEQ-ID NO: 82) and NS3a (H1) proteins (SEQ-ID NO: 105) failed to activate the system (FIG. 40B). Specifically, HEK-293 cells were co-transfected with the EGFP-NS3a (H1) sensor (SEQ-ID NOs: 52, 5&137) and expression vectors for either EGFP (SEQ-ID NO: 82), NS3a (H1) (SEQ-ID NO: 105) or EGFP-NS3a (H1) (SEQ-ID NO: 17). SEAP expression in the culture supernatant were profiled at 48 h after transfection. Furthermore, both fluorescence imaging (FIG. 41A) and flow-cytometric analysis (FIG. 41B) revealed that the MCP-LaG16/(ANR)₈-NSP3-based fusion protein sensor (SEQ-ID NOs: 52, 5&139: producing mCherry signals by NanoLuc-P2A-mCherry-mRNA containing MCP-specific poly (A)-surrogate) was highly target-specific, showing a strong correlation with EGFP signals produced by overexpressed EGFP-NS3a (H1), and there was no significant basal mCherry expression in EGFP-deficient cells (FIG. 41). Specifically, HEK-293 cells were co-transfected with plasmids encoding mCherry-mRNA containing MCP-specific poly (A)-surrogate (SEQ-ID NO: 139) and constitutive expression vectors for MCP-LaG16 (SEQ-ID NO: 52), (ANR)₈-NSP3 (SEQ-ID NO: 5) and EGFP-NS3a (H1) (SEQ-ID NO: 17). Transfection of pcDNA3.1 (+) (Invitrogen, CA; cat-no. V79020) instead of EGFP-NS3a (H1) expression vectors was used as a negative control. At 48 h post transfection, fluorescent images showing co-localized EGFP/mCherry signals were acquired (scale bar: 50 μm) before flow-cytometric analysis was performed (10,000 cells per group). Next, we used this STIF-based design framework to create a sensor for BCR-ABL, a representative cytosolic biomarker of chronic myelogenous leukemia (CML) (FIG. 40A). Such STIF-based sensor is highly selective for the fusion gene configuration (FIG. 40B); while reporter gene expression was dose-dependently activated by the BCR-ABL oncoprotein, the system remained unresponsive to overexpression of native BCR and ABL genes indicative for healthy cell signatures (FIG. 40C). Furthermore, we showed that overexpression of BCR-ABL (SEQ-ID NO: 301) indeed triggers assembly of the STIF components MCP-ABI (iDab) (SEQ-ID NO: 181: where ABI (iDab) (SEQ-ID NO: 223) is an ABL-specific intrabody) and CCmut3-NSP3 (SEQ-ID NO: 258: where CCmut3 (SEQ-ID NO: 222) is a synthetic BCR-specific coiled-coil domain), providing the basis for activation of STIF-dependent target gene translation (FIG. 40D). For functional assays (FIG. 40C), HEK-293 cells were co-transfected with a SEAP-producing BCR-ABL sensor (SEQ-ID NOs: 181, 258 &137) and expression vectors for either BCR (SEQ-ID NO: 227), ABL (SEQ-ID NO: 226) or BCR-ABL (SEQ-ID NO: 301). To verify BCR-ABL-specific activation of reporter mRNA circularization (FIG. 40D), HEK-293 cells were co-transfected with expression vectors for 3×FLAG-tagged MCP-ABI (iDab)₃ (SEQ-ID NO: 202), 3×HA-tagged CCmut3-NSP3 (SEQ-ID NO: 201) and BCR-ABL (+, SEQ-ID NO: 301) or pcDNA3.1 (+) (−, negative control; Invitrogen, CA; cat-no. V79020) at 48 h before immunoprecipitation. Target proteins in each lysate fraction before (input) and after immunoprecipitation (Flag-IP) were detected with anti-FLAG (Sigma-Aldrich; cat. no. F7425) and anti-HA antibodies (Cell Signaling Technology, Danvers, MA; cat. no. 3724) according to the descriptions in the Methods section above.

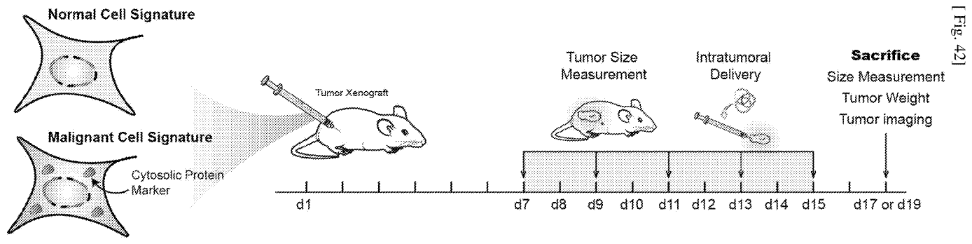

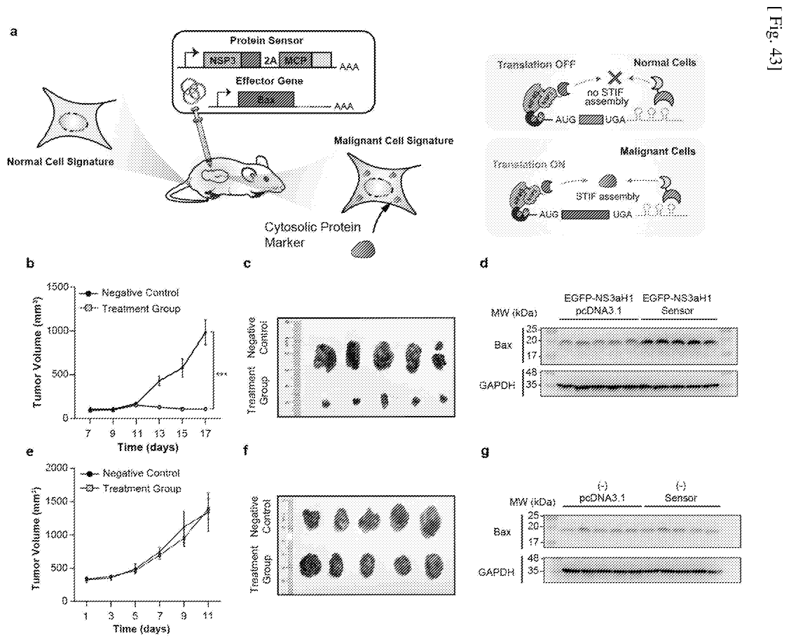

To showcase treatment potential of STIF-based protein sensors in vivo, we used an experimental model of cancer gene therapy. In brief, we created tumor xenografts by stably expressing EGFP-NS3a (H1) (SEQ-ID NO: 17) in epithelial B16-F10 cells, exemplifying a malignant cell signature featuring the presence of a specific target protein in the cytosol (FIG. 42). Such target protein can be representative for a fusion gene product during cancer (FIG. 40A) or for any other intracellular proteinaceous disease marker in general (FIG. 42). Experimental details for cell culture, transfection and stable cell line generation of B16-F10 related cells are described in the Methods section above. Seven days after implantation of B16-F10-derived xenografts, mice received daily intratumoral injections of plasmid mixtures encoding for an MCP-LaG16/(ANR)₈-NSP3-based fusion gene sensor driving translation and in situ production of a pro-apoptotic Bax protein (SEQ-ID NOs: 96&304) (FIG. 43A). Specifically, mice harboring subcutaneous B16-F10EGFP-NS3a (H1)-derived tumors received local injections of pcDNA3.1 (+) (negative control, n=5 mice per group) or plasmid DNA mixes comprising pSL831 (SEQ-ID NO: 145: mBax-mRNA containing MCP-specific poly (A)-surrogate) and constitutive expression vectors for MCP-LaG16 (SEQ-ID NO: 52) and (ANR) g-NSP3 (SEQ-ID NO: 5) (treatment group, n=5 mice per group). Daily changes of tumor size were assessed by calculating V=(length*width) 2/2 (FIG. 43B). Tumors were harvested at the final experimental day for weight analysis (FIG. 43C) and measurement of Bax protein levels by Western Blot using a rabbit anti-Bax antibody (Cell Signaling Technology Cat. no. 14796; Lot. No. 800) (FIG. 43D). Results show specific and self-sufficient elimination of EGFP-NS3a (H1)-transgenic tumors in mice treated with the genetic sensor, whereas mice not receiving the gene therapy (vehicle control) exhibited continuous and rapid tumor growth. Importantly, no significant activation of apoptosis was observed in mice implanted with native B16-F10 cells not expressing EGFP-NS3a (H1), indicating negligible background Bax expression under a potentially “normal” cell signature (FIGS. 43E-43G). Importantly, effective protein levels of Bax detected in tumors correlated with the cell lysis profile (FIGS. 43D, 43G), confirming target-specificity of trigger-inducible Bax expression in vivo. In the future, such fusion protein sensors could therefore form the basis of an “smartened” gene therapy providing self-sufficient detection and correction of pathologic cell conditions.

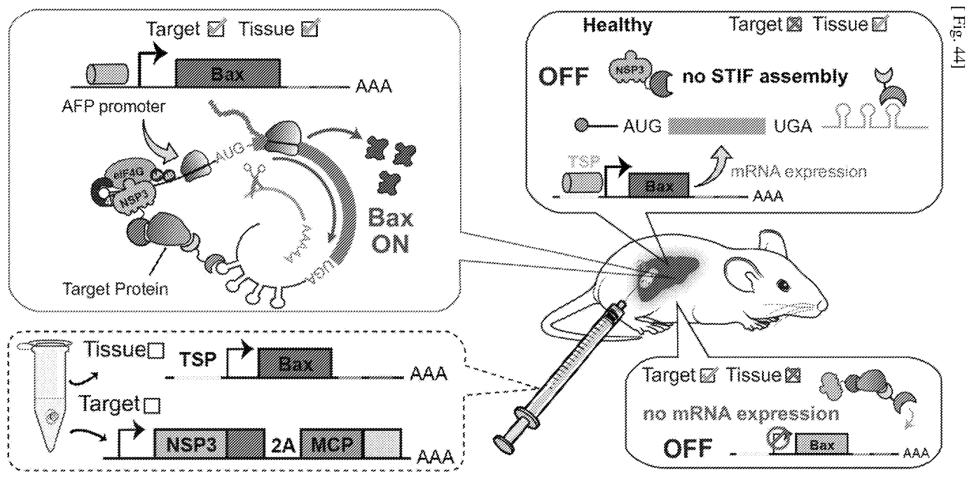

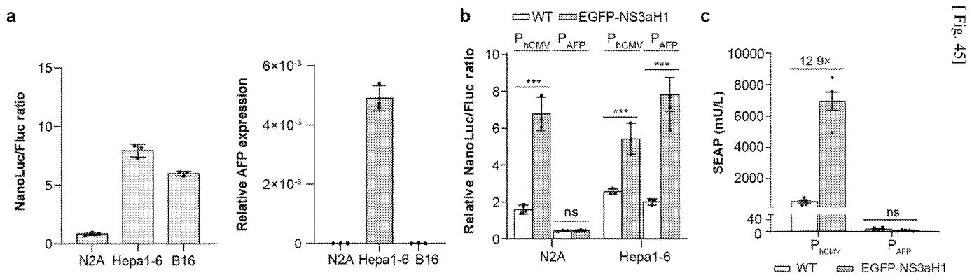

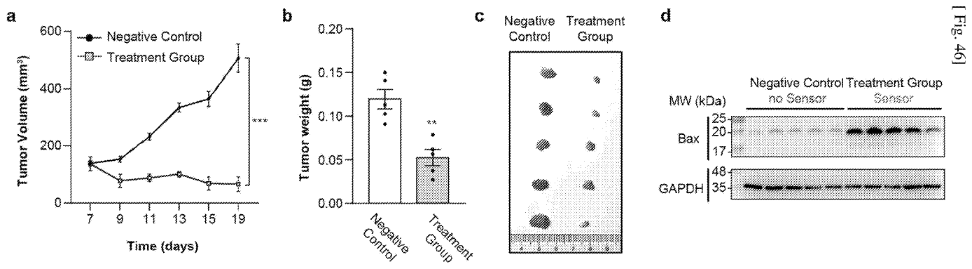

### Example 7: Therapeutic Biocomputer for Combinatorial Disease Profile Detection In Vivo

Whereas fusion proteins (e.g. native BCR-ABL or synthetic EGFP-NS3a (H1); Example 6) are prominent cases where a single intracellular target protein can unambiguously identify pathologic cell states, some diseases however typically lack such unique biomarkers. In these cases, a “true” disease-specific cellular signature must be resolved through a combined detection of various subordinate checkpoint signals. To showcase a scenario requiring such multiplexed cell-state detection in a most simplified format, we created a therapeutic biocomputer controlling strict tissue-specific detection of target proteins (FIG. 44). Although various tissue-specific promoters (TSPs) have been characterized in recent decades with the aim of achieving tissue-specific target gene expression, TSPs alone can hardly meet the requirements of precision targeting in therapeutic settings⁴⁷. For example, using the alpha-fetoprotein (AFP) promoter P_{AFP} as a model TSP (SEQ-ID NO: 129), we could indeed detect non-specific P_{AFP}-driven reporter gene expression in AFP-deficient cell lines (FIG. 45A). Specifically, N2A, Hepa1-6 and B16-F10 cells were co-transfected with a P_{MusAFP}-driven NanoLuc expression vector (SEQ-ID NO: 173) and a constitutive FLuc expression vector (SEQ-ID NO: 305). Experimental details for cell culture, transfection and stable cell line generation of N2A, Hepa1-6 and B16-F10 related cells are described in the Methods section above. At 48 h post transfection, luciferase levels (see analytical assays in Methods section above) and relative AFP expression levels were profiled using murine AFP-specific primers listed in Table S1 and normalized to the gene encoding murine ribosomal protein (Rplp0) (see qRT-PCR in Methods section above). Therefore, we designed a gene circuit running a two-input sensing algorithm that allows suicide gene expression to only occur in cells that harbor an intracellular target protein (e.g., EGFP-NS3a (H1)) and that reside within specific tissues (e.g., P_{AFP}), while tissues not expressing the target protein (i.e., healthy cells) or similar cells from unrelated tissues (e.g., on-target, off-tumor) would remain unaffected (FIG. 44). To test such a distinction logic in vitro, we first transiently transfected expression vectors for MCP-LaG16, (ANR)₈-NSP3 and P_{AFP}-driven reporter mRNA into neuronal Neuro-2A (N2A) and murine hepatoma-derived Hepa1-6 cells engineered for stable EGFP-NS3a (H1) expression. Specifically, native (WT) or stably EGFP-NS3a (H1)-transgenic N2A and Hepa1-6 cells were co-transfected with P_{MusAFP}-driven (SEQ-ID NO: 144) or constitutive expression vectors for NanoLuc-mRNA containing MCP-specific poly (A)-surrogate (SEQ-ID NO: 139) and constitutive expression vectors for MCP-LaG16 (SEQ-ID NO: 52), (ANR)₈-NSP3 (SEQ-ID NO: 5) and FLuc (SEQ-ID NO: 305). Luciferase levels were quantified at 48 h after transfection using a Luciferase Reporter Gene Assay Kit (Yeasen Biotechnology, Shanghai, China; cat. no. 11401ES60) according to experimental details described in the Methods section above. Results show that only Hepa1-6_{EGFP-NS3a (H1)} cells simultaneously fulfilled both criteria (tissue-AND target-specific gene expression) (FIG. 45B). To validate tissue-specific EGFP-NS3a (H1) sensing in mice, plasmids encoding P_{hCMV}-driven (SEQ-ID NOs: 5, 52, &137) and P_{MusAFP}-driven EGFP-NS3a (H1) sensors (SEQ-ID NOs: 5, 52, &146) and EGFP-NS3a (H1) expression vectors (producing SEQ-ID NO: 17) were hydrodynamically injected into the tail vein of C57BL/6 mice before SEAP levels in the bloodstream were measured after 24 h. Mice receiving pcDNA3.1 (+) instead of EGFP-NS3a (H1) expression vectors were used as negative controls. Thus, while a constitutively expressed EGFP-NS3a (H1) sensor showed significant basal activity in EGFP-NS3a (H1)-deficient tissues, aP_{MusAFP}-driven counterpart remained silent in wild-type mice expressing hepatic EGFP-NS3a (H1) due to the inactivity of P_{AFP} in healthy livers (FIG. 45C). Finally, using another xenograft model based on Hepa1-6EGFP-NS3a (H1)-derived tumors, we demonstrated P_{AFP}-driven EGFP-NS3a (H1)-dependent activation of apoptosis in vivo with self-sufficient tumor lysis correlating again with effective Bax expression (FIG. 46). Specifically, mice harboring subcutaneous Hepa1-6EGFP NS3a (H1)-derived tumors received local injections of pcDNA3.1 (+) (negative control, n=5 mice per group) or plasmid DNA mixes comprising pSL886 (SEQ-ID NO: 147: for P_{MusAFP}-driven expression of mBax-mRNA containing MCP-specific poly (A)-surrogate), pSL776 (SEQ-ID NO: 52: for expression of MCP-LaG16) and pSL582 (SEQ-ID NO: 5: for expression of (ANR) g-NSP3) (treatment group, n=5 mice per group). Daily changes of tumor size were assessed by calculating V=(length*width)²/2 (FIG. 46A). Tumors were weighed (FIG. 46B) and harvested for image acquisition (FIG. 46C) followed by measurement of Bax protein levels by Western Blot (FIG. 46D), which illustrates the results at the final experimental day.

In summary, these results indicate the potential of how translation-based protein sensors can play a central role in next-generation therapeutic gene circuits providing programmable, broadly adjustable and self-sufficient gene therapies for treatment of many human diseases in the future. By creating a model target protein EGFP-NS3a (H1) that exemplifies the molecular architecture of aberrant fusion proteins, we showcase the potential of this system for cancer treatments utilizing one or more fusion gene sensors. We also demonstrated its efficacy in a tumor xenograft model involving the kind of complexity and specificity issues that would be relevant in a clinical context. Our results suggest that STIF-enabled protein sensors can in principle be flexibly interconnected with other genetically encoded sensors to eventually achieve any desired custom combination of tissue- and target-specificity in vivo. Evidently, the STIF architecture is not limited to systematic and empirical design of highly specific fusion gene sensors for treatment of hitherto intractable cancers, but is amenable to the detection of any intracellular target signal of interest for which suitable sets of proteinaceous binder moieties (e.g. nanobodies) can be found. Thus, our invention can be used for treating various complex diseases through “designable medicines” created through synthetic biology-inspired bioengineering.

Supplementary Table 1. qRT-PCR Primers Used in this Study

| | |
|---|---|
| EGFP-fw | CCACATGAAGCAGCACGACTT |
| EGFP-rev | GGTGCGCTCCTGGACGTA |
| GAPDH-fw | ACATCGCTCAGACACCATG |
| GAPDH-rev | TGTAGTTGAGGTCAATGAA |
| murine AFP-fw | ATTCCTCCCAGTGCGTGACGGA |
| murine AFP-rev | TGCGTGCCAGCAGACACTGATG |
| Rplp0-fw | GAAACTGCTGCTCACATCCG |
| Rplp0-rev | GCTGGCACAGTGACCTCACACG |
| SEAP-fw | GGCTCTGTCCAAGACATACAA |
| SEAP-rev | GTCGTGTTGCACTGGTTAAAG |
| HusBax-fw | AGCTCTGAGCAGATCATGAAGA |
| HusBax-rev | AGTTGAAGTTGCCGTCAGAAAA |

TABLE S2
Plasmids designed and used in this study

| Plasmid | Description and Architecture | Contains SEQ-ID NOs | Related to Figure(s) |
|---|---|---|---|
| pCK53 | Mammalian cAMP-responsive SEAP expression vector (P_{CRE}-SEAP-pA). (Kemmer et al., 2011) | 172, 239 & 214 | FIG. 24, FIG. 26 & FIG. 33 |
| pDJ55 | Mammalian P_{hEF1α}-driven constitutive L7Ae-eIF4E^{K119A} expression vector (P_{hEF1α}-L7Ae-eIF4E^{K119A}-pA). | 44 & 83 | FIG. 2 & FIG. 5 |
| pLYL47 | Mammalian P_{hCMV}-driven constitutive MCP-VPg expression vector. (P_{hCMV}-MCP-VPg-pA). | 275 & 120 | FIG. 2 & FIG. 5 |
| pLYL62 | Mammalian cAMP-responsive (GNCR)₃-NSP3 expression vector (P_{CRE}-(GNCR)₃-NSP3-pA). | 162, 239 & 214 | FIG. 27 & FIG. 29 |
| pLYL63 | Mammalian VanR-specific (GNCR)₃-NSP3 expression vector (VanO₅-P_{hCMVmin}-(GNCR)₃-NSP3-pA). | 159, 230 & 237 | FIG. 27, FIG. 29, FIG. 30 & FIG. 32 |
| pLYL66 | Mammalian P_{CRE}-driven cAMP-responsive (ANR)₂-NSP3 expression vector (P_{CRE}-(ANR)₂-NSP3-pA). | 267 | |
| pLYL67 | Mammalian cAMP-responsive (ANR)₄-NSP expression vector (P_{CRE}-(ANR)₄-NSP3-pA). | 3, 160, 239 & 214 | FIG. 27, FIG. 29, FIG. 30, FIG. 32 & FIG. 33 |
| pLYL76 | Mammalian P_{hEF1α}-driven constitutive MOR9-1 expression vector (P_{hEF1α}-MOR9-1-pA). | 208 | FIG. 24, FIG. 27, FIG. 29, FIG. 30, FIG. 32 & |
| | | | FIG. 33 |
| pLYL85 | Mammalian VanR-specific (ANR)₄-NSP3 expression vector (VanO₅-Pₘᵢₙ-(ANR)₄-NSP3-pA). | 161, 3, 230 & 231 | FIG. 27 |
| pLYL87 | Mammalian VanR-specific (GNCR)₃-NSP3 expression vector (VanO₅-Pₘᵢₙ-(GNCR)₃-NSP3-pA). | 152 & 231 | FIG. 29 |
| pLYL95 | Mammalian VanR-specific (ANR)₈-NSP3 expression vector (VanO₅-Pₘᵢₙ-(ANR)₈-NSP3-pA). | 151, 230 & 231 | FIG. 29 |
| pLZ16 | Mammalian P_{hCMV}-driven constitutive PABP-L7Ae expression vector (P_{hCMV}-PABP-L7Ae-pA). | 65 & 108 | FIG. 2 & FIG. 5 |
| pLZ27 | Mammalian P_{hEF1α}-driven constitutive L7Ae-NSP3 expression vector (P_{hEF1α}-L7Ae-NSP3-pA). | 43 & 106 | FIG. 2 & FIG. 5 |
| pLZ42 | Mammalian P_{hEF1α}-driven constitutive FRB-NSP3 expression vector (P_{hEF1α}-FRB-NSP3-pA). | 26 & 87 | FIG. 10 |
| pLZ55 | Mammalian P_{hCMV}-driven constitutive L7Ae-CIB1 expression vector (P_{hCMV}-L7Ae-CIB1-pA). | 32 & 76 | FIG. 10 |
| pLZ68 | Mammalian P_{hCMV}-driven constitutive Cry2-NSP3 expression vector (P_{hCMV}-Cry2-NSP3-pA). | 10 & 78 | FIG. 10 |
| pLZ71 | Mammalian P_{hCMV}-driven constitutive (DNCR)₂-NSP3 expression vector (P_{hCMV}-(DNCR)₂-NSP3-pA). | 271 & 79 | |
| pLZ72 | Mammalian P_{hCMV}-driven constitutive (DNCR)₃-NSP3 expression vector (P_{hCMV}-(DNCR)₃-NSP3-pA). | 11 & 79 | FIG. 10 |
| pLZ73 | Mammalian P_{hCMV}-driven constitutive (GNCR)₂-NSP3 expression vector (P_{hCMV}-(GNCR)₂-NSP3-pA). | 29 & 90 | FIG. 14 |
| pLZ74 | Mammalian P_{hCMV}-driven constitutive (GNCR)₃-NSP3 expression vector (P_{hCMV}-(GNCR)₃-NSP3-pA). | 30 | FIG. 10, FIG. 14, FIG. 15, FIG. 16, FIG. 19, |
| | | | FIG. 20, FIG. 22, FIG. 23 & FIG. 29 |
| pLZ75 | Mammalian P_{hCMV}-driven constitutive L7Ae-(NS3a)₂ expression vector (P_{hCMV}-L7Ae-(NS3a)₂-pA). | 40, 92 & 104 | FIG. 14 |
| pLZ76 | Mammalian P_{hCMV}-driven constitutive L7Ae-(NS3a)₃ expression vector (P_{hCMV}-L7Ae-(NS3a)₃-pA). | 41, 92 & 104 | FIG. 10, FIG. 14, FIG. 15 & FIG. 19 |
| pLZ79 | Mammalian TetR-specific expression vector for shRNA-216-repressible SEAP-mRNA containing an | 168, 217 & 238 | FIG. 16 |
| | L7Ae-specific poly(A)-surrogate (P_{hCMV*-1}-SEAP-(C/Dbox)₂₄-BS (shRNA-216)₂-pA). | | |
| PLZ85 | Mammalian P_{hCMV}-driven constitutive (GNCR)₃-VP64 expression vector (P_{hCMV}-(GNCR)₃-VP64-pA). | 300 | FIG. 16 |
| PLZ88 | Mammalian P_{hCMV}-driven constitutive TetR-NS3a expression vector (P_{hCMV}-TetR-NS3a-pA). | 299, 104 & 117 | FIG. 16 |
| pLZ248 | Mammalian P_{hCMV}-driven constitutive L7Ae-VPg expression vector (P_{hCMV}-L7Ae-VPg-pA). | 281 & 120 | FIG. 2 & FIG. 5 |
| PLZ267 | Mammalian P_{SV40}-driven constitutive GEMS_{4xANR}(IL6RB) expression vector (P_{SV40}-GEMS_{4xANR}(IL6RB)-pA). | 187, 182 & 184 | FIG. 25 |
| pLZ268 | Mammalian P_{SV40}-driven constitutive GEMS_{ANR} expression vector (P_{SV40}-GEMS_{ANR}-PA). | 188, 182 & 184 | FIG. 26 |
| pLZ269 | Mammalian P_{SV40}-driven constitutive GEMS_{4xANR}(FGFR1) expression vector (P_{SV40}-GEMS_{4xANR}(FGFR1)-pA). | 189, 183 & 186 | FIG. 25 |
| pLZ271 | Mammalian P_{SV40}-driven constitutive GEMS_{4xANR}(VEGFR2) expression vector (P_{SV40}-GEMS_{4xANR}(VEGFR2)-pA). | 190, 183 & 185 | FIG. 25 |
| pLZ276 | Lentivirus transfer plasmid containing a constitutive P_{hEF1α}-driven expression unit for MOR9-1 and | 215, 236, 208, 229 | |
| | PuroR (LTR-P_{hEF1α}-MOR9-1-IRES-PuroR-LTR). | & 216 | |
| pLZ284 | Mammalian STAT3-specific SEAP expression vector ((O_{STAT3})₈-P_{hCMVmin}-SEAP-pA). | 169, 209 & 237 | FIG. 25 & FIG. 27 |
| PLZ285 | Mammalian cAMP-responsive GEMS_{GNCR} expression vector ((CRE)₄-Pₘᵢₙ-GEMS_{GNCR}-PA). | 158,213 & 231 | FIG. 27 & FIG. 30 |
| pLZ286 | Mammalian cAMP-responsive GEMS_{NS3aH1} expression vector ((CRE)₄-Pₘᵢₙ-GEMS_{NS3aH1}-pA). | 157. 317 & 231 | FIG. 27 & FIG. 30 |
| pLZ287 | Mammalian STAT3-specific mCherry expression vector ((O_{STAT3})₈-P_{hCMVmin}-mCherry-pA). | 150, 209 & 237 | FIG. 30 |
| pLZ310 | Mammalian cAMP-responsive GEMS_{ANR} expression vector ((CRE)₄-Pₘᵢₙ-GEMS_{ANR}-PA). | 154, 209, 317 & 231 | FIG. 27 & FIG. 30 |
| pLZ311 | Mammalian P_{hCMV}-driven constitutive DocS-VPg expression vector (P_{hCMV}-DocS-VPg-pA). | 254 & 120 | FIG. 9 |
| pLZ312 | Mammalian P_{hCMV}-driven constitutive DocS-eIF4E^{K119A} expression vector (P_{hCMV}-DocS-cIF4E^{K119A}-pA). | 257 & 83 | FIG. 9 |
| PLZ323 | Mammalian P_{SV40}-driven SEAP expression vector containing a MALAT1 and HHR motif in the 3′- | 308 | |
| | UTR (P_{SV40}-SEAP-MALAT1-HHR-pA). | | |
| pLZ324 | Mammalian P_{hCMV}-driven SEAP expression vector containing a MALAT1 motif in the 3′-UTR (P_{hCMV}-SEAP-MALAT1-pA). | 308 | |
| PLZ345 | Mammalian VanR-specific NanoLuc expression vector (VanO)₅-P_{hCMVmin}-NanoLuc-pA). | 170, 230 & 237 | FIG. 24 |
| PLZ368 | Mammalian STAT3-specific NanoLuc expression vector ((O_{STAT3})₈-P_{hCMVmin}-NanoLuc-pA). | 164, 101, 209 & 237 | FIG. 26 |
| pLZ411 | Mammalian VanR-specific GEMS_{GNCR} expression vector (VanO₅-P_{hCMVmin}-GEMS_{GNCR} -pA). | 313, 230 & 237 | FIG. 27 |
| pLZ412 | Mammalian VanR-specific GEMS_{NS3aH1} expression vector (VanO₅-P_{hCMVmin}-GEMS_{NS3aH1}-pA). | 156, 230 & 237 | FIG. 27 |
| PLZ413 | Mammalian VanR-specific GEMS_{ANR} expression vector (VanO₅-P_{hCMVmin}-GEMS_{ANR}-pA). | 155, 230 & 237 | FIG. 27 |
| pLZ417 | Mammalian P_{hCMV}-driven constitutive NLS-PcaV-StaPL-KRAB expression vector (P_{hCMV}-NLS-PcaV-StaPL-KRAB-pA). | 197, 16, 109 & 116 | FIG. 25 & FIG. 26 |
| PLZ418 | Mammalian P_{hCMV}-driven constitutive NLS-PeaV-StaPL-VP64 expression vector (P_{hCMV}-NLS-PcaV-StaPL-VP64-pA). | 198, 109 & 116 | FIG. 25 & FIG. 26 |
| PLZ419 | PcaV-repressible SEAP expression vector ((O_{PcaV})₅-P_{SV40}-SEAP-pA). | 166. 225 & 237 | FIG. 25 & FIG. 26 |
| pMF111 | Mammalian TetR-specific SEAP expression vector (P_{hCMV*-1}-SEAP-pA). | 168, 217 & 238 | FIG. 25 & FIG. 38 |
| pMX57 | Mammalian calcium-responsive P_{NFAT3}-driven SEAP expression vector (P_{NFAT3}-SEAP-pA). (Xie et al., 2016) | 167, 210, 218 & 231 | FIG. 25 |
| pMX116 | Mammalian P_{SV40}-driven SEAP expression vector containing λN-repressible HHR in the 3′-UTR (P_{SV40}-SEAP-HHR-pA). | 114 & 124 | FIG. 8 |
| pMX331 | Mammalian P_{hEF1α}-driven constitutive FKBP-L7Ae expression vector (P_{hEF1α}-FKBP-L7Ae-pA). | 25 & 86 | FIG. 10 |
| pPW1 | Mammalian P_{hCMV}-driven constitutive GID1-L7Ae expression vector (P_{hCMV}-GID1-L7Ae-pA). | 286 & 89 | |
| pPW2 | Mammalian P_{hCMV}-driven constitutive GAI-NSP3 expression vector (P_{hCMV}-GAI-NSP3-pA). | 289 & 88 | FIG. 10 |
| pPW3 | Mammalian P_{hCMV} driven constitutive L7Ae-(ABI)₃ expression vector (P_{hCMV}-L7Ae-(ABI)₃- pA). | 31 & 71 | FIG. 10 |
| pPW4 | Mammalian P_{hCMV}-driven constitutive NSP3-(PYL1)₃ expression vector (P_{hCMV}-NSP3-(PYL1)₃-pA). | 64 & 111 | FIG. 10 |
| pPW10 | Mammalian P_{hCMV}-driven constitutive ABI-NSP3 expression vector (P_{hCMV}-ABI-NSP3-pA). | 266 & 71 | |
| pPW11 | Mammalian P_{hCMV}-driven constitutive NSP3-ABI expression vector (P_{hCMV}-NSP3-ABI-pA). | 294 & 71 | |
| pPW12 | Mammalian P_{hCMV}-driven constitutive PYL1-L7Ae expression vector (P_{hCMV}-PYL1-L7Ae-pA). | 279 & 111 | |
| pPW13 | Mammalian P_{hCMV}-driven constitutive L7Ae-GAI expression vector (P_{hCMV}-L7Ae-GAI-pA). | 283 & 88 | |
| pPW14 | Mammalian P_{hCMV}-driven constitutive GAI-L7Ae expression vector (P_{hCMV}-GAI-L7Ae-pA). | 27 & 88 | FIG. 10 |
| pPW15 | Mammalian P_{hCMV}-driven constitutive L7Ae-GID1 expression vector (P_{hCMV}-L7Ae-GID1-pA). | 282 & 89 | |
| pPW16 | Mammalian P_{hCMV}-driven constitutive GID1-NSP3 expression vector (P_{hCMV}-GID1-NSP3-pA). | 287 & 89 | |
| pPW17 | Mammalian P_{hCMV}-driven constitutive NSP3-GID1 expression vector (P_{hCMV}-NSP3-GID1-pA). | 63 & 89 | FIG. 10 |
| pPW18 | Mammalian P_{hCMV}-driven constitutive NSP3-GAI expression vector (P_{hCMV}-NSP3-GAI-pA). | 293 & 88 | |
| pPW19 | Mammalian P_{hCMV}-driven constitutive (GAI)₃-L7Ae expression vector (P_{hCMV}-(GAI)₃-L7Ae-pA). | 285 & 88 | |
| pPW20 | Mammalian P_{hCMV}-driven constitutive NSP3-(GID1)₃ expression vector (P_{hCMV}-NSP3-(GID1)₃-pA). | 292 & 89 | |
| pPW21 | Mammalian P_{hCMV}-driven expression vector for reporter mRNA containing 24 tandem C/D-box | 127, 175 & 123 | FIG. 9 |
| | repeats placed downstream of SEAP- and upstream of NanoLuc-coding regions (P_{hCMV}-SEAP-(C/Dbox)₂₄-NanoLuc-pA). | | |
| pPW22 | Mammalian P_{hCMV}-driven constitutive ABI-MCP expression vector (P_{hCMV}-ABI-MCP-pA). | 259 & 71 | FIG. 10 |
| pPW23 | Mammalian P_{hCMV}-driven constitutive MCP-GID1 expression vector (P_{hCMV}-MCP-GID1-pA). | 242 & 89 | FIG. 10 |
| pPW24 | Mammalian P_{hCMV}-driven constitutive (PYL1)₃- NSP3 expression vector (P_{hCMV}-(PYL1)₃-NSP3-pA). | 67 & 111 | |
| pQZ8 | Mammalian P_{hCMV}-driven expression vector for SEAP mRNA containing 4 tandem C/D-box repeats | 127, 176 & 123 | FIG. 9 |
| | in the 5′-UTR (P_{hCMV}-(C/Dbox)₄-SEAP-pA). | | |
| pQZ111 | Mammalian P_{hCMV}-driven expression vector for NanoLuc-mRNA containing MCP-specific binding | 163, 101, 124 & 125 | FIG. 26 |
| | sites in the 3′-UTR (P_{hCMV}-NanoLuc-(MS2-box)₂₄-HHR-pA). | | |
| pQZ112 | Mammalian P_{hCMV}-driven expression vector for EGFP-mRNA containing MCP-specific binding sites | 149, 124, 125 | FIG. 27, FIG. 29, FIG. 30 & FIG. 32 |
| | in the 3′-UTR (P_{hCMV}-EGFP-(MS2-box)₂₄-HHR-pA). | | |
| pSL4 | Mammalian P_{hU6}-driven shRNA-216 expression vector (P_{hU6}-shRNA-216). | 128, 126 & 235 | FIG. 2, FIG. 3, FIG. 4, FIG. 11, FIG. 14, FIG. |
| | | | 15, FIG. 19, FIG. 35, FIG. 36 & FIG. 37 |
| pSL31 | Mammalian P_{hCMV}-driven expression vector for shRNA-216-repressible L7Ae-specific SEAP-mRNA | 131, 114, 122 & 123 | FIG. 2, FIG. 3 & FIG. 4 |
| | (P_{hCMV}-SEAP-(C/Dbox)₈-BS(shRNA-216)₂-pA). | | |
| pSL44 | Mammalian P_{hCMV}-driven constitutive Coh2-L7Ae expression vector (P_{hCMV}-Coh2-L7Ae-pA). | 8 & 77 | FIG. 2, FIG. 3 & FIG. 5 |
| pSL47 | Mammalian P_{hCMV}-driven constitutive PABP-DocS expression vector (P_{hCMV}-PABP-DocS-pA). | 207, 80 & 108 | FIG. 9 |
| pSL65 | Mammalian P_{hCMV}-driven constitutive L7Ae-Coh2 expression vector (P_{hCMV}-L7Ae-Coh2-pA). | 33 & 77 | FIG. 8 & FIG. 35 |
| pSL66 | Mammalian P_{hCMV}-driven constitutive DocS-NSP3 expression vector (P_{hCMV}-DocS-NSP3-pA). | 12 & 80 | FIG. 8, FIG. 9, FIG. 35 & FIG. 36 |
| pSL71 | Mammalian P_{hCMV}-driven constitutive Cry2-NSP3 expression vector (P_{hCMV}-Cry2-NSP3-pA). | 10 & 78 | FIG. 10 |
| pSL80 | Mammalian P_{hCMV}-driven expression vector for shRNA-216-repressible L7Ae-specific SEAP-mRNA | 132, 114, 122 & 123 | FIG. 2 |
| | (P_{hCMV}-SEAP-(C/Dbox)₁₆-BS (shRNA-216)₂-pA). | | |
| pSL81 | Mammalian P_{hCMV}-driven expression vector for shRNA-216-repressible L7Ae-specific SEAP-mRNA | 133, 114, 122 & 123 | FIG. 2 |
| | (P_{hCMV}-SEAP-(C/Dbox)₁₂-BS(shRNA-216)₂-pA). | | |
| pSL82 | Mammalian P_{hCMV}-driven constitutive L7Ae-(Coh2)₂ expression vector (P_{hCMV}-L7Ae-(Coh2)₂-pA). | 34 | FIG. 35 |
| pSL83 | Mammalian P_{hCMV}-driven constitutive L7Ae-(Coh2)₃ expression vector (P_{hCMV}-L7Ae-(Coh2)₃-pA). | 35 | FIG. 9, FIG. 35 & FIG. 36 |
| pSL85 | Mammalian P_{hCMV}-driven constitutive (DocS)₂-NSP3 expression vector (P_{hCMV}-(DocS)₂-NSP3-pA). | 13 | FIG. 35 & FIG. 36 |
| pSL86 | Mammalian P_{hCMV}-driven constitutive (DocS)₃-NSP3 expression vector (P_{hCMV}-(DocS)₃-NSP3-pA). | 14 | FIG. 8, FIG. 35 & FIG. 36 |
| pSL87 | Mammalian P_{hCMV}-driven constitutive DocS-eIF4G expression vector (P_{hCMV}-DocS-eIF4G-pA). | 256. 84 & 230 | FIG. 9 |
| pSL88 | Mammalian P_{hCMV}-driven expression vector for shRNA-216-repressible L7Ae-specific SEAP-mRNA | 134, 114, 122 & 123 | FIG. 2, FIG. 3, FIG. 14, |
| | (P_{hCMV}-SEAP-(C/Dbox)₂₄-BS(shRNA-216)₂-pA). | | FIG. 15, FIG. 19, FIG. 35, FIG. 36 & FIG. 37 |
| PSL95 | Mammalian P_{hCMV}-driven constitutive MCP-NSP3 expression vector (P_{hCMV}-MCP-NSP3-pA). | 59, 98 & 106 | FIG. 2 & FIG. 5 |
| pSL133 | Mammalian P_{hCMV}-driven constitutive scFv162-NSP3 expression vector (P_{hCMV}-ScFv162-NSP3-pA). | 70 & 112 | |
| pSL136 | Mammalian P_{hCMV}-driven constitutive L7Ae-scFv35 expression vector (P_{hCMV}-L7Ae-scFv35-pA). | 47 & 113 | FIG. 37 |
| pSL138 | Mammalian P_{hCMV}-driven constitutive (scFv162)₂-NSP3 expression vector (P_{hCMV}-(scFv162)₂-NSP3-pA). | 261 & 112 | |
| pSL139 | Mammalian P_{hCMV}-driven constitutive (scFv162)₃-NSP3 expression vector (P_{hCMV}-(scFv162)₃-NSP3-pA). | 261 & 112 | FIG. 37 |
| pSL154 | Mammalian P_{hCMV}-driven constitutive cIF4G-MCP expression vector (P_{hCMV}-CIF4G-MCP-pA). | 253 & 84 | FIG. 2 & FIG. 5 |
| pSL159 | Mammalian P_{hCMV}-driven constitutive TetR-VP64 expression vector (P_{hCMV}-TetR-VP64-pA). | 297, 117 & 119 | |
| PSL168 | Mammalian P_{hCMV}-driven constitutive L7Ae-pE59 expression vector (P_{hCMV}-L7Ae-pE59-pA). | 280 & 110 | |
| PSL169 | Mammalian P_{hCMV}-driven constitutive L7Ae-(pE59)₂ expression vector (P_{hCMV}-L7Ae-(pE59)₂-pA). | 46 & 110 | FIG. 11 |
| pSL171 | Mammalian P_{hCMV}-driven constitutive ERK2-NSP3 expression vector (P_{hCMV}-ERK2-NSP3-pA). | 277 & 85 | |
| pSL172 | Mammalian PcaV-specific SEAP expression vector ((O_{PcaV})₅-P_{hCMVmin}-SEAP-pA). | 165, 225 & 237 | FIG. 25 & FIG. 26 |
| pSL173 | Mammalian VanR-specific SEAP expression vector (VanO₅-P_{hCMVmin}-SEAP-pA). | 171, 230 & 237 | FIG. 24 & FIG. 33 |
| pSL175 | Mammalian P_{hCMV}-driven constitutive VanR-VP64 expression vector (P_{hCMV}-VanR-VP64-pA). | 296, 119 & 219 | FIG. 24, FIG. 27, FIG. 29, FIG. 30, FIG. 32 & |
| | | | FIG. 33 |
| pSL176 | Mammalian P_{hCMV}-driven constitutive PeaV-VP64 expression vector (P_{hCMV}-PeaV-VP64-pA). | 298 & 109 | |
| pSL189 | Mammalian P_{hCMV}-driven constitutive (ERK2)₂-NSP3 expression vector (P_{hCMV}-(ERK2)₂-NSP3-pA). | 24, 85 & 88 | FIG. 11 |
| pSL241 | Mammalian P_{hCMV}-driven constitutive Coh2-NSP3 expression vector (P_{hCMV}-Coh2-NSP3-pA). | 268 | FIG. 35 |
| pSL242 | Mammalian P_{hCMV}-driven constitutive (Coh2)₂-NSP3 expression vector (P_{hCMV}-(Coh2)₂-NSP3-pA). | 269 | FIG. 35 |
| pSL243 | Mammalian P_{hCMV}-driven constitutive (Coh2)₃-NSP3 expression vector (P_{hCMV}-(Coh2)₃-NSP3-pA). | 9 | FIG. 35 & FIG. 37 |
| pSL244 | Mammalian P_{hCMV}-driven constitutive (DocS)₃ expression vector (P_{hCMV}-(DocS)₃-pA). | 302 | FIG. 37 |
| pSL274 | Mammalian dual reporter vector containing a constitutive FLuc expression unit and a constitutive | 135, 101, 122 & 123 | FIG. 11 |
| | expression unit for shRNA-216-repressible NanoLuc-mRNA with an L7Ae-specific poly(A)- | | |
| | surrogate (P_{hCMV}-NanoLuc-(C/Dbox)₂₄-BS(shRNA-216)₂-pA::P_{hEF1α}-FLuc-pA). | | |
| pSL334 | Mammalian P_{hCMV}-driven constitutive λN-Coh2 expression vector (P_{hCMV}-λN-Coh2-pA). | 205 & 100 | FIG. 8 |
| pSL355 | Mammalian P_{hCMV}-driven expression vector for SEAP-mRNA containing an L7Ae-specific poly(A)- | 136, 114, 123 & 124 | FIG. 3, FIG. 5, FIG. 8, FIG. 9, FIG. 10 & FIG. 19 |
| | surrogate (P_{hCMV}-SEAP-(C/Dbox)₂₄-HHR-pA). | | |
| pSL435 | Mammalian P_{hCMV}-driven constitutive MCP-EGFP expression vector (P_{hCMV}-MCP-EGFP-pA). | 245 | FIG. 9 |
| pSL446 | AAV vector for P_{hCMV}-driven constitutive (GNCR)₃-NSP3 expression (P_{hCMV}-(GNCR)₃-NSP3-pA). | 240 & 30 | |
| pSL468 | Mammalian P_{hCMV}-driven expression vector for SEAP-mRNA containing an MCP-specific poly(A)- | 137, 114, 124 & 125 | FIG. 2, FIG. 3, FIG. 8, FIG. 9, FIG. 10, FIG. 15, |
| | surrogate (P_{hCMV}-SEAP-(MS2-box)₂₄-HHR-pA). | | FIG. 16, FIG. 18, FIG. 19, FIG. 22, FIG. 23, FIG. |
| | | | 26, FIG. 33, FIG. 35, FIG. 36, FIG. 37, FIG. 38, |
| | | | FIG. 39, FIG. 40 & FIG. 45 |
| pSL475 | Mammalian P_{hCMV}-driven constitutive L7Ae-(NS3a)₃-3xFLAG expression vector (P_{hCMV}-L7Ae-(NS3a)₃-3xFLAG-pA). | 48 | FIG. 17 |
| pSL476 | Mammalian P_{hCMV}-driven constitutive 3xHA-(GNCR)₃-NSP3 expression vector (P_{hCMV}-3xHA-(GNCR)₃-NSP3-pA). | 1, 127 & 224 | FIG. 17 |
| pSL479 | | 274 & 224 | |
| pSL497 | Mammalian P_{hCMV}-driven constitutive MCP-NS3a expression vector (P_{hCMV}-MCP-NS3a-pA). | 54, 98 & 104 | FIG. 10 & FIG. 22 |
| pSL500 | Mammalian P_{hCMV}-driven constitutive MCP-(NS3a)₂ expression vector (P_{hCMV}-MCP-(NS3a)₂-pA). | 290 & 104 | |
| pSL503 | Mammalian P_{hCMV}-driven constitutive MCP-(NS3a)₃ expression vector (P_{hCMV}-MCP-(NS3a)₃-pA). | | FIG. 16 |
| pSL511 | Mammalian P_{hCMV}-driven constitutive expression vector for SEAP-mRNA containing an MCP- | 127, 137, 124 & 125 | |
| | specific poly(A)-surrogate (P_{hCMV}-SEAP-(MS2-box)₂₄-HHR-pA). | | |
| pSL512 | AAV vector for P_{hCMV}-driven constitutive MCP-(NS3a)₃ expression (P_{hCMV}-MCP-(NS3a)₃-pA). | 127 & 55 | |
| pSL515 | Mammalian P_{hCMV}-driven expression vector for SEAP-mRNA containing MCP-specific poly(A)- | 179, 127, 124 & 125 | FIG. 2 & FIG. 3 |
| | surrogate with 8 tandem MS2-box repeats (P_{hCMV}-SEAP-(MS2-box)₈-HHR-pA). | | |
| pSL516 | Mammalian P_{hCMV}-driven expression vector for SEAP-mRNA containing an MCP-specific poly(A)- | 138, 114, 124 & 125 | FIG. 2 & FIG. 10 |
| | surrogate (P_{hCMV}-SEAP-(MS2-box)₁₆-HHR-pA). | | |
| pSL517 | Mammalian P_{hCMV}-driven constitutive SEAP expression vector (P_{hCMV}-SEAP-pA). | | FIG. 3 & FIG. 4 |
| pSL546 | Mammalian P_{hCMV}-driven constitutive MCP-NS3a(H1) expression vector (P_{hCMV}-MCP-NS3a(H1)-pA). | 56 & 105 | FIG. 22 & FIG. 23 |
| pSL547 | Mammalian P_{hCMV}-driven constitutive MCP-(NS3a(H1))₂ expression vector (P_{hCMV}-MCP-(NS3a(H1))₂-pA). | 57 & 105 | FIG. 23 |
| pSL548 | Mammalian P_{hCMV}-driven constitutive MCP-(NS3a(H1))₃ expression vector (P_{hCMV}-MCP-(NS3a(H1))₃-pA). | 58, 98 & 105 | FIG. 19, FIG. 20, FIG. 23, FIG. 26, FIG. 27, |
| | | | FIG. 29, FIG. 30, FIG. 32, FIG. 33 & FIG. 39 |
| pSL549 | Mammalian P_{hCMV}-driven constitutive (ANR)₄-NSP3 expression vector (P_{hCMV}-(ANR)₄-NSP3-pA). | 3 | FIG. 8, FIG. 22, FIG. 23, FIG. 33 & FIG. 39 |
| pSL580 | Mammalian cAMP-specific (ANR)₈-NSP3 expression vector (P_{CRE}-(ANR)₈-NSP3-pA). | 153, 239 & 214 | FIG. 29 |
| pSL581 | Mammalian P_{hCMV}-driven constitutive (ANR)₆-NSP3 expression vector (P_{hCMV}-(ANR)₆-NSP3-pA). | 4 | FIG. 39 |
| pSL582 | Mammalian P_{hCMV}-driven constitutive (ANR)₈-NSP3 expression vector (P_{hCMV}-(ANR)₈-NSP3-pA). | 5 | FIG. 26, FIG. 29, FIG. 38, FIG. 39, FIG. 40, |
| | | | FIG. 41, FIG. 43, FIG. 45 & FIG. 46 |
| pSL615 | Mammalian P_{hCMV}-driven constitutive Bcl-XL-NSP3 expression vector (P_{hCMV}-Bcl-XL-NSP3-pA). | 7 & 74 | FIG. 8 |
| pSL637 | Mammalian P_{hCMV}-driven constitutive MCP-(pE59)₂ expression vector (P_{hCMV}-MCP-(pE59)₂-pA). | 249 & 110 | FIG. 11 |
| pSL661 | Mammalian P_{hCMV}-driven constitutive L7Ae-LD3 expression vector (P_{hCMV}-L7Ae-LD3-pA). | 38 & 95 | FIG. 8 |
| pSL667 | Mammalian P_{hCMV}-driven constitutive L7Ae-LD1 expression vector (P_{hCMV}-L7Ae-LD1-pA). | 37 & 94 | FIG. 8 |
| pSL674 | Mammalian P_{hCMV}-driven constitutive MCP-Coh2 expression vector (P_{hCMV}-MCP-Coh2-pA). | 51 | FIG. 2, FIG. 3, FIG. 5, FIG. 8, FIG. 9 & FIG. 35 |
| pSL683 | Mammalian P_{hCMV}-driven constitutive expression vector for NanoLuc-P2A-mCherry-mRNA | 139, 97, 101, 124 & 125 | FIG. 11, FIG. 33, FIG. 41 & FIG. 45 |
| | containing an MCP-specific poly(A)-surrogate (P_{hCMV}-NanoLuc-P2A-mCherry-(MS2-box)₂₄-HHR-pA). | | |
| pSL684 | Mammalian PcaV-specific mCherry expression vector ((O_{PcaV})₅-P_{hCMVmin}-mCherry-pA). | 141, 97, 225 & 237 | FIG. 32 |
| pSL685 | Mammalian P_{hCMV}-driven constitutive expression vector for NanoLuc-P2A-mINS-mRNA containing | 140, 99, 101, 124 & 125 | FIG. 20 |
| | an MCP-specific poly(A)-surrogate (P_{hCMV}-NanoLuc-P2A-mINS-(MS2-box)₂₄-HHR-pA). | | |
| pSL688 | SB100X-specific transposon containing a constitutive expression unit for NanoLuc-P2A-mINS- | 127, 140, 233, 307, | |
| | mRNA with an MCP-specific poly(A)-surrogate and a constitutive expression unit for PuroR (ITR- | 99, 101, 124 & 125 | |
| | P_{RPBSA}-PuroR-pA:P_{hCMV}-NanoLuc-P2A-mINS-(MS2-box)₂₄-HHR-pA-ITR). | | |
| pSL703 | Mammalian P_{hCMV}-driven constitutive L7Ae-NS3a(H1) expression vector (P_{hCMV}-L7Ae-NS3a(H1)-pA). | 42 & 105 | FIG. 8 |
| pSL704 | Mammalian P_{hCMV}-driven constitutive ANR-NSP3 expression vector (P_{hCMV}-ANR-NSP3-pA). | 2 & 73 | FIG. 39 |
| pSL721 | SB100X-specific transposon containing constitutive expression units of ZeoR and MCP-(NS3a)₃ | 127, 55, 234, 228 & 104 | |
| | (ITR-P_{RPBSA}-ZeoR-PA:P_{hCMV}-MCP-(NS3a)₃-pA-ITR). | | |
| pSL722 | SB100X-specific transposon containing constitutive expression units of PuroR and (GNCR)₃-NSP3 | 127, 30, 234 & 229 | |
| | (ITR-P_{RPBSA}-PuroR-PA:P_{hCMV}-(GNCR)₃-NSP3-pA-ITR) | | |
| pSL754 | Mammalian P_{CRE}-driven cAMP-responsive NLS-PcaV-StaPLd-VP64 expression vector (P_{CRE}-NLS-PcaV-StaPLd-VP64-pA). | 130, 61, 103, 109, | FIG. 32 |
| | | 119, 239 & 214 | |
| pSL762 | Mammalian P_{hCMV}-driven constitutive 3xFLAG-L7Ae-NSP3 expression vector (P_{hCMV}-3xFLAG-L7Ae-NSP3-pA). | 49 & 221 | FIG. 6 |
| pSL763 | Mammalian P_{hCMV}-driven constitutive PABP-L7Ae-3xFLAG expression vector (P_{hCMV}-PABP-L7AC-3xFLAG-pA). | 66 & 221 | FIG. 3 & FIG. 6 |
| pSL767 | Mammalian P_{hCMV}-driven constitutive expression vector for SEAP-mRNA with self-elcavable polyA | 142 & 124 | FIG. 4 |
| | (P_{hCMV}-SEAP-HHR₂-pA). | | |
| pSL768 | Mammalian P_{hCMV}-driven constitutive expression vector for SEAP-mRNA with self-cleavable polyA | 143 & 124 | FIG. 4 |
| | (P_{hCMV}-SEAP-HHR₄-pA). | | |
| pSL775 | AAV vector for P_{hEF1α}-driven constitutive EGFP-NS3a(H1) expression (P_{hEF1α}-EGFP-NS3a(H1)-pA). | 17 | FIG. 38, FIG. 39, FIG. 40, FIG. 41 & FIG. 45 |
| pSL776 | Mammalian P_{hCMV}-driven constitutive MCP-LaG16 expression vector (P_{hCMV}-MCP-LaG16-pA). | 52, 204, 93 & 100 | FIG. 38, FIG. 39, FIG. 40, FIG. 41, FIG. 43, |
| | | | FIG. 45 & FIG. 46 |
| pSL777 | Mammalian P_{hCMV}-driven constitutive MCP-(LaG16)₂ expression vector (P_{hCMV}-MCP-(LaG16)₂-pA). | 53 & 93 | FIG. 39 |
| pSL781 | Mammalian P_{hCMV}-driven expression vector for 3xFLAG-FLuc-mRNA containing MCP-specific | 127, 177, 295, 124, | FIG. 5 |
| | poly(A)-surrogate with 24 tandem MS2-box repeats (P_{hCMV}-3xFLAG-FLuc-(MS2-box)₂₄-HHR-pA). | 125 & 221 | |
| pSL796 | Mammalian P_{hCMV}-driven constitutive SP-EGFP-NS3a(H1) expression vector (P_{hCMV}-SP-EGFP-NS3a(H1)-pA). | 22 & 115 | |
| pSL797 | Mammalian P_{hCMV}-driven constitutive NLS-EGFP-NS3a(H1) expression vector (P_{hCMV}-NLS-EGFP-NS3a(H1)-pA). | 21 | FIG. 38 |
| PSL798 | Mammalian P_{hCMV}-driven constitutive TM-EGFP-NS3a(H1) expression vector (P_{hCMV}-TM-EGFP-NS3a(H1)-pA). | 23 & 118 | FIG. 38 |
| pSL799 | Mammalian P_{hCMV}-driven constitutive EGFP-NS3a(H1)-CAAX expression vector (P_{hCMV}-EGFP-NS3a(H1)-CAAX-pA). | 19 & 75 | FIG. 38 |
| pSL813 | Mammalian P_{MusAFP}-driven expression vector for NanoLuc-P2A-mCherry-mRNA containing an | 144 & 129 | FIG. 45 |
| | MCP-specific poly(A)-surrogate (P_{MusAFP}-NanoLuc-P2A-mCherry-(MS2-box)₂₄-HHR-pA). | | |
| pSL816 | SB100X-specific transposon containing constitutive expression units for EGFP-NS3a(H1), BFP and | 236, 17, 234, 306, | |
| | PuroR (ITR-P_{hEF1α}-EGFP-NS3a(H1)-pA:P_{RPBSA}-BFP-P2A-PuroR-pA-ITR). | 107, 229 & 105 | |
| pSL818 | Mammalian P_{hCMV}-driven constitutive NS3a(H1) expression vector (P_{hCMV}-NS3a(H1)-pA). | 105 | FIG. 40 |
| pSL824 | Mammalian P_{hCMV}-driven constitutive NES-EGFP-NS3a(H1) expression vector (P_{hCMV}-NES-EGFP-NS3a(H1)-pA). | 20 & 102 | FIG. 38 |
| pSL831 | Mammalian P_{hCMV}-driven expression vector for mBax-mRNA containing MCP-specific poly(A)- | 145, 96, 124 & 125 | FIG. 43 |
| | surrogate with 24 tandem MS2-box repeats (P_{hCMV}-mBax-(MS2-box)₂₄-HHR-pA). | | |
| pSL832 | Mammalian P_{hCMV}-driven expression vector for hBax-mRNA containing MCP-specific poly(A)- | 304 | |
| | surrogate with 24 tandem MS2-box repeats (P_{hCMV}-hBax-(MS2-box)₂₄-HHR-pA). | | |
| pSL834 | Mammalian P_{hCMV}-driven constitutive TetR-LaG16 expression vector (P_{hCMV}-TetR-LaG16-pA). | 68, 93 & 117 | FIG. 38 |
| pSL836 | Mammalian P_{hCMV}-driven constitutive (ANR)₈-VP64 expression vector (P_{hCMV}-(ANR)₈-VP64-pA). | 6 | FIG. 38 |
| pSL857 | Mammalian P_{MusAFP}-driven expression vector for SEAP-mRNA containing an MCP-specific poly(A)- | 146 & 129 | FIG. 45 |
| | surrogate (P_{MusAFP}-SEAP-(MS2-box)₂₄-HHR-pA). | | |
| pSL860 | Mammalian P_{hCMV}-driven constitutive MCP-ABI(iDab)₃ expression vector (P_{hCMV}-MCP-ABI(iDab)₃-pA). | 181 & 223 | FIG. 40 |
| pSL863 | Mammalian P_{hCMV}-driven constitutive CCmut3-NSP3 expression vector (P_{hCMV}- CCmut3-NSP3-pA). | 258 & 222 | FIG. 40 |
| pSL875 | Mammalian P_{hCMV}-driven constitutive MCP-LaM8 AK47 expression vector (P_{hCMV}-MCP-LaM8_AK47-pA). | 241 & 220 | FIG. 10 |
| pSL876 | Mammalian P_{hCMV}-driven constitutive mCherry-NSP3 expression vector (P_{hCMV}- mCherry-NSP3-pA). | 250 | FIG. 10 |
| pSL886 | Mammalian P_{MusAFP}-driven expression vector for mBax-mRNA containing an MCP-specific poly(A)- | 147, 96 & 129 | FIG. 46 |
| | surrogate (P_{MusAFP}-mBax-(MS2-box)₂₄-HHR-pA). | | |
| pSL889 | Mammalian P_{SV40}-driven constitutive GEMS_{GNCR} expression vector (P_{SV40}-GEMS_{GNCR}-PA). | 191, 90, 182 & 184 | FIG. 25 & FIG. 26 |
| pSL890 | Mammalian P_{SV40}-driven constitutive GEMS_{NS3aH1} expression vector (P_{SV40}-GEMS_{NS3aH1}-PA). | 192, 105, 248, 182 | FIG. 25 & FIG. 26 |
| | | & 184 | |
| pSL891 | Mammalian P_{SV40}-driven constitutive GEMS_{GNCR}(FGFR1) expression vector (P_{SV40}-GEMS_{GNCR}(FGFR1)-pA). | 193, 183 & 186 | FIG. 25 |
| pSL892 | Mammalian P_{SV40}-driven constitutive GEMS_{NS3aH1}(FGFR1) expression vector (P_{SV40}-GEMS_{NS3aH1}(FGFR1)-pA). | 194, 105, 183 & 186 | FIG. 25 |
| pSL893 | Mammalian P_{SV40}-driven constitutive GEMS_{GNCR}(VEGFR2) expression vector (P_{SV40}-GEMS_{GNCR}(VEGFR2)-pA). | 195, 183 & 185 | FIG. 25 |
| pSL894 | Mammalian P_{SV40}-driven constitutive GEMS_{NS3aH1}(VEGFR2) expression vector (P_{SV40}-GEMS_{NS3aH1}(VEGFR2)-pA). | 196, 105, 183 & 185 | FIG. 25 |
| pSL901 | Mammalian P_{hCMV}-driven constitutive DrBPhP-NSP3 expression vector (P_{hCMV}-DrBPhP-NSP3-pA). | 15 & 81 | FIG. 10 |
| pSL917 | Mammalian P_{hCMV}-driven constitutive MCP-(Δff6_V18FΔN)₄ expression vector (P_{hCMV}-MCP-(Δff6_V18FΔN)4-pA). | 50 & 72 | FIG. 10 |
| pSL942 | Mammalian P_{hCMV}-driven constitutive EGFP-NSP3 expression vector (P_{hCMV}-EGFP-NSP3-pA). | 18 | FIG. 39 |
| pSL1003 | Mammalian P_{hCMV}-driven expression vector for NanoLuc-P2A-mINS-mRNA containing MCP- | 127, 174, 124 & 125 | FIG. 19 |
| | specific poly(A)-surrogate with 16 tandem MS2-box repeats (P_{hCMV}-NanoLuc-P2A-mINS-(MS2-box)₁₆-HHR-pA). | | |
| pSL1014 | Mammalian P_{hCMV}-driven constitutive BCR-ABL expression vector (P_{hCMV}-BCR-ABL-pA). | 301 | FIG. 40 |
| pSL1032 | AAV vector for P_{hCMV}-driven constitutive (GNCR)₃-NSP3 expression (P_{hCMV}-(GNCR)₃-NSP3-pA). | 127 & 30 | FIG. 15, FIG. 19 & FIG. 26 |
| pSL1042 | AAV vector for P_{hCMV}-driven constitutive MCP-(NS3a)₃ expression (P_{hCMV}-MCP-(NS3a)₃-pA). | 55 | FIG. 15 & FIG. 19 |
| pSL1045 | Mammalian P_{hCMV}-driven constitutive BCR expression vector (P_{hCMV}-BCR-pA). | 226 | FIG. 40 |
| pSL1046 | Mammalian P_{hCMV}-driven constitutive ABL1 expression vector (P_{hCMV}-ABL1-pA). | 227 | FIG. 40 |
| pSL1078 | Mammalian P_{hCMV}-driven constitutive L7Ae-EGFP expression vector (P_{hCMV}-L7Ae-EGFP-pA). | 251 | FIG. 9 |
| pSL1079 | Mammalian P_{hCMV}-driven constitutive MCP-(Coh2)₂ expression vector (P_{hCMV}-MCP-(Coh2)₂-pA). | 272 | FIG. 35 |
| pSL1080 | Mammalian P_{hCMV}-driven constitutive MCP-(Coh2)₃ expression vector (P_{hCMV}-MCP-(Coh2)₃-pA). | 273 | FIG. 35, FIG. 36 & FIG. 37 |
| pSL1083 | Mammalian P_{hCMV}-driven constitutive 3xFLAG-MCP-NSP3 expression vector (P_{hCMV}-3xFLAG-MCP-NSP3-pA). | 260 & 221 | FIG. 6 |
| pSL1084 | Mammalian P_{hCMV}-driven constitutive 3xFLAG-MCP expression vector (P_{hCMV}-3xFLAG-MCP-pA). | 264 & 221 | FIG. 6 |
| pSL1085 | P_{T7}-driven expression vector for in vitro transcription of MCP-(NS3a)₃ mRNA containing stabilized | 232, 311, 55, 309 | FIG. 18 |
| | 5′-UTR, 3′-UTR and an extended poly(A) signal (P_{T7}-5′UTR*-MCP-(NS3a)₃-3′UTR*-A₉₂). | & 310 | |
| PSL1091 | P_{T7}-driven expression vector for in vitro transcription of SEAP mRNA containing an extended | 232, 55 & 309 | FIG. 3 |
| | poly(A) signal (P_{T7}-MCP-(NS3a)₃-A₉₂-pA). | | |
| pSL1093 | Mammalian P_{hCMV}-driven constitutive 3xFLAG-MCP-NS3a expression vector (P_{hCMV}-3xFLAG-MCP-NS3a-pA). | 263 & 221 | FIG. 17 |
| pSL1096 | Mammalian P_{hCMV}-driven constitutive MCP-CIB1 expression vector (P_{hCMV}-MCP-CIB1-pA). | 247 & 76 | FIG. 10 |
| pSL1097 | Mammalian P_{hCMV}-driven constitutive MCP-FRB expression vector (P_{hCMV}-MCP-FRB-pA). | 243 & 87 | FIG. 10 |
| pSL1098 | Mammalian P_{hCMV}-driven constitutive FKBP-NSP3 expression vector (P_{hCMV}-FKBP-NSP3-pA). | 252 & 86 | FIG. 10 |
| pSL1099 | Mammalian P_{hCMV}-driven constitutive DocS-mCherry expression vector (P_{hCMV}-DocS-mCherry-pA). | 255 | FIG. 08 |
| pSL1101 | Mammalian P_{hCMV}-driven constitutive 3xFLAG-MCP-ABI(iDab)₃ expression vector (P_{hCMV}-3xFLAG-MCP-ABI(iDab)₃-pA). | 202 & 221 | FIG. 40 |
| pSL1102 | Mammalian P_{hCMV}-driven constitutive 3xHA-CCmut3-NSP3 expression vector (P_{hCMV}-3xHA-CCmut3-NSP3-pA). | 201 & 224 | FIG. 40 |
| pSL1284 | Mammalian P_{hCMV}-driven expression vector for SEAP-mRNA containing MCP-specific poly(A)- | 178, 127, 124 & 125 | FIG. 2 |
| | surrogate with 12 tandem MS2-box repeats (P_{hCMV}-SEAP-(MS2-box)₁₂-HHR-pA). | | |
| pSL1308 | Mammalian P_{hCMV}-driven expression vector for EGFP-mRNA containing MCP-specific poly(A)- | 149, 124 & 125 | FIG. 5. |
| | surrogate with 24 tandem MS2-box repeats (P_{hCMV}-EGFP-(MS2-box)₂₄-HHR-pA). | | |
| pSL1311 | Mammalian P_{hCMV} -driven constitutive MCP-DocS expression vector (P_{hCMV}-MCP-DocS-pA). | 246 | FIG. 35 |
| pSL1315 | Mammalian P_{hCMV} -driven constitutive PABP-MCP expression vector (P_{hCMV}-PABP-MCP-pA). | 206 & 108 | FIG. 2 & FIG. 5 |
| pSL1316 | Mammalian P_{hCMV} -driven constitutive MCP-eIF4E expression vector (P_{hCMV}-MCP-eIF4E-pA). | 244 & 314 | FIG. 2 & FIG. 5 |
| pSL1331 | Mammalian P_{hCMV}-driven expression vector for shRNA-216-repressible MCP-specific SEAP-mRNA | 180, 127, 122 & 125 | FIG. 2 |
| | (P_{hCMV}-SEAP-(MS2-box)₈- BS(shRNA-216)₂-pA). | | |
| PSLM54 | Mammalian P_{MusAFP}-driven NanoLuc expression vector (P_{MusAFP}-NanoLuc-pA). | 173 & 129 | FIG. 45 |
| PSLM97 | Mammalian P_{hCMV}-driven constitutive expression vector for SEAP-mRNA with self-cleavable polyA | 148 & 124 | FIG. 4 |
| | (P_{hCMV}-SEAP-HHR-pA). | | |
| pTetR-ELK1 | Mammalian P_{hCMV}-driven constitutive TetR-ELK1 expression vector (P_{hCMV}-TetR-ELK1-pA). | 199 & 211 | FIG. 25 |
| | (Keeley, Busch, Singh, & Abel, 2005) | | |
| pWH127 | Mammalian P_{hCMV}-driven constitutive cIF4G-2CaM-M13-L7Ae expression vector (P_{hCMV}-cIF4G- | 69, 84 & 312 | FIG. 2 & FIG. 5 |
| | 2CaM-M13-L7Ae-pA). | | |
| pWS164 | Mammalian P_{hCMV}-driven constitutive EGFP expression vector (P_{hCMV}-EGFP-pA). (ref. (Shao et al., 2017)) | 127 & 82 | FIG. 40 |
| PYF1 | Mammalian P_{hCMV}-driven constitutive NS3a-NSP3 expression vector (P_{hCMV}-NS3a-NSP3-pA). | 62 & 104 | FIG. 14 |
| pYF2 | Mammalian P_{hCMV}-driven constitutive DNCR-NSP3 expression vector (P_{hCMV}-DNCR-NSP3-pA). | 270 & 79 | |
| PYF3 | Mammalian P_{hCMV}-driven constitutive GNCR-NSP3 expression vector (P_{hCMV}-GNCR-NSP3-pA). | 28 & 90 | FIG. 10 & FIG. 14 |
| pYF5 | Mammalian P_{hCMV}-driven constitutive L7Ae-NS3a expression vector (P_{hCMV}-L7Ae-NS3a-pA). | 39, 92 & 104 | FIG. 8 & FIG. 14 |
| pYF6 | Mammalian P_{hCMV}-driven constitutive L7Ae-GNCR expression vector (P_{hCMV}-L7Ae-GNCR-pA). | 36 & 90 | FIG. 14 |
| pYW361 | P_{T7}-driven expression vector for in vitro transcription of (GNER)₃-NSP3 mRNA containing stabilized | 232, 311, 30, 310 | FIG. 18 |
| | 5′-UTR, 3′-UTR and an extended poly(A) signal (P_{T7}-5′UTR*-(GNCR)₃-NSP3-3′UTR*-A₉₂). | & 309 | |
| pYW99 | Mammalian P_{hCMV}-driven constitutive FLuc expression vector (P_{hCMV}-FLuc-pA). | 305 | FIG. 11 & FIG. 45 |

| Sequence | | | | | Related | |
|---|---|---|---|---|---|---|
| SEQ | | | | | to | |
| ID NO. | Name | | Type | Length | Plasmid(s) | Sequence |
| 1 | [symbol] | | Protein | 831 | [symbol] | [symbol] |
| 2 | [symbol] | | Protein | 362 | [symbol] | [symbol] |
| 3 | [symbol] | | Protein | 502 | [symbol] | [symbol] |
| 4 | [symbol] | | Protein | 596 | pSL581 | [symbol] |
| 5 | [symbol] | | Protein | 690 | pSL582 | [symbol] |
| 6 | [symbol] | | Protein | 437 | pSL836 | [symbol] |
| 7 | [symbol] | | Protein | 481 | pSL615 | [symbol] |
| 15 | [symbol] | | Protein | 1080 | pSL901 | [symbol] |
| 16 | KRAB | Krueppel: | Protein | 121 | pLZ417 | [symbol] |
| | | associatod box | | | | |
| | | protein of the | | | | |
| | | human kox-1 | | | | |
| | | gene | | | | |
| | | (Bellefroid el. | | | | |
| | | al., 1991). | | | | |
| 17 | [symbol] | | Protein | 449 | pSL816 | [symbol] |
| 18 | EGFP-NSP3 | | Protein | 564 | pSL942 | [symbol] |
| 19 | [symbol] | | Protein | 483 | pSL799 | [symbol] |
| 20 | [symbol] | | Protein | 463 | pSL824 | [symbol] |
| 21 | [symbol] | | Protein | 456 | pSL797 | [symbol] |
| 22 | [symbol] | | Protein | 466 | pSL796 | [symbol] |
| 23 | [symbol] | | Protein | 556 | pSL798 | [symbol] |
| 24 | [symbol] | | Protein | 1061 | pSL189 | [symbol] |
| 25 | FKBP-L7Ac | | Protein | 229 | pMX331 | [symbol] |
| 26 | FRB-NSP3 | | Protein | 433 | PLZ42 | [symbol] |
| 27 | [symbol] | | Protein | 222 | pPW14 | [symbol] |
| 28 | [symbol] | | Protein | 559 | PYF3 | [symbol] |
| 29 | [symbol] | | Protein | 805 | pLZ73 | [symbol] |
| 30 | [symbol] | | Protein | 1049 | [symbol] | [symbol] |
| 31 | L7Ae-(ABI)3 | | Protein | 1046 | pPW3 | [symbol] |
| 32 | [symbol] | | Protein | 300 | [symbol] | [symbol] |
| 33 | [symbol] | | Protein | 267 | pSL65 | [symbol] |
| 34 | [symbol] | | Protein | 417 | pSL82 | [symbol] |
| 35 | [symbol] | | Protein | 567 | pSL83 | [symbol] |
| 36 | [symbol] | | Protein | 364 | PYF6 | [symbol] |
| 37 | [symbol] | | Protein | 243 | pSL667 | [symbol] |
| 38 | [symbol] | | Protein | 282 | pSL661 | [symbol] |
| 39 | [symbol] | | Protein | 327 | pYF5 | [symbol] |
| 40 | [symbol] | | Protein | 535 | pLZ75 | [symbol] |
| 41 | [symbol] | | Protein | 743 | pLZ76 | [symbol] |
| 42 | [symbol] | | Protein | 329 | pSL703 | [symbol] |
| 43 | [symbol] | | Protein | 444 | PLZ27 | [symbol] |
| 44 | [symbol] | | Protein | 346 | [symbol] | [symbol] |
| 45 | [symbol] | | Protein | 442 | pSL543 | [symbol] |
| 46 | [symbol] | | Protein | 389 | pSL169 | [symbol] |
| 47 | [symbol] | | Protein | 398 | pSL136 | [symbol] |
| 48 | [symbol] | | Protein | 765 | pSL475 | [symbol] |
| 49 | [symbol] | | Protein | 466 | pSL762 | [symbol] |
| 50 | [symbol] | | Protein | 410 | PSL917 | [symbol] |
| 52 | [symbol] | | Protein | 414 | pS1776 | [symbol] |
| 53 | [symbol] | | Protein | 693 | pSL777 | [symbol] |
| 54 | [symbol] | | Protein | 338 | pSL497 | [symbol] |
| 55 | [symbol] | | Protein | 752 | [symbol] | [symbol] |
| 56 | [symbol] | | Protein | 340 | pSL546 | [symbol] |
| 57 | [symbol] | | Protein | 550 | PSL547 | [symbol] |
| 58 | [symbol] | | Protein | 760 | pSL548 | [symbol] |
| 59 | [symbol] | | Protein | 453 | pSL95 | [symbol] |
| 60 | [symbol] | | Protein | 453 | pSL560 | [symbol] |
| 61 | [symbol] | | Protein | 457 | pSL754 | [symbol] |
| 62 | [symbol] | | Protein | 522 | YF1 | [symbol] |
| 63 | [symbol] | | Protein | 668 | pPW17 | [symbol] |
| 64 | [symbol] | | Protein | 880 | pPW4 | [symbol] |
| 65 | [symbol] | | Protein | 767 | pLZ16 | [symbol] |
| 66 | [symbol] | | Protein | 789 | pSL763 | [symbol] |
| 67 | [symbol] | | Protein | 884 | pPW24 | [symbol] |
| 68 | [symbol] | | Protein | 490 | pSL834 | [symbol] |
| 69 | [symbol] | | Protein | 1227 | pWH127 | [symbol] |
| 70 | [symbol] | | Protein | 460 | pSL133 | [symbol] |
| 71 | [symbol] | Abscisic acid- | Protein | 300 | [symbol] | [symbol] |
| | | responsive | | | | |
| | | PYL1-binding | | | | |
| | | protein (ABA | | | | |
| | | insensitive | | | | |
| | | mutant) (Gao | | | | |
| | | et al., 2016). | | | | |
| 72 | [symbol] | DrBPhP- | Protein | 56 | pS1917 | [symbol] |
| | | specific | | | | |
| | | [symbol] | | | | |
| | | (Kuwasaki et | | | | |
| | | al, 2022). | | | | |
| 73 | [symbol] | apo NS3a | Protein | 21 | PSL704 | [symbol] |
| | | reader | | | | |
| | | (Cunningham- | | | | |
| | | Bryant et al., | | | | |
| | | 2019). | | | | |
| 74 | [symbol] | B-cell | Protein | 155 | pSL615 | [symbol] |
| | | lymphoma- | | | | |
| | | extra large | | | | |
| | | (Giordano- | | | | |
| | | Attianese et | | | | |
| | | al., 2020). | | | | |
| 75 | [symbol] | pronylation site | Protein | 24 | pSL799 | [symbol] |
| | | recognized by | | | | |
| | | [symbol] | | | | |
| | | [symbol] | | | | |
| | | [symbol] | | | | |
| | | [symbol] | | | | |
| 76 | [symbol] | [symbol] | Protein | 169 | [symbol] | [symbol] |
| | | [symbol] | | | | |
| | | [symbol] | | | | |
| | | [symbol] | | | | |
| | | [symbol] | | | | |
| | | [symbol] | | | | |
| 77 | [symbol] | [symbol] | Protein | 138 | [symbol] | [symbol] |
| 78 | Cry2 | [symbol] | Protein | 498 | [symbol] | [symbol] |
| | | [symbol] | | | | |
| | | [symbol] | | | | |
| | | 2 protein | | | | |
| | | (Kennedy ct | | | | |
| | | al., 2010). | | | | |
| 79 | DNCR | [symbol] | Protein | 229 | [symbol] | [symbol] |
| | | [symbol] | | | | |
| | | [symbol] | | | | |
| | | [symbol] | | | | |
| 80 | Docs | Clostridium | Protein | 69 | [symbol] | [symbol] |
| | | thermocellum | | | | |
| | | Cellulose S. | | | | |
| | | derived | | | | |
| | | Dockerin | | | | |
| | | (Barak et al., | | | | |
| | | 2005). | | | | |
| 81 | DrBPhP | [symbol] | Protein | 754 | pSL901 | [symbol] |
| | | [symbol] | | | | |
| | | [symbol] | | | | |
| | | bacterial | | | | |
| | | [symbol] | | | | |
| | | [symbol] | | | | |
| | | [symbol] | | | | |
| 82 | EGFP | enhanced green | Protein | 238 | pWS164 | [symbol] |
| | | fluorescent | | | | |
| | | protein | | | | |
| 83 | [symbol] | [symbol] | Protein | 216 | [symbol] | [symbol] |
| | | [symbol] | | | | |
| | | [symbol] | | | | |
| | | [symbol] | | | | |
| | | [symbol] | | | | |
| 84 | eIF4G | C-terminal part | Protein | 919 | [symbol] | [symbol] |
| | | [symbol] | | | | |
| | | [symbol] | | | | |
| | | [symbol] | | | | |
| | | [symbol] | | | | |
| 85 | ERK2 | extracellular | Protein | 359 | [symbol] | [symbol] |
| | | regulated | | | | |
| | | protein kinase | | | | |
| | | 2 (NCBI-ID: | | | | |
| | | NM_138957). | | | | |
| 86 | FKBP | FK506-binding | Protein | 107 | [symbol] | [symbol] |
| | | protein (also | | | | |
| | | known as | | | | |
| | | [symbol] | | | | |
| 87 | [symbol] | [symbol] | Protein | 96 | [symbol] | [symbol] |
| | | [symbol] | | | | |
| | | binding | | | | |
| | | domain of the | | | | |
| | | mammalian | | | | |
| | | target of | | | | |
| | | [symbol] | | | | |
| | | (mTOR) | | | | |
| | | kinas [symbol] c. | | | | |
| 88 | GAI | [symbol] | Protein | 91 | [symbol] | [symbol] |
| | | acid- | | | | |
| | | responsive | | | | |
| | | GID1-binding | | | | |
| | | DELLA | | | | |
| | | protein (Gao et. | | | | |
| | | al., 2016). | | | | |
| 89 | GID1 | Gibberellin | Protein | 344 | [symbol] | [symbol] |
| | | receptor GID | | | | |
| | | (Gao cial., | | | | |
| | | 2016) | | | | |
| 90 | GNCR | [symbol] | Protein | 233 | [symbol] | [symbol] |
| | | [symbol] | | | | |
| | | [symbol] | | | | |
| | | [symbol] | | | | |
| 91 | hNSP3 | human | Protein | 312 | [symbol] | [symbol] |
| | | [symbol] | | | | |
| | | [symbol] | | | | |
| | | nonstructural | | | | |
| | | protein 3 | | | | |
| 92 | [symbol] | [symbol] | Protein | 118 | [symbol] | [symbol] |
| | | [symbol] | | | | |
| | | protein | | | | |
| | | specifically | | | | |
| | | binding to the | | | | |
| | | [symbol] | | | | |
| | | [symbol] | | | | |
| | | [symbol] | | | | |
| | | [symbol] | | | | |
| | | [symbol] | | | | |
| 93 | [symbol] | high-affinity | Protein | 269 | [symbol] | [symbol] |
| | | anti-EGFP | | | | |
| | | nanobody | | | | |
| | | (Morsut et al., | | | | |
| | | 2016). | | | | |
| 94 | [symbol] | a synthetic | Protein | 109 | [symbol] | [symbol] |
| | | Bcl-XL- | | | | |
| | | specific protein | | | | |
| | | derived from | | | | |
| | | rat syntaxin 6 | | | | |
| | | (Giordano- | | | | |
| | | [symbol] | | | | |
| | | 2020). | | | | |
| 95 | LD3 | a synthetic | Protein | 149 | pSL661 | [symbol] |
| | | Bcl-XL- | | | | |
| | | specific protein | | | | |
| | | derived from | | | | |
| | | human focal | | | | |
| | | adhesion | | | | |
| | | targeting | | | | |
| | | domain of | | | | |
| | | human | | | | |
| | | apolipoprotein | | | | |
| | | E4 (Giordano- | | | | |
| | | [symbol] | | | | |
| | | 2020). | | | | |
| 96 | mBax | murine BCL2- | Protein | 192 | | [symbol] |
| | | associated X | | | | |
| | | pro-apoptotic | | | | |
| | | protein (NCBI- | | | | |
| | | ID: | | | | |
| | | NM 007527). | | | | |
| 97 | mCherry | mushroom | Protein | 236 | [symbol] | [symbol] |
| | | coral red | | | | |
| | | fluorescence | | | | |
| | | protein. | | | | |
| 98 | MCP | bacteriophage | Protein | 129 | [symbol] | [symbol] |
| | | MS2 coat | | | | |
| | | protein | | | | |
| | | (GenBank: | | | | |
| | | ASW25882.1). | | | | |
| 99 | [symbol] | modified furin- | Protein | 110 | [symbol] | [symbol] |
| | | cleavable | | | | |
| | | insulin variant | | | | |
| | | (Hay and | | | | |
| | | Docherty, | | | | |
| | | 2003). | | | | |
| 100 | N-Peptide | Bacteriophage | Protein | 36 | [symbol] | [symbol] |
| | | λ-derived N- | | | | |
| | | Peptide (S. | | | | |
| | | Auslander et | | | | |
| | | al., 2014). | | | | |
| 101 | NanoLuc | secreted | Protein | 200 | [symbol] | [symbol] |
| | | [symbol] | | | | |
| | | [symbol] | | | | |
| | | luciferase | | | | |
| | | containing a | | | | |
| | | IgK-derived | | | | |
| | | signal peptide. | | | | |
| 102 | [symbol] | mammalian | Protein | 14 | pSL824 | LQLPPLERLTLGSS |
| | | nuclear export | | | | |
| | | signal. | | | | |
| 103 | NLS | mammalian | Protein | 7 | [symbol] | [symbol] |
| | | nucleoplasmin- | | | | |
| | | derived nuclear | | | | |
| | | localization | | | | |
| | | signal. | | | | |
| 104 | NS3a | solubility | Protein | 196 | [symbol] | [symbol] |
| | | optimized | | | | |
| | | catalytically | | | | |
| | | active hepatitis | | | | |
| | | C virus | | | | |
| | | protease | | | | |
| | | (Foight ct al., | | | | |
| | | 2019) | | | | |
| 105 | [symbol] | catalytically | Protein | 196 | [symbol] | [symbol] |
| | | [symbol] | | | | |
| | | variant | | | | |
| | | (Cunningham- | | | | |
| | | Bryant et al., | | | | |
| | | 2019) | | | | |
| 106 | NSP3 | bovine | Protein | 312 | pLZ27, | [symbol] |
| | | rotavirus strain | | | pSL95 | |
| | | RF | | | | |
| | | nonstructural | | | | |
| | | protein 3 | | | | |
| | | (UniProtKB/ | | | | |
| | | Swiss-Prot: | | | | |
| | | Q86504.1): | | | | |
| 107 | P2A | Porcine | Protein | 20 | pSL816 | [symbol] |
| | | [symbol] | | | | |
| | | derived | | | | |
| | | ribosome | | | | |
| | | skipping | | | | |
| | | sequence | | | | |
| | | optimized for | | | | |
| | | [symbol] | | | | |
| | | expression in | | | | |
| | | mammalian | | | | |
| | | cells. | | | | |
| 108 | PABP | Poly(A)- | Protein | 635 | [symbol] | [symbol] |
| | | binding protein | | | | |
| | | (NCBI-ID: | | | | |
| | | XP_004402403.1) | | | | |
| 109 | [symbol] | Streptomyces | Protein | 153 | [symbol] | [symbol] |
| | | [symbol] | | | | |
| | | derived | | | | |
| | | repressor of the | | | | |
| | | [symbol] | | | | |
| | | [symbol] | | | | |
| | | (Yin et al., | | | | |
| | | 2019). | | | | |
| 110 | pE59 | DARPin | Protein | 123 | [symbol] | [symbol] |
| | | targeting. | | | | |
| | | phosphorylated | | | | |
| | | ERK2 | | | | |
| | | (Kummer et. | | | | |
| | | al, 2012). | | | | |
| 111 | PYL1 | [symbol] | Protein | 181 | [symbol] | [symbol] |
| | | responsive | | | | |
| | | ABI-binding | | | | |
| | | protein | | | | |
| | | [symbol] | | | | |
| | | resistance | | | | |
| | | (PYR)-like | | | | |
| | | protein) (Gao | | | | |
| | | et al., 2016). | | | | |
| 112 | scFv162 | nNS3-specific | Protein | 134 | [symbol] | [symbol] |
| | | scFv (Siciliano | | | | |
| | | et al. 2018); | | | | |
| 113 | scFv35 | nNS3-specific | Protein | 266 | pSL136 | [symbol] |
| | | scFv (Siciliano | | | | |
| | | et al., 2018). | | | | |
| 114 | SEAP | human | Protein | 519 | [symbol] | [symbol] |
| | | placental | | | | |
| | | secreted | | | | |
| | | alkaline | | | | |
| | | phosphatase | | | | |
| | | (Berger et al., | | | | |
| | | 1988). | | | | |
| 115 | SEAP-derived | SEAP-derived | Protein | 17 | PSL796 | [symbol] |
| | secretory | secretory | | | | |
| | signal | signal peptide. | | | | |
| | peptide (SP) | | | | | |
| 248 | NanoLuc- | NanoLuc- | Protein | 21 | pSL890 | [symbol] |
| | derived | derived | | | | |
| | secretory | secretory | | | | |
| | signal | signal peptide. | | | | |
| | peptide (SP) | | | | | |
| 116 | StaPLd | [symbol] | Protein | 236 | [symbol] | [symbol] |
| | | stabilizable | | | | |
| | | polypeptide | | | | |
| | | linkages based | | | | |
| | | on hepatitis C | | | | |
| | | virus protease | | | | |
| | | (Jacobs et al., | | | | |
| | | 2018). | | | | |
| 117 | TetR | Escherichia | Protein | 205 | [symbol] | [symbol] |
| | | coli [symbol] | | | | |
| | | derived | | | | |
| | | [symbol] | | | | |
| | | dependent | | | | |
| | | repressor of the | | | | |
| | | tetracycline | | | | |
| | | resistance gene | | | | |
| | | [symbol] | | | | |
| | | [symbol] | | | | |
| 118 | TM | N-terminal | Protein | 81 | PSL798 | [symbol] |
| | | transmembrane | | | | |
| | | domain | | | | |
| 119 | VP64 | [symbol] | Protein | 50 | [symbol] | [symbol] |
| | | of Herpes | | | | |
| | | simplex virus- | | | | |
| | | derived | | | | |
| | | transactivation | | | | |
| | | domain. | | | | |
| 120 | VPg | [symbol] | Protein | 117 | [symbol] | [symbol] |
| | | protein | | | | |
| | | [symbol] | | | | |
| | | [symbol] | | | | |
| 121 | boxB | [symbol] | RNA | 19 | [symbol] | GGGCCCUGAAGAAGGGCCC |
| | | N)-specific | | | | |
| | | aptamer motif | | | | |
| | | [symbol] | | | | |
| | | et al., 2014) | | | | |
| 122 | [symbol] | Binding site | RNA | 22 | [symbol] | UCACAGUUGCCAGUGAGAUUA |
| | | for shRNA- | | | | |
| | | 216. | | | | |
| 123 | C/D-box | L7AC-specific | RNA | 23 | [symbol] | GGGCGUGAUCCGAAAGGUGACCC |
| | | RNA aptamer | | | | |
| | | with a kink- | | | | |
| | | [symbol] | | | | |
| | | motif | | | | |
| | | (Nakanishi and | | | | |
| | | Saito, 2020). | | | | |
| 124 | HHR | [symbol] | RNA | 76 | [symbol] | [symbol] |
| | | N)-repressible | | | | |
| | | hammerhead | | | | |
| | | ribozyme (S. | | | | |
| | | [symbol] | | | | |
| | | al., 2014). | | | | |
| 125 | MS2-box (C- | MCP-specific | RNA | 13 | [symbol] | UGAGGAUCACOCA |
| | variant) | RNA aptamer | | | | |
| | | (C-variant) | | | | |
| | | (Nakanishi and | | | | |
| | | Saito, 2020). | | | | |
| 315 | MS2-box (U- | MCP-specific | RNA | 19 | [symbol] | ACAUGAGGAUUACCCAUGU |
| | variant) | RNA aptamer | | | | |
| | | (L-variant) | | | | |
| | | (Nakanishi and | | | | |
| | | Saito, 2020). | | | | |
| 126 | shRNA-216 | short hairpin | RNA | 60 | [symbol] | [symbol] |
| | | RNA no. 216. | | | | |
| 127 | [symbol] | human | DNA | 588 | [symbol] | [symbol] |
| | | [symbol] | | | | |
| | | [symbol] | | | | |
| | | curly promoter. | | | | |
| 128 | P_hU6 | humun U6 | DNA | 241 | pSLA | [symbol] |
| | | promoter. | | | | |
| 129 | P_MusAFP | | DNA | 283 | [symbol] | [symbol] |
| 130 | P_CRE-NLS- | | DNA | 1617 | pSL754 | [symbol] |
| | PcaV- [symbol] - | | | | | |
| | VP64 | | | | | |
| 131 | 5′-UTR- | | RNA | 2208 | PSL31 | [symbol] |
| | SEAP-(C/D- | | | | | |
| | Box)x8- | | | | | |
| | (shRNA- | | | | | |
| | 216)x2 | | | | | |
| 132 | 5′-UTR- | | RNA | 2709 | pSL80 | [symbol] |
| | SEAP-(C/D)- | | | | | |
| | Box)x16- | | | | | |
| | (shRNA- | | | | | |
| | 216)x2 | | | | | |
| 133 | 5′-UTR- | | RNA | 2424 | pSL81 | [symbol] |
| | SEAP-(C/D- | | | | | |
| | Box)x12- | | | | | |
| | (shRNA- | | | | | |
| | 216)x2 | | | | | |
| 134 | 5′-UTR- | | RNA | 3058 | pSL88 | [symbol] |
| | SEAP-(CD)- | | | | | |
| | Box)x24- | | | | | |
| | (shRNA- | | | | | |
| | 216)x2 | | | | | |
| 135 | 5′-UTR-NanoLuc-(C/D- | | RNA | 2133 | pSL274 | [symbol] |
| | Box)x24-(shRNA-216)x2: | | | | | |
| 136 | 5′- | | RNA | 3056 | PSL355 | [symbol] |
| | SEAP- | | | | | |
| | (C/D- | | | | | |
| | Box)x24- | | | | | |
| | HHR | | | | | |
| 137 | 5′- | | RNA | 2959 | pSL468, | [symbol] |
| | UTR- | | | | pSL511 | |
| | SEAP- | | | | | |
| | (MS2- | | | | | |
| | Box)x24- | | | | | |
| | HHR | | | | | |
| 138 | 5′ | | RNA | 2549 | pSL516 | [symbol] |
| | UTR- | | | | | |
| | SEAP- | | | | | |
| | (MS2- | | | | | |
| | Box)x16- | | | | | |
| | [symbol] | | | | | |
| 139 | 5′-UTR-NanoLuc-P2A- | | RNA | 2763 | pSL683 | [symbol] |
| | mCherry-(MS2-Box)x24-HHR | | | | | |
| 140 | 5′-UTR-NanoLoc-P2A-mINS- | | RNA | 2385 | [symbol] | [symbol] |
| | (MS2-Box)x24-HHR | | | | | |
| 141 | [symbol] | | DNA | 987 | pSL684 | [symbol] |
| | [symbol] | | | | | |
| | [symbol] | | | | | |
| | [symbol] | | | | | |
| | mCherry | | | | | |
| 142 | 5′- | | RNA | 1820 | pSL767 | [symbol] |
| | UTR- | | | | | |
| | SEAP- | | | | | |
| | (HHR)x2 | | | | | |
| 143 | 5′- | | RNA | 1981 | pSL768 | [symbol] |
| | UTR- | | | | | |
| | SEAP- | | | | | |
| | (HHR)x4 | | | | | |
| 144 | [symbol] | | DNA | 3050 | pSL813 | [symbol] |
| | (MS2-Box)x24-HHR | | | | | |
| 145 | 5′- | | RNA | 1965 | pSL831 | [symbol] |
| | UTR- | | | | | |
| | mBax- | | | | | |
| | (MS2- | | | | | |
| | Box)x24- | | | | | |
| | HHR | | | | | |
| 146 | P_AFP- | | DNA | 3265 | pSL857 | [symbol] |
| | SEAP- | | | | | |
| | (MS2- | | | | | |
| | Box)x24- | | | | | |
| | HHR | | | | | |
| 147 | P_AFP- | | DNA | 2268 | pSL886 | [symbol] |
| | mBax- | | | | | |
| | (MS2- | | | | | |
| | Box)x24- | | | | | |
| | HHR | | | | | |
| 148 | 5′-UTR- | | RNA | 1713 | pSLM97 | [symbol] |
| | SEAP- | | | | | |
| | [symbol] | | | | | |
| 149 | 5′- | | RNA | 2106 | [symbol] | [symbol] |
| | UTR- | | | | | |
| | EGFP- | | | | | |
| | (MS2- | | | | | |
| | Rox)x24- | | | | | |
| | HHR | | | | | |
| 150 | [symbol] | | DNA | 1135 | pLZ287 | [symbol] |
| | [symbol] | | | | | |
| | [symbol] | | | | | |
| | [symbol] | | | | | |
| | mCherry | | | | | |
| 151 | [symbol] | | DNA | 2223 | pLYL95 | [symbol] |
| | [symbol] | | | | | |
| | [symbol] | | | | | |
| | NSP3 | | | | | |
| 152 | [symbol] | | DNA | 3365 | pLYL87 | [symbol] |
| | [symbol] | | | | | |
| | [symbol] | | | | | |
| | [symbol] | | | | | |
| 153 | P_CRE- | | DNA | 2316 | pSL580 | [symbol] |
| | 8xANR- | | | | | |
| | NSP3 | | | | | |
| 154 | P_CRE- | | DNA | 2963 | pLZ310 | [symbol] |
| | GEMS_ | | | | | |
| | ANR | | | | | |
| 155 | [symbol] | | DNA | 3038 | pLZ413 | [symbol] |
| | [symbol] | | | | | |
| | [symbol] | | | | | |
| | min- | | | | | |
| | GEMS_ | | | | | |
| | ANR | | | | | |
| 156 | [symbol] | | DNA | 2483 | pLZ412 | [symbol] |
| | [symbol] | | | | | |
| | [symbol] | | | | | |
| | min- | | | | | |
| | GEMS_ | | | | | |
| | NS3a(H1) | | | | | |
| 157 | P_CRE- | | DNA | 2405 | pLZ286 | [symbol] |
| | GEMS_ | | | | | |
| | [symbol] | | | | | |
| 158 | P_CRE- | | DNA | 2516 | pLZ285 | [symbol] |
| | GEMS_ | | | | | |
| | GNCR | | | | | |
| 159 | [symbol] | | DNA | 3510 | pLYL63 | [symbol] |
| | [symbol] | | | | | |
| | [symbol] | | | | | |
| | min- | | | | | |
| | 3xGNCR- | | | | | |
| | NSP3 | | | | | |
| 160 | P_CRE- | | DNA | 1752 | PLYL67 | [symbol] |
| | 4xANR- | | | | | |
| | [symbol] | | | | | |
| 161 | [symbol] | | DNA | 1659 | [symbol] | [symbol] |
| | [symbol] | | | | | |
| | 4xANR- | | | | | |
| | NSP3 | | | | | |
| 162 | [symbol] | | DNA | 3502 | pLYL62 | [symbol] |
| | [symbol] | | | | | |
| | [symbol] | | | | | |
| 163 | 5′- | | RNA | 1989 | [symbol] | [symbol] |
| | UTR- | | | | | |
| | NanoLuc- | | | | | |
| | (MS2- | | | | | |
| | Box)x24- | | | | | |
| 164 | [symbol] | | DNA | 1003 | pLZ368 | [symbol] |
| | [symbol] | | | | | |
| | [symbol] | | | | | |
| | min- | | | | | |
| | NanoLuc | | | | | |
| 165 | [symbol] | | DNA | 1823 | pSL172 | [symbol] |
| | [symbol] | | | | | |
| | [symbol] | | | | | |
| | min- | | | | | |
| | SEAP | | | | | |
| 166 | [symbol] | | DNA | 1911 | pLZ419 | [symbol] |
| | [symbol] | | | | | |
| | [symbol] | | | | | |
| | SEAP | | | | | |
| 167 | P_NEAT | | DNA | 1801 | pMX57 | [symbol] |
| | 3-SEAP | | | | | |
| 168 | [symbol] | | DNA | 2034 | [symbol] | [symbol] |
| | [symbol] | | | | | |
| | SEAP | | | | | |
| 169 | [symbol] | | DNA | 1950 | pLZ284 | [symbol] |
| | [symbol] | | | | | |
| | [symbol] | | | | | |
| | min- | | | | | |
| | SEAP | | | | | |
| 170 | [symbol] | | DNA | 850 | pLZ345 | [symbol] |
| | [symbol] | | | | | |
| | [symbol] | | | | | |
| | min- | | | | | |
| | NanoLuc | | | | | |
| 171 | [symbol] | | DNA | 1789 | pSL173 | [symbol] |
| | [symbol] | | | | | |
| | [symbol] | | | | | |
| | min- | | | | | |
| | SEAP | | | | | |
| 172 | P_CRE- | | DNA | 1854 | pCK53 | [symbol] |
| | SEAP | | | | | |
| 173 | P_AFP- | | DNA | 897 | pSLM54 | [symbol] |
| | NanoLuc | | | | | |
| 174 | 5′-UTR- | | RNA | 1987 | pSL1003 | [symbol] |
| | NanoLuc- | | | | | |
| | P2A- | | | | | |
| | mCherry- | | | | | |
| | (MS2- | | | | | |
| | [symbol] | | | | | |
| | HHR | | | | | |
| 175 | 5′- | | RNA | 3579 | pPW21 | [symbol] |
| | UTR- | | | | | |
| | SEAP- | | | | | |
| | (C/D- | | | | | |
| | Box)x24- | | | | | |
| | Nanoluc | | | | | |
| 176 | 5′- | | RNA | 1921 | pQZ8 | [symbol] |
| | UTR- | | | | | |
| | (C/D | | | | | |
| | Box)x4- | | | | | |
| | SEAP | | | | | |
| 177 | 5′- | | RNA | 3105 | pSL781 | [symbol] |
| | UTR- | | | | | |
| | FLAG- | | | | | |
| | FLuc- | | | | | |
| | (MS2- | | | | | |
| | Box)x24- | | | | | |
| | HHR | | | | | |
| 178 | 5′- | | RNA | 2339 | pSL1284 | [symbol] |
| | UTR- | | | | | |
| | SEAP | | | | | |
| | (MS2- | | | | | |
| | Box)x12- | | | | | |
| | HHR | | | | | |
| 179 | 5′- | | RNA | 2128 | pSLS15 | [symbol] |
| | UTR- | | | | | |
| | SEAP- | | | | | |
| | (MS2- | | | | | |
| | Box)x8- | | | | | |
| | HHR | | | | | |
| 180 | 5′- | | RNA | 2164 | pSL1331 | [symbol] |
| | UTR- | | | | | |
| | SEAP- | | | | | |
| | (MS2- | | | | | |
| | Box)x8- | | | | | |
| | (shRNA- | | | | | |
| | 216)x2 | | | | | |
| 181 | MCP- | | Protein | 266 | pSL860 | [symbol] |
| | [symbol] | | | | | |
| | [symbol] | | | | | |
| 182 | GEMS_ | modified F93A | Protein | 251 | [symbol] | [symbol] |
| | TM | mutant of | | | | |
| | | Erythropoietin | | | | |
| | | receptor (EpoR) | | | | |
| | | containing three | | | | |
| | | [symbol] | | | | |
| | | [symbol] | | | | |
| | | [symbol] | | | | |
| | | [symbol] | | | | |
| 183 | [symbol] | [symbol] | Protein | 248 | [symbol] | [symbol] |
| | | mutant of | | | | |
| | | Erythropoictin | | | | |
| | | receptor (EpoR) | | | | |
| | | (Scheller et | | | | |
| | | al., 2018). | | | | |
| 184 | IL- | modified | Protein | 276 | [symbol] | [symbol] |
| | 6RB_m | intracellular | | | | |
| | | signal | | | | |
| | | transduction | | | | |
| | | domuins of | | | | |
| | | interleukin 6 | | | | |
| | | receptor | | | | |
| | | [symbol] | | | | |
| | | [symbol] | | | | |
| | | [symbol] | | | | |
| | | [symbol] | | | | |
| | | [symbol] | | | | |
| | | [symbol] | | | | |
| | | 2018). | | | | |
| 185 | VEGFR2_ | [symbol] | Protein | 571 | [symbol] | [symbol] |
| | int | [symbol] | | | | |
| | | [symbol] | | | | |
| | | [symbol] | | | | |
| | | [symbol] | | | | |
| | | [symbol] | | | | |
| 186 | [symbol] | [symbol] | Protein | 423 | [symbol] | [symbol] |
| | | [symbol] | | | | |
| | | [symbol] | | | | |
| | | [symbol] | | | | |
| | | [symbol] | | | | |
| 187 | [symbol] | | Protein | 746 | pLZ267 | [symbol] |
| | [symbol] | | | | | |
| | [symbol] | | | | | |
| 188 | [symbol] | | Protein | 934 | pLZ268 | [symbol] |
| | [symbol] | | | | | |
| | [symbol] | | | | | |
| 189 | [symbol] | | Protein | 890 | pLZ269 | [symbol] |
| | [symbol] | | | | | |
| | [symbol] | | | | | |
| 190 | [symbol] | | Protein | 1033 | pLZ271 | [symbol] |
| | [symbol] | | | | | |
| | [symbol] | | | | | |
| 191 | [symbol] | | Protein | 786 | [symbol] | [symbol] |
| | [symbol] | | | | | |
| | [symbol] | | | | | |
| 192 | [symbol] | | Protein | 749 | pSL890 | [symbol] |
| | [symbol] | | | | | |
| | [symbol] | | | | | |
| 193 | [symbol] | | Protein | 930 | pSL891 | [symbol] |
| | [symbol] | | | | | |
| | [symbol] | | | | | |
| 194 | [symbol] | | Protein | 893 | pSL892 | [symbol] |
| | [symbol] | | | | | |
| | [symbol] | | | | | |
| 195 | [symbol] | | Protein | 1077 | pSL893 | [symbol] |
| | [symbol] | | | | | |
| | [symbol] | | | | | |
| 196 | [symbol] | | Protein | 1040 | pSL894 | [symbol] |
| | [symbol] | | | | | |
| | [symbol] | | | | | |
| 197 | [symbol] | | Protein | 555 | pLZ417 | [symbol] |
| | [symbol] | | | | | |
| | [symbol] | | | | | |
| | KRAB | | | | | |
| 198 | [symbol] | | Protein | 456 | pLZ418 | [symbol] |
| | [symbol] | | | | | |
| | [symbol] | | | | | |
| | VP64 | | | | | |
| 199 | [symbol] | [symbol] | Protein | 338 | [symbol] | [symbol] |
| | [symbol] | [symbol] | | | | |
| | | [symbol] | | | | |
| | | [symbol] | | | | |
| | | [symbol] | | | | |
| 200 | [symbol] | V29I mutant of | Protein | 116 | pSL565 | [symbol] |
| | [symbol] | MCP. | | | | |
| 201 | CCmut3- | | Protein | 424 | pSL1102 | [symbol] |
| | NSP3- | | | | | |
| | [symbol] | | | | | |
| 202 | 3xFLAG- | | Protein | 288 | pSL1101 | [symbol] |
| | MCP- | | | | | |
| | [symbol] | | | | | |
| | [symbol] | | | | | |
| 203 | [symbol] | | Protein | 312 | pPW9 | [symbol] |
| | PYL1 | | | | | |
| 204 | [symbol] | | Protein | 297 | pSL776 | [symbol] |
| | mCherry | | | | | |
| 205 | [symbol] | | Protein | 170 | pSL334 | [symbol] |
| | Coh2 | | | | | |
| 206 | PABP- | | Protein | 775 | pSL1315 | [symbol] |
| | MCP | | | | | |
| 207 | PABP- | | Protein | 718 | pSLA7 | [symbol] |
| | [symbol] | | | | | |
| 208 | MOR9-1 | [symbol] | Protein | 344 | [symbol] | [symbol] |
| | | [symbol] | | | | |
| | | [symbol] | | | | |
| | | [symbol] | | | | |
| 209 | [symbol] | STAT3-specific | DNA | 50 | [symbol] | [symbol] |
| | [symbol] | response | | | | |
| | | element. | | | | |
| 210 | P_NFAT | [symbol] | DNA | 241 | pMX57 | [symbol] |
| | 3 | [symbol] | | | | |
| | | [symbol] | | | | |
| | | [symbol] | | | | |
| | | [symbol] | | | | |
| | | [symbol] | | | | |
| | | [symbol] | | | | |
| | | [symbol] | | | | |
| 211 | ELK1 | [symbol] | Protein | 132 | [symbol] | [symbol] |
| | | [symbol] | | | | |
| | | [symbol] | | | | |
| | | [symbol] | | | | |
| | | [symbol] | | | | |
| | | [symbol] | | | | |
| | | [symbol] | | | | |
| 212 | [symbol] | [symbol] | DNA | 130 | [symbol] | [symbol] |
| | [symbol] | [symbol] | | | | |
| | | [symbol] | | | | |
| | | [symbol] | | | | |
| 316 | [symbol] | [symbol] | DNA | 130 | [symbol] | [symbol] |
| | [symbol] | [symbol] | | | | |
| | | [symbol] | | | | |
| | | [symbol] | | | | |
| 213 | CRE | CAMP-response | DNA | 8 | pLZ285 | [symbol] |
| | | [symbol] | | | | |
| | | [symbol] | | | | |
| 317 | CRE_m | [symbol] | DNA | 8 | [symbol] | [symbol] |
| | (RIP) | [symbol] | | | | |
| | | [symbol] | | | | |
| | | [symbol] | | | | |
| 214 | P_TAL | [symbol] | DNA | 147 | [symbol] | [symbol] |
| | | [symbol] | | | | |
| | | [symbol] | | | | |
| | | [symbol] | | | | |
| | | [symbol] | | | | |
| 215 | 5′-LTR | Lentivirus long | DNA | 634 | pLZ276 | [symbol] |
| | | terminal | | | | |
| | | repeats. | | | | |
| 216 | TRFS | internal | RNA | 574 | pLZ276 | [symbol] |
| | | ribosome | | | | |
| | | entry site. | | | | |
| 217 | tetO | TetR-specific | DNA | 19 | [symbol] | [symbol] |
| | | [symbol] | | | | |
| | | [symbol] | | | | |
| 218 | [symbol] | NFAT response | DNA | 18 | pMX57 | [symbol] |
| | [symbol] | [symbol] | | | | |
| | | [symbol] | | | | |
| | | [symbol] | | | | |
| | | 1994). | | | | |
| 219 | VanR | [symbol] | Protein | 243 | pSL175 | [symbol] |
| | | [symbol] | | | | |
| | | [symbol] | | | | |
| | | [symbol] | | | | |
| | | [symbol] | | | | |
| | | [symbol] | | | | |
| | | [symbol] | | | | |
| 220 | [symbol] | [symbol] | Protein | 265 | [symbol] | [symbol] |
| | [symbol] | [symbol] | | | | |
| | | [symbol] | | | | |
| 221 | FLAG | [symbol] | Protein | 22 | [symbol] | [symbol] |
| | | [symbol] | | | | |
| | | [symbol] | | | | |
| | | [symbol] | | | | |
| 222 | CCmut3 | [symbol] | Protein | 71 | pSL863 | [symbol] |
| | | [symbol] | | | | |
| | | [symbol] | | | | |
| | | [symbol] | | | | |
| 223 | [symbol] | [symbol] | Protein | 122 | pSL860 | [symbol] |
| | [symbol] | [symbol] | | | | |
| | | [symbol] | | | | |
| | | [symbol] | | | | |
| 224 | 3xHA | [symbol] | Protein | 27 | [symbol] | [symbol] |
| | | [symbol] | | | | |
| 225 | [symbol] | [symbol] | DNA | 20 | [symbol] | [symbol] |
| | | [symbol] | | | | |
| | | [symbol] | | | | |
| 226 | ABL1 | Tyrosine-protein | Protein | 1130 | [symbol] | [symbol] |
| | | [symbol] | | | | |
| | | [symbol] | | | | |
| | | [symbol] | | | | |
| | | [symbol] | | | | |
| 227 | BCR | Breakpoint | Protein | 1271 | PSL1046 | [symbol] |
| | | cluster | | | | |
| | | region protein | | | | |
| | | encoded by the | | | | |
| | | human BCR gene | | | | |
| | | on | | | | |
| | | chromosome 22. | | | | |
| 228 | ZeoR | gene conferring | Protein | 124 | [symbol] | [symbol] |
| | | [symbol] | | | | |
| 229 | PuroR | gene conferring | Protein | 199 | [symbol] | [symbol] |
| | | puromycin | | | | |
| | | resistance. | | | | |
| 230 | VanO | VanR-specific. | DNA | 12 | [symbol] | [symbol] |
| | | operator site | | | | |
| | | [symbol] | | | | |
| | | [symbol] | | | | |
| 231 | P_min | [symbol] | DNA | 32 | [symbol] | [symbol] |
| | | [symbol] | | | | |
| 232 | P_T7 | [symbol] | DNA | 20 | [symbol] | [symbol] |
| | | [symbol] | | | | |
| | | [symbol] | | | | |
| | | [symbol] | | | | |
| 233 | P_SV40 | simian virus 40 | DNA | 330 | [symbol] | [symbol] |
| | | constitutive | | | | |
| | | promoter. | | | | |
| 234 | P_RPBSA | [symbol] | DNA | 612 | [symbol] | [symbol] |
| | | [symbol] | | | | |
| | | [symbol] | | | | |
| | | [symbol] | | | | |
| 235 | P_hPGK | human | DNA | 501 | [symbol] | [symbol] |
| | | [symbol] | | | | |
| | | [symbol] | | | | |
| | | [symbol] | | | | |
| 236 | [symbol] | human elongation | DNA | 1059 | [symbol] | [symbol] |
| | | [symbol] | | | | |
| 237 | [symbol] | [symbol] | DNA | 144 | [symbol] | [symbol] |
| | min | [symbol] | | | | |
| 238 | [symbol] | [symbol] | DNA | 437 | [symbol] | [symbol] |
| | [symbol] | [symbol] | | | | |
| | | [symbol] | | | | |
| | | [symbol] | | | | |
| | | [symbol] | | | | |
| | | [symbol] | | | | |
| | | [symbol] | | | | |
| 239 | P_CRE | [symbol] | DONA | 240 | [symbol] | [symbol] |
| | | [symbol] | | | | |
| | | [symbol] | | | | |
| | | [symbol] | | | | |
| | | [symbol] | | | | |
| | | [symbol] | | | | |
| 240 | P_CAG | [symbol] | DNA | 584 | pSL446 | [symbol] |
| | | [symbol] | | | | |
| | | [symbol] | | | | |
| | | [symbol] | | | | |
| | | [symbol] | | | | |
| | | [symbol] | | | | |
| | | [symbol] | | | | |
| 241 | [symbol] | | Protein | 408 | pSL875 | [symbol] |
| | [symbol] | | | | | |
| | [symbol] | | | | | |
| 242 | MCP- | | Protein | 486 | pPW23 | [symbol] |
| | GID1 | | | | | |
| 243 | MCP- | | Protein | 236 | pSL1097 | [symbol] |
| | FRB | | | | | |
| 244 | MCP- | | Protein | 358 | PSL1316 | [symbol] |
| | [symbol] | | | | | |
| 245 | MGP- | | Protein | 379 | pSL435 | [symbol] |
| | EGFP | | | | | |
| 246 | MCP- | | Protein | 214 | pSL1311 | [symbol] |
| | [symbol] | | | | | |
| 247 | MCP- | | Protein | 309 | pSL1096 | [symbol] |
| | [symbol] | | | | | |
| 249 | MCP- | | Protein | 402 | pSL637 | [symbol] |
| | [symbol] | | | | | |
| 250 | mCherry- | | Protein | 561 | pSL876 | [symbol] |
| | NSP3 | | | | | |
| 251 | L7Ae- | | Protein | 370 | pSL1078 | [symbol] |
| | [symbol] | | | | | |
| 252 | FKBP- | | Protein | 433 | PSL1098 | [symbol] |
| | [symbol] | | | | | |
| 253 | [symbol] | | Protein | 1059 | PSL154 | [symbol] |
| | [symbol] | | | | | |
| 254 | [symbol] | | Protein | 192 | PLZ311 | [symbol] |
| | [symbol] | | | | | |
| 255 | [symbol] | | Protein | 319 | pSL1099 | [symbol] |
| | mCherry | | | | | |
| 256 | [symbol] | | Protein | 999 | pSL87 | [symbol] |
| | [symbol] | | | | | |
| 257 | [symbol] | | Protein | 291 | [symbol] | [symbol] |
| | eIF4E | | | | | |
| 258 | [symbol] | | Protein | 397 | pSL863 | [symbol] |
| | [symbol] | | | | | |
| 259 | ABI- | | Protein | 442 | pPW22 | [symbol] |
| | MCP | | | | | |
| 260 | 3xFLAG- | | Protein | 475 | pSL1083 | [symbol] |
| | MCP- | | | | | |
| | NSP3 | | | | | |
| 261 | [symbol] | | Protein | 607 | pSL138 | [symbol] |
| | [symbol] | | | | | |
| 262 | [symbol] | | Protein | 754 | [symbol] | [symbol] |
| | [symbol] | | | | | |
| 263 | 3xFLAG- | | Protein | 360 | [symbol] | [symbol] |
| | MCP- | | | | | |
| | NS3a | | | | | |
| 264 | 3xFLAG- | | Protein | 152 | [symbol] | [symbol] |
| | MCP | | | | | |
| 265 | [symbol] | | Protein | 431 | pPW8 | [symbol] |
| | [symbol] | | | | | |
| 266 | [symbol] | | Protein | 625 | pPW10 | [symbol] |
| | [symbol] | | | | | |
| 267 | [symbol] | | Protein | 408 | [symbol] | [symbol] |
| | [symbol] | | | | | |
| 268 | Coh2- | | Protein | 464 | pSL241 | [symbol] |
| | NSP3 | | | | | |
| 269 | [symbol] | | Protein | 614 | pSL242 | [symbol] |
| | [symbol] | | | | | |
| 270 | [symbol] | | Protein | 555 | pYF2 | [symbol] |
| | [symbol] | | | | | |
| 271 | [symbol] | | Protein | 797 | pLZ71 | [symbol] |
| | [symbol] | | | | | |
| 272 | [symbol] | | Protein | 432 | pSL1079 | [symbol] |
| | [symbol] | | | | | |
| 273 | [symbol] | | Protein | 582 | [symbol] | [symbol] |
| | [symbol] | | | | | |
| 274 | [symbol] | | Protein | 586 | pSL479 | [symbol] |
| | [symbol] | | | | | |
| | [symbol] | | | | | |
| 275 | [symbol] | | Protein | 243 | PLY147 | [symbol] |
| | [symbol] | | | | | |
| 276 | [symbol] | | Protein | 235 | pSL565 | [symbol] |
| | [symbol] | | | | | |
| 277 | [symbol] | | Protein | 685 | pSLI71 | [symbol] |
| | [symbol] | | | | | |
| 278 | PYL1- | | Protein | 507 | pPW6 | [symbol] |
| | NSP3 | | | | | |
| 279 | [symbol] | | Protein | 313 | pPW12 | [symbol] |
| | [symbol] | | | | | |
| 280 | [symbol] | | Protein | 254 | pSL168 | [symbol] |
| | [symbol] | | | | | |
| 281 | [symbol] | | Protein | 245 | [symbol] | [symbol] |
| | [symbol] | | | | | |
| 282 | GID1 | | Protein | 474 | pPW15 | [symbol] |
| 283 | [symbol] | | Protein | 219 | pPW13 | [symbol] |
| | [symbol] | | | | | |
| 284 | [symbol] | | Protein | 430 | pPW5 | [symbol] |
| | [symbol] | | | | | |
| 285 | [symbol] | | Protein | 397 | [symbol] | [symbol] |
| | [symbol] | | | | | |
| 286 | [symbol] | | Protein | 475 | [symbol] | [symbol] |
| | [symbol] | | | | | |
| 287 | GID1- | | Protein | 669 | [symbol] | [symbol] |
| | NSP3 | | | | | |
| 288 | [symbol] | | Protein | 439 | PSLM79 | [symbol] |
| | (Coh2)2- | | | | | |
| | 3xFLAG | | | | | |
| 289 | [symbol] | | Protein | 416 | [symbol] | [symbol] |
| | [symbol] | | | | | |
| 290 | [symbol] | | Protein | 546 | [symbol] | [symbol] |
| | [symbol] | | | | | |
| 291 | [symbol] | | Protein | 507 | pPW7 | [symbol] |
| | [symbol] | | | | | |
| 292 | NSP3- | | Protein | 1368 | pPW20 | [symbol] |
| | [symbol] | | | | | |
| 293 | [symbol] | | Protein | 415 | pPW18 | [symbol] |
| | [symbol] | | | | | |
| 294 | [symbol] | | Protein | 624 | pPW11 | [symbol] |
| | [symbol] | | | | | |
| 295 | [symbol] | | Protein | 572 | pSL781 | [symbol] |
| | [symbol] | | | | | |
| | [symbol] | | | | | |
| 296 | VanR- | | Protein | 304 | pSL175 | [symbol] |
| | VP64 | | | | | |
| 297 | TetR- | | Protein | 266 | pSL159 | [symbol] |
| | VP64 | | | | | |
| 298 | [symbol] | | Protein | 214 | [symbol] | [symbol] |
| | VP64 | | | | | |
| 299 | TetR- | | Protein | 414 | [symbol] | [symbol] |
| | [symbol] | | | | | |
| 300 | [symbol] | | Protein | 804 | [symbol] | [symbol] |
| | [symbol] | | | | | |
| 301 | BCR- | | Protein | 2031 | pSL1014 | [symbol] |
| | [symbol] | | | | | |
| 302 | [symbol] | | Protein | 234 | [symbol] | [symbol] |
| 303 | [symbol] | [symbol] | Protein | 201 | pSL107 | [symbol] |
| | | [symbol] | | | | |
| | | [symbol] | | | | |
| | | [symbol] | | | | |
| | | [symbol] | | | | |
| | | [symbol] | | | | |
| 304 | [symbol] | [symbol] | Protein | 221 | pSL832 | [symbol] |
| | | [symbol] | | | | |
| | | [symbol] | | | | |
| | | [symbol] | | | | |
| | | [symbol] | | | | |
| 305 | FLuc | Firefly | Protein | 550 | PYW99 | [symbol] |
| | | Luciferase | | | | |

[symbol]

indicates data missing or illegible when filed

Ausländer, D., Ausländer, S., Charpin-El Hamri, G., Sedlmayer, F., Müller, M., Frey, O., Hierlemann, A., Stelling, J., Fussenegger, M., 2014. A Synthetic Multifunctional Mammalian pH Sensor and CO2 Transgene-Control Device. Mol Cell 55, 397-408. https://doi.org/10.1016/j.molcel.2014.06.007
Ausländer, S., Ausländer, D., Müller, M., Wieland, M., Fussenegger, M., 2012. Programmable single-cell mammalian biocomputers. Nature 487, 123-127. https://doi.org/10.1038/nature11149
Ausländer, S., Stücheli, P., Rehm, C., Ausländer, D., Hartig, J. S., Fussenegger, M., 2014. A general design strategy for protein-responsive riboswitches in mammalian cells. Nat Methods 11, 1154-1160. https://doi.org/10.1038/nmeth.3136
Barak, Y., Handelsman, T., Nakar, D., Mechaly, A., Lamed, R., Shoham, Y., Bayer, E. A., 2005. Matching fusion protein systems for affinity analysis of two interacting families of proteins: the cohesin-dockerin interaction. J Mol Recognit 18, 491-501. https://doi.org/10.1002/jmr.749
Bellefroid, E. J., Poncelet, D. A., Lecocq, P. J., Revelant, O., Martial, J. A., 1991. The evolutionarily conserved Krüppel-associated box domain defines a subfamily of eukaryotic multifingered proteins. Proc National Acad Sci 88, 3608-3612. https://doi.org/10.1073/pnas.88.9.3608
Berger, J., Hauber, J., Hauber, R., Geiger, R., Cullen, B. R., 1988. Secreted placental alkaline phosphatase: a powerful new quantitative indicator of gene expression in eukaryotic cells. Gene 66, 1-10. https://doi.org/10.1016/0378-1119 (88) 90219-3
Cafferty, S. M., Temmerman, J. D., Kitada, T., Becraft, J. R., Weiss, R., Irvine, D. J., Devreese, M., Baere, S. D., Combes, F., Sanders, N. N., 2021. In Vivo Validation of a Reversible Small Molecule-Based Switch for Synthetic Self-Amplifying mRNA Regulation. Mol Ther 29, 1164-1173. https://doi.org/10.1016/j.ymthe.2020.11.010
Cella, F., Wroblewska, L., Weiss, R., Siciliano, V., 2018. Engineering protein-protein devices for multilayered regulation of mRNA translation using orthogonal proteases in mammalian cells. Nat Commun 9, 4392. https://doi.org/10.1038/s41467-018-06825-7
Cunningham-Bryant, D., Dieter, E. M., Foight, G. W., Rose, J. C., Loutey, D. E., Maly, D. J., 2019. A Chemically Disrupted Proximity System for Controlling Dynamic Cellular Processes. J Am Chem Soc 141, 3352-3355. https://doi.org/10.1021/jacs.8b12382
DeCaprio, J., Kohl, T. O., 2020. Immunoprecipitation. Cold Spring Harb Protoc 2020, pdb. top098509. https://doi.org/10.1101/pdb.top098509
DiAndreth, B., Wauford, N., Hu, E., Palacios, S., Weiss, R., 2022. PERSIST platform provides programmable RNA regulation using CRISPR endoRNases. Nat Commun 13, 2582. https://doi.org/10.1038/s41467-022-30172-3
Dykstra, P. B., Kaplan, M., Smolke, C. D., 2022. Engineering synthetic RNA devices for cell control. Nat Rev Genet 1-14. https://doi.org/10.1038/s41576-021-00436-7
Foight, G. W., Wang, Z., Wei, C. T., Greisen, P. J., Warner, K. M., Cunningham-Bryant, D., Park, K., Brunette, T. J., Sheffler, W., Baker, D., Maly, D. J., 2019. Multi-input chemical control of protein dimerization for programming graded cellular responses. Nat Biotechnol 37, 1209-1216. https://doi.org/10.1038/s41587-0 19-0242-8
Fukunaga, K., Yokobayashi, Y., 2021. Directed evolution of orthogonal RNA-RBP pairs through library-vs-library in vitro selection. Nucleic Acids Res gkab527-. https://doi.org/10.1093/nar/gkab527
Gao, Y., Xiong, X., Wong, S., Charles, E. J., Lim, W. A., Qi, L. S., 2016. Complex transcriptional modulation with orthogonal and inducible dCas9 regulators. Nat Methods 13, 1043-1049. https://doi.org/10.1038/nmeth.4042
Gerstberger, S., Hafner, M., Tuschl, T., 2014. A census of human RNA-binding proteins. Nat. Rev. Genet. 15, 829-845. https://doi.org/10.1038/nrg3813
Giordano-Attianese, G., Gainza, P., Gray-Gaillard, E., Cribioli, E., Shui, S., Kim, S., Kwak, M.-J., Vollers, S., Osorio, A. D. J. C., Reichenbach, P., Bonet, J., Oh, B.-H., Irving, M., Coukos, G., Correia, B. E., 2020. A computationally designed chimeric antigen receptor provides a small-molecule safety switch for T-cell therapy. Nat Biotechnol 38, 426-432. https://doi.org/10.1038/s41587-019-0403-9
Gitzinger, M., Kemmer, C., Fluri, D. A., El-Baba, M. D., Weber, W., Fussenegger, M., 2012. The food additive vanillic acid controls transgene expression in mammalian cells and mice. Nucleic Acids Res 40, e37-e37. https://doi.org/10.1093/nar/gkr1251
Gossen, M., Bujard, H., 1992. Tight control of gene expression in mammalian cells by tetracycline-responsive promoters. Proc National Acad Sci 89, 5547-5551. https://doi.org/10.1073/pnas.89.12.5547
Gray, N. K., Coller, J. M., Dickson, K. S., Wickens, M., 2000. Multiple portions of poly (A)-binding protein stimulate translation in vivo. Embo J 19, 4723-4733. https://doi.org/10.1093/emboj/19.17.4723
Gregorio, E. D., Preiss, T., Hentze, M. W., 1999. Translation driven by an eIF4G core domain in vivo. EMBO J 18, 4865-4874. https://doi.org/10.1093/emboj/18.17.4865
Groft, C. M., Burley, S. K., 2002. Recognition of eIF4G by Rotavirus NSP3 Reveals a Basis for mRNA Circularization. Mol Cell 9, 1273-1283. https://doi.org/10.1016/s1097-2765 (02) 00555-5
Hay, C., Docherty, K., 2003. Enhanced expression of a furin-cleavable proinsulin. J Mol Endocrinol 31, 597-607. https://doi.org/10.1677/jme.0.0310597
Jackson, R. J., Hellen, C. U. T., Pestova, T. V., 2010. The mechanism of eukaryotic translation initiation and principles of its regulation. Nat Rev Mol Cell Bio 11, 113-127. https://doi.org/10.1038/nrm2838
Jacobs, C. L., Badiee, R. K., Lin, M. Z., 2018. StaPLs: versatile genetically encoded modules for engineering drug-inducible proteins. Nat Methods 15, 523-526. https://doi.org/10.1038/s41592-018-0041-z
Keeley, M. B., Busch, J., Singh, R., Abel, T., 2005. TetR hybrid transcription factors report cell signaling and are inhibited by doxycycline. Biotechniques 39, 529-536. https://doi.org/10.2144/000112002
Kemmer, C., Fluri, D. A., Witschi, U., Passeraub, A., Gutzwiller, A., Fussenegger, M., 2011. A designer network coordinating bovine artificial insemination by ovulation-triggered release of implanted sperms. J Control Release 150, 23-29. https://doi.org/10.1016/j.jconrel.2010.11.016
Kennedy, M. J., Hughes, R. M., Peteya, L. A., Schwartz, J. W., Ehlers, M. D., Tucker, C. L., 2010. Rapid blue-light-mediated induction of protein interactions in living cells. Nat Methods 7, 973-975. https://doi.org/10.1038/nmeth.1524
Kim, T. H., Dekker, J., 2018. ChIP-Quantitative Polymerase Chain Reaction (ChIP-qPCR). Cold Spring Harb Protoc 2018, pdb. prot082628. https://doi.org/10.1101/pdb.prot082628
Kowarz, E., Löscher, D., Marschalek, R., 2015. Optimized Sleeping Beauty transposons rapidly generate stable transgenic cell lines. Biotechnol J 10, 647-653. https://doi.org/10.1002/biot.201400821
Kummer, L., Parizek, P., Rube, P., Millgramm, B., Prinz, A., Mittl, P. R. E., Kaufholz, M., Zimmermann, B., Herberg, F. W., Plückthun, A., 2012. Structural and functional analysis of phosphorylation-specific binders of the kinase ERK from designed ankyrin repeat protein libraries. Proc National Acad Sci 109, E2248-E2257. https://doi.org/10.1073/pnas.1205399109
Kuwasaki, Y., Suzuki, K., Yu, G., Yamamoto, S., Otabe, T., Kakihara, Y., Nishiwaki, M., Miyake, K., Fushimi, K., Bekdash, R., Shimizu, Y., Narikawa, R., Nakajima, T., Yazawa, M., Sato, M., 2022. A red light-responsive photoswitch for deep tissue optogenetics. Nat Biotechnol 1-8. https://doi.org/10.1038/s41587-0 22-01351-w
Li, C., Samulski, R. J., 2020. Engineering adeno-associated virus vectors for gene therapy. Nat Rev Genet 21, 255-272. https://doi.org/10.1038/s41576-019-0205-4
Mitchell, S. F., Parker, R., 2015. Modifications on Translation Initiation. Cell 163, 796-798. https://doi.org/10.1016/j.cell.2015.10.056
Mitelman, F., Johansson, B., Mertens, F., 2007. The impact of translocations and gene fusions on cancer causation. Nat Rev Cancer 7, 233-245. https://doi.org/10.1038/nrc2091
Morsut, L., Roybal, K. T., Xiong, X., Gordley, R. M., Coyle, S. M., Thomson, M., Lim, W. A., 2016. Engineering Customized Cell Sensing and Response Behaviors Using Synthetic Notch Receptors. Cell 164, 780-791. https://doi.org/10.1016/j.cell.2016.01.012
Nakanishi, H., Saito, H., 2020. Caliciviral protein-based artificial translational activator for mammalian gene circuits with RNA-only delivery. Nat Commun 11, 1297. https://doi.org/10.1038/s41467-020-15061-x
Nakanishi, H., Saito, H., Itaka, K., 2022. Versatile Design of Intracellular Protein-Responsive Translational Regulation System for Synthetic mRNA. Acs Synth Biol 11, 1077-1085. https://doi.org/10.1021/acssynbio.1c00567
Pardee, K., Green, A. A., Ferrante, T., Cameron, D. E., DaleyKeyser, A., Yin, P., Collins, J. J., 2014. Paper-Based Synthetic Gene Networks. Cell 159, 940-954. https://doi.org/10.1016/j.cell.2014.10.004
Parr, C. J. C., Wada, S., Kotake, K., Kameda, S., Matsuura, S., Sakashita, S., Park, S., Sugiyama, H., Kuang, Y., Saito, H., 2020. N 1-Methylpseudouridine substitution enhances the performance of synthetic mRNA switches in cells. Nucleic Acids Res 48, e35-e35. https://doi.org/10.1093/nar/gkaa070
Passmore, L. A., Coller, J., 2021. Roles of mRNA poly (A) tails in regulation of eukaryotic gene expression. Nat Rev Mol Cell Bio 1-14. https://doi.org/10.1038/s41580-021-00417-y
Peng, H., Latifi, B., Müller, S., Lupták, A., Chen, I. A., 2021. Self-cleaving ribozymes: substrate specificity and synthetic biology applications. RSC Chem. Biol. 2, 1370-1383. https://doi.org/10.1039/d0cb00207k
Piron, M., Delaunay, T., Grosclaude, J., Poncet, D., 1999. Identification of the RNA-Binding, Dimerization, and eIF4GI-Binding Domains of Rotavirus Nonstructural Protein NSP3. J Virol 73, 5411-5421. https://doi.org/10.1128/jvi.73.7.5411-5421.1999
Roberts, J. M., Beck, J. D., Pollock, T. B., Bendixsen, D. P., Hayden, E. J., 2023. RNA sequence to structure analysis from comprehensive pairwise mutagenesis of multiple self-cleaving ribozymes. eLife 12, e80360. https://doi.org/10.7554/elife.80360
Rooney, J. W., Hodge, M. R., McCaffrey, P. G., Rao, A., Glimcher, L. H., 1994. A common factor regulates both Th1- and Th2-specific cytokine gene expression. Embo J 13, 625-633. https://doi.org/10.1002/j.1460-2075.1994.tb06300.x
Royall, E., Locker, N., 2016. Translational Control during Calicivirus Infection. Viruses 8, 104. https://doi.org/10.3390/v8040104
Saito, H., Kobayashi, T., Hara, T., Fujita, Y., Hayashi, K., Furushima, R., Inoue, T., 2010. Synthetic translational regulation by an L7Ae-kink-turn RNP switch. Nat Chem Biol 6, 71-78. https://doi.org/10.1038/nchembio.273
Sassone-Corsi, P., 1995. Transcription Factors Responsive to camp. Annu Rev Cell Dev Bi 11, 355-377. https://doi.org/10.1146/annurev.cb.11.110195.002035
Saxena, P., Heng, B. C., Bai, P., Folcher, M., Zulewski, H., Fussenegger, M., 2016. A programmable synthetic lineage-control network that differentiates human IPSCs into glucose-sensitive insulin-secreting beta-like cells. Nat Commun 7, 11247. https://doi.org/10.1038/ncomms11247
Scheller, L., Strittmatter, T., Fuchs, D., Bojar, D., Fussenegger, M., 2018. Generalized extracellular molecule sensor platform for programming cellular behavior. Nat Chem Biol 14, 723-729. https://doi.org/10.1038/s41589-018-0046-z
Schoenberg, D. R., Baron-Benhamou, J., Gehring, N. H., Kulozik, A. E., Hentze, M. W., 2004. mRNA Processing and Metabolism, Methods and Protocols. Methods Mol Biology Clifton N J 257, 135-153. https://doi.org/10.1385/1-59259-750-5:135
Shao, J., Xue, S., Yu, G., Yu, Y., Yang, X., Bai, Y., Zhu, S., Yang, L., Yin, J., Wang, Y., Liao, S., Guo, S., Xie, M., Fussenegger, M., Ye, H., 2017. Smartphone-controlled optogenetically engineered cells enable semiautomatic glucose homeostasis in diabetic mice. Sci Transl Med 9, eaa12298. https://doi.org/10.1126/scitranslmed.aa12298
Siciliano, V., DiAndreth, B., Monel, B., Beal, J., Huh, J., Clayton, K. L., Wroblewska, L., Mckeon, A., Walker, B. D., Weiss, R., 2018. Engineering modular intracellular protein sensor-actuator devices. Nat Commun 9, 1881. https://doi.org/10.1038/s41467-018-03984-5
Slomovic, S., Pardee, K., Collins, J. J., 2015. Synthetic biology devices for in vitro and in vivo diagnostics. Proc National Acad Sci 112, 14429-14435. https://doi.org/10.1073/pnas.1508521112
Tang, T. T., Stowell, J. A., Hill, C. H., Passmore, L. A., 2019. The intrinsic structure of poly (A) RNA determines the specificity of Pan2 and Caf1 deadenylases. Nat Struct Mol Biol 26, 433-442. https://doi.org/10.1038/s41594-019-0227-9
Wang, H., Ye, H., Xie, M., Daoud El-Baba, M., Fussenegger, M., 2015. Cosmetics-triggered percutaneous remote control of transgene expression in mice. Nucleic Acids Res 43, e91-e91. https://doi.org/10.1093/nar/gkv326
Weber, W., Link, N., Fussenegger, M., 2006. A genetic redox sensor for mammalian cells. Metab Eng 8, 273-280. https://doi.org/10.1016/j.ymben.2005.12.004
Wroblewska, L., Kitada, T., Endo, K., Siciliano, V., Stillo, B., Saito, H., Weiss, R., 2015. Mammalian synthetic circuits with RNA binding proteins delivered by RNA. Nat Biotechnol 33, 839-841. https://doi.org/10.1038/nbt.3301
Wu, J., Wang, M., Yang, X., Yi, C., Jiang, J., Yu, Y., Ye, H., 2020. A non-invasive far-red light-induced split-Cre recombinase system for controllable genome engineering in mice. Nat Commun 11, 3708. https://doi.org/10.1038/s41467-0 20-17530-9
Xie, M., Ye, H., Wang, H., Hamri, G. C.-E., Lormeau, C., Saxena, P., Stelling, J., Fussenegger, M., 2016. β-cell-mimetic designer cells provide closed-loop glycemic control. Science 354, 1296-1301. https://doi.org/10.1126/science.aaf4006
Xie, Z., Wroblewska, L., Prochazka, L., Weiss, R., Benenson, Y., 2011. Multi-Input RNAi-Based Logic Circuit for Identification of Specific Cancer Cells. Science 333, 1307-1311. https://doi.org/10.1126/science.1205527
Yin, J., Yang, L., Mou, L., Dong, K., Jiang, J., Xue, S., Xu, Y., Wang, X., Lu, Y., Ye, H., 2019. Agreen tea-triggered genetic control system for treating diabetes in mice and monkeys. Sci Transl Med 11, eaav8826. https://doi.org/10.1126/scitranslmed.aav8826
Yu, A.-M., Choi, Y. H., Tu, M.-J., 2020. RNA Drugs and RNA Targets for Small Molecules: Principles, Progress, and Challenges. Pharmacol Rev 72, 862-898. https://doi.org/10.1124/pr. 120.019554
Zhong, G., Wang, H., He, W., Li, Y., Mou, H., Tickner, Z. J., Tran, M. H., Ou, T., Yin, Y., Diao, H., Farzan, M., 2020. A reversible RNA on-switch that controls gene expression of AAV-delivered therapeutics in vivo. Nat Biotechnol 38, 169-175. https://doi.org/10.1038/s41587-019-0357-y

1. A gene regulation system that can express the target gene mRNA by regulation, which comprises
(i) Synthetic translation initiation Factors (STIFs), and an mRNA construct that comprises the mRNA encoding the target protein; or
(ii) An nucleic acid that encodes the STIF(s) and a nucleic acid that encodes the mRNA construct that comprises the mRNA encoding the target protein, e.g., said nucleic acid is DNA or RNA.
wherein the STIF comprises or consists of at least one eIFBP (eIF4F-binding proteins), and at least one RBP (RNA binding proteins).

2. The system of claim 1, wherein the eIFBP is selected from PABP, NSP3, VPg and anyone member of eIF4F, such as eIF4A, eIF4B, eIF4E or eIF4G; and/or the RBP is selected from L7Ae or MCP or λ-N.

3. The system of claim 2, wherein
the PABP comprises the amino acid sequence of SEQ ID NO: 108 or an amino acid sequence having at least 85%, 90%, 91%, 92%, 93%, 94%, 95%, 96%, 97%, 98% or 99% sequence identity to SEQ ID NO: 108, or consists of said amino acid sequence;
the NSP3 comprises the amino acid sequence of SEQ ID NO: 91 or 106 or an amino acid sequence having at least 85%, 90%, 91%, 92%, 93%, 94%, 95%, 96%, 97%, 98% or 99% sequence identity to SEQ ID NO: 91 or 106, or consists of said amino acid sequence;
the VPg comprises the amino acid sequence of SEQ ID NO: 120 or an amino acid sequence having at least 85%, 90%, 91%, 92%, 93%, 94%, 95%, 96%, 97%, 98% or 99% sequence identity to SEQ ID NO: 120, or consists of said amino acid sequence;
the eIF4G comprises the amino acid sequence of SEQ ID NO: 84 or an amino acid sequence having at least 85%, 90%, 91%, 92%, 93%, 94%, 95%, 96%, 97%, 98% or 99% sequence identity to SEQ ID NO: 84, or consists of said amino acid sequence;
the eIF4E comprises the amino acid sequence of SEQ ID NO: 83 or 314 or an amino acid sequence having at least 85%, 90%, 91%, 92%, 93%, 94%, 95%, 96%, 97%, 98% or 99% sequence identity to SEQ ID NO: 83 or 314, or an amino acid sequence having at least 85%, 90%, 91%, 92%, 93%, 94%, 95%, 96%, 97%, 98% or 99% sequence identity to SEQ ID NO: 83 and having a substitution of K119A or consists of said amino acid sequence;
the L7Ae comprises the amino acid sequence of SEQ ID NO: 92 or an amino acid sequence having at least 85%, 90%, 91%, 92%, 93%, 94%, 95%, 96%, 97%, 98% or 99% sequence identity to SEQ ID NO: 92, or consists of said amino acid sequence;
the MCP comprises the amino acid sequence of SEQ ID NO: 98 or an amino acid sequence having at least 85%, 90%, 91%, 92%, 93%, 94%, 95%, 96%, 97%, 98% or 99% sequence identity to SEQ ID NO: 98, or consists of said amino acid sequence. In a further embodiment, the MCP is an MCP variant, which has V29I substitution compared to the MCP; or
the λ-N comprises the amino acid sequence of SEQ ID NO: 100 or an amino acid sequence having at least 85%, 90%, 91%, 92%, 93%, 94%, 95%, 96%, 97%, 98% or 99% sequence identity to SEQ ID NO: 100, or consists of said amino acid sequence.

4. The system of anyone of claims 1 to 3, wherein the configuration of the fusion protein, from N-terminus to C-terminus, is eIFBP-RBP or RBP-eIFBP, and optionally with some further protein domains (e.g., 2CaM-M13, or tag) inserted or with a tag at N-terminus or C-terminus, preferably,
the STIF comprises or consists of a fusion protein L7Ae-NSP3; 3×flag-L7Ae-NSP3; L7Ae-hNSP3; L7Ae-eIF4E; MCP-NSP3; MCP-hNSP3; PABP-L7Ae; PABP-L7Ae-3×FLAG; eIF4G-2CaM-M13-L7Ae; PABP-MCP; MCP-eIF4E; eIF4G-MCP; 3×FLAG-MCP-NSP3; MCP-VPg; MCP^{V29I}-VPg; or L7Ae-VPg.

5. The system of anyone of claims 1 to 3, wherein the STIF comprises or consists of two recombinant fusion proteins A and B, wherein
wherein protein A can be RBP-Y and protein B can be Y′-eIFBP, or
wherein protein A can be RBP-Y and protein B can be eIFBP-Y′, or
wherein protein A can be Y-RBP and protein B can be Y′-eIFBP, or
wherein protein A can be Y-RBP and protein B can be eIFBP-Y′, or
wherein protein A can be eIFBP-Y and protein B can be Y′-RBP, or
wherein protein A can be eIFBP-Y and protein B can be RBP-Y′, or
wherein protein A can be Y-eIFBP and protein B can be Y′-RBP, or
wherein protein A can be Y-eIFBP and protein B can be RBP-Y′, or
wherein protein A can be RBP-Y′ and protein B can be Y-eIFBP, or
wherein protein A can be RBP-Y′ and protein B can be eIFBP-Y, or
wherein protein A can be Y′-RBP and protein B can be Y-eIFBP, or
wherein protein A can be Y′-RBP and protein B can be eIFBP-Y, or
wherein protein A can be eIFBP-Y′ and protein B can be Y-RBP, or
wherein protein A can be eIFBP-Y′ and protein B can be RBP-Y, or
wherein protein A can be Y′-eIFBP and protein B can be Y-RBP, or
wherein protein A can be Y′-eIFBP and protein B can be RBP-Y;
wherein Y and Y′ can interact with each other constitutively, or by trigger agent or signal or by a further protein Y″.

6. The system of claim 5, wherein the protein A or B can comprise multiple tandem repeats of Y or Y′, for example, 1-5 repeats, e.g., 1, 2, 3, 4, or 5 repeats.

7. The system of claim 5 or 6, wherein the Y and Y′ constitutively interact with each other, preferably, protein Y is DocS and protein Y′ is Coh2; or protein Y is Coh2 and protein Y′ is DocS; protein Y is NS3a (H1) and protein Y′ is ANR; or protein Y is ANR and protein Y′ is NS3a (H1); protein Y is Bcl-XL and protein Y′ is LD1 or LD3; or protein Y is LD1 or LD3 and protein Y′ is Bcl-XL; protein Y is EGFP and protein Y′ is LaG16; or protein Y is LaG16 and protein Y′ is EGFP; protein Y is CCmut3 and protein Y′ is BCR; or protein Y is BCR and protein Y′ is CCmut3; protein Y is ABI (iDab) and protein Y′ is ABL1; or protein Y is ABL1 and protein Y′ is ABI (iDab); protein Y is antibody or antigen binding fragments (such as VHH or scFv) that specifically binds to an antigen and protein Y′ is the antigen; or protein Y is the antigen and protein Y′ is the antibody or antigen binding fragments (such as VHH or scFv), for example, the antigen is NS3 or fragment thereof, e.g., N-terminus of NS3.

8. The system of claim 5 or 6, wherein the Y and Y′ is interacted with each other by trigger-induction or trigger-repression with an agent, for example, protein Y is ABI and protein Y′ is PYL1; or protein Y is PYL1 and protein Y′ is ABI, for example, the protein-protein interaction (binding) between ABI and PYL1 is trigger-inducible by Abscisic acid; protein Y is Aff6 and protein Y′ is DrBPhP; or protein Y is DrBPhP and protein Y′ is Aff6, for example, the protein-protein interaction (binding) between Aff6 and DrBPhP is trigger-inducible by red light; protein Y is ANR and protein Y′ is NS3a (H1); or protein Y is NS3a (H1) and protein Y′ is ANR, for example, the protein-protein interaction (binding) between ANR and NS3a (H1) is trigger-repressible by Grazoprevir; protein Y is CIB1 and protein Y′ is Cry2; or protein Y is Cry2 and protein Y′ is CIB1, for example, the protein-protein interaction (binding) between CIB1 and Cry2 is trigger-inducible by blue light; protein Y is DNCR and protein Y′ is NS3a or NS3a (H1); or protein Y is NS3a or NS3a (H1) and protein Y′ is DNCR, for example, the protein-protein interaction (binding) between DNCR and NS3a or NS3a (H1) is trigger-inducible by Danoprevir; protein Y is FKBP and protein Y′ is FRB; or protein Y is FRB and protein Y′ is FKBP, for example, the protein-protein interaction (binding) between FKBP and FRB is trigger-inducible by Rapamycin; protein Y is GAI and protein Y′ is GID1; or protein Y is GID1 and protein Y′ is GAI, for example, the protein-protein interaction (binding) between GAI and GID1 is trigger-inducible by Gibberellic acid; protein Y is GNCR and protein Y′ is NS3a or NS3a (H1); or protein Y is NS3a or NS3a (H1) and protein Y′ is GNCR, for example, the protein-protein interaction (binding) between GNCR and NS3a or NS3a (H1) is trigger-inducible by Grazoprevir; or protein Y is LaM8AK47 and protein Y′ is mCherry; or protein Y is mCherry and protein Y′ is LaM8AK47, for example, the protein-protein interaction (binding) between LaM8AK47 and mCherry is trigger-inducible by blue lightx.

9. The system of claim 5 or 6, wherein the Y and Y′ can interact with each other specifically by Signaling, wherein
protein Y is ERK2 and protein Y′ is pE59; or protein Y is pE59 and protein Y′ is ERK2, for example, the protein-protein interaction (binding) between ERK2 and pE59 is trigger-inducible by MAPK signaling (e.g., agents that activate the MAPK signaling, such as MAPK, EGF).

10. The system of claim 5 or 6, wherein Y and Y′ can interact with each other via another protein Y″, when Y″ exist, Y and Y′ can associate with target protein Y″ through a specific Y:Y″:Y′-mediated interaction, preferably, protein Y and protein Y′ are two different scFvs that binds specifically to protein Y″, e.g., specifically binds to different domains or epitopes of protein Y″.

11. The system of claim 10, wherein the Y″ is selected from
(i) a disease-specific cellular signature, such as oncoprotein, e.g., a fusion protein or protein complex that specifically expressed in tumor cell or tumor tissue, e.g., BCR-ARL, or infection specific protein, such as HCV or HCV specific protein (e.g., NS3 protein);
(ii) a fusion protein of Y and Y″, or a fusion protein comprising both the binding domain of Y and the binding domain of Y′, wherein Y and Y′ can constitutively interact with each other, e.g., EGFP-NS3a (H1) fusition protein;
(iii) intracellular or secreted protein containing one or multiple domains;
(iv) antigen that has at least two or more epitopes or domains.

12. The system of anyone of claims 1-11, wherein the mRNA construct comprising a coding region and an RNA segment binding by STIF, and the coding region is flanked by the 5′-UTR and 3′-UTR.

13. The system of claim 12, wherein the coding region is any RNA sequence starting with nucleotides AUG and terminating with nucleotide sequences UAG, UAA or UGA, ore example, the coding region encodes target protein, preferably, the target protein can be selected from therapeutic protein such as insulin e.g., human insulin or pro-apoptotic protein such as BAX (such as human Bax), protein that can be detected with its expression, e.g., a marker protein or reporter such as SEAP, luciferase, fluorescent protein such as GFP or EGFP.

14. The system of claim 12 or 13, wherein the RNA segment binding by STIF is poly-A signal or a poly (A)-surrogate, wherein the poly (A) surrogate can be any segment that contains or consists of one or more n aptamer repeats binding to a specific RBP, and is placed into the 3′-UTR or 5′-UTR of said mRNA.

15. The system of claim 14, wherein the aptamer is selected from C/D-box, MS2-box, or boxB.

16. The system of claim 15, wherein
the poly (A) surrogate is tandem repeats of the L7Ae-specific C/D-box aptamer e.g., (C/D-box) n if the RBP of STIF is L7Ae, or MCP-specific MS2-box aptamer (MS2-box) n or λ-N-specific aptamer (boxB) n if the RBP of STIF is MCP, wherein n can be any number between 1 to 1000, e.g., 5-30, e.g., 8, 12, 16, or 24.

17. The system of claim 16, wherein
the C/D-box comprises or consists of the amino acid sequence of SEQ ID NO: 123;
the MS2-box comprises or consists of the amino acid sequence of SEQ ID NO: 125 or 315; or
the boxB comprises or consists of the amino acid sequence of SEQ ID NO: 121.

18. The system of claims 14 to 17, wherein the mRNA construct further comprises an RNA cleavage site enabling pre-programmed poly (A)-removal, preferably, the site is located between the aptamer and the poly (A) and placed into the 3′-UTR.

19. The system of claim 18, wherein the cleavage is performed by RNA interference and the RNA cleavage site is a siRNA binding site or multiple copies thereof, a shRNA binding site or multiple copies or a miRNA binding site or multiple copies thereof.

20. The system of claim 19, wherein the system further comprises a construct that express shRNA-216 to cleave the polyA, and wherein the RNA cleavage site may comprises one or multiple n repeats of (BS (shRNA-216)) n, wherein n can be any number between 1 and 100 and preferably n can be any number between 1 and 4, preferably, the BS (shRNA-216) comprises or consists of SEQ ID NO: 122, and/or the shRNA-216 comprises or consists of SEQ ID NO: 126.

21. The system of claim 18, wherein the cleavage is performed by ribozyme and the cleavage site is a ribozyme, for example, the ribozyme is a self-cleaving ribozyme or multiple copies thereof or a fragment thereof.

22. The system of claim 21, wherein the self-cleaving ribozyme is the hammerhead ribozyme (HHR) n, wherein n can be any number between 1 and 100 and preferably n can be any number between 1 and 4, for example, the HHR comprises or consists of SEQ ID NO: 124.

23. The system of claim 12, wherein the mRNA construct the construct comprises, from N-terminus to C-terminus, 5′UTR, the coding region, Poly (A)-surrogate, cleavage site and 3′UTR, preferably, the combination of Poly (A)-surrogate and the cleavage site is selected from
(i). (C/D-box)₈ (BS (shRNA-216))₂
(ii). (C/D-box)₁₆ (BS (shRNA-216))₂
(iii). (C/D-box)₁₂ (BS (shRNA-216))₂
(iv). (C/D-box)₂₄ (BS (shRNA-216))₂
(v). (C/D-box)₂₄ (BS (shRNA-216))₂
(vi). (C/D-box)₂₄ HHR
(vii). (MS2-box)₂₄ HHR
(viii). (MS2-box)₁₆ HHR
(ix). (MS2-box)₂₄ HHR
(x). (MS2-box)₂₄ HHR
(xi). none (HHR)₂
(xii). none (HHR)₄
(xiii). (MS2-box)₂₄ HHR
(xiv). none HHR
(xv). (MS2-box)₂₄ HHR
(xvi). (MS2-box)₂₄ HHR
(xvii). (MS2-box)₁₆ HHR
(xviii). (C/D-box)₂₄ none
(xix). (C/D-box)₄ none
(xx). (MS2-box)₂₄ HHR
(xxi). (MS2-box)₁₂ HHR
(xxii). (MS2-box)₈ HHR
(xxiii). (MS2-box)₈ (BS (shRNA-216))₂.

24. The system of claims 12 to 23, wherein the mRNA construct can further comprise 5′-cap or 5′-cap surrogates, preferably, the 5′-cap surrogate can be the poly (A) surrogate as defined in anyone of claims 14-17.

25. The system of claim 25, wherein the mRNA construct can further comprise the cleavage as defined in anyone of claims 19-22.

26. The system of claim 12, wherein the system is triggered by Grazoprevir, wherein the STIF comprises or consists of two recombinant fusion proteins A and B, and wherein
(i) protein A is L7Ae-(NS3a) n configuration and protein B is (GNCR) n-NSP3, or protein A is L7Ae-(GNCR) n configuration and protein B is (NS3a) n-NSP3, wherein n is an integer from 1 to 10; and the mRNA construct comprises, from N-terminus to C-terminus, 5′ UTR, coding region, (C/D-box) n (e.g., n=1-30, e.g., 24), (BS (shRNA-216)) n (e.g., n=1, 2 or 3, e.g, 2) and 3′ UTR; or
(ii) protein A is MCP-(NS3a)ₙ configuration and protein B is (GNCR)ₙ-NSP3, or protein A is MCP-(GNCR)ₙ configuration and protein B is (NS3a) n-NSP3, wherein n is an integer from 1 to 10; and the mRNA construct comprises, from N-terminus to C-terminus, 5′ UTR, coding region, (MS2-box) n (e.g., n=1-30, e.g., 24), (BS (shRNA-216)) n (e.g., n=1, 2 or 3, e.g, 2) or (HHR)ₙ (e.g., n=1, 2, 3 or 4, e.g, 1) and 3′ UTR;
and the system further comprises a construct that expresses shRNA-216;
preferably, the coding region encodes insulin, e.g., human insulin.

27. A nucleic acid encoding STIF and/or the mRNA construct of the system of anyone of claims 1 to 26, wherein the nucleic acid is DNA or RNA.

28. A vector comprising the nucleic acid of claim 27, wherein the vector is an expression vector, such as a eukaryotic expression vector, such as pcDNA3.1 or AAV.

29. A pharmaceutical composition or kit or abiotic materials comprising the system of anyone of claims 1-26.

30. A pharmaceutical combination comprising the system of anyone of claims 5-26, and one or more other agents, e.g., agent that trigger or induce or repress the expression of the system, e.g., protein Y″, Abscisic acid, Grazoprevir, Danoprevir, MAPK, Rapamycin, Gibberellic acid; or other therapeutic agents.

31. A method for preventing or treating a disease, comprising administering the gene regulation system of anyone of claims 1-26 or the pharmaceutical composition of claim 29 or pharmaceutical combination of claim 30 to a subject need thereof.

32. A use of the system of anyone of claims 1-26 in Biocomputation in vitro or in vivo.

33. A method for diagnosing a disease, comprising using the system of anyone of claims 10-26 to detect the disease signature of the disease, wherein the disease signature is the Y″ that can be bound by Y and Y′ of the STIF.

34. A method to detect a protein in vitro, including using the system of anyone of claims 10-26 to detect a protein that served as Y″ bound by both Y and Y′ of the STIF, preferably, the system is comprised in abiotic materials, e.g., paper discs.
